(19) United States
(12) Patent Application Publication
(10) Pub. No.: US 2007/0135442 A1
(43) Pub. Date: Jun. 14, 2007

(54) Chroman compounds

(75) Inventors: Peter Bernstein, Wilmington, DE (US); Daniel Hill, Wilmington, DE (US); Margaret Schooler, Wilmington, DE (US); Ashok Shenvi, Wilmington, DE (US); Robert Jacobs, Durham, NC (US)

Correspondence Address: ASTRA ZENECA PHARMACEUTICALS LP;GLOBAL INTELLECTUAL PROPERTY, 1800 CONCORD PIKE, WILMINGTON, DE, 19850-5437, US

(73) Assignee: AstraZeneca AB, Sodertalje (SE)

(21) Appl. No.: 11/592,543

(22) Filed: Nov. 3, 2006

Related U.S. Application Data
(60) Provisional application No. 60/734,036, filed on Nov. 4, 2005.

(51) Int. Cl. A61K 31/506; A61K 31/496; C07D 413/14; C07D 405/02

(52) U.S. Cl. 514/252.12; 514/254.11; 514/254.02; 514/254.03; 544/295; 544/367

(57) ABSTRACT

Compounds according to Formula (I) below: 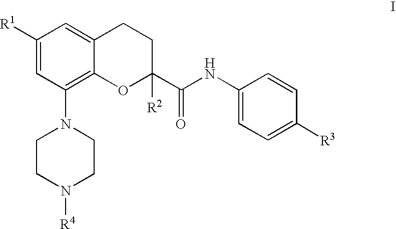
wherein R¹, R², R³, and R⁴ are as defined in the specification, pharmaceutically-acceptable salts, methods of making, pharmaceutical compositions containing and methods for using the same.

### FIELD OF THE INVENTION

This invention relates to novel chroman derivatives, methods for their preparation, pharmaceutical compositions containing them and their use in therapy.

### BACKGROUND OF THE INVENTION

Alterations in serotonin (5-hydroxytryptamine or 5-HT) activity has been implicated in many psychiatric disorders including but not limited to depression, generalized anxiety, eating disorders, dementia, panic disorder, and sleep disorders. Furthermore serotonin has been implicated in gastrointestinal disorders, cardiovascular regulation, motor disorders, endocrine disorders, vasospasm and sexual dysfunction. Serotonin receptors have been subdivided into at least 14 subtypes, see Barnes and Sharp, Neuropharmacology, 1999, 38, 1083-1152. These various subtypes are responsible for serotonin's action in many pathophysiological conditions. The 5-HT₁ families of receptors have high affinity for serotonin and consist of five related receptors. This family includes the 5-HT_{1B} and 5-HT_{1D} receptor subtypes. Compounds that interact with the 5-HT₁ families are known to have therapeutic potential in the above-mentioned disorders and diseases. In particular, compounds that are 5-HT_{1B} and 5-HT_{1D} antagonist have been known to be antidepressant and anxiolytic agents. The present invention discloses several 5-HT_{1B} antagonists that are useful for the treatment of anxiety disorders or mood disorders such as depression or dementia and other cognitive disorders such as Alzheimer's disease.

Provided herein are compounds in accord with Formula I: 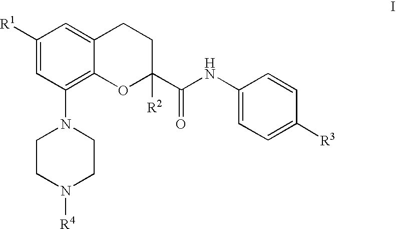
wherein:
R¹ is F or OCH₃ or OH;
R² is H, or CH₃;
R³ is a heterocycle where the heterocycle is optionally substituted with one of the following —CH₃, —CH₂CH₃, —CH₂CH₂CH₃, —CH(CH₃)₂, —CH₂OCH₃, or —OCH₂CH₃; and
R⁴ is H, —CH₃, CH₂CF₃, —CH₂CH₂OCH₃, CH₂CH₂CH₂CH₃, —CH₂CH₂CH₃, —CH(CH₃)₂,
or —CH₂CH₃

or a pharmaceutically-acceptable salt thereof.

Also provided are methods of using the compounds of formula I to treat diseases and conditions associated with a wide range of diseases or disorders in which 5-HT receptors are considered to have a role. Also provided are uses of the compounds of formula I as medicaments, uses of the compounds of formula I in the manufacture of medicaments and uses of the compounds of formula I for diagnostic and analytic purposes. Also provided are various administration methods either alone or in combination with other therapeutically active compounds or substances. Also provided are processes and intermediates used to prepare the compounds of formula I. Also provided are pharmaceutical compositions containing the compounds of formula I.

Definitions:

If used herein, the following terms have the following meanings:

The term “amine” or “amino” refers to radicals of the general formula —NRR′, wherein R and R′ are independently selected from hydrogen or a hydrocarbyl radical.

The term “hydrocarbyl” refers to any structure comprising only carbon and hydrogen atoms up to 14 carbon atoms.

The term “alkyl” used alone or as a suffix or prefix, refers to straight or branched chain hydrocarbyl radicals comprising 1 to about 12 carbon atoms.

The term “aromatic” refers to hydrocarbyl radicals having one or more polyunsaturated carbon rings having aromatic character, (e.g., 4n+2 delocalized electrons) and comprising 6 up to about 14 carbon atoms.

The term “aryl” refers to aromatic radicals including both monocyclic aromatic radicals comprising 6 carbon atoms and polycyclic aromatic radicals comprising up to about 14 carbon atoms.

The term “halo” or “halogen” refers to fluorine, chlorine, bromine and iodine radicals.

The term “heterocycle” or “heterocyclic” or “heterocyclic moiety” refers to ring-containing monovalent and divalent radicals having one or more heteroatoms, independently selected from N, O and S, as part of the ring structure and comprising at least 3 and up to about 20 atoms in the rings preferably 5 and 6 membered rings. Heterocyclic moieties may be saturated or unsaturated, containing one or more double bonds, and heterocyclic moieties may contain more than one ring.

The term “heteroaryl” refers to heterocyclic monovalent and divalent radicals having aromatic character.

Heterocyclic moieties include for example monocyclic moieties such as: aziridine, azetidine, dioxolane, dioxoimidazolidin, imidazolidine, oxetane, oxyrane, oxopyrrolidin, oxopyrimidine, oxooxazolidine, oxadiazole, oxazole, oxotetrahydropyrimidine, methyloxazole, pyrazolidine, pyrrolidine, pyrroline, thiirane, thietane, sulfolane 2,3-dihydrofuran, 2,5-dihydrofuran tetrahydrofuran, thiophane, piperidine, 1,2,3,6-tetrahydropyridine, piperazine, morpholine, thiomorpholine, pyran, thiopyran, 2,3-dihydropyran, tetrahydropyran, 1,4-dihydropyridine, 1,4-dioxane, 1,3-dioxane, dioxane, homopiperidine, 2,3,4,7-tetrahydro-1H-azepine homopiperazine, 1,3-dioxepane, 4,7-dihydro-1,3-dioxepin, and hexamethylene oxide. In addition heterocyclic moieties include heteroaryl rings such as: methyloxazole, pyridyl, pyrazinyl, pyrimidinyl, pyridazinyl, thienyl, furyl, pyrrolyl, imidazolyl, thiazolyl, oxazolyl, pyrazolyl, isothiazolyl, isoxazolyl, 1,2,3-triazolyl, tetrazolyl, 1,2,3-thiadiazolyl, 1,2,3-oxadiazolyl, 1,2,4-triazolyl, 1,2,4-thiadiazolyl, 1,2,4-oxadiazolyl, methyl-1,2,4-oxadiazolyl, 1,3,4-triazolyl, 1,3,4-thiadiazolyl, and 1,3,4 oxadiazolyl. Additionally, heterocyclic moieties encompass polycyclic moieties such as: morpholinophenyl, indole, indoline, quinoline, tetrahydroquinoline, isoquinoline, tetrahydroisoquinoline, 1,4-benzodioxan, coumarin, dihydrocoumarin, benzofuran, 2,3-dihydrobenzofuran, 1,2-benzisoxazole, benzothiophene, benzoxazole, benzthiazole, benzimidazole, benztriazole, thioxanthine, carbazole, carboline, acridine, pyrolizidine, and quinolizidine.

In addition to the polycyclic heterocycles described above, heterocyclic moieties include polycyclic heterocyclic moieties wherein the ring fusion between two or more rings comprises more than one bond common to both rings and more than two atoms common to both rings. Examples of such bridged heterocycles include quinuclidine, diazabicyclo[2.2.1]heptane and 7-oxabicyclo[2.2.1]heptane.

The term “anxiety disorders” includes but is not limited to one or more of the following, panic disorder, panic disorder without agoraphobia, panic disorder with agoraphobia, agoraphobia without history of panic disorder, specific phobia, social phobia, social anxiety disorder, obsessive-compulsive disorder, posttraumatic stress disorder, acute stress disorder, generalized anxiety disorder and generalized anxiety disorder due to a general medical condition.

The term “cognitive disorders” includes but is not limited to Alzheimer's disease, dementia, and dementia due to Alzheimer's disease, dementia due to Parkinson's disease.

The term “mammal” includes all air-breathing animals including humans.

The term “mood disorders” includes but is not limited to one or more of the following, depressive disorders, including but not limited to major depressive disorder and dysthymic disorder and b) bipolar depression and/or bipolar mania including but not limited to bipolar I, including but not limited to those with manic, depressive or mixed episodes, and bipolar II, c) cyclothymic disorder, mood disorder due to a general medical condition and manic episodes associated with bipolar disorder and mixed episodes associated with bipolar disorder.

The above conditions and disorder are defined for example in the American Psychiatric Association: Diagnostic and Statistical Manual of Mental Disorders, Fourth Edition, Text Revision, Washington, D.C., American Psychiatric Association, 2000.

### DETAILED DESCRIPTION OF THE INVENTION

A further aspect of the invention provides a compound according to Formula I wherein R¹ is F.

A further aspect of the invention provides a compound according to Formula I wherein R¹ is OCH₃.

A further aspect of the invention provides a compound according to Formula I wherein R¹ is OH.

A further aspect of the invention provides a compound according to Formula I wherein R² is H.

A further aspect of the invention provides a compound according to Formula I wherein R² is CH₃.

A further aspect of the invention provides a compound according to Formula I wherein R³ is oxadiazole optionally substituted with —C₁₋₄alkyl, or —CH₂OCH₃.

A further aspect of the invention provides a compound according to Formula I wherein R³ is oxotetrahydropyrimidine optionally substituted with —CH₃.

A further aspect of the invention provides a compound according to Formula I wherein R³ is oxooxazolidine.

A further aspect of the invention provides a compound according to Formula I wherein R³ is oxotetrahydropyrimidine optionally substituted with —CH₃.

A further aspect of the invention provides a compound according to Formula I wherein R³ is dioxoimidazolidin optionally substituted with —CH₃.

A further aspect of the invention provides a compound according to Formula I wherein R³ is oxopyrrolidin.

A further aspect of the invention provides a compound according to Formula I wherein R³ is piperidine optionally substituted with —OCH₂CH₃

A further aspect of the invention provides a compound according to Formula I wherein R³ is oxazole.

A further aspect of the invention provides a compound according to Formula I wherein R³ is morpholine.

A further aspect of the invention provides a compound according to Formula I wherein R¹ is F or OCH₃;

R² is H;

R³ is a heterocycle optionally substituted with one of the following —CH₃, —CH₂CH₃, —CH₂CH₂CH₃, —CH(CH₃)₂, —CH₂OCH₃, or —OCH₂CH₃; and

R⁴ is —CH₃;

or a pharmaceutically-acceptable salt thereof.

A further aspect of the invention provides a compound according to Formula I wherein R¹ is F;

R² is H;

R³ is a heterocycle optionally substituted with one of the following —CH₃, —CH₂CH₃, —CH₂CH₂CH₃, —CH(CH₃)₂, —CH₂OCH₃, or —OCH₂CH₃; and

R⁴ is —CH₃;

or a pharmaceutically-acceptable salt thereof.

A further aspect of the invention provides a compound according to Formula I wherein R¹ is OCH₃;

R² is H;

R³ is a heterocycle optionally substituted with one of the following —CH₃, —CH₂CH₃, —CH₂CH₂CH₃, —CH(CH₃)₂, —CH₂OCH₃, or —OCH₂CH₃; and

R⁴ is —CH₃;

or a pharmaceutically-acceptable salt thereof.

A further aspect of the invention provides a compound according to Formula I wherein R¹ is F or OCH₃;

R² is H;

R³ is oxadiazole optionally substituted with one of the following —CH₃, —CH₂CH₃, CH(CH₃)₂, or —CH₂OCH₃; and

R⁴ is —CH₃;

or a pharmaceutically-acceptable salt thereof.

A further aspect of the invention provides a compound according to Formula I wherein R¹ is F;

R² is H;

R³ is oxadiazole optionally substituted with one of the following —CH₃, —CH₂CH₃, CH(CH₃)₂, or —CH₂OCH₃; and

R⁴ is —CH₃;

or a pharmaceutically-acceptable salt thereof.

A further aspect of the invention provides a compound according to Formula I wherein R¹ is OCH₃;

R² is H;

R³ is oxadiazole optionally substituted with one of the following —CH₃, —CH₂CH₃, CH(CH₃)₂, or —CH₂OCH₃; and

R⁴ is —CH₃;

or a pharmaceutically-acceptable salt thereof.

A further aspect of the invention provides a compound according to Formula I wherein R¹ is F or OCH₃;

R² is H;

R³ is oxooxazolidine or —CH₂— oxooxazolidine; and

R⁴ is —CH₃;

or a pharmaceutically-acceptable salt thereof.

A further aspect of the invention provides a compound according to Formula I wherein R¹ is F;

R² is H;

R³ is oxooxazolidine or —CH₂— oxooxazolidine; and

R⁴ is —CH₃;

or a pharmaceutically-acceptable salt thereof.

A further aspect of the invention provides a compound according to Formula I wherein R¹ is OCH₃;

R² is H;

R³ is oxooxazolidine or —CH₂— oxooxazolidine; and;

R⁴ is —CH₃;

or a pharmaceutically-acceptable salt thereof.

A further aspect of the invention provides a compound according to Formula I wherein R¹ is F or OCH₃;

R² is H;

R³ is oxotetrahydropyrimidine optionally substituted with —CH₃; and

R⁴ is —CH₃;

or a pharmaceutically-acceptable salt thereof.

A further aspect of the invention provides a compound according to Formula I wherein R¹ is F;

R² is H;

R³ is oxotetrahydropyrimidine optionally substituted with —CH₃; and

R⁴ is —CH₃;

or a pharmaceutically-acceptable salt thereof.

A further aspect of the invention provides a compound according to Formula I wherein R¹ is OCH₃;

R² is H;

R³ is oxotetrahydropyrimidine optionally substituted with —CH₃; and

R⁴ is —CH₃;

or a pharmaceutically-acceptable salt thereof.

A further aspect of the invention provides a compound according to Formula I wherein R¹ is F or OCH₃;

R² is H;

R³ is dioxoimidazolidin optionally substituted with CH₃; and

R⁴ is —CH₃;

or a pharmaceutically-acceptable salt thereof.

A further aspect of the invention provides a compound according to Formula I wherein R¹ is F;

R² is H;

R³ is dioxoimidazolidin optionally substituted with CH₃; and

R⁴ is —CH₃;

or a pharmaceutically-acceptable salt thereof.

A further aspect of the invention provides a compound according to Formula I wherein R¹ is OCH₃;

R² is H;

R³ is dioxoimidazolidin optionally substituted with CH₃; and

R⁴ is —CH₃;

or a pharmaceutically-acceptable salt thereof.

A further aspect of the invention provides a compound according to Formula I wherein R¹ is F or OCH₃;

R² is H;

R³ is oxopyrrolidin; and

R⁴ is —CH₃;

or a pharmaceutically-acceptable salt thereof.

A further aspect of the invention provides a compound according to Formula I wherein R¹ is F;

R² is H;

R³ is oxopyrrolidin; and

R⁴ is —CH₃;

or a pharmaceutically-acceptable salt thereof.

A further aspect of the invention provides a compound according to Formula I wherein R¹ is OCH₃;

R² is H;

R³ is oxopyrrolidin; and

R⁴ is —CH₃;

or a pharmaceutically-acceptable salt thereof.

A further aspect of the invention provides a compound according to Formula I wherein R¹ is F or OCH₃;

R² is H;

R³ is piperidine optionally substituted with —OCH₂CH₃; and

R⁴ is —CH₃;

or a pharmaceutically-acceptable salt thereof.

A further aspect of the invention provides a compound according to Formula I wherein R¹ is F;

R² is H;

R³ is piperidine optionally substituted with —OCH₂CH₃; and

R⁴ is —CH₃;

or a pharmaceutically-acceptable salt thereof.

A further aspect of the invention provides a compound according to Formula I wherein R¹ is OCH₃;

R² is H;

R³ is piperidine optionally substituted with —OCH₂CH₃; and

R⁴ is —CH₃;

or a pharmaceutically-acceptable salt thereof.

A further aspect of the invention provides a compound according to Formula I wherein R¹ is F or OCH₃;

R² is H;

R³ is oxazole; and

R⁴ is —CH₃;

or a pharmaceutically-acceptable salt thereof.

A further aspect of the invention provides a compound according to Formula I wherein R¹ is F;

R² is H;

R³ is oxazole; and

R⁴ is —CH₃;

or a pharmaceutically-acceptable salt thereof.

A further aspect of the invention provides a compound according to Formula I wherein R¹ is OCH₃; R² is H;

R³ is oxazole; and

R⁴ is —CH₃;

or a pharmaceutically-acceptable salt thereof.

A further aspect of the invention provides a compound according to Formula I wherein R¹ is F or OCH₃;

R² is H;

R³ is morpholine; and

R⁴ is —CH₃;

or a pharmaceutically-acceptable salt thereof.

A further aspect of the invention provides a compound according to Formula I wherein R¹ is F;

R² is H;

R³ is morpholine;

R⁴ is —CH₃;

or a pharmaceutically-acceptable salt thereof.

A further aspect of the invention provides a compound according to Formula I wherein R¹ is OCH₃;

R² is H;

R³ is morpholine; and

R⁴ is —CH₃;

or a pharmaceutically-acceptable salt thereof.

A further aspect of the invention provides a compound according to Formula I selected from:
6-methoxy-8-(4-methylpiperazin-1-yl)-N-(4-(2-oxopyrrolidin-1-yl)phenyl)chroman-2-carboxamide;
(+)-6-methoxy-8-(4-methylpiperazin-1-yl)-N-(4-(2-oxopyrrolidin-1-yl)phenyl)chroman-2-carboxamide; (Isomer 1)
(−)-6-methoxy-8-(4-methylpiperazin-1-yl)-N-(4-(2-oxopyrrolidin-1-yl)phenyl)chroman-2-carboxamide; (Isomer 2)
6-methoxy-8-(4-methylpiperazin-1-yl)-N-(4-(2-oxotetrahydropyrimidin-1(2H)-yl)phenyl)chroman-2-carboxamide;
(+)-6-methoxy-8-(4-methylpiperazin-1-yl)-N-(4-(2-oxotetrahydropyrimidin-1(2H)-yl)phenyl)chroman-2-carboxamide; (Isomer 1)
(−)-6-methoxy-8-(4-methylpiperazin-1-yl)-N-(4-(2-oxotetrahydropyrimidin-1(2H)-yl)phenyl)chroman-2-carboxamide; (Isomer 2)
6-methoxy-N-(4-(3-methyl-2-oxotetrahydropyrimidin-1(2H)-yl)phenyl)-8-(4-methylpiperazin-1-yl)chroman-2-carboxamide;
(+)-6-methoxy-N-(4-(3-methyl-2-oxotetrahydropyrimidin-1(2H)-yl)phenyl)-8-(4-methylpiperazin-1-yl)chroman-2-carboxamide; (Isomer 1)
(−)-6-methoxy-N-(4-(3-methyl-2-oxotetrahydropyrimidin-1(2H)-yl)phenyl)-8-(4-methylpiperazin-1-yl)chroman-2-carboxamide; (Isomer 2)
6-fluoro-N-(4-(3-methyl-2-oxotetrahydropyrimidin-1(2H)-yl)phenyl)-8-(4-methylpiperazin-1-yl)chroman-2-carboxamide;
(+)-6-fluoro-N-(4-(3-methyl-2-oxotetrahydropyrimidin-1(2H)-yl)phenyl)-8-(4-methylpiperazin-1-yl)chroman-2-carboxamide; (Isomer 1)
(−)-6-fluoro-N-(4-(3-methyl-2-oxotetrahydropyrimidin-1(2H)-yl)phenyl)-8-(4-methylpiperazin-1-yl)chroman-2-carboxamide; (Isomer 2)
6-methoxy-8 (4-methylpiperazin-1-yl)-N-(4-(2-oxooxazolidin-3-yl)phenyl)chroman-2-carboxamide;
(+)-6-methoxy-8-(4-methylpiperazin-1-yl)-N-(4-(2-oxooxazolidin-3-yl)phenyl)chroman-2-carboxamide; (Isomer 1)
(−)-6-methoxy-8-(4-methylpiperazin-1-yl)-N-(4-(2-oxooxazolidin-3-yl)phenyl)chroman-2-carboxamide; (Isomer 2)
6-methoxy-N-(4-(3-methyl-2,5-dioxoimidazolidin-1-yl)phenyl)-8-(4-methylpiperazin-1-yl)chroman-2-carboxamide;
(+)-6-methoxy-N-(4-(3-methyl-2,5-dioxoimidazolidin-1-yl)phenyl)-8-(4-methylpiperazin-1-yl)chroman-2-carboxamide; (Isomer 1)
(−)-6-methoxy-N-(4-(3-methyl-2,5-dioxoimidazolidin-1-yl)phenyl)-8-(4-methylpiperazin-1-yl)chroman-2-carboxamide; (Isomer 2)
N-(4-(2,5-dioxoimidazolidin-1-yl)phenyl)-6-methoxy-8-(4-methylpiperazin-1-yl)chroman-2-carboxamide;
(+)-N-(4-(2,5-dioxoimidazolidin-1-yl)phenyl)-6-methoxy-8-(4-methylpiperazin-1-yl)chroman-2-carboxamide; (Isomer 1)
(−)-N-(4-(2,5-dioxoimidazolidin-1-yl)phenyl)-6-methoxy-8-(4-methylpiperazin-1-yl)chroman-2-carboxamide; (Isomer 2)
6-fluoro-8-(4-methylpiperazin-1-yl)-N-(4-(2-oxooxazolidin-3-yl)phenyl)chroman-2-carboxamide;
(+)-6-fluoro-8-(4-methylpiperazin-1-yl)-N-(4-(2-oxooxazolidin-3-yl)phenyl)chroman-2-carboxamide; (Isomer 1)
(−)-6-fluoro-8-(4-methylpiperazin-1-yl)-N-(4-(2-oxooxazolidin-3-yl)phenyl)chroman-2-carboxamide; (Isomer 2)
6-fluoro-8-(4-methylpiperazin-1-yl)-N-(4-(2-oxopyrrolidin-1-yl)phenyl)chroman-2-carboxamide;
(+)-6-fluoro-8-(4-methylpiperazin-1-yl)-N-(4-(2-oxopyrrolidin-1-yl)phenyl)chroman-2-carboxamide; (Isomer 1);
(−)-6-fluoro-8-(4-methylpiperazin-1-yl)-N-(4-(2-oxopyrrolidin-1-yl)phenyl)chroman-2-carboxamide; (Isomer 2)
6-fluoro-N-(4-(3-methyl-2,5-dioxoimidazolidin-1-yl)phenyl)-8-(4-methylpiperazin-1-yl)chroman-2-carboxamide;
(+)-6-fluoro-N-(4-(3-methyl-2,5-dioxoimidazolidin-1-yl)phenyl)-8-(4-methylpiperazin-1-yl)chroman-2-carboxamide; (Isomer 1)
(−)-6-fluoro-N-(4-(3-methyl-2,5-dioxoimidazolidin-1-yl)phenyl)-8-(4-methylpiperazin-1-yl)chroman-2-carboxamide; (Isomer 2)
N-(4-(2,5-dioxoimidazolidin-1-yl)phenyl)-6-fluoro-8-(4-methylpiperazin-1-yl)chroman-2-carboxamide;
(+)-N-(4-(2,5-dioxoimidazolidin-1-yl)phenyl)-6-fluoro-8-(4-methylpiperazin-1-yl)chroman-2-carboxamide; (Isomer 1)
(−)-N-(4-(2,5-dioxoimidazolidin-1-yl)phenyl)-6-fluoro-8-(4-methylpiperazin-1-yl)chroman-2-carboxamide; (Isomer 2)
6-fluoro-8-(4-methylpiperazin-1-yl)-N-(4-(2-oxotetrahydropyrimidin-1(2H)-yl)phenyl)chroman-2-carboxamide;
(+)-6-fluoro-8-(4-methylpiperazin-1-yl)-N-(4-(2-oxotetrahydropyrimidin-1 (2H)-yl)phenyl)chroman-2-carboxamide; (Isomer 1)
(−)-6-fluoro-8-(4-methylpiperazin-1-yl)-N-(4-(2-oxotetrahydropyrimidin-1(2H)-yl)phenyl)chroman-2-carboxamide; (Isomer 2)
6-methoxy-N-(4-(3-(methoxymethyl)-1,2,4-oxadiazol-5-yl)phenyl)-8-(4-methylpiperazin-1-yl)chroman-2-carboxamide;
(+)-6-methoxy-N-(4-(3-(methoxymethyl)-1,2,4-oxadiazol-5-yl)phenyl)-8-(4-methylpiperazin-1-yl)chroman-2-carboxamide; (Isomer 1)
(−)-6-methoxy-N-(4-(3-(methoxymethyl)-1,2,4-oxadiazol-5-yl)phenyl)-8-(4-methylpiperazin-1-yl)chroman-2-carboxamide; (Isomer 2)
6-fluoro-N-(4-(3-(methoxymethyl)-1,2,4-oxadiazol-5-yl)phenyl)-8-(4-methylpiperazin-1-yl)chroman-2-carboxamide;
(+)-6-fluoro-N-(4-(3-(methoxymethyl)-1,2,4-oxadiazol-5-yl)phenyl)-8-(4-methylpiperazin-1-yl)chroman-2-carboxamide; (Isomer 1)
(−)-6-fluoro-N-(4-(3-(methoxymethyl)-1,2,4-oxadiazol-5-yl)phenyl)-8-(4-methylpiperazin-1-yl)chroman-2-carboxamide; (Isomer 2)
6-methoxy-N-(4-(3-methyl-1,2,4-oxadiazol-5-yl)phenyl)-8-(4-methylpiperazin-1-yl)chroman-2-carboxamide;
(+)-6-methoxy-N-(4-(3-methyl-1,2,4-oxadiazol-5-yl)phenyl)-8-(4-methylpiperazin-1-yl)chroman-2-carboxamide; (Isomer 1)
(−)-6-methoxy-N-(4-(3-methyl-1,2,4-oxadiazol-5-yl)phenyl)-8-(4-methylpiperazin-1-yl)chroman-2-carboxamide; (Isomer 2)
6-fluoro-N-(4-(3-methyl-1,2,4-oxadiazol-5-yl)phenyl)-8-(4-methylpiperazin-1-yl)chroman-2-carboxamide;
(+)-6-fluoro-N-(4-(3-methyl-1,2,4-oxadiazol-5-yl)phenyl)-8-(4-methylpiperazin-1-yl)chroman-2-carboxamide; (Isomer 1)
(−)-6-fluoro-N-(4-(3-methyl-1,2,4-oxadiazol-5-yl)phenyl)-8-(4-methylpiperazin-1-yl)chroman-2-carboxamide; (Isomer 2)
N-(4-(4-ethoxypiperidin-1-yl)phenyl)-6-fluoro-8-(4-methylpiperazin-1-yl)chroman-2-carboxamide;
(+)-N-(4-(4-ethoxypiperidin-1-yl)phenyl)-6-fluoro-8-(4-methylpiperazin-1-yl)chroman-2-carboxamide; (Isomer 1)
(−)-N-(4-(4-ethoxypiperidin-1-yl)phenyl)-6-fluoro-8-(4-methylpiperazin-1-yl)chroman-2-carboxamide; (Isomer 2)
6-methoxy-N-(4-(5-methyl-1,2,4-oxadiazol-3-yl)phenyl)-8-(4-methylpiperazin-1-yl)chroman-2-carboxamide;
(+)-6-methoxy-N-(4-(5-methyl-1,2,4-oxadiazol-3-yl)phenyl)-8-(4-methylpiperazin-1-yl)chroman-2-carboxamide; (Isomer 1)
(−)-6-methoxy-N-(4-(5-methyl-1,2,4-oxadiazol-3-yl)phenyl)-8-(4-methylpiperazin-1-yl)chroman-2-carboxamide; (Isomer 2)
N-(4-(5-ethyl-1,2,4-oxadiazol-3-yl)phenyl)-6-methoxy-8-(4-methylpiperazin-1-yl)chroman-2-carboxamide;
(+)-N-(4-(5-ethyl-1,2,4-oxadiazol-3-yl)phenyl)-6-methoxy-8-(4-methylpiperazin-1-yl)chroman-2-carboxamide; (Isomer 1)
(−)-N-(4-(5-ethyl-1,2,4-oxadiazol-3-yl)phenyl)-6-methoxy-8-(4-methylpiperazin-1-yl)chroman-2-carboxamide; (Isomer 2)
N-(4-(5-isopropyl-1,2,4-oxadiazol-3-yl)phenyl)-6-methoxy-8-(4-methylpiperazin-1-yl)chroman-2-carboxamide;
(−)-N-(4-(5-isopropyl-1,2,4-oxadiazol-3-yl)phenyl)-6-methoxy-8-(4-methylpiperazin-1-yl)chroman-2-carboxamide; (Isomer 2)
(+)-N-(4-(5-isopropyl-1,2,4-oxadiazol-3-yl)phenyl)-6-methoxy-8-(4-methylpiperazin-1-yl)chroman-2-carboxamide; (Isomer 1)
6-methoxy-N-(4-(5-(methoxymethyl)-1,2,4-oxadiazol-3-yl)phenyl)-8-(4-methylpiperazin-1-yl)chroman-2-carboxamide;
(+)-6-methoxy-N-(4-(5-(methoxymethyl)-1,2,4-oxadiazol-3-yl)phenyl)-8-(4-methylpiperazin-1-yl)chroman-2-carboxamide; (Isomer 1)
(−)-6-methoxy-N-(4-(5-(methoxymethyl)-1,2,4-oxadiazol-3-yl)phenyl)-8-(4-methylpiperazin-1-yl)chroman-2-carboxamide; (Isomer 2)
6-fluoro-N-(4-(5-methyl-1,2,4-oxadiazol-3-yl)phenyl)-8-(4-methylpiperazin-1-yl)chroman-2-carboxamide;
(+)-6-fluoro-N-(4-(5-methyl-1,2,4-oxadiazol-3-yl)phenyl)-8-(4-methylpiperazin-1-yl)chroman-2-carboxamide; (Isomer 1)
(−)-6-fluoro-N-(4-(5-methyl-1,2,4-oxadiazol-3-yl)phenyl)-8-(4-methylpiperazin-1-yl)chroman-2-carboxamide; (Isomer 2)
N-(4-(5-ethyl-1,2,4-oxadiazol-3-yl)phenyl)-6-fluoro-8-(4-methylpiperazin-1-yl)chroman-2-carboxamide;
(+)-N-(4-(5-ethyl-1,2,4-oxadiazol-3-yl)phenyl)-6-fluoro-8-(4-methylpiperazin-1-yl)chroman-2-carboxamide; (Isomer 1)
(−)-N-(4-(5-ethyl-1,2,4-oxadiazol-3-yl)phenyl)-6-fluoro-8-(4-methylpiperazin-1-yl)chroman-2-carboxamide; (Isomer 2)
6-fluoro-N-(4-(5-isopropyl-1,2,4-oxadiazol-3-yl)phenyl)-8-(4-methylpiperazin-1-yl)chroman-2-carboxamide;
(+)-6-fluoro-N-(4-(5-isopropyl-1,2,4-oxadiazol-3-yl)phenyl)-8-(4-methylpiperazin-1-yl)chroman-2-carboxamide; (Isomer 1)
(−)-6-fluoro-N-(4-(5-isopropyl-1,2,4-oxadiazol-3-yl)phenyl)-8-(4-methylpiperazin-1-yl)chroman-2-carboxamide; (Isomer 2)
N-(4-(4-ethoxypiperidin-1-yl)phenyl)-6-methoxy-8-(4-methylpiperazin-1-yl)chroman-2-carboxamide;
(−)-N-(4-(4-ethoxypiperidin-1-yl)phenyl)-6-methoxy-8-(4-methylpiperazin-1-yl)chroman-2-carboxamide; (Isomer 2)
(+)-N-(4-(4-ethoxypiperidin-1-yl)phenyl)-6-methoxy-8-(4-methylpiperazin-1-yl)chroman-2-carboxamide; (Isomer 1)
6-methoxy-N-(4-(4-methyloxazol-2-yl)phenyl)-8-(4-methylpiperazin-1-yl)chroman-2-carboxamide;
(+)-6-methoxy-N-(4-(4-methyloxazol-2-yl)phenyl)-8-(4-methylpiperazin-1-yl)chroman-2-carboxamide; (Isomer 1)
(−)-6-methoxy-N-(4-(4-methyloxazol-2-yl)phenyl)-8-(4-methylpiperazin-1-yl)chroman-2-carboxamide; (Isomer 2)
6-methoxy-N-(4-morpholinophenyl)-8-(4-(2,2,2-trifluoroethyl)piperazin-1-yl)chroman-2-carboxamide;
(+)-6-methoxy-N-(4-morpholinophenyl)-8-(4-(2,2,2-trifluoroethyl)piperazin-1-yl)chroman-2-carboxamide; (Isomer 1)
(−)-6-methoxy-N-(4-morpholinophenyl)-8-(4-(2,2,2-trifluoroethyl)piperazin-1-yl)chroman-2-carboxamide; (Isomer 2)
6-methoxy-8-(4-(2-methoxyethyl)piperazin-1-yl)-N-(4-morpholinophenyl)chroman-2-carboxamide;
(+)-6-methoxy-8-(4-(2-methoxyethyl)piperazin-1-yl)-N-(4-morpholinophenyl)chroman-2-carboxamide; (Isomer 1)
(−)-6-methoxy-8-(4-(2-methoxyethyl)piperazin-1-yl)-N-(4-morpholinophenyl)chroman-2-carboxamide; (Isomer 2)
8-(4-butylpiperazine-1-yl)-6-methoxy-N-(4-morpholinophenyl)chroman-2-carboxamide;
(+)-8-(4-butylpiperazine-1-yl)-6-methoxy-N-(4-morpholinophenyl)chroman-2-carboxamide; (Isomer 1)
(−)-8-(4-butylpiperazine-1-yl)-6-methoxy-N-(4-morpholinophenyl)chroman-2-carboxamide; (Isomer 2)
6-methoxy-N-(4-morpholinophenyl)-8-(4-propylpiperazin-1-yl)chroman-2-carboxamide;
(+)-6-methoxy-N-(4-morpholinophenyl)-8-(4-propylpiperazin-1-yl)chroman-2-carboxamide; (Isomer 1)
(−)-6-methoxy-N-(4-morpholinophenyl)-8-(4-propylpiperazin-1-yl)chroman-2-carboxamide; (Isomer 2)
8-(4-isopropylpiperazin-1-yl)-6-methoxy-N-(4-morpholinophenyl)chroman-2-carboxamide;
(+)-8-(4-isopropylpiperazin-1-yl)-6-methoxy-N-(4-morpholinophenyl)chroman-2-carboxamide; (Isomer 1)
(−)-8-(4-isopropylpiperazin-1-yl)-6-methoxy-N-(4-morpholinophenyl)chroman-2-carboxamide; (Isomer 2)
8-(4-ethylpiperazin-1-yl)-6-methoxy-N-(4-morpholinophenyl)chroman-2-carboxamide;
(+)-8-(4-ethylpiperazin-1-yl)-6-methoxy-N-(4-morpholinophenyl)chroman-2-carboxamide; (Isomer 1)
(−)-8-(4-ethylpiperazin-1-yl)-6-methoxy-N-(4-morpholinophenyl)chroman-2-carboxamide; (Isomer 2)
6-methoxy-N-(4-morpholinophenyl)-8-(piperazin-1-yl)chroman-2-carboxamide;
(+)-6-methoxy-N-(4-morpholinophenyl)-8-(piperazin-1-yl)chroman-2-carboxamide; (Isomer 1)
(−)-6-methoxy-N-(4-morpholinophenyl)-8-(piperazin-1-yl)chroman-2-carboxamide; (Isomer 2)
6-methoxy-2-methyl-8-(4-methylpiperazin-1-yl)-N-(4-morpholinophenyl)chroman-2-carboxamide;
(+)-6-methoxy-2-methyl-8-(4-methylpiperazin-1-yl)-N-(4-morpholinophenyl)chroman-2-carboxamide; (Isomer 1)
(−)-6-methoxy-2-methyl-8-(4-methylpiperazin-1-yl)-N-(4-morpholinophenyl)chroman-2-carboxamide; (Isomer 2)
6-hydroxy-8-(4-methylpiperazin-1-yl)-N-(4-morpholinophenyl)chroman-2-carboxamide;
(+)-6-hydroxy-8-(4-methylpiperazin-1-yl)-N-(4-morpholinophenyl)chroman-2-carboxamide; (Isomer 1)
(−)-6-hydroxy-8-(4-methylpiperazin-1-yl)-N-(4-morpholinophenyl)chroman-2-carboxamide; (Isomer 2)

or a pharmaceutically-acceptable salt thereof.

A further aspect of the invention provides a compound according to Formula I selected from:
(+)-6-methoxy-8-(4-methylpiperazin-1-yl)-N-(4-(2-oxopyrrolidin-1-yl)phenyl)chroman-2-carboxamide; (Isomer 1)
(+)-6-methoxy-8-(4-methylpiperazin-1-yl)-N-(4-(2-oxotetrahydropyrimidin-1 (2H)-yl)phenyl)chroman-2-carboxamide; (Isomer 1)
(+)-6-methoxy-N-(4-(3-methyl-2-oxotetrahydropyrimidin-1 (2H)-yl)phenyl)-8-(4-methylpiperazin-1-yl)chroman-2-carboxamide; (Isomer 1)
(+)-6-fluoro-N-(4-(3-methyl-2-oxotetrahydropyrimidin-1 (2H)-yl)phenyl)-8-(4-methylpiperazin-1-yl)chroman-2-carboxamide; (Isomer 1)
(+)-6-methoxy-8-(4-methylpiperazin-1-yl)-N-(4-(2-oxooxazolidin-3-yl)phenyl)chroman-2-carboxamide; (Isomer 1)
(+)-6-methoxy-N-(4-(3-methyl-2,5-dioxoimidazolidin-1-yl)phenyl)-8-(4-methylpiperazin-1-yl)chroman-2-carboxamide; (Isomer 1)
(+)-N-(4-(2,5-dioxoimidazolidin-1-yl)phenyl)-6-methoxy-8-(4-methylpiperazin-1-yl)chroman-2-carboxamide; (Isomer 1)
(+)-6-fluoro-8-(4-methylpiperazin-1-yl)-N-(4-(2-oxooxazolidin-3-yl)phenyl)chroman-2-carboxamide; (Isomer 1)
(+)-6-fluoro-8-(4-methylpiperazin-1-yl)-N-(4-(2-oxopyrrolidin-1-yl)phenyl)chroman-2-carboxamide; (Isomer 1)
(+)-6-fluoro-N-(4-(3-methyl-2,5-dioxoimidazolidin-1-yl)phenyl)-8-(4-methylpiperazin-1-yl)chroman-2-carboxamide; (Isomer 1)
(+)-N-(4-(2,5-dioxoimidazolidin-1-yl)phenyl)-6-fluoro-8-(4-methylpiperazin-1-yl)chroman-2-carboxamide; (Isomer 1)
(+)-6-fluoro-8-(4-methylpiperazin-1-yl)-N-(4-(2-oxotetrahydropyrimidin-1 (2H)-yl)phenyl)chroman-2-carboxamide; (Isomer 1)
(+)-6-methoxy-N-(4-(3-(methoxymethyl)-1,2,4-oxadiazol-5-yl)phenyl)-8-(4-methylpiperazin-1-yl)chroman-2-carboxamide; (Isomer 1)
(+)-6-fluoro-N-(4-(3-(methoxymethyl)-1,2,4-oxadiazol-5-yl)phenyl)-8-(4-methylpiperazin-1-yl)chroman-2-carboxamide; (Isomer 1)
(+)-6-methoxy-N-(4-(3-methyl-1,2,4-oxadiazol-5-yl)phenyl)-8-(4-methylpiperazin-1-yl)chroman-2-carboxamide; (Isomer 1)
(+)-6-fluoro-N-(4-(3-methyl-1,2,4-oxadiazol-5-yl)phenyl)-8-(4-methylpiperazin-1-yl)chroman-2-carboxamide; (Isomer 1)
(+)-N-(4-(4-ethoxypiperidin-1-yl)phenyl)-6-fluoro-8-(4-methylpiperazin-1-yl)chroman-2-carboxamide; (Isomer 1)
(+)-6-methoxy-N-(4-(5-methyl-1,2,4-oxadiazol-3-yl)phenyl)-8-(4-methylpiperazin-1-yl)chroman-2-carboxamide; (Isomer 1)
(+)-N-(4-(5-ethyl-1,2,4-oxadiazol-3-yl)phenyl)-6-methoxy-8-(4-methylpiperazin-1-yl)chroman-2-carboxamide; (Isomer 1)
(+)-N-(4-(5-isopropyl-1,2,4-oxadiazol-3-yl)phenyl)-6-methoxy-8-(4-methylpiperazin-1-yl)chroman-2-carboxamide; (Isomer 1)
(+)-6-methoxy-N-(4-(5-(methoxymethyl)-1,2,4-oxadiazol-3-yl)phenyl)-8-(4-methylpiperazin-1-yl)chroman-2-carboxamide; (Isomer 1)
(+)-6-fluoro-N-(4-(5-methyl-1,2,4-oxadiazol-3-yl)phenyl)-8-(4-methylpiperazin-1-yl)chroman-2-carboxamide; (Isomer 1)
(+)-N-(4-(5-ethyl-1,2,4-oxadiazol-3-yl)phenyl)-6-fluoro-8-(4-methylpiperazin-1-yl)chroman-2-carboxamide; (Isomer 1)
(+)-6-fluoro-N-(4-(5-isopropyl-1,2,4-oxadiazol-3-yl)phenyl)-8-(4-methylpiperazin-1-yl)chroman-2-carboxamide; (Isomer 1)
(+)-N-(4-(4-ethoxypiperidin-1-yl)phenyl)-6-methoxy-8-(4-methylpiperazin-1-yl)chroman-2-carboxamide; (Isomer 1)
(+)-6-methoxy-N-(4-(4-methyloxazol-2-yl)phenyl)-8-(4-methylpiperazin-1-yl)chroman-2-carboxamide; (Isomer 1)
(+)-6-methoxy-N-(4-morpholinophenyl)-8-(4-(2,2,2-trifluoroethyl)piperazin-1-yl)chroman-2-carboxamide; (Isomer 1)
(+)-6-methoxy-8-(4-(2-methoxyethyl)piperazin-1-yl)-N-(4-morpholinophenyl)chroman-2-carboxamide; (Isomer 1)
(+)-8-(4-butylpiperazine-1-yl)-6-methoxy-N-(4-morpholinophenyl)chroman-2-carboxamide; (Isomer 1)
(+)-6-methoxy-N-(4-morpholinophenyl)-8-(4-propylpiperazin-1-yl)chroman-2-carboxamide; (Isomer 1)
(+)-8-(4-isopropylpiperazin-1-yl)-6-methoxy-N-(4-morpholinophenyl)chroman-2-carboxamide; (Isomer 1)
(+)-8-(4-ethylpiperazin-1-yl)-6-methoxy-N-(4-morpholinophenyl)chroman-2-carboxamide; (Isomer 1)
(+)-6-methoxy-N-(4-morpholinophenyl)-8-(piperazin-1-yl)chroman-2-carboxamide; (Isomer 1)
(+)-6-methoxy-2-methyl-8-(4-methylpiperazin-1-yl)-N-(4-morpholinophenyl)chroman-2-carboxamide; (Isomer 1) and
(+)-6-hydroxy-8-(4-methylpiperazin-1-yl)-N-(4-morpholinophenyl)chroman-2-carboxamide; (Isomer 1)
   or a pharmaceutically-acceptable salt thereof.

A further aspect of the invention provides a method of treating anxiety disorders in a mammal comprising administering to such mammal an effective amount of a compound of formula I 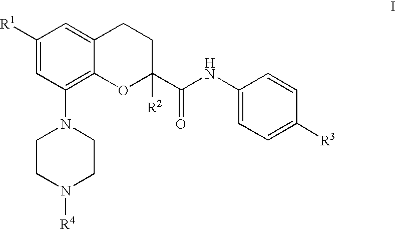
wherein:
R¹ is F or OCH₃ or OH;
R² is H, or CH₃;
R³ is a heterocycle optionally substituted with one of the following —CH₃, —CH₂CH₃, —CH₂CH₂CH₃, —CH(CH₃)₂, —CH₂OCH₃, or —OCH₂CH₃; and
R⁴ is H, —CH₃, CH₂CF₃, —CH₂CH₂OCH₃, CH₂CH₂CH₂CH₃, —CH₂CH₂CH₃, —CH(CH₃)₂, or —CH₂CH₃
or a pharmaceutically-acceptable salt thereof.

A further aspect of the invention provides a method of treating mood disorders in a mammal comprising administering to such mammal an effective amount of a compound of formula I.

A further aspect of the invention provides a method of treating cognitive disorders in a mammal comprising administering to such mammal an effective amount of a compound of formula I.

A further aspect of the invention provides a method of treating anxiety disorders in a mammal comprising administering to such mammal an effective amount of a compound selected from:
6-methoxy-8-(4-methylpiperazin-1-yl)-N-(4-(2-oxopyrrolidin-1-yl)phenyl)chroman-2-carboxamide;
(+)-6-methoxy-8-(4-methylpiperazin-1-yl)-N-(4-(2-oxopyrrolidin-1-yl)phenyl)chroman-2-carboxamide; (Isomer 1)
(−)-6-methoxy-8-(4-methylpiperazin-1-yl)-N-(4-(2-oxopyrrolidin-1-yl)phenyl)chroman-2-carboxamide; (Isomer 2)
6-methoxy-8-(4-methylpiperazin-1-yl)-N-(4-(2-oxotetrahydropyrimidin-1(2H)-yl)phenyl)chroman-2-carboxamide;
(+)-6-methoxy-8-(4-methylpiperazin-1-yl)-N-(4-(2-oxotetrahydropyrimidin-1(2H)-yl)phenyl)chroman-2-carboxamide; (Isomer 1)
(−)-6-methoxy-8-(4-methylpiperazin-1-yl)-N-(4-(2-oxotetrahydropyrimidin-1(2H)-yl)phenyl)chroman-2-carboxamide; (Isomer 2)
6-methoxy-N-(4-(3-methyl-2-oxotetrahydropyrimidin-1(2H)-yl)phenyl)-8-(4-methylpiperazin-1-yl)chroman-2-carboxamide;
(+)-6-methoxy-N-(4-(3-methyl-2-oxotetrahydropyrimidin-1(2H)-yl)phenyl)-8-(4-methylpiperazin-1-yl)chroman-2-carboxamide; (Isomer 1)
(−)-6-methoxy-N-(4-(3-methyl-2-oxotetrahydropyrimidin-1(2H)-yl)phenyl)-8-(4-methylpiperazin-1-yl)chroman-2-carboxamide; (Isomer 2)
6-fluoro-N-(4-(3-methyl-2-oxotetrahydropyrimidin-1(2H)-yl)phenyl)-8-(4-methylpiperazin-1-yl)chroman-2-carboxamide;
(+)-6-fluoro-N-(4-(3-methyl-2-oxotetrahydropyrimidin-1 (2H)-yl)phenyl)-8-(4-methylpiperazin-1-yl)chroman-2-carboxamide; (Isomer 1)
6-methoxy-8-(4-methylpiperazin-1-yl)-N-(4-(2-oxooxazolidin-3-yl)phenyl)chroman-2-carboxamide;
(+)-6-methoxy-8-(4-methylpiperazin-1-yl)-N-(4-(2-oxooxazolidin-3-yl)phenyl)chroman-2-carboxamide; (Isomer 1)
(−)-6-methoxy-8-(4-methylpiperazin-1-yl)-N-(4-(2-oxooxazolidin-3-yl)phenyl)chroman-2-carboxamide; (Isomer 2)
6-methoxy-N-(4-(3-methyl-2,5-dioxoimidazolidin-1-yl)phenyl)-8-(4-methylpiperazin-1-yl)chroman-2-carboxamide;
(+)-6-methoxy-N-(4-(3-methyl-2,5-dioxoimidazolidin-1-yl)phenyl)-8-(4-methylpiperazin-1-yl)chroman-2-carboxamide; (Isomer 1)
(−)-6-methoxy-N-(4-(3-methyl-2,5-dioxoimidazolidin-1-yl)phenyl)-8-(4-methylpiperazin-1-yl)chroman-2-carboxamide; (Isomer 2)
N-(4-(2,5-dioxoimidazolidin-1-yl)phenyl)-6-methoxy-8-(4-methylpiperazin-1-yl)chroman-2-carboxamide;
(+)-N-(4-(2,5-dioxoimidazolidin-1-yl)phenyl)-6-methoxy-8-(4-methylpiperazin-1-yl)chroman-2-carboxamide; (Isomer 1)
(−)-N-(4-(2,5-dioxoimidazolidin-1-yl)phenyl)-6-methoxy-8-(4-methylpiperazin-1-yl)chroman-2-carboxamide; (Isomer 2)
6-fluoro-8-(4-methylpiperazin-1-yl)-N-(4-(2-oxooxazolidin-3-yl)phenyl)chroman-2-carboxamide;
(+)-6-fluoro-8-(4-methylpiperazin-1-yl)-N-(4-(2-oxooxazolidin-3-yl)phenyl)chroman-2-carboxamide; (Isomer 1)
(−)-6-fluoro-8-(4-methylpiperazin-1-yl)-N-(4-(2-oxooxazolidin-3-yl)phenyl)chroman-2-carboxamide; (Isomer 2)
6-fluoro-8-(4-methylpiperazin-1-yl)-N-(4-(2-oxopyrrolidin-1-yl)phenyl)chroman-2-carboxamide;
(+)-6-fluoro-8-(4-methylpiperazin-1-yl)-N-(4-(2-oxopyrrolidin-1-yl)phenyl)chroman-2-carboxamide; (Isomer 1)
(−)-6-fluoro-8-(4-methylpiperazin-1-yl)-N-(4-(2-oxopyrrolidin-1-yl)phenyl)chroman-2-carboxamide; (Isomer 2)
6-fluoro-N-(4-(3-methyl-2,5-dioxoimidazolidin-1-yl)phenyl)-8-(4-methylpiperazin-1-yl)chroman-2-carboxamide;
(+)-6-fluoro-N-(4-(3-methyl-2,5-dioxoimidazolidin-1-yl)phenyl)-8-(4-methylpiperazin-1-yl)chroman-2-carboxamide; (Isomer 1)
(−)-6-fluoro-N-(4-(3-methyl-2,5-dioxoimidazolidin-1-yl)phenyl)-8-(4-methylpiperazin-1-yl)chroman-2-carboxamide; (Isomer 2)
N-(4-(2,5-dioxoimidazolidin-1-yl)phenyl)-6-fluoro-8-(4-methylpiperazin-1-yl)chroman-2-carboxamide;
(+)-N-(4-(2,5-dioxoimidazolidin-1-yl)phenyl)-6-fluoro-8-(4-methylpiperazin-1-yl)chroman-2-carboxamide; (Isomer 1)
(−)-N-(4-(2,5-dioxoimidazolidin-1-yl)phenyl)-6-fluoro-8-(4-methylpiperazin-1-yl)chroman-2-carboxamide; (Isomer 2)
6-fluoro-8-(4-methylpiperazin-1-yl)-N-(4-(2-oxotetrahydropyrimidin-1(2H)-yl)phenyl)chroman-2-carboxamide;
(+)-6-fluoro-8-(4-methylpiperazin-1-yl)-N-(4-(2-oxotetrahydropyrimidin-1(2H)-yl)phenyl)chroman-2-carboxamide; (Isomer 1)
(−)-6-fluoro-8-(4-methylpiperazin-1-yl)-N-(4-(2-oxotetrahydropyrimidin-1(2H)-yl)phenyl)chroman-2-carboxamide; (Isomer 2)
6-methoxy-N-(4-(3-(methoxymethyl)-1,2,4-oxadiazol-5-yl)phenyl)-8-(4-methylpiperazin-1-yl)chroman-2-carboxamide;
(+)-6-methoxy-N-(4-(3-(methoxymethyl)-1,2,4-oxadiazol-5-yl)phenyl)-8-(4-methylpiperazin-1-yl)chroman-2-carboxamide; (Isomer 1)
(−)-6-methoxy-N-(4-(3-(methoxymethyl)-1,2,4-oxadiazol-5-yl)phenyl)-8-(4-methylpiperazin-1-yl)chroman-2-carboxamide; (Isomer 2)
6-fluoro-N-(4-(3-(methoxymethyl)-1,2,4-oxadiazol-5-yl)phenyl)-8-(4-methylpiperazin-1-yl)chroman-2-carboxamide;
(+)-6-fluoro-N-(4-(3-(methoxymethyl)-1,2,4-oxadiazol-5-yl)phenyl)-8-(4-methylpiperazin-1-yl)chroman-2-carboxamide; (Isomer 1)
(−)-6-fluoro-N-(4-(3-(methoxymethyl)-1,2,4-oxadiazol-5-yl)phenyl)-8-(4-methylpiperazin-1-yl)chroman-2-carboxamide; (Isomer 2)
6-methoxy-N-(4-(3-methyl-1,2,4-oxadiazol-5-yl)phenyl)-8-(4-methylpiperazin-1-yl)chroman-2-carboxamide;
(+)-6-methoxy-N-(4-(3-methyl-1,2,4-oxadiazol-5-yl)phenyl)-8-(4-methylpiperazin-1-yl)chroman-2-carboxamide; (Isomer 1)
(−)-6-methoxy-N-(4-(3-methyl-1,2,4-oxadiazol-5-yl)phenyl)-8-(4-methylpiperazin-1-yl)chroman-2-carboxamide; (Isomer 2)
6-fluoro-N-(4-(3-methyl-1,2,4-oxadiazol-5-yl)phenyl)-8-(4-methylpiperazin-1-yl)chroman-2-carboxamide;
(+)-6-fluoro-N-(4-(3-methyl-1,2,4-oxadiazol-5-yl)phenyl)-8-(4-methylpiperazin-1-yl)chroman-2-carboxamide; (Isomer 1)
(−)-6-fluoro-N-(4-(3-methyl-1,2,4-oxadiazol-5-yl)phenyl)-8-(4-methylpiperazin-1-yl)chroman-2-carboxamide; (Isomer 2)
N-(4-(4-ethoxypiperidin-1-yl)phenyl)-6-fluoro-8-(4-methylpiperazin-1-yl)chroman-2-carboxamide;
(+)-N-(4-(4-ethoxypiperidin-1-yl)phenyl)-6-fluoro-8-(4-methylpiperazin-1-yl)chroman-2-carboxamide; (Isomer 1)
(−)-N-(4-(4-ethoxypiperidin-1-yl)phenyl)-6-fluoro-8-(4-methylpiperazin-1-yl)chroman-2-carboxamide; (Isomer 2);
(−)-; (Isomer 2)
(+)-; (Isomer 1)
(+)-(Isomer 1)
(−)-(Isomer 2)
(+)-(Isomer 1)
(−)-(Isomer 2)
(+)-(Isomer 1)
(−)-(Isomer 2)
6-methoxy-N-(4-(5-methyl-1,2,4-oxadiazol-3-yl)phenyl)-8-(4-methylpiperazin-1-yl)chroman-2-carboxamide;
(+)-6-methoxy-N-(4-(5-methyl-1,2,4-oxadiazol-3-yl)phenyl)-8-(4-methylpiperazin-1-yl)chroman-2-carboxamide; (Isomer 1)
(−)-6-methoxy-N-(4-(5-methyl-1,2,4-oxadiazol-3-yl)phenyl)-8-(4-methylpiperazin-1-yl)chroman-2-carboxamide; (Isomer 2)
N-(4-(5-ethyl-1,2,4-oxadiazol-3-yl)phenyl)-6-methoxy-8-(4-methylpiperazin-1-yl)chroman-2-carboxamide;
(+)-N-(4-(5-ethyl-1,2,4-oxadiazol-3-yl)phenyl)-6-methoxy-8-(4-methylpiperazin-1-yl)chroman-2-carboxamide; (Isomer 1)
(−)-N-(4-(5-ethyl-1,2,4-oxadiazol-3-yl)phenyl)-6-methoxy-8-(4-methylpiperazin-1-yl)chroman-2-carboxamide; (Isomer 2)
N-(4-(5-isopropyl-1,2,4-oxadiazol-3-yl)phenyl)-6-methoxy-8-(4-methylpiperazin-1-yl)chroman-2-carboxamide;
(−)-N-(4-(5-isopropyl-1,2,4-oxadiazol-3-yl)phenyl)-6-methoxy-8-(4-methylpiperazin-1-yl)chroman-2-carboxamide; (Isomer 2)
(+)-N-(4-(5-isopropyl-1,2,4-oxadiazol-3-yl)phenyl)-6-methoxy-8-(4-methylpiperazin-1-yl)chroman-2-carboxamide; (Isomer 1)
6-methoxy-N-(4-(5-(methoxymethyl)-1,2,4-oxadiazol-3-yl)phenyl)-8-(4-methylpiperazin-1-yl)chroman-2-carboxamide;
(+)-6-methoxy-N-(4-(5-(methoxymethyl)-1,2,4-oxadiazol-3-yl)phenyl)-8-(4-methylpiperazin-1-yl)chroman-2-carboxamide; (Isomer 1)
(−)-6-methoxy-N-(4-(5-(methoxymethyl)-1,2,4-oxadiazol-3-yl)phenyl)-8-(4-methylpiperazin-1-yl)chroman-2-carboxamide; (Isomer 2)
6-fluoro-N-(4-(5-methyl-1,2,4-oxadiazol-3-yl)phenyl)-8-(4-methylpiperazin-1-yl)chroman-2-carboxamide;
(+)-6-fluoro-N-(4-(5-methyl-1,2,4-oxadiazol-3-yl)phenyl)-8-(4-methylpiperazin-1-yl)chroman-2-carboxamide; (Isomer 1)
(−)-6-fluoro-N-(4-(5-methyl-1,2,4-oxadiazol-3-yl)phenyl)-8-(4-methylpiperazin-1-yl)chroman-2-carboxamide; (Isomer 2)
N-(4-(5-ethyl-1,2,4-oxadiazol-3-yl)phenyl)-6-fluoro-8-(4-methylpiperazin-1-yl)chroman-2-carboxamide;
(+)-N-(4-(5-ethyl-1,2,4-oxadiazol-3-yl)phenyl)-6-fluoro-8-(4-methylpiperazin-1-yl)chroman-2-carboxamide; (Isomer 1)
(−)-N-(4-(5-ethyl-1,2,4-oxadiazol-3-yl)phenyl)-6-fluoro-8-(4-methylpiperazin-1-yl)chroman-2-carboxamide; (Isomer 2)
6-fluoro-N-(4-(5-isopropyl-1,2,4-oxadiazol-3-yl)phenyl)-8-(4-methylpiperazin-1-yl)chroman-2-carboxamide;
(+)-6-fluoro-N-(4-(5-isopropyl-1,2,4-oxadiazol-3-yl)phenyl)-8-(4-methylpiperazin-1-yl)chroman-2-carboxamide; (Isomer 1)
(−)-6-fluoro-N-(4-(5-isopropyl-1,2,4-oxadiazol-3-yl)phenyl)-8-(4-methylpiperazin-1-yl)chroman-2-carboxamide; (Isomer 2)
N-(4-(4-ethoxypiperidin-1-yl)phenyl)-6-methoxy-8-(4-methylpiperazin-1-yl)chroman-2-carboxamide;
(−)-N-(4-(4-ethoxypiperidin-1-yl)phenyl)-6-methoxy-8-(4-methylpiperazin-1-yl)chroman-2-carboxamide; (Isomer 2)
(+)-N-(4-(4-ethoxypiperidin-1-yl)phenyl)-6-methoxy-8-(4-methylpiperazin-1-yl)chroman-2-carboxamide; (Isomer 1)
6-methoxy-N-(4-(4-methyloxazol-2-yl)phenyl)-8-(4-methylpiperazin-1-yl)chroman-2-carboxamide;
(+)-6-methoxy-N-(4-(4-methyloxazol-2-yl)phenyl)-8-(4-methylpiperazin-1-yl)chroman-2-carboxamide; (Isomer 1)
(−)-6-methoxy-N-(4-(4-methyloxazol-2-yl)phenyl)-8-(4-methylpiperazin-1-yl)chroman-2-carboxamide; (Isomer 2)
6-methoxy-N-(4-morpholinophenyl)-8-(4-(2,2,2-trifluoroethyl)piperazin-1-yl)chroman-2-carboxamide;
(+)-6-methoxy-N-(4-morpholinophenyl)-8-(4-(2,2,2-trifluoroethyl)piperazin-1-yl)chroman-2-carboxamide; (Isomer 1)
(−)-6-methoxy-N-(4-morpholinophenyl)-8-(4-(2,2,2-trifluoroethyl)piperazin-1-yl)chroman-2-carboxamide; (Isomer 2)
6-methoxy-8-(4-(2-methoxyethyl)piperazin-1-yl)-N-(4-morpholinophenyl)chroman-2-carboxamide;
(+)-6-methoxy-8-(4-(2-methoxyethyl)piperazin-1-yl)-N-(4-morpholinophenyl)chroman-2-carboxamide; (Isomer 1)
(−)-6-methoxy-8-(4-(2-methoxyethyl)piperazin-1-yl)-N-(4-morpholinophenyl)chroman-2-carboxamide; (Isomer 2)
8-(4-butylpiperazine-1-yl)-6-methoxy-N-(4-morpholinophenyl)chroman-2-carboxamide;
(+)-8-(4-butylpiperazine-1-yl)-6-methoxy-N-(4-morpholinophenyl)chroman-2-carboxamide; (Isomer 1)
(−)-8-(4-butylpiperazine-1-yl)-6-methoxy-N-(4-morpholinophenyl)chroman-2-carboxamide; (Isomer 2)
6-methoxy-N-(4-morpholinophenyl)-8-(4-propylpiperazin-1-yl)chroman-2-carboxamide;
(+)-6-methoxy-N-(4-morpholinophenyl)-8-(4-propylpiperazin-1-yl)chroman-2-carboxamide; (Isomer 1)
(−)-6-methoxy-N-(4-morpholinophenyl)-8-(4-propylpiperazin-1-yl)chroman-2-carboxamide; (Isomer 2)
8-(4-isopropylpiperazin-1-yl)-6-methoxy-N-(4-morpholinophenyl)chroman-2-carboxamide;
(+)-8-(4-isopropylpiperazin-1-yl)-6-methoxy-N-(4-morpholinophenyl)chroman-2-carboxamide; (Isomer 1)
(−)-8-(4-isopropylpiperazin-1-yl)-6-methoxy-N-(4-morpholinophenyl)chroman-2-carboxamide; (Isomer 2)
8-(4-ethylpiperazin-1-yl)-6-methoxy-N-(4-morpholinophenyl)chroman-2-carboxamide;
(+)-8-(4-ethylpiperazin-1-yl)-6-methoxy-N-(4-morpholinophenyl)chroman-2-carboxamide; (Isomer 1)
(−)-8-(4-ethylpiperazin-1-yl)-6-methoxy-N-(4-morpholinophenyl)chroman-2-carboxamide; (Isomer 2)
6-methoxy-N-(4-morpholinophenyl)-8-(piperazin-1-yl)chroman-2-carboxamide;
(+)-6-methoxy-N-(4-morpholinophenyl)-8-(piperazin-1-yl)chroman-2-carboxamide; (Isomer 1)
(−)-6-methoxy-N-(4-morpholinophenyl)-8-(piperazin-1-yl)chroman-2-carboxamide; (Isomer 2)
6-methoxy-2-methyl-8-(4-methylpiperazin-1-yl)-N-(4-morpholinophenyl)chroman-2-carboxamide;
(+)-6-methoxy-2-methyl-8-(4-methylpiperazin-1-yl)-N-(4-morpholinophenyl)chroman-2-carboxamide; (Isomer 1)
(−)-6-methoxy-2-methyl-8-(4-methylpiperazin-1-yl)-N-(4-morpholinophenyl)chroman-2-carboxamide; (Isomer 2)
6-hydroxy-8-(4-methylpiperazin-1-yl)-N-(4-morpholinophenyl)chroman-2-carboxamide;
(+)-6-hydroxy-8-(4-methylpiperazin-1-yl)-N-(4-morpholinophenyl)chroman-2-carboxamide; (Isomer 1), and
(−)-6-hydroxy-8-(4-methylpiperazin-1-yl)-N-(4-morpholinophenyl)chroman-2-carboxamide; (Isomer 2)
   or a pharmaceutically-acceptable salt thereof.
A further aspect of the invention provides a method of treating mood disorders in a mammal comprising administering to such mammal an effective amount of a compound selected from:
6-methoxy-8-(4-methylpiperazin-1-yl)-N-(4-(2-oxopyrrolidin-1-yl)phenyl)chroman-2-carboxamide;
(+)-6-methoxy-8-(4-methylpiperazin-1-yl)-N-(4-(2-oxopyrrolidin-1-yl)phenyl)chroman-2-carboxamide; (Isomer 1)
(−)-6-methoxy-8-(4-methylpiperazin-1-yl)-N-(4-(2-oxopyrrolidin-1-yl)phenyl)chroman-2-carboxamide; (Isomer 2)
6-methoxy-8-(4-methylpiperazin-1-yl)-N-(4-(2-oxotetrahydropyrimidin-1(2H)-yl)phenyl)chroman-2-carboxamide;
(+)-6-methoxy-8-(4-methylpiperazin-1-yl)-N-(4-(2-oxotetrahydropyrimidin-1(2H)-yl)phenyl)chroman-2-carboxamide; (Isomer 1)
(−)-6-methoxy-8-(4-methylpiperazin-1-yl)-N-(4-(2-oxotetrahydropyrimidin-1(2H)-yl)phenyl)chroman-2-carboxamide; (Isomer 2)
6-methoxy-N-(4-(3-methyl-2-oxotetrahydropyrimidin-1(2H)-yl)phenyl)-8-(4-methylpiperazin-1-yl)chroman-2-carboxamide;
(+)-6-methoxy-N-(4-(3-methyl-2-oxotetrahydropyrimidin-1(2H)-yl)phenyl)-8-(4-methylpiperazin-1-yl)chroman-2-carboxamide; (Isomer 1)
(−)-6-methoxy-N-(4-(3-methyl-2-oxotetrahydropyrimidin-1(2H)-yl)phenyl)-8-(4-methylpiperazin-1-yl)chroman-2-carboxamide; (Isomer 2)
6-fluoro-N-(4-(3-methyl-2-oxotetrahydropyrimidin-1(2H)-yl)phenyl)-8-(4-methylpiperazin-1-yl)chroman-2-carboxamide;
(+)-6-fluoro-N-(4-(3-methyl-2-oxotetrahydropyrimidin-1(2H)-yl)phenyl)-8-(4-methylpiperazin-1-yl)chroman-2-carboxamide; (Isomer 1)
6-methoxy-8-(4-methylpiperazin-1-yl)-N-(4-(2-oxooxazolidin-3-yl)phenyl)chroman-2-carboxamide;
(+)-6-methoxy-8-(4-methylpiperazin-1-yl)-N-(4-(2-oxooxazolidin-3-yl)phenyl)chroman-2-carboxamide; (Isomer 1)
(−)-6-methoxy-8-(4-methylpiperazin-1-yl)-N-(4-(2-oxooxazolidin-3-yl)phenyl)chroman-2-carboxamide; (Isomer 2)
6-methoxy-N-(4-(3-methyl-2,5-dioxoimidazolidin-1-yl)phenyl)-8-(4-methylpiperazin-1-yl)chroman-2-carboxamide;
(+)-6-methoxy-N-(4-(3-methyl-2,5-dioxoimidazolidin-1-yl)phenyl)-8-(4-methylpiperazin-1-yl)chroman-2-carboxamide; (Isomer 1)
(−)-6-methoxy-N-(4-(3-methyl-2,5-dioxoimidazolidin-1-yl)phenyl)-8-(4-methylpiperazin-1-yl)chroman-2-carboxamide; (Isomer 2)
N-(4-(2,5-dioxoimidazolidin-1-yl)phenyl)-6-methoxy-8-(4-methylpiperazin-1-yl)chroman-2-carboxamide;
(+)-N-(4-(2,5-dioxoimidazolidin-1-yl)phenyl)-6-methoxy-8-(4-methylpiperazin-1-yl)chroman-2-carboxamide; (Isomer 1)
(−)-N-(4-(2,5-dioxoimidazolidin-1-yl)phenyl)-6-methoxy-8-(4-methylpiperazin-1-yl)chroman-2-carboxamide; (Isomer 2)
6-fluoro-8-(4-methylpiperazin-1-yl)-N-(4-(2-oxooxazolidin-3-yl)phenyl)chroman-2-carboxamide;
(+)-6-fluoro-8-(4-methylpiperazin-1-yl)-N-(4-(2-oxooxazolidin-3-yl)phenyl)chroman-2-carboxamide; (Isomer 1)
(−)-6-fluoro-8-(4-methylpiperazin-1-yl)-N-(4-(2-oxooxazolidin-3-yl)phenyl)chroman-2-carboxamide; (Isomer 2)
6-fluoro-8-(4-methylpiperazin-1-yl)-N-(4-(2-oxopyrrolidin-1-yl)phenyl)chroman-2-carboxamide;
(+)-6-fluoro-8-(4-methylpiperazin-1-yl)-N-(4-(2-oxopyrrolidin-1-yl)phenyl)chroman-2-carboxamide; (Isomer 1)
(−)-6-fluoro-8-(4-methylpiperazin-1-yl)-N-(4-(2-oxopyrrolidin-1-yl)phenyl)chroman-2-carboxamide; (Isomer 2)
6-fluoro-N-(4-(3-methyl-2,5-dioxoimidazolidin-1-yl)phenyl)-8-(4-methylpiperazin-1-yl)chroman-2-carboxamide;
(+)-6-fluoro-N-(4-(3-methyl-2,5-dioxoimidazolidin-1-yl)phenyl)-8-(4-methylpiperazin-1-yl)chroman-2-carboxamide; (Isomer 1)
(−)-6-fluoro-N-(4-(3-methyl-2,5-dioxoimidazolidin-1-yl)phenyl)-8-(4-methylpiperazin-1-yl)chroman-2-carboxamide; (Isomer 2)
N-(4-(2,5-dioxoimidazolidin-1-yl)phenyl)-6-fluoro-8-(4-methylpiperazin-1-yl)chroman-2-carboxamide;
(+)-N-(4-(2,5-dioxoimidazolidin-1-yl)phenyl)-6-fluoro-8-(4-methylpiperazin-1-yl)chroman-2-carboxamide; (Isomer 1)
(−)-N-(4-(2,5-dioxoimidazolidin-1-yl)phenyl)-6-fluoro-8-(4-methylpiperazin-1-yl)chroman-2-carboxamide; (Isomer 2)
6-fluoro-8-(4-methylpiperazin-1-yl)-N-(4-(2-oxotetrahydropyrimidin-1(2H)-yl)phenyl)chroman-2-carboxamide;
(+)-6-fluoro-8-(4-methylpiperazin-1-yl)-N-(4-(2-oxotetrahydropyrimidin-1(2H)-yl)phenyl)chroman-2-carboxamide; (Isomer 1)
(−)-6-fluoro-8-(4-methylpiperazin-1-yl)-N-(4-(2-oxotetrahydropyrimidin-1(2H)-yl)phenyl)chroman-2-carboxamide; (Isomer 2)
6-methoxy-N-(4-(3-(methoxymethyl)-1,2,4-oxadiazol-5-yl)phenyl)-8-(4-methylpiperazin-1-yl)chroman-2-carboxamide;
(+)-6-methoxy-N-(4-(3-(methoxymethyl)-1,2,4-oxadiazol-5-yl)phenyl)-8-(4-methylpiperazin-1-yl)chroman-2-carboxamide; (Isomer 1)
(−)-6-methoxy-N-(4-(3-(methoxymethyl)-1,2,4-oxadiazol-5-yl)phenyl)-8-(4-methylpiperazin-1-yl)chroman-2-carboxamide; (Isomer 2)
6-fluoro-N-(4-(3-(methoxymethyl)-1,2,4-oxadiazol-5-yl)phenyl)-8-(4-methylpiperazin-1-yl)chroman-2-carboxamide;
(+)-6-fluoro-N-(4-(3-(methoxymethyl)-1,2,4-oxadiazol-5-yl)phenyl)-8-(4-methylpiperazin-1-yl)chroman-2-carboxamide; (Isomer 1)
(−)-6-fluoro-N-(4-(3-(methoxymethyl)-1,2,4-oxadiazol-5-yl)phenyl)-8-(4-methylpiperazin-1-yl)chroman-2-carboxamide; (Isomer 2)
6-methoxy-N-(4-(3-methyl-1,2,4-oxadiazol-5-yl)phenyl)-8-(4-methylpiperazin-1-yl)chroman-2-carboxamide;
(+)-6-methoxy-N-(4-(3-methyl-1,2,4-oxadiazol-5-yl)phenyl)-8-(4-methylpiperazin-1-yl)chroman-2-carboxamide; (Isomer 1)
(−)-6-methoxy-N-(4-(3-methyl-1,2,4-oxadiazol-5-yl)phenyl)-8-(4-methylpiperazin-1-yl)chroman-2-carboxamide; (Isomer 2)
6-fluoro-N-(4-(3-methyl-1,2,4-oxadiazol-5-yl)phenyl)-8-(4-methylpiperazin-1-yl)chroman-2-carboxamide;
(+)-6-fluoro-N-(4-(3-methyl-1,2,4-oxadiazol-5-yl)phenyl)-8-(4-methylpiperazin-1-yl)chroman-2-carboxamide; (Isomer 1)
(−)-6-fluoro-N-(4-(3-methyl-1,2,4-oxadiazol-5-yl)phenyl)-8-(4-methylpiperazin-1-yl)chroman-2-carboxamide; (Isomer 2)
N-(4-(4-ethoxypiperidin-1-yl)phenyl)-6-fluoro-8-(4-methylpiperazin-1-yl)chroman-2-carboxamide;
(+)-N-(4-(4-ethoxypiperidin-1-yl)phenyl)-6-fluoro-8-(4-methylpiperazin-1-yl)chroman-2-carboxamide; (Isomer 1)
(−)-N-(4-(4-ethoxypiperidin-1-yl)phenyl)-6-fluoro-8-(4-methylpiperazin-1-yl)chroman-2-carboxamide; (Isomer 2)
6-methoxy-N-(4-(5-methyl-1,2,4-oxadiazol-3-yl)phenyl)-8-(4-methylpiperazin-1-yl)chroman-2-carboxamide;
(+)-6-methoxy-N-(4-(5-methyl-1,2,4-oxadiazol-3-yl)phenyl)-8-(4-methylpiperazin-1-yl)chroman-2-carboxamide; (Isomer 1)
(−)-6-methoxy-N-(4-(5-methyl-1,2,4-oxadiazol-3-yl)phenyl)-8-(4-methylpiperazin-1-yl)chroman-2-carboxamide; (Isomer 2)
N-(4-(5-ethyl-1,2,4-oxadiazol-3-yl)phenyl)-6-methoxy-8-(4-methylpiperazin-1-yl)chroman-2-carboxamide;
(+)-N-(4-(5-ethyl-1,2,4-oxadiazol-3-yl)phenyl)-6-methoxy-8-(4-methylpiperazin-1-yl)chroman-2-carboxamide; (Isomer 1)
(−)-N-(4-(5-ethyl-1,2,4-oxadiazol-3-yl)phenyl)-6-methoxy-8-(4-methylpiperazin-1-yl)chroman-2-carboxamide; (Isomer 2)
N-(4-(5-isopropyl-1,2,4-oxadiazol-3-yl)phenyl)-6-methoxy-8-(4-methylpiperazin-1-yl)chroman-2-carboxamide;
(−)-N-(4-(5-isopropyl-1,2,4-oxadiazol-3-yl)phenyl)-6-methoxy-8-(4-methylpiperazin-1-yl)chroman-2-carboxamide; (Isomer 2)
(+)-N-(4-(5-isopropyl-1,2,4-oxadiazol-3-yl)phenyl)-6-methoxy-8-(4-methylpiperazin-1-yl)chroman-2-carboxamide; (Isomer 1)
6-methoxy-N-(4-(5-(methoxymethyl)-1,2,4-oxadiazol-3-yl)phenyl)-8-(4-methylpiperazin-1-yl)chroman-2-carboxamide;
(+)-6-methoxy-N-(4-(5-(methoxymethyl)-1,2,4-oxadiazol-3-yl)phenyl)-8-(4-methylpiperazin-1-yl)chroman-2-carboxamide; (Isomer 1)
(−)-6-methoxy-N-(4-(5-(methoxymethyl)-1,2,4-oxadiazol-3-yl)phenyl)-8-(4-methylpiperazin-1-yl)chroman-2-carboxamide; (Isomer 2)
6-fluoro-N-(4-(5-methyl-1,2,4-oxadiazol-3-yl)phenyl)-8-(4-methylpiperazin-1-yl)chroman-2-carboxamide;
(+)-6-fluoro-N-(4-(5-methyl-1,2,4-oxadiazol-3-yl)phenyl)-8-(4-methylpiperazin-1-yl)chroman-2-carboxamide; (Isomer 1)
(−)-6-fluoro-N-(4-(5-methyl-1,2,4-oxadiazol-3-yl)phenyl)-8-(4-methylpiperazin-1-yl)chroman-2-carboxamide; (Isomer 2)
N-(4-(5-ethyl-1,2,4-oxadiazol-3-yl)phenyl)-6-fluoro-8-(4-methylpiperazin-1-yl)chroman-2-carboxamide;
(+)-N-(4-(5-ethyl-1,2,4-oxadiazol-3-yl)phenyl)-6-fluoro-8-(4-methylpiperazin-1-yl)chroman-2-carboxamide; (Isomer 1)
(−)-N-(4-(5-ethyl-1,2,4-oxadiazol-3-yl)phenyl)-6-fluoro-8-(4-methylpiperazin-1-yl)chroman-2-carboxamide; (Isomer 2)
6-fluoro-N-(4-(5-isopropyl-1,2,4-oxadiazol-3-yl)phenyl)-8-(4-methylpiperazin-1-yl)chroman-2-carboxamide;
(+)-6-fluoro-N-(4-(5-isopropyl-1,2,4-oxadiazol-3-yl)phenyl)-8-(4-methylpiperazin-1-yl)chroman-2-carboxamide; (Isomer 1)
(−)-6-fluoro-N-(4-(5-isopropyl-1,2,4-oxadiazol-3-yl)phenyl)-8-(4-methylpiperazin-1-yl)chroman-2-carboxamide; (Isomer 2)
N-(4-(4-ethoxypiperidin-1-yl)phenyl)-6-methoxy-8-(4-methylpiperazin-1-yl)chroman-2-carboxamide;
(−)-N-(4-(4-ethoxypiperidin-1-yl)phenyl)-6-methoxy-8-(4-methylpiperazin-1-yl)chroman-2-carboxamide; (Isomer 2)
(+)-N-(4-(4-ethoxypiperidin-1-yl)phenyl)-6-methoxy-8-(4-methylpiperazin-1-yl)chroman-2-carboxamide; (Isomer 1)
6-methoxy-N-(4-(4-methyloxazol-2-yl)phenyl)-8-(4-methylpiperazin-1-yl)chroman-2-carboxamide;
(+)-6-methoxy-N-(4-(4-methyloxazol-2-yl)phenyl)-8-(4-methylpiperazin-1-yl)chroman-2-carboxamide; (Isomer 1)
(−)-6-methoxy-N-(4-(4-methyloxazol-2-yl)phenyl)-8-(4-methylpiperazin-1-yl)chroman-2-carboxamide; (Isomer 2)
6-methoxy-N-(4-morpholinophenyl)-8-(4-(2,2,2-trifluoroethyl)piperazin-1-yl)chroman-2-carboxamide;
(+)-6-methoxy-N-(4-morpholinophenyl)-8-(4-(2,2,2-trifluoroethyl)piperazin-1-yl)chroman-2-carboxamide; (Isomer 1)
(−)-6-methoxy-N-(4-morpholinophenyl)-8-(4-(2,2,2-trifluoroethyl)piperazin-1-yl)chroman-2-carboxamide; (Isomer 2)
6-methoxy-8-(4-(2-methoxyethyl)piperazin-1-yl)-N-(4-morpholinophenyl)chroman-2-carboxamide;
(+)-6-methoxy-8-(4-(2-methoxyethyl)piperazin-1-yl)-N-(4-morpholinophenyl)chroman-2-carboxamide; (Isomer 1)
(−)-6-methoxy-8-(4-(2-methoxyethyl)piperazin-1-yl)-N-(4-morpholinophenyl)chroman-2-carboxamide; (Isomer 2)
8-(4-butylpiperazine-1-yl)-6-methoxy-N-(4-morpholinophenyl)chroman-2-carboxamide;
(+)-8-(4-butylpiperazine-1-yl)-6-methoxy-N-(4-morpholinophenyl)chroman-2-carboxamide; (Isomer 1)
(−)-8-(4-butylpiperazine-1-yl)-6-methoxy-N-(4-morpholinophenyl)chroman-2-carboxamide; (Isomer 2)
6-methoxy-N-(4-morpholinophenyl)-8-(4-propylpiperazin-1-yl)chroman-2-carboxamide;
(+)-6-methoxy-N-(4-morpholinophenyl)-8-(4-propylpiperazin-1-yl)chroman-2-carboxamide; (Isomer 1)
(−)-6-methoxy-N-(4-morpholinophenyl)-8-(4-propylpiperazin-1-yl)chroman-2-carboxamide; (Isomer 2)
8-(4-isopropylpiperazin-1-yl)-6-methoxy-N-(4-morpholinophenyl)chroman-2-carboxamide;
(+)-8-(4-isopropylpiperazin-1-yl)-6-methoxy-N-(4-morpholinophenyl)chroman-2-carboxamide; (Isomer 1)
(−)-8-(4-isopropylpiperazin-1-yl)-6-methoxy-N-(4-morpholinophenyl)chroman-2-carboxamide; (Isomer 2)
8-(4-ethylpiperazin-1-yl)-6-methoxy-N-(4-morpholinophenyl)chroman-2-carboxamide;
(+)-8-(4-ethylpiperazin-1-yl)-6-methoxy-N-(4-morpholinophenyl)chroman-2-carboxamide; (Isomer 1)
(−)-8-(4-ethylpiperazin-1-yl)-6-methoxy-N-(4-morpholinophenyl)chroman-2-carboxamide; (Isomer 2)
6-methoxy-N-(4-morpholinophenyl)-8-(piperazin-1-yl)chroman-2-carboxamide;
(+)-6-methoxy-N-(4-morpholinophenyl)-8-(piperazin-1-yl)chroman-2-carboxamide; (Isomer 1)
(−)-6-methoxy-N-(4-morpholinophenyl)-8-(piperazin-1-yl)chroman-2-carboxamide; (Isomer 2)
6-methoxy-2-methyl-8-(4-methylpiperazin-1-yl)-N-(4-morpholinophenyl)chroman-2-carboxamide;
(+)-6-methoxy-2-methyl-8-(4-methylpiperazin-1-yl)-N-(4-morpholinophenyl)chroman-2-carboxamide; (Isomer 1)
(−)-6-methoxy-2-methyl-8-(4-methylpiperazin-1-yl)-N-(4-morpholinophenyl)chroman-2-carboxamide; (Isomer 2)
6-hydroxy-8-(4-methylpiperazin-1-yl)-N-(4-morpholinophenyl)chroman-2-carboxamide;
(+)-6-hydroxy-8-(4-methylpiperazin-1-yl)-N-(4-morpholinophenyl)chroman-2-carboxamide; (Isomer 1), and
(−)-6-hydroxy-8-(4-methylpiperazin-1-yl)-N-(4-morpholinophenyl)chroman-2-carboxamide; (Isomer 2)
   or a pharmaceutically-acceptable salt thereof.

A further aspect of the invention provides a method of treating cognitive disorders in a mammal comprising administering to such mammal an effective amount of a compound selected from:
6-methoxy-8-(4-methylpiperazin-1-yl)-N-(4-(2-oxopyrrolidin-1-yl)phenyl)chroman-2-carboxamide;
(+)-6-methoxy-8-(4-methylpiperazin-1-yl)-N-(4-(2-oxopyrrolidin-1-yl)phenyl)chroman-2-carboxamide; (Isomer 1)
(−)-6-methoxy-8-(4-methylpiperazin-1-yl)-N-(4-(2-oxopyrrolidin-1-yl)phenyl)chroman-2-carboxamide; (Isomer 2)
6-methoxy-8-(4-methylpiperazin-1-yl)-N-(4-(2-oxotetrahydropyrimidin-1(2H)-yl)phenyl)chroman-2-carboxamide;
(+)-6-methoxy-8-(4-methylpiperazin-1-yl)-N-(4-(2-oxotetrahydropyrimidin-1(2H)-yl)phenyl)chroman-2-carboxamide; (Isomer 1)
(−)-6-methoxy-8-(4-methylpiperazin-1-yl)-N-(4-(2-oxotetrahydropyrimidin-1 (2H)-yl)phenyl)chroman-2-carboxamide; (Isomer 2)
6-methoxy-N-(4-(3-methyl-2-oxotetrahydropyrimidin-1 (2H)-yl)phenyl)-8-(4-methylpiperazin-1-yl)chroman-2-carboxamide;
(+)-6-methoxy-N-(4-(3-methyl-2-oxotetrahydropyrimidin-1(2H)-yl)phenyl)-8-(4-methylpiperazin-1-yl)chroman-2-carboxamide; (Isomer 1)
(−)-6-methoxy-N-(4-(3-methyl-2-oxotetrahydropyrimidin-1(2H)-yl)phenyl)-8-(4-methylpiperazin-1-yl)chroman-2-carboxamide; (Isomer 2)
6-fluoro-N-(4-(3-methyl-2-oxotetrahydropyrimidin-1(2H)-yl)phenyl)-8-(4-methylpiperazin-1-yl)chroman-2-carboxamide;
(+)-6-fluoro-N-(4-(3-methyl-2-oxotetrahydropyrimidin-1(2H)-yl)phenyl)-8-(4-methylpiperazin-1-yl)chroman-2-carboxamide; (Isomer 1)
6-methoxy-8-(4-methylpiperazin-1-yl)-N-(4-(2-oxooxazolidin-3-yl)phenyl)chroman-2-carboxamide;
(+)-6-methoxy-8-(4-methylpiperazin-1-yl)-N-(4-(2-oxooxazolidin-3-yl)phenyl)chroman-2-carboxamide; (Isomer 1)
(−)-6-methoxy-8-(4-methylpiperazin-1-yl)-N-(4-(2-oxooxazolidin-3-yl)phenyl)chroman-2-carboxamide; (Isomer 2)
6-methoxy-N-(4-(3-methyl-2,5-dioxoimidazolidin-1-yl)phenyl)-8-(4-methylpiperazin-1-yl)chroman-2-carboxamide;
(+)-6-methoxy-N-(4-(3-methyl-2,5-dioxoimidazolidin-1-yl)phenyl)-8-(4-methylpiperazin-1-yl)chroman-2-carboxamide; (Isomer 1)
(−)-6-methoxy-N-(4-(3-methyl-2,5-dioxoimidazolidin-1-yl)phenyl)-8-(4-methylpiperazin-1-yl)chroman-2-carboxamide; (Isomer 2)
N-(4-(2,5-dioxoimidazolidin-1-yl)phenyl)-6-methoxy-8-(4-methylpiperazin-1-yl)chroman-2-carboxamide;
(+)-N-(4-(2,5-dioxoimidazolidin-1-yl)phenyl)-6-methoxy-8-(4-methylpiperazin-1-yl)chroman-2-carboxamide; (Isomer 1)
(−)-N-(4-(2,5-dioxoimidazolidin-1-yl)phenyl)-6-methoxy-8-(4-methylpiperazin-1-yl)chroman-2-carboxamide; (Isomer 2)
6-fluoro-8-(4-methylpiperazin-1-yl)-N-(4-(2-oxooxazolidin-3-yl)phenyl)chroman-2-carboxamide;
(+)-6-fluoro-8-(4-methylpiperazin-1-yl)-N-(4-(2-oxooxazolidin-3-yl)phenyl)chroman-2-carboxamide; (Isomer 1)
(−)-6-fluoro-8-(4-methylpiperazin-1-yl)-N-(4-(2-oxooxazolidin-3-yl)phenyl)chroman-2-carboxamide; (Isomer 2)
6-fluoro-8-(4-methylpiperazin-1-yl)-N-(4-(2-oxopyrrolidin-1-yl)phenyl)chroman-2-carboxamide;
(+)-6-fluoro-8-(4-methylpiperazin-1-yl)-N-(4-(2-oxopyrrolidin-1-yl)phenyl)chroman-2-carboxamide; (Isomer 1)
(−)-6-fluoro-8-(4-methylpiperazin-1-yl)-N-(4-(2-oxopyrrolidin-1-yl)phenyl)chroman-2-carboxamide; (Isomer 2)
6-fluoro-N-(4-(3-methyl-2,5-dioxoimidazolidin-1-yl)phenyl)-8-(4-methylpiperazin-1-yl)chroman-2-carboxamide;
(+)-6-fluoro-N-(4-(3-methyl-2,5-dioxoimidazolidin-1-yl)phenyl)-8-(4-methylpiperazin-1-yl)chroman-2-carboxamide; (Isomer 1)
(−)-6-fluoro-N-(4-(3-methyl-2,5-dioxoimidazolidin-1-yl)phenyl)-8-(4-methylpiperazin-1-yl)chroman-2-carboxamide; (Isomer 2)
N-(4-(2,5-dioxoimidazolidin-1-yl)phenyl)-6-fluoro-8-(4-methylpiperazin-1-yl)chroman-2-carboxamide;
(+)-N-(4-(2,5-dioxoimidazolidin-1-yl)phenyl)-6-fluoro-8-(4-methylpiperazin-1-yl)chroman-2-carboxamide; (Isomer 1)
(−)-N-(4-(2,5-dioxoimidazolidin-1-yl)phenyl)-6-fluoro-8-(4-methylpiperazin-1-yl)chroman-2-carboxamide; (Isomer 2)
6-fluoro-8-(4-methylpiperazin-1-yl)-N-(4-(2-oxotetrahydropyrimidin-1(2H)-yl)phenyl)chroman-2-carboxamide;
(+)-6-fluoro-8-(4-methylpiperazin-1-yl)-N-(4-(2-oxotetrahydropyrimidin-1(2H)-yl)phenyl)chroman-2-carboxamide; (Isomer 1)
(−)-6-fluoro-8-(4-methylpiperazin-1-yl)-N-(4-(2-oxotetrahydropyrimidin-1(2H)-yl)phenyl)chroman-2-carboxamide; (Isomer 2)
6-methoxy-N-(4-(3-(methoxymethyl)-1,2,4-oxadiazol-5-yl)phenyl)-8-(4-methylpiperazin-1-yl)chroman-2-carboxamide;
(+)-6-methoxy-N-(4-(3-(methoxymethyl)-1,2,4-oxadiazol-5-yl)phenyl)-8-(4-methylpiperazin-1-yl)chroman-2-carboxamide; (Isomer 1)
(−)-6-methoxy-N-(4-(3-(methoxymethyl)-1,2,4-oxadiazol-5-yl)phenyl)-8-(4-methylpiperazin-1-yl)chroman-2-carboxamide; (Isomer 2)
6-fluoro-N-(4-(3-(methoxymethyl)-1,2,4-oxadiazol-5-yl)phenyl)-8-(4-methylpiperazin-1-yl)chroman-2-carboxamide;
(+)-6-fluoro-N-(4-(3-(methoxymethyl)-1,2,4-oxadiazol-5-yl)phenyl)-8-(4-methylpiperazin-1-yl)chroman-2-carboxamide; (Isomer 1)
(−)-6-fluoro-N-(4-(3-(methoxymethyl)-1,2,4-oxadiazol-5-yl)phenyl)-8-(4-methylpiperazin-1-yl)chroman-2-carboxamide; (Isomer 2)
6-methoxy-N-(4-(3-methyl-1,2,4-oxadiazol-5-yl)phenyl)-8-(4-methylpiperazin-1-yl)chroman-2-carboxamide;
(+)-6-methoxy-N-(4-(3-methyl-1,2,4-oxadiazol-5-yl)phenyl)-8-(4-methylpiperazin-1-yl)chroman-2-carboxamide; (Isomer 1)
(−)-6-methoxy-N-(4-(3-methyl-1,2,4-oxadiazol-5-yl)phenyl)-8-(4-methylpiperazin-1-yl)chroman-2-carboxamide; (Isomer 2)
6-fluoro-N-(4-(3-methyl-1,2,4-oxadiazol-5-yl)phenyl)-8-(4-methylpiperazin-1-yl)chroman-2-carboxamide;
(+)-6-fluoro-N-(4-(3-methyl-1,2,4-oxadiazol-5-yl)phenyl)-8-(4-methylpiperazin-1-yl)chroman-2-carboxamide; (Isomer 1)
(−)-6-fluoro-N-(4-(3-methyl-1,2,4-oxadiazol-5-yl)phenyl)-8-(4-methylpiperazin-1-yl)chroman-2-carboxamide; (Isomer 2)
N-(4-(4-ethoxypiperidin-1-yl)phenyl)-6-fluoro-8-(4-methylpiperazin-1-yl)chroman-2-carboxamide;
(+)-N-(4-(4-ethoxypiperidin-1-yl)phenyl)-6-fluoro-8-(4-methylpiperazin-1-yl)chroman-2-carboxamide; (Isomer 1)
(−)-N-(4-(4-ethoxypiperidin-1-yl)phenyl)-6-fluoro-8-(4-methylpiperazin-1-yl)chroman-2-carboxamide; (Isomer 2)
6-methoxy-N-(4-(5-methyl-1,2,4-oxadiazol-3-yl)phenyl)-8-(4-methylpiperazin-1-yl)chroman-2-carboxamide;
(+)-6-methoxy-N-(4-(5-methyl-1,2,4-oxadiazol-3-yl)phenyl)-8-(4-methylpiperazin-1-yl)chroman-2-carboxamide; (Isomer 1)
(−)-6-methoxy-N-(4-(5-methyl-1,2,4-oxadiazol-3-yl)phenyl)-8-(4-methylpiperazin-1-yl)chroman-2-carboxamide; (Isomer 2)
N-(4-(5-ethyl-1,2,4-oxadiazol-3-yl)phenyl)-6-methoxy-8-(4-methylpiperazin-1-yl)chroman-2-carboxamide;
(+)-N-(4-(5-ethyl-1,2,4-oxadiazol-3-yl)phenyl)-6-methoxy-8-(4-methylpiperazin-1-yl)chroman-2-carboxamide; (Isomer 1)
(−)-N-(4-(5-ethyl-1,2,4-oxadiazol-3-yl)phenyl)-6-methoxy-8-(4-methylpiperazin-1-yl)chroman-2-carboxamide; (Isomer 2)
N-(4-(5-isopropyl-1,2,4-oxadiazol-3-yl)phenyl)-6-methoxy-8-(4-methylpiperazin-1-yl)chroman-2-carboxamide;
(−)-N-(4-(5-isopropyl-1,2,4-oxadiazol-3-yl)phenyl)-6-methoxy-8-(4-methylpiperazin-1-yl)chroman-2-carboxamide; (Isomer 2)
(+)-N-(4-(5-isopropyl-1,2,4-oxadiazol-3-yl)phenyl)-6-methoxy-8-(4-methylpiperazin-1-yl)chroman-2-carboxamide; (Isomer 1)
6-methoxy-N-(4-(5-(methoxymethyl)-1,2,4-oxadiazol-3-yl)phenyl)-8-(4-methylpiperazin-1-yl)chroman-2-carboxamide;
(+)-6-methoxy-N-(4-(5-(methoxymethyl)-1,2,4-oxadiazol-3-yl)phenyl)-8-(4-methylpiperazin-1-yl)chroman-2-carboxamide; (Isomer 1)
(−)-6-methoxy-N-(4-(5-(methoxymethyl)-1,2,4-oxadiazol-3-yl)phenyl)-8-(4-methylpiperazin-1-yl)chroman-2-carboxamide; (Isomer 2)
6-fluoro-N-(4-(5-methyl-1,2,4-oxadiazol-3-yl)phenyl)-8-(4-methylpiperazin-1-yl)chroman-2-carboxamide;
(+)-6-fluoro-N-(4-(5-methyl-1,2,4-oxadiazol-3-yl)phenyl)-8-(4-methylpiperazin-1-yl)chroman-2-carboxamide; (Isomer 1)
(−)-6-fluoro-N-(4-(5-methyl-1,2,4-oxadiazol-3-yl)phenyl)-8-(4-methylpiperazin-1-yl)chroman-2-carboxamide; (Isomer 2)
N-(4-(5-ethyl-1,2,4-oxadiazol-3-yl)phenyl)-6-fluoro-8-(4-methylpiperazin-1-yl)chroman-2-carboxamide;
(+)-N-(4-(5-ethyl-1,2,4-oxadiazol-3-yl)phenyl)-6-fluoro-8-(4-methylpiperazin-1-yl)chroman-2-carboxamide; (Isomer 1)
(−)-N-(4-(5-ethyl-1,2,4-oxadiazol-3-yl)phenyl)-6-fluoro-8-(4-methylpiperazin-1-yl)chroman-2-carboxamide; (Isomer 2)
6-fluoro-N-(4-(5-isopropyl-1,2,4-oxadiazol-3-yl)phenyl)-8-(4-methylpiperazin-1-yl)chroman-2-carboxamide;
(+)-6-fluoro-N-(4-(5-isopropyl-1,2,4-oxadiazol-3-yl)phenyl)-8-(4-methylpiperazin-1-yl)chroman-2-carboxamide; (Isomer 1)
(−)-6-fluoro-N-(4-(5-isopropyl-1,2,4-oxadiazol-3-yl)phenyl)-8-(4-methylpiperazin-1-yl)chroman-2-carboxamide; (Isomer 2)
N-(4-(4-ethoxypiperidin-1-yl)phenyl)-6-methoxy-8-(4-methylpiperazin-1-yl)chroman-2-carboxamide;
(−)-N-(4-(4-ethoxypiperidin-1-yl)phenyl)-6-methoxy-8-(4-methylpiperazin-1-yl)chroman-2-carboxamide; (Isomer 2)
(+)-N-(4-(4-ethoxypiperidin-1-yl)phenyl)-6-methoxy-8-(4-methylpiperazin-1-yl)chroman-2-carboxamide; (Isomer 1)
6-methoxy-N-(4-(4-methyloxazol-2-yl)phenyl)-8-(4-methylpiperazin-1-yl)chroman-2-carboxamide;
(+)-6-methoxy-N-(4-(4-methyloxazol-2-yl)phenyl)-8-(4-methylpiperazin-1-yl)chroman-2-carboxamide; (Isomer 1)
(−)-6-methoxy-N-(4-(4-methyloxazol-2-yl)phenyl)-8-(4-methylpiperazin-1-yl)chroman-2-carboxamide; (Isomer 2)
6-methoxy-N-(4-morpholinophenyl)-8-(4-(2,2,2-trifluoroethyl)piperazin-1-yl)chroman-2-carboxamide;
(+)-6-methoxy-N-(4-morpholinophenyl)-8-(4-(2,2,2-trifluoroethyl)piperazin-1-yl)chroman-2-carboxamide; (Isomer 1)
(−)-6-methoxy-N-(4-morpholinophenyl)-8-(4-(2,2,2-trifluoroethyl)piperazin-1-yl)chroman-2-carboxamide; (Isomer 2)
6-methoxy-8-(4-(2-methoxyethyl)piperazin-1-yl)-N-(4-morpholinophenyl)chroman-2-carboxamide;
(+)-6-methoxy-8-(4-(2-methoxyethyl)piperazin-1-yl)-N-(4-morpholinophenyl)chroman-2-carboxamide; (Isomer 1)
(−)-6-methoxy-8-(4-(2-methoxyethyl)piperazin-1-yl)-N-(4-morpholinophenyl)chroman-2-carboxamide; (Isomer 2)
8-(4-butylpiperazine-1-yl)-6-methoxy-N-(4-morpholinophenyl)chroman-2-carboxamide;
(+)-8-(4-butylpiperazine-1-yl)-6-methoxy-N-(4-morpholinophenyl)chroman-2-carboxamide; (Isomer 1)
(−)-8-(4-butylpiperazine-1-yl)-6-methoxy-N-(4-morpholinophenyl)chroman-2-carboxamide; (Isomer 2)
6-methoxy-N-(4-morpholinophenyl)-8-(4-propylpiperazin-1-yl)chroman-2-carboxamide;
(+)-6-methoxy-N-(4-morpholinophenyl)-8-(4-propylpiperazin-1-yl)chroman-2-carboxamide; (Isomer 1)
(−)-6-methoxy-N-(4-morpholinophenyl)-8-(4-propylpiperazin-1-yl)chroman-2-carboxamide; (Isomer 2)
8-(4-isopropylpiperazin-1-yl)-6-methoxy-N-(4-morpholinophenyl)chroman-2-carboxamide;
(+)-8-(4-isopropylpiperazin-1-yl)-6-methoxy-N-(4-morpholinophenyl)chroman-2-carboxamide; (Isomer 1)
(−)-8-(4-isopropylpiperazin-1-yl)-6-methoxy-N-(4-morpholinophenyl)chroman-2-carboxamide; (Isomer 2)
8-(4-ethylpiperazin-1-yl)-6-methoxy-N-(4-morpholinophenyl)chroman-2-carboxamide;
(+)-8-(4-ethylpiperazin-1-yl)-6-methoxy-N-(4-morpholinophenyl)chroman-2-carboxamide; (Isomer 1)
(−)-8-(4-ethylpiperazin-1-yl)-6-methoxy-N-(4-morpholinophenyl)chroman-2-carboxamide; (Isomer 2)
6-methoxy-N-(4-morpholinophenyl)-8-(piperazin-1-yl)chroman-2-carboxamide;
(+)-6-methoxy-N-(4-morpholinophenyl)-8-(piperazin-1-yl)chroman-2-carboxamide; (Isomer 1)
(−)-6-methoxy-N-(4-morpholinophenyl)-8-(piperazin-1-yl)chroman-2-carboxamide; (Isomer 2)
6-methoxy-2-methyl-8-(4-methylpiperazin-1-yl)-N-(4-morpholinophenyl)chroman-2-carboxamide;
(+)-6-methoxy-2-methyl-8-(4-methylpiperazin-1-yl)-N-(4-morpholinophenyl)chroman-2-carboxamide; (Isomer 1)
(−)-6-methoxy-2-methyl-8-(4-methylpiperazin-1-yl)-N-(4-morpholinophenyl)chroman-2-carboxamide; (Isomer 2)
6-hydroxy-8-(4-methylpiperazin-1-yl)-N-(4-morpholinophenyl)chroman-2-carboxamide;
(+)-6-hydroxy-8-(4-methylpiperazin-1-yl)-N-(4-morpholinophenyl)chroman-2-carboxamide; (Isomer 1), and
(−)-6-hydroxy-8-(4-methylpiperazin-1-yl)-N-(4-morpholinophenyl)chroman-2-carboxamide; (Isomer 2)
   or a pharmaceutically-acceptable salt thereof.

A further aspect of the invention provides a method of treating anxiety disorders in a mammal comprising administering to such mammal an effective amount of a compound selected from:
(+)-6-methoxy-8-(4-methylpiperazin-1-yl)-N-(4-(2-oxopyrrolidin-1-yl)phenyl)chroman-2-carboxamide; (Isomer 1)
(+)-6-methoxy-8-(4-methylpiperazin-1-yl)-N-(4-(2-oxotetrahydropyrimidin-1(2H)-yl)phenyl)chroman-2-carboxamide; (Isomer 1)
(+)-6-methoxy-N-(4-(3-methyl-2-oxotetrahydropyrimidin-1(2H)-yl)phenyl)-8-(4-methylpiperazin-1-yl)chroman-2-carboxamide; (Isomer 1)
(+)-6-fluoro-N-(4-(3-methyl-2-oxotetrahydropyrimidin-1(2H)-yl)phenyl)-8-(4-methylpiperazin-1-yl)chroman-2-carboxamide; (Isomer 1)
(+)-6-methoxy-8-(4-methylpiperazin-1-yl)-N-(4-(2-oxooxazolidin-3-yl)phenyl)chroman-2-carboxamide; (Isomer 1)
(+)-6-methoxy-N-(4-(3-methyl-2,5-dioxoimidazolidin-1-yl)phenyl)-8-(4-methylpiperazin-1-yl)chroman-2-carboxamide; (Isomer 1)
(+)-N-(4-(2,5-dioxoimidazolidin-1-yl)phenyl)-6-methoxy-8-(4-methylpiperazin-1-yl)chroman-2-carboxamide; (Isomer 1)
(+)-6-fluoro-8-(4-methylpiperazin-1-yl)-N-(4-(2-oxooxazolidin-3-yl)phenyl)chroman-2-carboxamide; (Isomer 1)
(+)-6-fluoro-8-(4-methylpiperazin-1-yl)-N-(4-(2-oxopyrrolidin-1-yl)phenyl)chroman-2-carboxamide; (Isomer 1)
(+)-6-fluoro-N-(4-(3-methyl-2,5-dioxoimidazolidin-1-yl)phenyl)-8-(4-methylpiperazin-1-yl)chroman-2-carboxamide; (Isomer 1)
(+)-N-(4-(2,5-dioxoimidazolidin-1-yl)phenyl)-6-fluoro-8-(4-methylpiperazin-1-yl)chroman-2-carboxamide; (Isomer 1)
(+)-6-fluoro-8-(4-methylpiperazin-1-yl)-N-(4-(2-oxotetrahydropyrimidin-1(2H)-yl)phenyl)chroman-2-carboxamide; (Isomer 1)
(+)-6-methoxy-N-(4-(3-(methoxymethyl)-1,2,4-oxadiazol-5-yl)phenyl)-8-(4-methylpiperazin-1-yl)chroman-2-carboxamide; (Isomer 1)
(+)-6-fluoro-N-(4-(3-(methoxymethyl)-1,2,4-oxadiazol-5-yl)phenyl)-8-(4-methylpiperazin-1-yl)chroman-2-carboxamide; (Isomer 1)
(+)-6-methoxy-N-(4-(3-methyl-1,2,4-oxadiazol-5-yl)phenyl)-8-(4-methylpiperazin-1-yl)chroman-2-carboxamide; (Isomer 1)
(+)-6-fluoro-N-(4-(3-methyl-1,2,4-oxadiazol-5-yl)phenyl)-8-(4-methylpiperazin-1-yl)chroman-2-carboxamide; (Isomer 1)
(+)-N-(4-(4-ethoxypiperidin-1-yl)phenyl)-6-fluoro-8-(4-methylpiperazin-1-yl)chroman-2-carboxamide; (Isomer 1)
(+)-6-methoxy-N-(4-(5-methyl-1,2,4-oxadiazol-3-yl)phenyl)-8-(4-methylpiperazin-1-yl)chroman-2-carboxamide; (Isomer 1)
(+)-N-(4-(5-ethyl-1,2,4-oxadiazol-3-yl)phenyl)-6-methoxy-8-(4-methylpiperazin-1-yl)chroman-2-carboxamide; (Isomer 1)
(+)-N-(4-(5-isopropyl-1,2,4-oxadiazol-3-yl)phenyl)-6-methoxy-8-(4-methylpiperazin-1-yl)chroman-2-carboxamide; (Isomer 1)
(+)-6-methoxy-N-(4-(5-(methoxymethyl)-1,2,4-oxadiazol-3-yl)phenyl)-8-(4-methylpiperazin-1-yl)chroman-2-carboxamide; (Isomer 1)
(+)-6-fluoro-N-(4-(5-methyl-1,2,4-oxadiazol-3-yl)phenyl)-8-(4-methylpiperazin-1-yl)chroman-2-carboxamide; (Isomer 1)
(+)-N-(4-(5-ethyl-1,2,4-oxadiazol-3-yl)phenyl)-6-fluoro-8-(4-methylpiperazin-1-yl)chroman-2-carboxamide; (Isomer 1)
(+)-6-fluoro-N-(4-(5-isopropyl-1,2,4-oxadiazol-3-yl)phenyl)-8-(4-methylpiperazin-1-yl)chroman-2-carboxamide; (Isomer 1)
(+)-N-(4-(4-ethoxypiperidin-1-yl)phenyl)-6-methoxy-8-(4-methylpiperazin-1-yl)chroman-2-carboxamide; (Isomer 1)
(+)-6-methoxy-N-(4-(4-methyloxazol-2-yl)phenyl)-8-(4-methylpiperazin-1-yl)chroman-2-carboxamide; (Isomer 1)
(+)-6-methoxy-N-(4-morpholinophenyl)-8-(4-(2,2,2-trifluoroethyl)piperazin-1-yl)chroman-2-carboxamide; (Isomer 1)
(+)-6-methoxy-8-(4-(2-methoxyethyl)piperazin-1-yl)-N-(4-morpholinophenyl)chroman-2-carboxamide; (Isomer 1)
(+)-8-(4-butylpiperazine-1-yl)-6-methoxy-N-(4-morpholinophenyl)chroman-2-carboxamide; (Isomer 1)
(+)-6-methoxy-N-(4-morpholinophenyl)-8-(4-propylpiperazin-1-yl)chroman-2-carboxamide; (Isomer 1)
(+)-8-(4-isopropylpiperazin-1-yl)-6-methoxy-N-(4-morpholinophenyl)chroman-2-carboxamide; (Isomer 1)
(+)-8-(4-ethylpiperazin-1-yl)-6-methoxy-N-(4-morpholinophenyl)chroman-2-carboxamide; (Isomer 1)
(+)-6-methoxy-N-(4-morpholinophenyl)-8-(piperazin-1-yl)chroman-2-carboxamide; (Isomer 1)
(+)-6-methoxy-2-methyl-8-(4-methylpiperazin-1-yl)-N-(4-morpholinophenyl)chroman-2-carboxamide; (Isomer 1), and
(+)-6-hydroxy-8-(4-methylpiperazin-1-yl)-N-(4-morpholinophenyl)chroman-2-carboxamide; (Isomer 1)
   or a pharmaceutically-acceptable salt thereof.

A further aspect of the invention provides a method of treating mood disorders in a mammal comprising administering to such mammal an effective amount of a compound selected from:
(+)-6-methoxy-8-(4-methylpiperazin-1-yl)-N-(4-(2-oxopyrrolidin-1-yl)phenyl)chroman-2-carboxamide; (Isomer 1)
(+)-6-methoxy-8-(4-methylpiperazin-1-yl)-N-(4-(2-oxotetrahydropyrimidin-1(2H)-yl)phenyl)chroman-2-carboxamide; (Isomer 1)
(+)-6-methoxy-N-(4-(3-methyl-2-oxotetrahydropyrimidin-1(2H)-yl)phenyl)-8-(4-methylpiperazin-1-yl)chroman-2-carboxamide; (Isomer 1)
(+)-6-fluoro-N-(4-(3-methyl-2-oxotetrahydropyrimidin-1(2H)-yl)phenyl)-8-(4-methylpiperazin-1-yl)chroman-2-carboxamide; (Isomer 1)
(+)-6-methoxy-8-(4-methylpiperazin-1-yl)-N-(4-(2-oxooxazolidin-3-yl)phenyl)chroman-2-carboxamide; (Isomer 1)
(+)-6-methoxy-N-(4-(3-methyl-2,5-dioxoimidazolidin-1-yl)phenyl)-8-(4-methylpiperazin-1-yl)chroman-2-carboxamide; (Isomer 1)
(+)-N-(4-(2,5-dioxoimidazolidin-1-yl)phenyl)-6-methoxy-8-(4-methylpiperazin-1-yl)chroman-2-carboxamide; (Isomer 1)
(+)-6-fluoro-8-(4-methylpiperazin-1-yl)-N-(4-(2-oxooxazolidin-3-yl)phenyl)chroman-2-carboxamide; (Isomer 1)
(+)-6-fluoro-8-(4-methylpiperazin-1-yl)-N-(4-(2-oxopyrrolidin-1-yl)phenyl)chroman-2-carboxamide; (Isomer 1)
(+)-6-fluoro-N-(4-(3-methyl-2,5-dioxoimidazolidin-1-yl)phenyl)-8-(4-methylpiperazin-1-yl)chroman-2-carboxamide; (Isomer 1)
(+)-N-(4-(2,5-dioxoimidazolidin-1-yl)phenyl)-6-fluoro-8-(4-methylpiperazin-1-yl)chroman-2-carboxamide; (Isomer 1)
(+)-6-fluoro-8-(4-methylpiperazin-1-yl)-N-(4-(2-oxotetrahydropyrimidin-1(2H)-yl)phenyl)chroman-2-carboxamide; (Isomer 1)
(+)-6-methoxy-N-(4-(3-(methoxymethyl)-1,2,4-oxadiazol-5-yl)phenyl)-8-(4-methylpiperazin-1-yl)chroman-2-carboxamide; (Isomer 1)
(+)-6-fluoro-N-(4-(3-(methoxymethyl)-1,2,4-oxadiazol-5-yl)phenyl)-8-(4-methylpiperazin-1-yl)chroman-2-carboxamide; (Isomer 1)
(+)-6-methoxy-N-(4-(3-methyl-1,2,4-oxadiazol-5-yl)phenyl)-8-(4-methylpiperazin-1-yl)chroman-2-carboxamide; (Isomer 1)
(+)-6-fluoro-N-(4-(3-methyl-1,2,4-oxadiazol-5-yl)phenyl)-8-(4-methylpiperazin-1-yl)chroman-2-carboxamide; (Isomer 1)
(+)-N-(4-(4-ethoxypiperidin-1-yl)phenyl)-6-fluoro-8-(4-methylpiperazin-1-yl)chroman-2-carboxamide; (Isomer 1)
(+)-6-methoxy-N-(4-(5-methyl-1,2,4-oxadiazol-3-yl)phenyl)-8-(4-methylpiperazin-1-yl)chroman-2-carboxamide; (Isomer 1)
(+)-N-(4-(5-ethyl-1,2,4-oxadiazol-3-yl)phenyl)-6-methoxy-8-(4-methylpiperazin-1-yl)chroman-2-carboxamide; (Isomer 1)
(+)-N-(4-(5-isopropyl-1,2,4-oxadiazol-3-yl)phenyl)-6-methoxy-8-(4-methylpiperazin-1-yl)chroman-2-carboxamide; (Isomer 1)
(+)-6-methoxy-N-(4-(5-(methoxymethyl)-1,2,4-oxadiazol-3-yl)phenyl)-8-(4-methylpiperazin-1-yl)chroman-2-carboxamide; (Isomer 1)
(+)-6-fluoro-N-(4-(5-methyl-1,2,4-oxadiazol-3-yl)phenyl)-8-(4-methylpiperazin-1-yl)chroman-2-carboxamide; (Isomer 1)
(+)-N-(4-(5-ethyl-1,2,4-oxadiazol-3-yl)phenyl)-6-fluoro-8-(4-methylpiperazin-1-yl)chroman-2-carboxamide; (Isomer 1)
(+)-6-fluoro-N-(4-(5-isopropyl-1,2,4-oxadiazol-3-yl)phenyl)-8-(4-methylpiperazin-1-yl)chroman-2-carboxamide; (Isomer 1)
(+)-N-(4-(4-ethoxypiperidin-1-yl)phenyl)-6-methoxy-8-(4-methylpiperazin-1-yl)chroman-2-carboxamide; (Isomer 1)
(+)-6-methoxy-N-(4-(4-methyloxazol-2-yl)phenyl)-8-(4-methylpiperazin-1-yl)chroman-2-carboxamide; (Isomer 1)
(+)-6-methoxy-N-(4-morpholinophenyl)-8-(4-(2,2,2-trifluoroethyl)piperazin-1-yl)chroman-2-carboxamide; (Isomer 1)
(+)-6-methoxy-8-(4-(2-methoxyethyl)piperazin-1-yl)-N-(4-morpholinophenyl)chroman-2-carboxamide; (Isomer 1)
(+)-8-(4-butylpiperazine-1-yl)-6-methoxy-N-(4-morpholinophenyl)chroman-2-carboxamide; (Isomer 1)
(+)-6-methoxy-N-(4-morpholinophenyl)-8-(4-propylpiperazin-1-yl)chroman-2-carboxamide; (Isomer 1)
(+)-8-(4-isopropylpiperazin-1-yl)-6-methoxy-N-(4-morpholinophenyl)chroman-2-carboxamide; (Isomer 1)
(+)-8-(4-ethylpiperazin-1-yl)-6-methoxy-N-(4-morpholinophenyl)chroman-2-carboxamide; (Isomer 1)
(+)-6-methoxy-N-(4-morpholinophenyl)-8-(piperazin-1-yl)chroman-2-carboxamide; (Isomer 1)
(+)-6-methoxy-2-methyl-8-(4-methylpiperazin-1-yl)-N-(4-morpholinophenyl)chroman-2-carboxamide; (Isomer 1), and
(+)-6-hydroxy-8-(4-methylpiperazin-1-yl)-N-(4-morpholinophenyl)chroman-2-carboxamide; (Isomer 1)
   or a pharmaceutically-acceptable salt thereof.

A further aspect of the invention provides a method of treating cognitive disorders in a mammal comprising administering to such mammal an effective amount of a compound selected from:
(+)-6-methoxy-8-(4-methylpiperazin-1-yl)-N-(4-(2-oxopyrrolidin-1-yl)phenyl)chroman-2-carboxamide; (Isomer 1)
(+)-6-methoxy-8-(4-methylpiperazin-1-yl)-N-(4-(2-oxotetrahydropyrimidin-1(2H)-yl)phenyl)chroman-2-carboxamide; (Isomer 1)
(+)-6-methoxy-N-(4-(3-methyl-2-oxotetrahydropyrimidin-1(2H)-yl)phenyl)-8-(4-methylpiperazin-1-yl)chroman-2-carboxamide; (Isomer 1)
(+)-6-fluoro-N-(4-(3-methyl-2-oxotetrahydropyrimidin-1(2H)-yl)phenyl)-8-(4-methylpiperazin-1-yl)chroman-2-carboxamide; (Isomer 1)
(+)-6-methoxy-8-(4-methylpiperazin-1-yl)-N-(4-(2-oxooxazolidin-3-yl)phenyl)chroman-2-carboxamide; (Isomer 1)
(+)-6-methoxy-N-(4-(3-methyl-2,5-dioxoimidazolidin-1-yl)phenyl)-8-(4-methylpiperazin-1-yl)chroman-2-carboxamide; (Isomer 1)
(+)-N-(4-(2,5-dioxoimidazolidin-1-yl)phenyl)-6-methoxy-8-(4-methylpiperazin-1-yl)chroman-2-carboxamide; (Isomer 1)
(+)-6-fluoro-8-(4-methylpiperazin-1-yl)-N-(4-(2-oxooxazolidin-3-yl)phenyl)chroman-2-carboxamide; (Isomer 1)
(+)-6-fluoro-8-(4-methylpiperazin-1-yl)-N-(4-(2-oxopyrrolidin-1-yl)phenyl)chroman-2-carboxamide; (Isomer 1)
(+)-6-fluoro-N-(4-(3-methyl-2,5-dioxoimidazolidin-1-yl)phenyl)-8-(4-methylpiperazin-1-yl)chroman-2-carboxamide; (Isomer 1)
(+)-N-(4-(2,5-dioxoimidazolidin-1-yl)phenyl)-6-fluoro-8-(4-methylpiperazin-1-yl)chroman-2-carboxamide; (Isomer 1)
(+)-6-fluoro-8-(4-methylpiperazin-1-yl)-N-(4-(2-oxotetrahydropyrimidin-1(2H)-yl)phenyl)chroman-2-carboxamide; (Isomer 1)
(+)-6-methoxy-N-(4-(3-(methoxymethyl)-1,2,4-oxadiazol-5-yl)phenyl)-8-(4-methylpiperazin-1-yl)chroman-2-carboxamide; (Isomer 1)
(+)-6-fluoro-N-(4-(3-(methoxymethyl)-1,2,4-oxadiazol-5-yl)phenyl)-8-(4-methylpiperazin-1-yl)chroman-2-carboxamide; (Isomer 1)
(+)-6-methoxy-N-(4-(3-methyl-1,2,4-oxadiazol-5-yl)phenyl)-8-(4-methylpiperazin-1-yl)chroman-2-carboxamide; (Isomer 1)
(+)-6-fluoro-N-(4-(3-methyl-1,2,4-oxadiazol-5-yl)phenyl)-8-(4-methylpiperazin-1-yl)chroman-2-carboxamide; (Isomer 1)
(+)-N-(4-(4-ethoxypiperidin-1-yl)phenyl)-6-fluoro-8-(4-methylpiperazin-1-yl)chroman-2-carboxamide; (Isomer 1)
(+)-6-methoxy-N-(4-(5-methyl-1,2,4-oxadiazol-3-yl)phenyl)-8-(4-methylpiperazin-1-yl)chroman-2-carboxamide; (Isomer 1)
(+)-N-(4-(5-ethyl-1,2,4-oxadiazol-3-yl)phenyl)-6-methoxy-8-(4-methylpiperazin-1-yl)chroman-2-carboxamide; (Isomer 1)
(+)-N-(4-(5-isopropyl-1,2,4-oxadiazol-3-yl)phenyl)-6-methoxy-8-(4-methylpiperazin-1-yl)chroman-2-carboxamide; (Isomer 1)
(+)-6-methoxy-N-(4-(5-(methoxymethyl)-1,2,4-oxadiazol-3-yl)phenyl)-8-(4-methylpiperazin-1-yl)chroman-2-carboxamide; (Isomer 1)
(+)-6-fluoro-N-(4-(5-methyl-1,2,4-oxadiazol-3-yl)phenyl)-8-(4-methylpiperazin-1-yl)chroman-2-carboxamide; (Isomer 1)
(+)-N-(4-(5-ethyl-1,2,4-oxadiazol-3-yl)phenyl)-6-fluoro-8-(4-methylpiperazin-1-yl)chroman-2-carboxamide; (Isomer 1)
(+)-6-fluoro-N-(4-(5-isopropyl-1,2,4-oxadiazol-3-yl)phenyl)-8-(4-methylpiperazin-1-yl)chroman-2-carboxamide; (Isomer 1)
(+)-N-(4-(4-ethoxypiperidin-1-yl)phenyl)-6-methoxy-8-(4-methylpiperazin-1-yl)chroman-2-carboxamide; (Isomer 1)
(+)-6-methoxy-N-(4-(4-methyloxazol-2-yl)phenyl)-8-(4-methylpiperazin-1-yl)chroman-2-carboxamide; (Isomer 1)
(+)-6-methoxy-N-(4-morpholinophenyl)-8-(4-(2,2,2-trifluoroethyl)piperazin-1-yl)chroman-2-carboxamide; (Isomer 1)
(+)-6-methoxy-8-(4-(2-methoxyethyl)piperazin-1-yl)-N-(4-morpholinophenyl)chroman-2-carboxamide; (Isomer 1)
(+)-8-(4-butylpiperazine-1-yl)-6-methoxy-N-(4-morpholinophenyl)chroman-2-carboxamide; (Isomer 1)
(+)-6-methoxy-N-(4-morpholinophenyl)-8-(4-propylpiperazin-1-yl)chroman-2-carboxamide; (Isomer 1)
(+)-8-(4-isopropylpiperazin-1-yl)-6-methoxy-N-(4-morpholinophenyl)chroman-2-carboxamide; (Isomer 1)
(+)-8-(4-ethylpiperazin-1-yl)-6-methoxy-N-(4-morpholinophenyl)chroman-2-carboxamide; (Isomer 1)
(+)-6-methoxy-N-(4-morpholinophenyl)-8-(piperazin-1-yl)chroman-2-carboxamide; (Isomer 1)
(+)-6-methoxy-2-methyl-8-(4-methylpiperazin-1-yl)-N-(4-morpholinophenyl)chroman-2-carboxamide; (Isomer 1), and
(+)-6-hydroxy-8-(4-methylpiperazin-1-yl)-N-(4-morpholinophenyl)chroman-2-carboxamide; (Isomer 1)
   or a pharmaceutically-acceptable salt thereof.

A further aspect of the invention provides the use of a compound according to formula I in the preparation of a medicament for the treatment of anxiety disorders or mood disorders or cognitive disorders.

A further aspect of the invention provides a pharmaceutical composition comprising a compound of formula I or a pharmaceutically acceptable salt thereof and a pharmaceutically acceptable carrier.

A further aspect of the invention provides a process for the preparation of a compound according to formula I or a pharmaceutically acceptable salt thereof, which process comprises reacting a compound of formula (II): 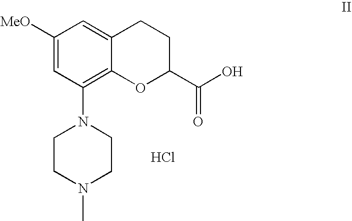
with a compound of formula (III): 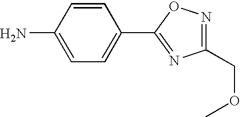
and isolating the desired isomer thereof.

Compounds of the invention have a chiral center at the 2-position of the chroman structure. Such compounds exist in S- and R-forms illustrated below:

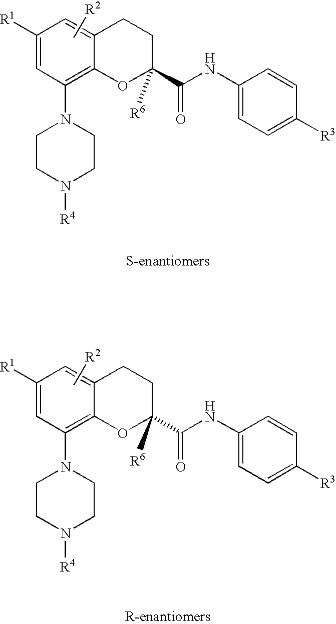

Such forms may be fractionated by chiral chromatography and it has been found for the compounds fractionated that a first-emerging dextrorotatory compound has greater antagonist activity than a second-emerging laevorotatory compound. While not wishing to be bound by any theory it is currently believed that the (+) isomers are the (R) enantiomers and the (−) isomers are the (S) enantiomers. Thus, while dextrorotatory, (D) or (+), and laevorotatory, (L) or (−) compounds, are compounds of the invention, particular compounds of the invention are dextrorotatory, (D) or (+) or (R), compounds.

Particular compounds described herein illustrate, but do not limit the invention, other compounds within the scope of the invention will be apparent to those of skill in the art upon contemplation of the processes, methods and compounds described herein.

The compounds provided herein are useful in the form as a free base, but may also be provided in the form of a pharmaceutically acceptable salt, and/or in the form of a pharmaceutically acceptable hydrate. For example pharmaceutically acceptable salts of compounds of Formula I, include those derived from mineral acids such as for example: methane sulfonic acid, hydrochloric acid, nitric acid, phosphoric acid, sulfuric acid, hydrobromic acid, hydroiodic acid, nitrous acid, and phosphorous acid. Pharmaceutically acceptable salts may also be developed with organic acids including aliphatic mono and dicarboxylates and aromatic acids.

Other pharmaceutically acceptable salts of compounds of the present invention include for example sulfate, pyrosulfate, bisulfate, bisulfite, nitrate, and phosphate.

Compounds of Formula I can be made by processes known in the chemical arts for the production of structurally analogous compounds. Accordingly, the compounds of this invention may be prepared by employing procedures known in the literature starting from known compounds or readily prepared intermediates. For example, intermediate compounds designated as hydrochlorides also contain in most instances one equivalent of lithium chloride. For example, the core bicyclic, heterocyclic structure may be made by first preparing a chromone, quinolone or quinoline. The compounds of the present invention are made by the general procedure for amide coupling, that is, by coupling an anime with an acid using known coupling procedures. The amines used in the current invention if not commercially available may be made by known techniques. For example as a first step in the process of making compound of Formula I, a nitro compound may be reduced to an amine. The nitro compound may be a nitrophenyl compound. The resulting amines may be reacted with an acid.

Provided herein are synthetic methods for the preparation of precursor compounds or use in practicing aspects of the present invention.

It will be appreciated by those skilled in the art that certain compounds of the present invention contain for example asymmetrically substituted carbon, and accordingly may exist in and be isolated in, optically-active and racemic forms. Some compounds may exhibit polymorphism, thus it is to be understood that the present invention encompasses racemic, optically active, polymorphic or stereoisomeric forms, or mixtures thereof, which forms possess properties useful in the treatment of the disorders set forth below. Preparation of optically active forms is well known in the art (for example by resolution of racemic forms by recrystallization techniques, synthesis from optically active starting materials, chiral synthesis, or by chromatographic separation using a chiral stationary phase.

Compounds of Formula I have been found to be 5-HT_{1B} antagonists. The compounds of Formula I, and their pharmaceutically acceptable salts, may also be used in a method for the treatment of anxiety disorders, cognitive disorders, or mood disorders. The treatment of such disorders comprises administering to a warm-blooded animal, preferably a mammal, more preferably a human, in need of such treatment, an effective amount of a compound of Formula I or a pharmaceutically acceptable salt of said compound.

Further provided is the use of a compound of Formula I in the preparation of a medicament for the treatment of a disorder such as migraine in a warm-blooded animal, preferably a mammal, more preferably a human, suffering from such disorder.

The invention further provides a pharmaceutical composition suitable for the treatment of the above describe disorders comprising administering to a warm-blooded animal having such disorder an effective amount of a pharmaceutical composition of a compound of Formula I, or a pharmaceutically acceptable salt.

The invention also provides a pharmaceutical composition comprising a compound of Formula I, as defined herein, or a pharmaceutically acceptable salt, in combination with a pharmaceutically acceptable carrier. Preferred compounds of Formula I, for use in the compositions of the invention are as described above.

Compounds described herein demonstrate binding affinities (observed Ki values), in an assay described herein, of better than 10 μM. Selected compounds of the present invention are found to be active antagonists with activity of less than 100 μM/kg. In addition, selected compounds of the present invention demonstrate 5-HT_{1B} antagonist activity by reversing 5-HT_{1B} agonist-induced hypothermia in the guinea pig.

The compounds described herein may be provided or delivered in a form suitable for oral use, for example in a tablet, lozenge, hard and soft capsule, aqueous solution, oily solution, emulsion, and suspension. The compounds may be also be provided for topical administration, for example, as a cream, ointment, gel, spray, or aqueous solutions, oily solutions, emulsions or suspensions. The compounds described herein may also be provided in a form suitable for nasal administration for example, as a nasal spray, nasal drops, or dry powder. The compositions may also be administered to the vagina or rectum in the form of a suppository. The compounds described herein may also be administered parentally, for example by intravenous, intravesicular, subcutaneous, or intramuscular injection or infusion. The compounds may be administered by insufflation (for example as a finely divided powder). The compounds may also be administered transdermally or sublingually. The compounds of the invention may accordingly be obtained by conventional procedures using conventional pharmaceutical excipients, well known in the art. Thus, compositions intended for oral use may contain, for example, one or more coloring, sweetening, flavoring and/or preservative agents.

The amount of active ingredient that is combined with one or more excipients to produce a single dosage form will necessarily vary depending upon the host treated and the particular route of administration. The size of the dose for therapeutic or prophylactic purposes of a compound of the Formula I, will naturally vary according to the nature and severity of the conditions, the age and sex of the animal or patient and the route of administration, according to well known principles of medicine. Various assays and in vivo tests are known for determining the utility of the compounds in the disorders noted above and specifically as agonists and antagonists of 5-HT_{1B} and 5-HT_{1D} receptors.

A compound of formula (I) or a pharmaceutically acceptable salt, solvate or in vivo hydrolysable ester thereof, or a pharmaceutical composition or formulation comprising a compound of formula (I) is administered concurrently, simultaneously, sequentially or separately with another compound or compounds selected from the following:

(i) antidepressants such as amitriptyline, amoxapine, bupropion, citalopram, clomipramine, desipramine, doxepin duloxetine, elzasonan, escitalopram, fluvoxamine, fluoxetine, gepirone, imipramine, ipsapirone, maprotiline, nortriptyline, nefazodone, paroxetine, phenelzine, protriptyline, reboxetine, sertraline, sibutramine, thionisoxetine, tranylcypromaine, trazodone, trimipramine, venlafaxine and equivalents including but not limited to pharmaceutically acceptable salts and pharmaceutically active isomer(s) and metabolite(s) thereof.

(ii) atypical antipsychotics including for example quetiapene and lithium and equivalents including but not limited to pharmaceutically acceptable salts pharmaceutically and active isomer(s) and metabolite(s) thereof.

(iii) antipsychotics including for example amisulpride, aripiprazole, asenapine, benzisoxidil, bifeprunox, carbamazepine, clozapine, chlorpromazine, debenzapine, divalproex, duloxetine, eszopiclone, haloperidol, iloperidone, lamotrigine, loxapine, mesoridazine, olanzapine, paliperidone, perlapine, perphenazine, phenothiazine, phenylbutlypiperidine, pimozide, prochlorperazine, risperidone, sertindole, sulpiride, suproclone, suriclone, thioridazine, trifluoperazine, trimetozine, valproate, valproic acid, zopiclone, zotepine, ziprasidone and equivalents including but not limited to pharmaceutically acceptable salts and pharmaceutically active isomer(s) and metabolite(s) thereof.

(iv) anxiolytics including for example alnespirone, azapirones, benzodiazepines, barbiturates such as adinazolam, alprazolam, balezepam, bentazepam, bromazepam, brotizolam, buspirone, clonazepam, clorazepate, chlordiazepoxide, cyprazepam, diazepam, diphenhydramine, estazolam, fenobam, flunitrazepam, flurazepam, fosazepam, lorazepam, lormetazepam, meprobamate, midazolam, nitrazepam, oxazepam, prazepam, quazepam, reclazepam, tracazolate, trepipam, temazepam, triazolam, uldazepam, zolazepam and equivalents including but not limited to pharmaceutically acceptable salts and pharmaceutically active isomer(s) and metabolite(s) thereof.

(v) anticonvulsants including for example carbamazepine, topiramate, valproate, lamotrigine, gabapentin and equivalents including but not limited to pharmaceutically acceptable salts and pharmaceutically active isomer(s) and metabolite(s) thereof.

(vi) Alzheimer's therapies including for example donepezil, memantine, tacrine and equivalents including but not limited to pharmaceutically acceptable salts and pharmaceutically active isomer(s) and metabolite(s) thereof.

(vii) Parkinson's therapies including for example deprenyl, L-dopa, Requip, Mirapex, MAOB inhibitors such as selegine and rasagiline, comP inhibitors such as Tasmar, A-2 inhibitors, dopamine reuptake inhibitors, NMDA antagonists, Nicotine agonists, Dopamine agonists and inhibitors of neuronal nitric oxide synthase and equivalents including but not limited to pharmaceutically acceptable salts and pharmaceutically active isomer(s) and metabolite(s) thereof.

(ix) stroke therapies including for example abciximab, activase, CEROVIVE™ (NXY-059), citicoline, crobenetine, desmoteplase, repinotan, traxoprodil and equivalents including but not limited to pharmaceutically acceptable salts and pharmaceutically active isomer(s) and metabolite(s) thereof.

(x) urinary incontinence and/or overactive bladder therapies including for example darifenacin, falvoxate, oxybutynin, propiverine, robalzotan, solifenacin, tispium, tolterodine and equivalents including but not limited to pharmaceutically acceptable salts and pharmaceutically active isomer(s) and metabolite(s) thereof.

(xi) neuropathic pain therapies including for example gabapentin, lidoderm, pregablin and equivalents including but not limited to pharmaceutically acceptable salts and pharmaceutically active isomer(s) and metabolite(s) thereof.

(xii) nociceptive pain therapies such as celecoxib, etoricoxib, lumiracoxib, rofecoxib, valdecoxib, diclofenac, loxoprofen, naproxen, paracetamol and equivalents including but not limited to pharmaceutically acceptable salts and pharmaceutically active isomer(s) and metabolite(s) thereof.

(xiii) insomnia therapies including for example allobarbital, alonimid, amobarbital, benzoctamine, butabarbital, capuride, chloral, cloperidone, clorethate, dexclamol, eszopiclone, ethchlorvynol, etomidate, glutethimide, halazepam, hydroxyzine, mecloqualone, melatonin, mephobarbital, methaqualone, midaflur, nisobamate, pentobarbital, phenobarbital, propofol, roletamide, triclofos, secobarbital, zaleplon, zolpidem and equivalents including but not limited to pharmaceutically acceptable salts and pharmaceutically active isomer(s) and metabolite(s) thereof.

Such combination products employ the compounds of this invention within the dosage range described herein and the other pharmaceutically active agent within approved dosage ranges and/or the dosage described in the publication reference.

The utility of the compounds for example to treat anxiety disorders or mood disorders such as depression or dementia and other cognitive disorders such as Alzheimer's disease may be shown via a learned helplessness test in guinea pigs, which is used extensively as correlative to antidepressant activity in humans. The learned helplessness test may be carried out as follows: Seventy male Hartley guinea pigs, each weighing about 350-425 gm are fed ad lib, and are housed under a 12-hour light/dark cycle. The procedure consists of two phases: The induction phase and the avoidance-training phase. In the induction phase, subjects are placed into standard shuttle cages (20 L×16 W×21 centimeters H), which are fitted with a grid floor. Electrical stimulation (1.25 mA, 10 sec duration) is delivered to the floor of the cage every 90-sec during 1 hour daily sessions. Subjects have no opportunity to escape or to avoid shocks. Induction is conducted for 2 consecutive days.

In avoidance training, testing may be conducted in the shuttle cages, except that the subjects are not returned to the same chamber in which induction had occurred. Additionally, all cages are fitted with a partition with an arch in the center of the cage, through which animals can pass between the left and right halves of the cage. The procedure employed is a standard shuttle avoidance procedure in which a compound, conditioned stimulus (a 10-sec presentation of a tone and turning on of a lamp on the side of the cage that the guinea pig was occupying) serves to indicate presentation of electrical current to the floor of the cage. Shock is presented for a 5 sec period, 5 sec after initiation of the conditioned stimulus. Entry into the opposite side of the shuttle cage via the arched partition prior to shock onset results in the end of the trial (avoidance response). If shock is delivered, entry into the opposite side of the cage results in termination of the shock and CS (escape). Reversal of learned helplessness in the induction subjects correlates to antidepressant activity of the test compound. Avoidance training, 45-min in duration, is conducted on 2 consecutive days, beginning 48 hr after the final induction session. Seventy subjects are assigned to 1 of 6 groups of 11-12 animals. The groups are as follows:

1) No induction group. The subjects are placed into the shuttle cages but are not given inescapable shock, the animals are subsequently trained in the avoidance procedure and the vehicle is administered;

Induction vehicle control group;

Imipramine 17.8 mg/kg;

0.3 mg/kg compounds;

1 mg/kg compounds; and

5 mg/kg compounds.

Groups 2-6 are given induction and avoidance training sessions. Injections are administered immediately following induction sessions and 1 hour prior to avoidance training sessions. A second injection is administered 7-8 hours following the first injection, for a total of 9 injections administered over 5 days. No injections are administered following the final avoidance training session.

Compounds of the present invention may be administered in a volume of 1 mL/kg bwt. Imipramine is dissolved in DI water. The compounds are dissolved in DI water, to which was added a few drops of lactic acid (pH 5.5). The vehicle control is DI water prepared with lactic acid to the same pH as the-treated groups.

The primary dependent variable is escape failure during avoidance training. 2-way analysis of variance (ANOVA) is used to assess overall treatment effect, with Dunn's post hoc analysis used to compare the vehicle-treated group with the drug-treated groups. The no-induction group is used to gauge whether learned helplessness is established, by comparison to the vehicle treated group.

Other assays that may be used to measure for example affinity of compounds of the present invention for 5-HT_{1B} and 5-HT_{1D} receptors are described in J. Med. Chem. 41:1218-1235, 1228 (1998) and J. Med. Chem. 42:4981-5001, (1999) and incorporated by reference herein. These assays may be used with some modifications: Frozen membrane preparations of a stably transfected chinese hamster ovary (CHO) cell line expressing 5-HT_{1B} receptors and 5-HT_{1D} receptors may be thawed rapidly, briefly vortexed, and diluted in assay buffer (AB) containing 50 mM Tris-HCl, 4 mM MgCl₂, 4 mM CaCl₂, 1 mM EDTA, and adjusted to pH 7.4 with NaOH. Final protein concentrations are −0.185 mg/ml for 5-HT_{1B}, and 0.4 mg/ml for 5-HT_{1D} membranes. Test compounds are evaluated in competition assays using [³H]-GR125743 (Amersham). The ligand concentration in both assays was 0.27 nM. Kd for [³H]-GR125743 may vary from 0.15 nM to 0.25 nM. The 5-HT_{1B} and 5-HT_{1D} assays are performed simultaneously on one 96-well assay plate, one drug/compound per plate. Ten serial dilutions (1 uM to 4 pM, final concentration) of compound are prepared in DMSO from 10 mM stock solutions. Incubation mixtures are prepared in quadruplicate in 96-deep well assay plates (Matrix 1 ml). Final assay volumes per well are 10 μl compound/nonspecific; 100 μl membranes; 100 μl [3H]-GR125743; and 790 μl AB. Specific binding is defined by using 10-μM Methiothepin. The assay plates are shaken for 5 min., and then incubated for an additional 55 min. Then the assay plates are filtered through Beckman GF/B filters (soaked >2 hrs. in PEI) using a Packard Filtermate 196. Filters are washed 2× with 1 ml ice-cold wash buffer (5 mM Tris-HCl-pH7.4 with NaOH). After the filters are dried, 35 μl of Microscint20 is added to each well. The plates are then counted on a Packard TopCount to determine CPM's per well. Ki values are determined for each test compound utilizing the graphic and analytical software package, GraphPad Prism. Compounds are then ranked in order of potency, and selectivity for 5-HT_{1B} over 5-HT_{1D} receptors.

A method that may be used to determine a compound's affinity for 5-HT_{1B} and 5-HT_{1D} receptors is a guinea pig cortical test. This assay is described in detail by Roberts, et al, Br. J. Pharmacol. 1996, 117, 384-388. The test is carried out as follows: Guinea pigs are decapitated and the cortici is dissected out, weighed and homogenized in 50 mM Tris-HCl, pH 7.7 with an Ultra-Turrax followed by centrifugation for 10 min at 48000×g and 5° C. The pellet is resuspended and recentrifuged. The final pellet is suspended in 0.32 M sucrose buffer to a concentration of 0.5 g original wet weight per mL and stored frozen at −70° C. The radioligand binding assay is carried out as follows: [³H]GR125743 saturation studies are tested in duplicate with 3-4 mg w.w. per tube in 5 mL buffer (50 mM Tris, 4 mM CaCl2, 4 mM MgCl2 and 1 mM EDTA at pH 7.7), and a concentration range of 0.012-2 nM (10-12 concentrations) for the radioligand. Non-specific binding is determined in the presence of 10 mM methiothepin. In competition experiments 4-8 mg w.w. per tube and a radioligand concentration of 0.2 nM are used with 10-12 concentrations of the competing drug. The assays are run for 24 hours at 30° C. and terminated by rapid filtration through Whatman GF/B filters (pretreated with 0.1% polyethyleneimine) using a Brandel cell harvester. Bovine serum albumin (0.1%) is added to the washing buffer to reduce non-specific binding. Data from the experiments may be analyzed using the iterative non-linear curve-fitting program LIGAND. The K_{d} values obtained from the saturation studies are used in the calculation of the Ki values by the LIGAND program. The K_{d} value of [³H]GR125743 may result in a measurement of 46±4 pM and the Bₘₐₓ in a measurement of 4.9±0.2 pmol/g w.w.

A GTPγS binding assay may be used to determine whether a compound is a 5-HT_{1B} or 5-HT_{1D} agonist or antagonist. One assay available measures agonist stimulated GTP binding for example as set forth by Lazareno, S. (1999) Methods in Molecular Biology 106: 231-245. Frozen membranes may be thawed, briefly sonicated, and diluted to 167 μg/ml protein in assay buffer containing 20 mM HEPES, 100 mM NaCl, 1 mM MgCL₂ and 1 μM GDP, pH adjusted to 7.4 with NaOH. Diluted membranes are briefly homogenized with a Polytron and allowed to equilibrate at room temperature for at least 15 minutes before use. Serial dilutions (10 μM to 1 pM, final concentration) of test compounds are prepared in buffer with and without 100 nM 5-HT (final concentration) from 10 mM DMSO stock solutions. Incubation mixtures are prepared in quadruplicate in 96-well, deep-well plates and consisted of 180 μL of membranes (30 μg protein) and 40 μL of compound with or without 5-HT. After an incubation period of 15 minutes at room temperature, 20 μ-L of [³⁵S]GTPγS (NEN; 100 pM final concentration) is added to begin the assay. Mixtures are shaken for 2 minutes and incubated at room temperature for an additional 28 minutes. The reaction is stopped by rapid filtration through Beckman GF/B glass fiber filters using a 96-well Packard cell harvester. Filters are washed four times with 1 mL ice-cold water. The filter plates are nominally dried and 30 μL of scintillation cocktail (MicroScint 40, Packard) is added to each well. CPMs for each well is determined using a TopCount Scintillation Counter (Packard). Maximum stimulation of [³⁵S]GTPγS binding is defined in the presence of 100 nM 5-HT. Basal [³⁵ S]GTPγS binding is defined in buffer alone. IC₅₀ values are defined as the concentration of compound at which 50% of the 100 nM 5-HT response was obtained. Maximal intrinsic activity (IA) of a compound is defined as the percent maximal 5-HT-induced stimulation by 10 μM compound in the absence of 5-HT. As an inter-assay standard, a concentration response curve of 5-HT (1 μM to 1 pM final) in the absence of compounds was included in each assay and an EC₅₀ was determined.

The following reference examples illustrate the making of intermediates in the synthesis of the compounds of the present invention, and are not intended to limit the invention in any manner.

The resolution of racemic compounds is achieved by a variety of methods including: resolution of racemic forms by recrystallization techniques, synthesis from optically active starting materials, chiral synthesis, HPLC chromatographic separation using a chiral stationary phase and supercritical fluid chromatography (SFC) using a chiral stationary phase.

Preparative HPLC was performed using either 21×250 mm columns (˜20 mL/min flow rates) for samples up to 300 mg or 50×500 mm (˜150 mL/min flow rates) for samples above 300 mg. A variety of chiral stationary phases (Chiralpak AD, 10 micron, for example) and a variety of solvent mixtures are used and are described in the following synthetic details. UV detection was either single or multi-wavelength set at 220, 254 and 280 nm.

Preparative SFC was achieved using a Berger auto prep 2 instrument using 21.2×250 mm columns. UV detection was 280 nm. A variety of chiral stationary phases (Chiralpak AD-H, 5 micron for example) and a variety of solvent mixtures are used and are described in the following synthetic details. Flow rate was 50 mL/min.

### REFERENCE EXAMPLE 1

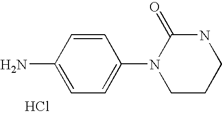

### Preparation of 1-(4-Aminophenyl)-tetrahydro-pyrimidin-2-one hydrochloride

### REFERENCE EXAMPLE 1A

### 1-(3-Chloropropyl)-3-(4-nitrophenyl) urea

A solution of 4-nitrophenylisocyanate (1.70 g, 10.4 mmol) in methylene chloride (100 mL) was cooled to 0° C., and 3-chloropropylamine hydrochloride (1.41 g, 10.8 mmol) was added. N,N-diisopropylethylamine (3.90 mL, 22.4 mmol) was added dropwise, and the reaction mixture was warmed to room temperature with stirring overnight. The reaction mixture was poured into water (50 mL), and the phases were separated. The aqueous portion was extracted with 1:4 methanol/methylene chloride (100 mL). The title compound began to crystallize in the organic phase and was collected by filtration. The organic filtrate was dried (Na₂SO₄), filtered, and concentrated to a yellow solid. This solid was triturated from 1:1:5 hexane/methylene chloride/ether, filtered and combined with the first crop of product. After drying under high vacuum, the title compound was obtained as 2.12 g (79%) of yellow solid.

LC/MS (M+1) m/z=258. ¹H NMR (300 MHz, DMSO) δ 9.26 (s, 1H), 8.13 (d, J=9.2 Hz, 2H), 7.62 (d, J=9.3 Hz, 2H), 6.55 (t, J=5.7 Hz, 1H), 3.68 (t, J=6.5 Hz, 2H), 3.24 (q, J=6.4 Hz, 2H), 1.91 (quintet, J=6.6 Hz, 2H).

### REFERENCE EXAMPLE 1B

### 1-(4-Nitrophenyl)-tetrahydro-pyrimidin-2-one

A solution of 1-(3-chloropropyl)-3-(4-nitrophenyl) urea (2.11 g, 8.19 mmol) in anhydrous tetrahydrofuran (50 mL) was cooled to 0° C. Potassium t-butoxide (2.20 g, 19.6 mmol) was added, and the reaction mixture stirred 15 min at 0° C. before warming to room temperature. After 7.5 h the reaction mixture was partitioned between ethyl acetate (50 mL) and water (50 mL). Upon addition of brine (50 mL), the phases were separated, and the aqueous phase was brought to pH 4 by addition of 1M aqueous HCl. The aqueous phase was extracted with ethyl acetate (2×100 mL), and the combined extracts were dried (Na₂SO₄), filtered and concentrated. Upon trituration from 1:1:5 methanol/hexane/ether, a yellow-orange solid was obtained (1.27 g, 70%).

LC/MS (M+1) m/z=222. ¹H NMR (300 MHz, DMSO) δ 8.15 (dd, J=12.5, 3.2 Hz, 2H), 7.62 (dd, J=7.2, 2.1 Hz, 2H), 6.99 (s, 1H), 3.74 (t, J=5.7 Hz, 2H), 3.24 (td, J=5.9, 2.4 Hz, 2H), 1.98 (quintet, J=5.8 Hz, 2H).

### REFERENCE EXAMPLE 1C

### 1-(4-Aminophenyl)-tetrahydro-pyrimidin-2-one hydrochloride

1-(4-Nitrophenyl)-tetrahydro-pyrimidin-2-one (4.04 g, 18.3 mmol) was added to a suspension of 10% palladium on carbon (0.393 g, 0.370 mmol) in glacial acetic acid (180 mL) and subjected to hydrogenation at 50 psi for 2 h at room temperature. The suspension was filtered through a pad of diatomaceous earth, which was washed with methanol and 1:1 methanol/methylene chloride. The filtrate was concentrated in vacuo, and the resulting solid dried under high vacuum at room temperature overnight giving 4.59 g tan solid. The solid was taken up in anhydrous methanol, treated with HCl (2.0M in ether, 30 mL), and the hydrochloride salt was precipitated by addition of anhydrous ether. The salt was filtered under a nitrogen atmosphere then dried under high vacuum at 60 C for 3 h giving 4.07 g (98%).

LC/MS (M+1) m/z=192. ¹H NMR (300 MHz, DMSO) δ 9.93 (s, 1H), 7.37 (d, J=8.8 Hz, 2H), 7.25 (d, J=8.8 Hz, 2H), 6.62 (s, 1H), 3.61 (t, J=5.7 Hz, 2H), 3.23 (t, J=5.9 Hz, 2H), 1.95 (quintet, J=5.8 Hz, 2H).

### REFERENCE EXAMPLE 2

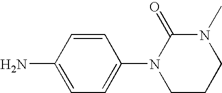

### Preparation of 1-(4-amino-phenyl)-3-methyl-tetrahydro-pyrimidin-2-one

### REFERENCE EXAMPLE 2A

### 1-Methyl-3-(4-nitro-phenyl)-tetrahydro-pyrimidin-2-one

A solution of 1-(4-nitrophenyl)-tetrahydro-pyrimidin-2-one (0.615 g, 2.78 mmol) in anhydrous tetrahydrofuran (28 mL) was cooled to 0 C, and sodium hydride (60% dispersion in mineral oil, 0.278 g, 6.95 mmol) was added. After 45 min iodomethane (0.52 mL, 8.3 mmol) was added via syringe, and the mixture was brought to room temperature for 1 h. The reaction was quenched by addition of water (30 mL), and then extracted with ethyl acetate (2×30 mL). The combined organic extracts were washed with brine (30 mL), dried (Na₂SO₄), filtered, and concentrated in vacuo to a yellow solid. The material was triturated from 9:1 ether/hexane 0.582 g (89%) of the title compound as yellow needles.

LC/MS (M+1) m/z: 236. ¹H NMR (300 MHz, DMSO) δ 8.14 (dd, J=7.1, 2.1 Hz, 2H), 7.55 (dd, J=7.1, 2.1 Hz, 2H), 3.76 (t, J=5.7 Hz, 2H), 3.36 (t, J=6.1 Hz, 2H), 2.90 (s, 3H), 2.05 (quintet, J=5.9 Hz, 2H).

### REFERENCE EXAMPLE 2B

### 1-(4-amino-phenyl)-3-methyl-tetrahydro-pyrimidin-2-one

1-Methyl-3-(4-nitro-phenyl)-tetrahydro-pyrimidin-2-one (0.692 g, 2.94 mmol) was added to a suspension of 10% palladium on carbon (47.6 mg, 0.447 mmol) in absolute ethanol (80 mL) and subjected to hydrogenation at 50 psi for overnight at room temperature. The reaction mixture was filtered through a pad of Diatomaceous earth, washing with ethanol, and the filtrate was concentrated in vacuo. The solid was triturated from ether, and the title compound was isolated as 0.499 g (83%) of a pale yellow solid.

LC/MS (M+1) m/z: 206. ¹H NMR (300 MHz, DMSO) δ 6.83 (d, J=8.7 Hz, 2H), 6.48 (d, J=8.7 Hz, 2H), 4.91 (s, 2H), 3.48 (t, J=5.8 Hz, 2H), 3.29 (t, J=6.0 Hz, 2H), 2.81 (s, 3H), 1.97 (quintet, J=5.9 Hz, 2H).

### REFERENCE EXAMPLE 3

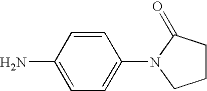

### Preparation of 1-(4-amino-phenyl)-pyrrolidin-2-one. This material was prepared using the following literature procedure: Reppe, et al. Justus Liebigs Ann. Chem. (1955), 596.

### REFERENCE EXAMPLE 4

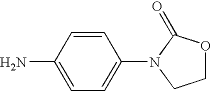

### Preparation of 3-(4-aminophenyl)-oxazolidin-2-one. This material was prepared using the following literature procedure: Artico, et al. Farmaco Sci. Ed. (1969), 179.

### REFERENCE EXAMPLE 5

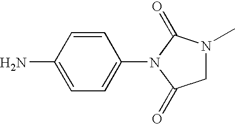

### Preparation of 3-(4-Aminophenyl)-1-methyl-imidazolidine-2,4-dione

1-Methyl-3-(4-nitrophenyl)-imidazolidine-2,4-dione (1.41 g, 6.01 mmol, prepared as described by Pojarlieff, et al. J. Chem. Soc., Perkin Trans. 2 (1996), 2479.) was added to a suspension of 10% palladium on carbon (82.0 mg, 0.0770 mmol) in ethanol (80 mL) and subjected to hydrogenation at 50 psi for 2 h at room temperature. The reaction mixture was filtered through a pad of Diatomaceous earth, washed with ethanol, and the filtrate was concentrated in vacuo. The solid was triturated from ether, and the title compound was isolated as a yellow solid (0.840 g 68%). Additional product (0.218 g, 18%) was isolated from further washing of the Diatomaceous earth pad with 1:9 methanol/methylene chloride.

LC/MS (M+1) m/z: 206. ¹H NMR (300 MHz, DMSO) δ 6.89 (dd, J=6.6, 2.1 Hz, 2H), 6.58 (dd, J=6.7, 2.0 Hz, 2H), 5.24 (s, 2H), 4.04 (s, 2H), 2.89 (s, 3H).

### REFERENCE EXAMPLE 6

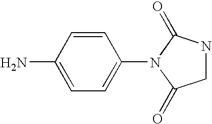

### Preparation of 3-(4-aminophenyl)-imidazolidine-2,4-dione

This material was prepared using the following literature procedure: Ryczek, J. Heterocyclic Chem. 39 (2002), 997.

### REFERENCE EXAMPLE 7

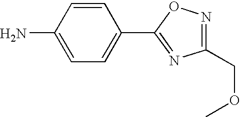

### Preparation of 4-[3-(methoxymethyl)-1,2,4-oxadiazol-5-yl]aniline

### REFERENCE EXAMPLE 7A

### (2E)-1-methoxyacetone O-(4-nitrobenzoyl)oxime

To a solution of (1Z)-N′-hydroxy-2-methoxyethanimidamide (1.58 g, 15.2 mmol) in DCM (100 mL) was added DIPEA (5.29 mL, 30.4 mmol) followed by 4-nitrobenzoylchloride (3.66 g, 19.7 mmol) at 0° C. The reaction mixture was stirred at 0° C. for 1 h and room temperature over night. DCM was evaporated and EtOAc was added. The organic layer was washed with water (2×) and saturated NaCl (1×), dried over MgSO4, filtered and concentrated to give yellow solid (1.475 g, 38% yield). LC/MS (M+1) m/z=254.

### REFERENCE EXAMPLE 7B

### 3-(methoxymethyl)-5-(4-nitrophenyl)-1,2,4-oxadiazole

To the solution of (2E)-1-methoxyacetone O-(4-nitrobenzoyl)oxime (1.475 g, 5.8 mmol) in CH₃CN (25 mL) was added tetrabutylammonium fluoride (3.0 mL, 2.9 mmol). The mixture was stirred at room temperature over night. EtOAc was added. The organic layer was washed with water (2×) and saturated NaCl (1×), dried over MgSO₄, filtered and concentrated to give yellow solid (1.237 g, 90% yield).

### REFERENCE EXAMPLE 7C

### 4-[3-(methoxymethyl)-1,2,4-oxadiazol-5-yl]aniline

To the solution of 3-(methoxymethyl)-5-(4-nitrophenyl)-1,2,4-oxadiazole (1.132 g, 4.8 mmol) in dioxane (11 mL) was added a hot solution (75° C.) of Na₂S.9H₂O (2.66 g, 11 mmol) in water (11 mL) at 80° C. The reaction mixture was stirred at 80° C. for 45 min and cooled to room temperature. Solvent was evaporated and the residue was purified by silica gel chromatography eluting with DCM to give light yellow solid (423 mg, 43% yield). LC/MS (M+1) m/z: 206.

### REFERENCE EXAMPLE 8

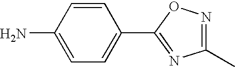

### Preparation of 4-(3-methyl-1,2,4-oxadiazol-5-yl]aniline

### REFERENCE EXAMPLE 8A

### (1E)-N′-[(4-nitrobenzoyl)oxy]ethanimidamide

To the solution of (1E)-N′-hydroxyethanimidamide (1.00 g, 13.5 mmol) in DCM (100 mL) was added DIPEA (4.70 mL, 27.0 mmol) followed by 4-nitrobenzoylchloride (3.26 g, 17.6 mmol) at 0° C. The reaction mixture was stirred at 0° C. for 1 h and room temperature over night. DCM was evaporated and EtOAc was added. The organic layer was washed with water (2×) and saturated NaCl (1×), dried over MgSO4, filtered and concentrated to give yellow solid (1.50 g, 50% yield). LC/MS (M+1) m/z: 224.

### REFERENCE EXAMPLE 8B

### 3-methyl-5-(4-nitrophenyl)-1,2,4-oxadiazole

To the solution of (1E)-N′-[(4-nitrobenzoyl)oxy]ethanimidamide (1.5 g, 6.72 mmol) in CH₃CN (20 mL) was added tetrabutylammonium fluoride (1M in THF, 6.72 mL, 6.72 mmol). The mixture was stirred at room temperature for 20 min. EtOAc was added. The organic layer was washed with water (2×) and saturated NaCl (1×), dried over MgSO4, filtered and concentrated. The residue was purified by silica gel chromatography eluting with DCM to give white solid (1.296 g, 94% yield).

### REFERENCE EXAMPLE 8C

### 4-(3-methyl-1,2,4-oxadiazol-5-yl)aniline

To the solution of 3-methyl-5-(4-nitrophenyl)-1,2,4-oxadiazole (1.088 g, 5.3 mmol) in acetic acid (90 mL) was added Fe (2.66 g, 47.7 mmol) at 0° C. The reaction mixture was stirred at room temperature for 2 h. Mixture was decanted away from Fe by holding a large stir bar in hand. Solvent was evaporated and the residue was dissolved in 9:1 DCM/EtOAc (500 mL). Silica amine (13 g) was then added. The mixture was stirred at room temperature for 1.5 h, filtered through diatomaceous earth, washed with DCM, concentrated to give white solid (367 mg, 40% yield). LC/MS (M+1) m/z: 176.

### REFERENCE EXAMPLE 9

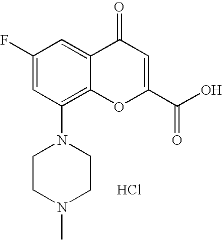

### Preparation of 6-Fluoro-8-(4-methyl-piperazin-1-yl)-4-oxo-4-H-chromene-2-carboxylic acid hydrochloride. This material was prepared using the procedure described in AstraZeneca patent application no. WO2003037872.

### REFERENCE EXAMPLE 10

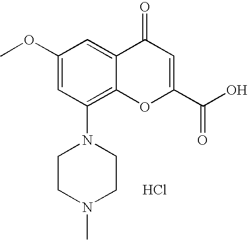

### Preparation of 6-Methoxy-8-(4-methyl-piperazin-1-yl)-4-oxo-4-H-chromene-2-carboxylic acid hydrochloride. This material was prepared using the procedure described in AstraZeneca patent application no. WO2003037872.

### REFERENCE EXAMPLE 11

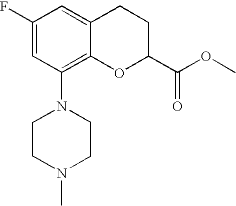

Resolution of (rac)-6-fluoro-8-(4-methyl-piperazin-1-yl)-chroman-2-carboxylic acid methyl ester to yield (+)-6-fluoro-8-(4-methyl-piperazin-1-yl)-chroman-2-carboxylic acid methyl ester.

A solution of (rac)-6-fluoro-8-(4-methyl-piperazin-1-yl)-chroman-2-carboxylic acid methyl ester (Reference Example 17, 82.54 g) in methanol (300 mL) was treated with a solution of D-tartaric acid (42.24 g) in methanol (300 mL) and stirred rapidly for 16 h. The resulting solid was collected by filtration, redissolved in boiling methanol (600 mL), stirred for 16 h and the solid was filtered. This process was repeated for a total of 4 times to obtain the resolved tartrate salt of the title compound (30.29 g). A sample of this material was converted to the free base and analyzed by chiral HPLC (Chiralpak AD, 10% EtOH/Hexane, rt=7.75 min.) to determine the chiral purity of the material to be 95% ee. (The % ee enrichment varied slightly from batch to batch. Also, the ee could be further enriched by additional crystallizations.)

A suspension of the resolved tartrate salt (30.29 g) in ethyl acetate (400 mL) was treated with 10% potassium carbonate solution and the organic layer was dried over anhydrous potassium carbonate, concentrated under reduced pressure to afford the title compound (20 g, 48% yield).

### REFERENCE EXAMPLE 12

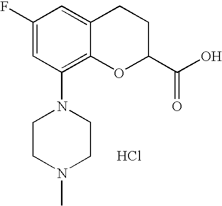

### Preparation of (+)-6-fluoro-8-(4-methyl-piperazin-1-yl)-chroman-2-carboxylic acid hydrochloride

(+)-6-Fluoro-8-(4-methyl-piperazin-1-yl)-chroman-2-carboxylic acid methyl ester (Reference Example 11, 20.0 g) was dissolved in tetrahydrofuran (500 mL), treated with a solution of lithium hydroxide (3.12 g) in water (100 mL). Upon stirring for 1 h at room temperature the reaction mixture was acidified with 4M HCl in dioxane (33 mL) and concentrated under reduced pressure. The resulting oil was treated consecutively twice with tetrahydrofuran and once with acetonitrile (300 mL) and concentrated under reduced pressure. The material thus obtained was suspended in rapidly stirred acetonitrile (400 mL), treated with 4M HCl in dioxane (65 mL) and stirred for 1 h. The solid was filtered, resuspended in ethyl ether (300 mL) and filtered, dried under reduced pressure to obtain the desired product (25.7 g)

LC/MS: 295 (M+1). ¹H NMR (300.132 MHz, DMSO) δ 11.25(s, 2H) 6.64-6.55 (m, 2H), 4.83 (dd, J=6.3, 4.0 Hz, 1H), 3.92 (d, J=12.8 Hz, 1H), 3.45-3.35 (m, 3H), 3.22-3.11 (m, 3H), 2.92 (t, J=11.3 Hz, 1H), 2.83-2.73 (m, 4H), 2.68-2.57 (m, 2H), 2.19-1.99 (m, 2H).

### REFERENCE EXAMPLE 14

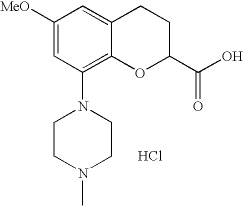

### Preparation of (Rac)-6-Methoxy-8-(4-methyl-piperazin-1-yl)-chroman-2-carboxylic acid hydrochloride

A solution of 6-Methoxy-8-(4-methyl-piperazin-1-yl)-4-oxo-4-H-chromene-2-carboxylic acid hydrochloride (Reference Example 10, 100 g) in acetic acid (1200 mL) was hydrogenated using 10% Pd/C (25 g) at 50 PSI hydrogen pressure at 60 C for 5 days. At the end of this period the reaction mixture was filtered through a pad of diatomaceous earth (wash with acetic acid [6×150 mL]) and concentrated under reduced pressure yielding a thick amber syrup. The diatomaceous earth pad was then washed with THF (5×150 mL). These washes were combined with the amber syrup and solvent removed under vacuo. Anhydrous toluene (4×500 mL) was added and the solution was again concentrated in vacuo. The syrup thus obtained was stirred with ethyl ether (1000 mL) for 1 h and then stored undisturbed overnight. The solid was collected by vacuum filtration, washed with ether (2×200 mL) and dried under vacuum at 50 C to yield a light beige powder (185.4 g). This beige powder was suspended in rapidly stirred acetonitrile (2500 mL). To this rapidly stirring suspension was added 4N HCl in dioxane (560 mL) dropwise over 15 minutes, and the resulting suspension stirred overnight. The off-white solid was collected by filtration, washed with acetonitrile (3×200 mL), dried under vacuum at 55 C overnight yielding the title compound (167.8 g, 87% yield as the mono-HCl salt). LC/MS: 307 (M+1).

### REFERENCE EXAMPLE 15

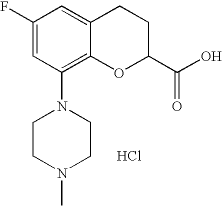

### Preparation of (Rac)-6-Fluoro-8-(4-methyl-piperazin-1-yl)-chroman-2-carboxylic acid hydrochloride

This material was prepared as described in Reference Example 14 but using 6-Fluoro-8-(4-methyl-piperazin-1-yl)-4-oxo-4-H-chromene-2-carboxylic acid hydrochloride (Reference Example 9, 150 g) instead of 6-Methoxy-8-(4-methyl-piperazin-1-yl)-4-oxo-4-H-chromene-2-carboxylic acid hydrochloride and the reaction temperature was raised to 70 C yielding the title compound (141 g, 87% yield as the mono-HCl salt). LC/MS: 295 (M+1).

### REFERENCE EXAMPLE 16

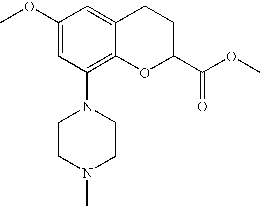

### Preparation of (Rac)-6-Methoxy-8-(4-methyl-piperazin-1-yl)-chroman-2-carboxylic methyl ester

Concentrated sulfuric acid (1.0 mL) was added to a solution containing (Rac)-6-Methoxy-8-(4-methyl-piperazin-1-yl)-chroman-2-carboxylic acid hydrochloride (Reference Example 14, 8.49 g) in anhydrous methanol (80 mL) and heated to reflux for 3 h. Reaction was cooled to room temp and solvent was removed under vacuo. Enough saturated aqueous sodium bicarbonate was added to give pH >8. Transfer to separatory funnel and extract with ethyl acetate (2×200 mL). The ethyl acetate extracts were washed with saturated sodium bicarbonate (75 mL) then brine (75 ml), dried over magnesium sulfate and filtered. Solvent was removed under vacuum yielding an orange solid. Solid was washed with hexane (75 mL) and dried yielding the title compound as an off-white solid (7.02 g, 99% yield). LC/MS: 321 (M+1).

### REFERENCE EXAMPLE 17

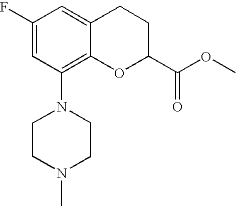

### Preparation of (Rac)-6-Fluoro-8-(4-methyl-piperazin-1-yl)-chroman-2-carboxylic methyl ester

This material was prepared as described in Reference Example 16 but using (Rac)-6-fluoro-8-(4-methyl-piperazin-1-yl)-chroman-2-carboxylic acid hydrochloride (Reference Example 15) instead of (Rac)-6-Methoxy-8-(4-methyl-piperazin-1-yl)-chroman-2-carboxylic acid hydrochloride yielding the title compound as an off-white solid. LC/MS: 309 (M+1).

### REFERENCE EXAMPLE 22

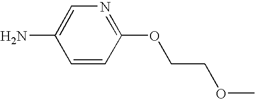

### Preparation of 2-(Methoxyethoxy)-4-aminopyridine

2-Methoxyethanol (5 ml) was treated with NaH (0.57 g, 14.3 mmol) at rt in several portions over 10 minutes. The reaction is exothermic! After stirring at rt for 20 min, 2-chloro-4-nitropyridine (1.5 g, 9.5 mmol) was added in two portions. After stirring at rt for 20 min, the reaction was poured into water and the product was filtered off and air-dried. This solid was suspended in MeOH, treated with 10% Pd/C (0.2 g) and the reaction hydrogenated on a Parr Shaker at 50 psi for 2 h. The reaction was then filtered and the solvent removed at reduced pressure to give the product as an off white solid (1.8 g, 95%). LC/MS: 169 (M+H, 100%). ¹H-NMR (300 MHz, DMSO-d₆) [symbol] 8.12 (s, 1H), 7.14 (d, J=8.6 Hz, 1H), 6.66 (d, J=8.6 Hz, 1H), 5.50 (bs, 1H), 4.47 (d, J=8.4 Hz, 1H), 3.74 (d, J=8.4 Hz, 1H), 3.44 (s, 3H).

### REFERENCE EXAMPLE 23

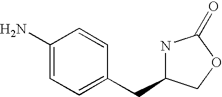

### Preparation of (R)-4-(4-amino-benzyl)-oxazolidin-2-one

This aniline was synthesized using the procedure described in J. Med. Chem. 1997, 40, 2347-2362.

### REFERENCE EXAMPLE 24

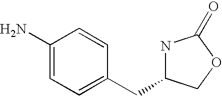

### Preparation of (S)-4-(4-amino-benzyl)-oxazolidin-2-one

This aniline was synthesized using the procedure described in J. Med. Chem. 1997, 40, 2347-2362.

### REFERENCE EXAMPLE 25

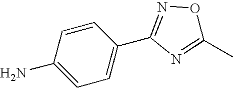

### Preparation of 4-(5-Methyl-[1,2,4]oxadiazol-3-yl)-phenylamine

### REFERENCE EXAMPLE 25A

### N-Hydroxy-4-nitro-benzamidine

To a stirred solution of 4-nitrobenzonitrile (3.17 g, 21.4 mmol) in absolute ethanol (100 mL) was added hydroxylamine (5 mL of a 50% w/w solution in water, 81 mmol). Mixture heated to reflux for 2.5 hour and then cooled to RT overnight. Ethanol was evaporated under reduced pressure; the residue was mixed with 100 mL water, and stirred for 30 minutes. The bright yellow solid was filtered, washed with water (2×25 mL), and then dried overnight under high vacuum (3.67 g, 88% yield, ˜93% pure).

¹H NMR (300.132 MHz, DMSO-d₆) δ 10.12 (s, 1H), 8.24 (d, J=8.4 Hz, 2H), 7.95 (d, J=8.4 Hz, 2H), 6.04 (s, 2H).

### REFERENCE EXAMPLE 25B

### (E)-N′-acetoxy-4-nitrobenzimidamide

To a stirred solution of N-Hydroxy-4-nitro-benzamidine (600 mg, 3.31 mmol) in DCM (10 mL) cooled to 0° C. was added triethylamine (1.2 mL, 8.61 mmol) followed by acetyl chloride (0.30 mL, 4.3 mmol). Mixture was stirred for 1.25 h at 0° C. then warmed to room temperature for 3 h. Reaction was diluted with EtOAc (50 mL) and extracted with water (50 mL). Organic extract was dried over MgSO₄, filtered, and evaporated under reduced pressure to give a solid which was recrystallized from 95% EtOH (170 mg, 23% yield). MS: m/z 224 (M+H).

### REFERENCE EXAMPLE 25C

### 5-Methyl-3-(4-nitro-phenyl)-[1,2,4]oxadiazole

To a solution of (E)-N′-acetoxy-4-nitrobenzimidamide (446 mg, 2.00 mmol) in DCM (10 mL) was added tetrabutylammonium fluoride (6.0 mL, 6.0 mmol). After 2 h the mixture was diluted with 1:1 EtOAc/Et₂O (125 mL) and extracted with water (4×30 mL), 20% aqueous K₂CO₃ (30 mL), and brine (30 mL). Organic extract was dried over Na₂SO₄, filtered, and evaporated under reduced pressure to give a solid which was purified by flash chromatography on silica; DCM eluent (393 mg, 96%). ¹H NMR (300.132 MHz, CDCl₃) δ 8.38-8.31 (m, 2H), 8.29-8.23 (m, 2H), 2.70 (s, 3H).

### REFERENCE EXAMPLE 25D

### 4-(5-Methyl-[1,2,4]oxadiazol-3-yl)-phenylamine

To a solution of 5-methyl-3-(4-nitro-phenyl)-[1,2,4]oxadiazole (389 mg, 1.90 mmol) in 7 mL HOAc was added iron (1.03 g, 18.4 mmol). After 1 h a heavy precipitate formed and more HOAc (7 mL) was added; reaction was complete after an additional hour. The mixture was decanted away from the elemental iron, which was washed with HOAc (3×5 mL). HOAc was evaporated under reduced pressure to give a solid which was dissolved in 100 mL 9:1 DCM:EtOAc. To this was added 5 g Si-Amine (silicycle part #R52030B, 1.46 mmol/g). Mixture was stirred for 1.5 h, filtered through diatomaceous earth; filtrate was evaporated under reduced pressure to give a solid (284 mg, 86%, 92% pure). MS: m/z 217 (M+H+ MeCN).

### REFERENCE EXAMPLE 26

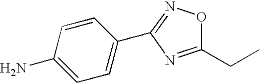

### Preparation of 4-(5-Ethyl-[1,2,4]oxadiazol-3-yl)-phenylamine

### REFERENCE EXAMPLE 26A

### (E)-4-nitro-N′-(propionyloxy)benzimidamide

Prepared in an analogous fashion to that described in Reference Example 25b using propionyl chloride in place of acetyl chloride. Product was recrystallized from EtOH/Hexane (483 mg, 62% yield). MS: m/z 238 (M+H).

### REFERENCE EXAMPLE 26B

### 5-Ethyl-3-(4-nitro-phenyl)-[1,2,4]oxadiazole

Prepared in an analogous fashion to that described in Reference Example 25c to give a solid, which was purified by chromatography on silica, DCM eluent (440 mg, 96%). ¹H NMR (300.132 MHz, CDCl3) δ 8.38-8.31 (m, 2H), 8.29-8.23 (m, 2H), 3.00 (q, J=7.5 Hz, 2H), 1.48 (t, J=7.5 Hz, 3H).

### REFERENCE EXAMPLE 26C

### 4-(5-Ethyl-[1,2,4]oxadiazol-3-yl)-phenylamine

Prepared in an analogous fashion to that described in Reference Example 25d to give a solid (340 mg, 89%, 86% pure). MS: m/z 231 (M+H+ MeCN).

### REFERENCE EXAMPLE 27

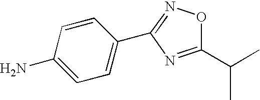

### Preparation of 4-(5-Isopropyl-[1,2,4]oxadiazol-3-yl)-phenylamine

### REFERENCE EXAMPLE 27A

### (E)-4-nitro-N′-(isobutyryloxy)benzimidamide

Prepared in an analogous fashion to that described in Reference Example 25b using isobutyryl chloride in place of acetyl chloride. Product was recrystallized from EtOH/Hexane (262 mg, 32% yield). MS: m/z 252 (M+H).

### REFERENCE EXAMPLE 27B

### 5-Isopropyl-3-(4-nitro-phenyl)-[1,2,4]oxadiazole

Prepared in an analogous fashion to that described in Reference Example 25c to give a solid, which was purified by chromatography on silica, DCM eluent (394 mg, 96%). ¹H NMR (300.132 MHz, CDCl₃) δ 8.36-8.31 (m, 2H), 8.30-8.25 (m, 2H), 3.32 (septet, J=6.9 Hz, 1H), 1.48 (d, J=6.9 Hz, 6H).

### REFERENCE EXAMPLE 27C

### 4-(5-Isopropyl-[1,2,4]oxadiazol-3-yl)-phenylamine

Prepared in an analogous fashion to that described in Reference Example 25d to give a solid (266 mg, 78%, 90% pure). MS: m/z 245 (M+H+ MeCN).

### REFERENCE EXAMPLE 28

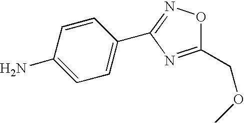

### Preparation of 4-(5-Methoxymethyl-[1,2,4]oxadiazol-3-yl)-phenylamine

### REFERENCE EXAMPLE 28A

### (E)-4-nitro-N′-(methoxyacetoxy)-4-nitrobenzimidamide

Prepared in an analogous fashion to that described in Reference Example 25b using methoxyacetyl chloride in place of acetyl chloride. product was recrystallized from DCM/Hexane (209 mg, 25% yield). MS: m/z 254 (M+H).

### REFERENCE EXAMPLE 28B

### 5-Methoxymethyl-3-(4-nitro-phenyl)-[1,2,4]oxadiazole

Prepared in an analogous fashion to that described in Reference Example 25c to give a solid which was purified by chromatography on silica; DCM eluent (95 mg, 61%); ¹H NMR (300.132 MHz, CDCl₃) δ 8.38-8.28 (m, 4H), 4.78 (s, 2H), 3.58 (s, 3H).

### REFERENCE EXAMPLE 28C

### 4-(5-Methoxymethyl-[1,2,4]oxadiazol-3-yl)-phenylamine

Prepared in an analogous fashion to that described in Reference Example 25d to give a solid (192 mg, 86%, 85% pure). MS: m/z 247 (M+H+ MeCN).

### REFERENCE EXAMPLE 30

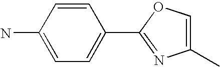

### Preparation of 4-(4-Methyl-oxazol-2-yl)-phenylamine

This aniline was prepared as described in JACS, 1937, 59, 2262-2264.

### REFERENCE EXAMPLE 35

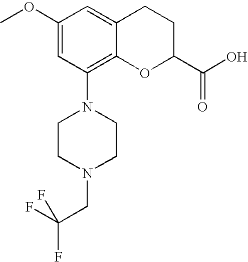

### Preparation of 6-Methoxy-8-[4-(2,2,2-trifluoro-ethyl)-piperazin-1-yl]-chroman-2-carboxylic acid

### REFERENCE EXAMPLE 35A

### 6-Methoxy-4-oxo-8-[4-(2,2,2-trifluoro-ethyl)-piperazin-1-yl]-4H-chromene-2-carboxylic acid ethyl ester

To a reaction flask was added 1-(2,2,2-trifluoro-ethyl)-piperazine (Reference Example 36, 284 mg, 1.69 mmol), 8-bromo-6-methoxy-4-oxo-4H-chromene-2-carboxylic acid ethyl ester (Reference Example 37, 570 mg, 1.74 mmol), toluene (10 mL), racemic-2,2′-bis(diphenylphosphino)-1,1′-binaphthyl (100 mg, 0.16 mmol), cesium carbonate (880 mg, 2.70 mmol), and palladium dba (70 mg, 0.076 mmol). Flask was evacuated and backfilled with N₂ (3 cycles). Reaction was heated to 100° C. for 18 h, cooled to rt, mixed with 50 mL 20% aqueous K₂CO₃, and extracted with DCM (4×30 mL). Extracts were combined, dried over Na₂SO₄, filtered, and evaporated under reduced pressure to give an oil. Product was purified by chromatography on silica, 200:1 to 100:1 to 50:1 to 25:1 DCM/Hexane: MeOH (w/0.25% aq conc. NH₃). This gave 319 mg of product (46%), MS: m/z 415 (M+H).

### REFERENCE EXAMPLE 35B

### 6-Methoxy-4-oxo-8-[4-(2,2,2-trifluoro-ethyl)-piperazin-1-yl]-4H-chromene-2-carboxylic acid

To a solution of 6-methoxy-4-oxo-8-[4-(2,2,2-trifluoro-ethyl)-piperazin-1-yl]-4H-chromene-2-carboxylic acid ethyl ester (Reference Example 35a, 319 mg, 0.77 mmoles) in 6 mL of 5:1 THF: Methanol was added LiOH (62 mg, 1.48 mmol) dissolved in 1 mL Water. After 15 minutes the pH was then adjusted to ˜1 and solvents were evaporated under reduced pressure to give a yellow solid that was pumped down overnight. This gave 320 mg of product (89% as the HCl/LiCl salt). MS: m/z 387 (M+H).

### REFERENCE EXAMPLE 35C

### 6-Methoxy-8-[4-(2,2,2-trifluoro-ethyl)-piperazin-1-yl]-chroman-2-carboxylic acid

6-Methoxy-4-oxo-8-[4-(2,2,2-trifluoro-ethyl)-piperazin-1-yl]-4H-chromene-2-carboxylic acid salt with HCl/LiCl (Reference Example 35b, 320 mg, 0.69 mmol) was dissolved in 250 mL of HOAc and charged with 240 mg 10% Pd/C. Flask was attached to the Parr shaker, evacuated/backfilled with hydrogen (3 cycles). Mixture was heated to 75° C. and agitated for 8 h under 60-psi hydrogen. Reaction was cooled to RT, filtered through diatomaceous earth, and evaporated under reduced pressure. The resulting brown oil was pumped down under high vacuum overnight. This gave 196 mg (63%), MS: m/z 375 (M+H).

### REFERENCE EXAMPLE 36

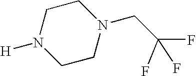

### Preparation of 1-(2,2,2-Trifluoro-ethyl)-piperazine. This material was prepared as described in JOC, 1966, 31, 3867-3868

### REFERENCE EXAMPLE 37

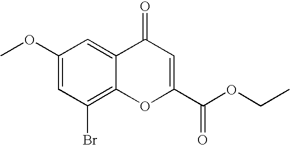

### Preparation of 8-bromo-6-methoxy-4-oxo-4H-chromene-2-carboxylic acid ethyl ester

This material was prepared using the procedure described in AstraZeneca patent application WO2003037872A 1-(Reference Example 2).

### REFERENCE EXAMPLE 38

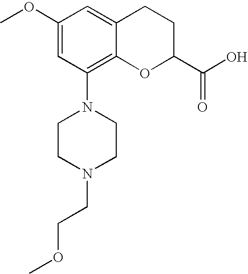

### Preparation of 6-Methoxy-8-[4-(2-methoxy-ethyl)-piperazin-1-yl]-chroman-2-carboxylic acid

### REFERENCE EXAMPLE 38A

### 6-Methoxy-8-[4-(2-methoxy-ethyl)-piperazin-1-yl]-4-oxo-4H-chromene-2-carboxylic acid ethyl ester

8-Bromo-6-methoxy-4-oxo-4H-chromene-2-carboxylic acid ethyl ester (Reference Example 37, 680 mg, 2.08 mmol) was coupled with 1-(2-methoxy-ethyl)-piperazine (320 mg, 2.22 mmol) in an analogous fashion to that described in Reference Example 35a to give 292 mg (36%) of product; MS: m/z 391 (M+H).

### REFERENCE EXAMPLE 38B

### 6-Methoxy-8-[4-(2-methoxy-ethyl)-piperazin-1-yl]-4-oxo-4H-chromene-2-carboxylic acid

6-Methoxy-8-[4-(2-methoxy-ethyl)-piperazin-1-yl]-4-oxo-4H-chromene-2-carboxylic acid ethyl ester (Reference Example 38a) was hydrolyzed using a procedure similar (reaction time was 30 minutes) to that described in Reference Example 35b to give 292 mg of product (essentially quantitative as the HCl/LiCl salt); MS: m/z 363 (M+H).

### REFERENCE EXAMPLE 38C

### 6-Methoxy-8-[4-(2-methoxy-ethyl)-piperazin-1-yl]-chroman-2-carboxylic acid

6-Methoxy-8-[4-(2-methoxy-ethyl)-piperazin-1-yl]-4-oxo-4H-chromene-2-carboxylic acid (Reference Example 38b) was reduced as described in Reference Example 35c except the reaction time was 50 h. This gave 310 mg of product (essentially quantitative). MS: m/z 351 (M+H).

### REFERENCE EXAMPLE 40

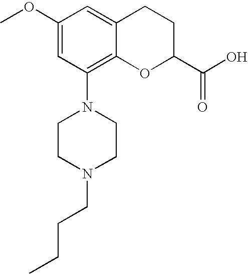

### Preparation of 8-(4-Butyl-piperazin-1-yl)-6-methoxy-chroman-2-carboxylic acid

### REFERENCE EXAMPLE 40A

### 8-(4-Butyl-piperazin-1-yl)-6-methoxy-4-oxo-4H-chromene-2-carboxylic acid ethyl ester

8-Bromo-6-methoxy-4-oxo-4H-chromene-2-carboxylic acid ethyl ester (Reference Example 37, 654 mg, 2.00 mmol) was coupled with 1-butyl-piperazine (360 mg, 2.53 mmol) in an analogous fashion to that described in Reference Example 35a. Compound needed additional purification by Prep RPHPLC. [Conditions: 25-90% MeCN (w/0.1% TFA) over 20 minutes, 7 min equilibration at 25% MeCN, 5 min flush at 90% MeCN. Used a 2″ diameter radial compression C8 dynamax 60 Å column]. Residue after evaporation was mixed with 15 mL 20% aqueous K₂CO₃ and extracted with DCM (3×30 mL). Extracts were combined, dried over Na₂SO₄, filtered, and evaporated under reduced pressure to give 307 mg (41%) of product; ¹H NMR (300.132 MHz, CDCl₃) δ 7.16 (d, J=2.9 Hz, 1H), 7.09 (s, 1H), 6.83 (d, J=2.9 Hz, 1H), 4.45 (q, J=7.1 Hz, 2H), 3.88 (s, 3H), 3.35-3.20 (m, 4H), 2.79-2.66 (m, 4H), 2.44 (t, J=7.6 Hz, 2H), 1.44 (t, J=7.1 Hz, 3H), 1.60-1.30 (m, 4H), 0.95 (t, J=7.2 Hz, 3H).

### REFERENCE EXAMPLE 40B

### 8-(4-Butyl-piperazin-1-yl)-6-methoxy-chroman-2-carboxylic acid

8-(4-Butyl-piperazin-1-yl)-6-methoxy-4-oxo-4H-chromene-2-carboxylic acid ethyl ester (Reference Example 40a, 307 mg, 0.79 mmol) was dissolved in 200 mL of HOAc and charged with 300 mg 10% Pd/C. Flask was attached to the Parr shaker and evacuated/backfilled with hydrogen (3 cycles). Mixture was heated to 75° C. and agitated for 5 days under 60-psi hydrogen. Reaction was cooled to room temperature, filtered through diatomaceous earth, and evaporated under reduced pressure. The resulting brown oil was triturated in diethyl ether, filtered, washed with more diethyl ether, and pumped down under high vacuum overnight. This gave 151 mg (55%), MS: m/z 349 (M+H).

### REFERENCE EXAMPLE 41

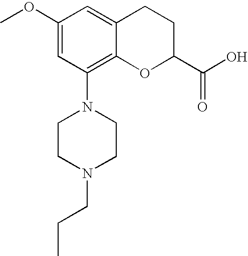

### Preparation of 8-(4-Propyl-piperazin-1-yl)-6-methoxy-chroman-2-carboxylic acid

### REFERENCE EXAMPLE 41A

### 6-Methoxy-4-oxo-8-(4-propyl-piperazin-1-yl)-4H-chromene-2-carboxylic acid ethyl ester

8-Bromo-6-methoxy-4-oxo-4H-chromene-2-carboxylic acid ethyl ester (Reference Example 37, 701 mg, 2.14 mmol) was coupled with 1-propyl-piperazine (300 mg, 2.34 mmol) in an analogous fashion to that described in Reference Example 40a to give 312 mg (40%) of product; ¹H NMR (300.132 MHz, CDCl₃) δ 7.16 (d, J=2.9 Hz, 1H), 7.09 (s, 1H), 6.83 (d, J=3.4 Hz, 1H), 4.45 (q, J=7.1 Hz, 2H), 3.88 (s, 3H), 3.35-3.20 (m, 4H), 2.80-2.65 (m, 4H), 2.42 (t, J=7.5 Hz, 2H), 1.66-1.49 (m, 2H), 1.44 (t, J=7.1 Hz, 3H), 0.95 (t, J=7.3 Hz, 3H).

### REFERENCE EXAMPLE 41B

### 6-methoxy-8-(4-propyl-piperazin-1-yl)-chroman-2-carboxylic acid

6-Methoxy-4-oxo-8-(4-propyl-piperazin-1-yl)-4H-chromene-2-carboxylic acid ethyl ester (Reference Example 41a) was reduced as described in Reference Example 40b. This gave 104 mg of product (36%), MS: m/z 335 (M+H).

### REFERENCE EXAMPLE 42

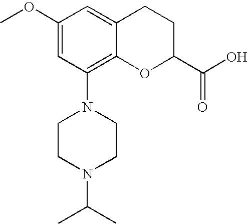

### Preparation of 8-(4-Isopropyl-piperazin-1-yl)-6-methoxy-chroman-2-carboxylic acid

### REFERENCE EXAMPLE 42A

### 6-Methoxy-4-oxo-8-(4-isopropyl-piperazin-1-yl)-4H-chromene-2-carboxylic acid ethyl ester

8-Bromo-6-methoxy-4-oxo-4H-chromene-2-carboxylic acid ethyl ester (Reference Example 37, 696 mg, 2.13 mmol) was coupled with 1-isopropyl-piperazine (273 mg, 2.13 mmol) in an analogous fashion to that described in Reference Example 40a to give 380 mg of product (48%); ¹H NMR (300.132 MHz, CDCl₃) δ 7.16 (d, J=2.9 Hz, 1H), 7.10 (s, 1H), 6.83 (d, J=2.9 Hz, 1H), 4.45 (q, J=7.1 Hz, 2H), 3.88 (s, 3H), 3.35-3.20 (m, 4H), 2.88-2.70 (m, 5H), 1.45 (t, J=7.1 Hz, 3H), 1.12 (d, J=6.5 Hz, 6H).

### REFERENCE EXAMPLE 42B

### 6-Methoxy-8-(4-isopropyl-piperazin-1-yl)-chroman-2-carboxylic acid

6-Methoxy-4-oxo-8-(4-isopropyl-piperazin-1-yl)-4H-chromene-2-carboxylic acid ethyl ester (Reference Example 42A) was reduced as described in Reference Example 40B. This gave 242 mg of product (72%); MS: m/z 334 (M+H).

### REFERENCE EXAMPLE 43

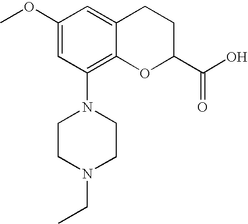

### Preparation of 8-(4-Ethyl-piperazin-1-yl)-6-methoxy-chroman-2-carboxylic acid

### REFERENCE EXAMPLE 43A

### 6-Methoxy-4-oxo-8-(4-ethyl-piperazin-1-yl)-4H-chromene-2-carboxylic acid ethyl ester

8-Bromo-6-methoxy-4-oxo-4H-chromene-2-carboxylic acid ethyl ester (Reference Example 37, 690 mg, 2.11 mmol) was coupled with 1-ethyl-piperazine (243 mg, 2.13 mmol) in an analogous fashion to that described in Reference Example 40a to give 270 mg of product (35%); ¹H NMR (300.132 MHz, CDCl₃) δ 7.16 (d, J=2.9 Hz, 1H), 7.10 (s, 1H), 6.83 (d, J=2.9 Hz, 1H), 4.45 (q, J=7.1 Hz, 2H), 3.88 (s, 3H), 3.36-3.22 (m, 4H), 2.82-2.66 (m, 4H), 2.53 (q, J=7.1 Hz, 2H), 1.44 (t, J=7.1 Hz, 3H), 1.15 (t, J=7.2 Hz, 3H).

### REFERENCE EXAMPLE 43B

### 6-methoxy-8-(4-ethyl-piperazin-1-yl)-chroman-2-carboxylic acid

6-Methoxy-4-oxo-8-(4-ethyl-piperazin-1-yl)-4H-chromene-2-carboxylic acid ethyl ester Reference Example 43a) was reduced as described in Reference Example 40b. This gave 231 mg of product (72%); MS: m/z 321 (M+H).

### REFERENCE EXAMPLE 44

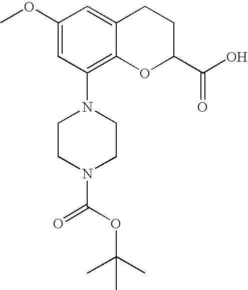

### Preparation of 4-(2-Carboxy-6-methoxy-chroman-8-yl)-piperazine-1-carboxylic acid tert-butyl ester

### REFERENCE EXAMPLE 44A

### 8-(4-Benzyl-piperazin-1-yl)-6-methoxy-4-oxo-4H-chromene-2-carboxylic acid ethyl ester

8-Bromo-6-methoxy-4-oxo-4H-chromene-2-carboxylic acid ethyl ester (Reference Example 37, 1.34 g, 4.10 mmol) was coupled with 1-benzyl-piperazine (1.34 g, 4.26 mmol) in an analogous fashion to that described in Reference Example 40a to give 1.21 g of product (70%). ¹H NMR (300.132 MHz, CDCl₃) δ 7.42-7.22 (m, 5H), 7.16 (d, J=2.9 Hz, 1H), 7.09 (s, 1H), 6.82 (d, J=2.9 Hz, 1H), 4.44 (q, J=7.1 Hz, 2H), 3.87 (s, 3H), 3.62 (s, 2H), 3.35-3.18 (m, 4H), 2.83-2.65 (m, 4H), 1.43 (t, J=7.1 Hz, 3H).

### REFERENCE EXAMPLE 44B

### 6-Methoxy-8-piperazin-1-yl-chroman-2-carboxylic acid

6-Methoxy-4-oxo-8-(4-benzyl-piperazin-1-yl)-4H-chromene-2-carboxylic acid ethyl ester (Reference Example 44A) was reduced as described in Reference Example 40b. Exception: reaction product was purified by RPHPLC as described in Reference Example 40a. This gave 225 mg of product (34%), MS: m/z 293 (M+H).

### REFERENCE EXAMPLE 44C

### 4-(2-Carboxy-6-methoxy-chroman-8-yl)-piperazine-1-carboxylic acid tert-butyl ester

To a stirred solution of 6-methoxy-8-piperazin-1-yl-chroman-2-carboxylic acid (Reference Example 44B, 297 mg, 1.23 mmol) and Et₃N (320 mL, 2.30 mmol) in 10 mL of DCM was added di-tert-butyl dicarbonate (272 mg, 1.24 mmol). Mixture was stirred for 18 h, diluted with 1 N HCl (10 mL), and extracted with DCM (3×20 mL). Organic layers were dried over Na₂SO₄, filtered, and solvent was evaporated under reduced pressure. Product was purified by chromatography on silica (DCM:MeOH 20:1 [symbol] 10:1) to give 190 mg of product. MS: m/z 393 (M+H).

### REFERENCE EXAMPLE 45

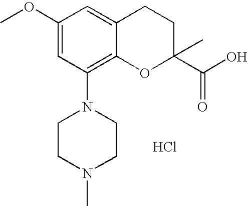

### Preparation of 6-Methoxy-2-methyl-8-(4-methyl-piperazin-1-yl)-chroman-2-carboxylic acid hydrochloride

### REFERENCE EXAMPLE 45A

### 2-Bromo-4-methoxy-phenol. This material was prepared as described in Journal of the Chemical Society (Resumed), 1927, 74-77

### REFERENCE EXAMPLE 45B

### 8-Bromo-6-methoxy-2-methyl-chroman-2-carboxylic acid ethyl ester

A mixture of 2-bromo-4-methoxy-phenol (Reference Example 45a, 6.1 g, 30 mmol), 2-methyl-acrylic acid ethyl ester (17.1 g, 150 mmol), dibutyl-amine (0.49 g, 3.7 mmol), paraformaldehyde (0.98 g, 32 mmol), and HOAc (0.91 g, 15.2 mmol) was heated to reflux for 24 h. At this point additional paraformaldehyde (0.98 g, 32 mmol) was added; mixture was heated to reflux for an additional 20 h, and cooled to room temperature. Volatile components were removed under high vacuum, residue was dissolved in EtOAc (100 mL), extracted with 1N aqueous HCl (50 mL), 10% aqueous NaHCO₃ (2×50 mL), and brine (50 mL). Organic layer was dried over Na₂SO₄, filtered, and evaporated leaving brown oil. product was purified by chromatography on silica (hexane:EtOAc, 6:1 to 4:1) to give 4.15 g (42%) of product. MS: m/z 329/331 (M+H).

### REFERENCE EXAMPLE 45C

### 6-Methoxy-2-methyl-8-(4-methyl-piperazin-1-yl)-chroman-2-carboxylic acid ethyl ester

To a reaction flask was added 1-methyl-piperazine (1.02 g, 10.2 mmol), 8-bromo-6-methoxy-2-methyl-chroman-2-carboxylic acid ethyl ester (Reference Example 45b, 2.80 g, 8.51 mmol), toluene (30 mL), racemic-2,2′-bis(diphenylphosphino)-1,1′-binaphthyl (317 mg, 0.51 mmol), cesium carbonate (3.88 g, 11.9 mmol), and palladium dba (156 mg, 0.17 mmol). Reaction was heated to reflux for 24 h, cooled to rt, and diluted with EtOAc. This was extracted with 1M aqueous K₂CO₃ (2×100 mL), and brine (100 mL). Extract was dried over Na₂SO₄, decolorized (Darco C-60), filtered, and evaporated under reduced pressure to leave brown oil. Product was purified by chromatography on silica, 99:1 to 98:2 to 95:5 DCM:MeOH (w/0.1% aq conc. NH₃). This gave 2.15 g of product (73%). ¹H NMR (300.132 MHz, CDCl₃) δ 6.36 (d, J=2.8 Hz, 1H), 6.18 (d, J=2.8 Hz, 1H), 4.23-4.04 (m, 2H), 3.72 (s, 3H), 3.66-3.41 (m, 2H), 2.98-2.81 (m, 2H), 2.80-2.52 (m, 6H), 2.44-2.28 (m, 1H), 2.35 (s, 1H), 1.90 (ddd, J=13.4, 11.0, 6.7 Hz, 3H), 1.64 (s, 3H), 1.20 (t, J=7.1 Hz, 3H).

### REFERENCE EXAMPLE 45D

### 6-Methoxy-2-methyl-8-(4-methyl-piperazin-1-yl)-chroman-2-carboxylic acid

To a solution of 6-methoxy-2-methyl-8-(4-methyl-piperazin-1-yl)-chroman-2-carboxylic acid ethyl ester (Reference Example 45c, 1.08 g, 3.10 mmol) in 12 mL of 5:1 THF:Methanol was added LiOH (130 mg, 3.10 mmol) dissolved in 2 mL Water. After 20 h the pH was adjusted to −2 w/1 N HCl. Solvent was evaporated under reduced pressure to give a tan solid. Water was added (10 mL), and solid was filtered. This gave 320 mg of product (33% as the HCl salt), MS: m/z 321 (M+H). Filtrate was evaporated under reduced pressure to yield additional product as the HCl/LiCl salt (680 mg, 63%) bringing the total yield to 96%.

### REFERENCE EXAMPLE 48

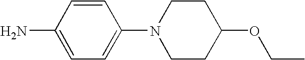

### Preparation of 4-(4-ethoxy-piperidin-1-yl)phenylamine

### REFERENCE EXAMPLE 48A

### 4-Ethoxy-piperidine-1-carboxylic acid tert-butyl ester

To a stirred solution of 4-hydroxy-piperidine-1-carboxylic acid tert-butyl ester (2.57 g, 12.8 mmol) in 30 mL DMF cooled to 0° C. was added sodium hydride (0.55 g, 13.8 mmol, 60% mineral oil dispersion) portionwise over 15 min. Ethyl iodide (1.99 g, 12.8 mmol) was then added slowly (caution: H₂ evolution). When the addition was complete reaction was warmed to rt overnight (18 h). Reaction was quenched with 100 mL water and extracted with ether/EtOAc (3×50 mL). Extracts were combined, washed with water (3×50 mL), 20% aqueous K₂CO₃ (50 mL), and brine (50 mL). Organic layer was dried over MgSO₄, filtered, and evaporated under reduced pressure to give an oil. Product was purified by chromatography on silica 100% Hexane to 10:1 to 4:1 Hexane:EtOAc to give 1.90 g (65%) of clear oil. ¹H NMR (300.132 MHz, CDCl₃) δ 3.92-3.68 (m, 2H), 3.51 (q, J=7.1 Hz, 2H), 3.43 (tt, J=8.5, 4.1 Hz, 1H), 3.05 (ddd, J=13.5, 10.2, 4.0 Hz, 2H), 1.92-1.73 (m, 2H), 1.59-1.37 (m, 2H), 1.45 (s, 9H), 1.20 (t, J=7.1 Hz, 3H).

### REFERENCE EXAMPLE 48B

### 4-Ethoxy-piperidine

To a solution of 4-ethoxy-piperidine-1-carboxylic acid tert-butyl ester (reference example 48a, 2.13 g, 9.29 mmol) in dichloromethane (10 mL) was added trifluoroacetic acid (4 mL). Reaction was stirred for 2 h, solvents were evaporated under reduced pressure, reside was mixed with 20 mL 20% K₂CO₃ (aq), and extracted with DCM (3×50 mL). Extracts were combined, dried over Na₂SO₄, filtered, and evaporated under reduced pressure to give 1.64 g (quantitative) of product; ¹H NMR (300.132 MHz, CDCl3) δ 4.35 (s, 1H), 3.51 (q, J=7.0 Hz, 2H), 3.41 (tt, J=8.7, 4.2 Hz, 1H), 3.19-3.05 (m, 2H), 2.72 (ddd, J=12.5, 9.3, 3.2 Hz, 2H), 2.02-1.87 (m, 2H), 1.53 (dtd, J=12.8, 8.7, 3.7 Hz, 2H), 1.20 (t, J=6.8 Hz, 3H).

### REFERENCE EXAMPLE 48C

### 4-Ethoxy-1-(4-nitro-phenyl)-piperidine

To a solution of 4-ethoxy-piperidine (Reference Example 48b, 1.64 g, <9.3 mmol) in triethylamine (3.63 g, 35.9 mmol) was added 4-fluoronitrobenzene (1.73 g, 12.3 mmol). The mixture was heated to 90° C. for 3 h, and cooled to room temperature. The reaction was poured over ice/water, resulting slurry was stirred for 1.5 h, and extracted with 2:1 diethyl ether:DCM (3×50 mL). Extracts were combined, washed with water (2×50 mL), 50 mL 20% K₂CO₃ (aq), and brine (50 mL). Organic extract was dried over Na₂SO₄, filtered, and evaporated under reduced pressure to give an oil. Product was purified by chromatography on silica 100% Hexane to 20:1 to 10:1 to 4:1 Hexane:EtOAc to give 2.36 g (99%) of an oil which crystallized under high vac; MS: m/z 251 (M+H).

### REFERENCE EXAMPLE 48D

### 4-(4-Ethoxy-piperidin-1-yl)-phenylamine

4-Ethoxy-1-(4-nitro-phenyl)-piperidine (Reference Example 48c, 345 mg, 1.36 mmol) was dissolved in 100 mL absolute EtOH and charged with 30 mg 10% Pd/C. Flask was attached to the Parr shaker, evacuated/backfilled with hydrogen (3 cycles). Mixture was agitated for 1.5 at room temperature under 50 psi, filtered through diatomaceous earth, and evaporated under reduced pressure. The yield was essentially quantitative (320 mg) and material was used as is without purification; ¹H NMR (300.132 MHz, CDCl₃) δ 6.83 (d, J=8.6 Hz, 2H), 6.64 (d, J=8.7 Hz, 2H), 3.54 (q, J=7.0 Hz, 2H), 3.70-3.50 (m, 5H), 2.85-2.65 (m, 2H), 2.07-1.90 (m, 2H), 1.53 (dtd, J=12.9, 9.5, 3.7 Hz, 2H), 1.22 (t, J=7.0 Hz, 3H).

### EXAMPLES

### Example 3

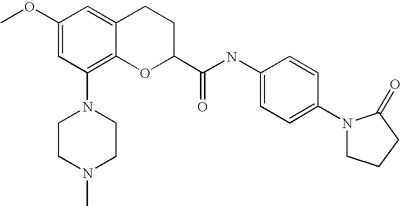

### 6-Methoxy-8-(4-methyl-piperazin-1-yl)-N-[4-(2-oxo-pyrrolidin-1-yl)-phenyl]-chroman-2-carboxamide

(rac)-6-Methoxy-8-(4-methyl-piperazin-1-yl)chroman-2-carboxylic acid hydrochloride (Reference Example 14, 1.15 mmol) was suspended in anhydrous methylene chloride (10 mL) with 1 drop of anhydrous DMF and cooled to 0 C. Oxalyl chloride (1.0 mL, 11.5 mmol) was added via syringe, and the solution stirred at 0 C for 1 h. The solvent was, removed in vacuo, and the resulting solid dried under high vacuum at room temperature for 1 h before suspending in anhydrous methylene chloride (5 mL). After cooling to 0 C, 1-(4-amino-phenyl)-pyrrolidin-2-one (Reference Example 3, 0.401 g, 2.28 mmol) in anhydrous methylene chloride (5 mL) was added, followed by dropwise addition of triethylamine (1.10 mL, 7.89 mmol). The mixture was stirred for 20 min at 0 C, then warmed to room temperature. The mixture was filtered and the filtrate was concentrated in vacuo. The product was passed through a column of basic alumina (ethyl acetate, 1:5 methylene chloride/ethyl acetate, 1:1 methylene chloride/ethyl acetate, 1:20 methanol/methylene chloride) yielding 235.4 mg (44%) after trituration from 1:5:10 ethyl acetate/ether/hexane.

LC/MS (M+1) m/z: 465. ¹H NMR (300 MHz, DMSO) δ 9.66 (s, 1H), 7.61 (td, J=9.3, 2.7 Hz, 4H), 6.30 (s, 2H), 4.65 (dd, J=9.2, 3.1 Hz, 1H), 3.81 (t, J=7.0 Hz, 2H), 3.66 (s, 3H), 3.14-2.89 (m, 4H), 2.89-2.64 (m, 2H), 2.48-2.40 (m, 6H), 2.29-2.20 (m, 1H), 2.19 (s, 3H), 2.05 (quintet, J=7.6 Hz, 2H), 1.98-1.92 (m, 1H).

The enantiomers (202 mg) were separated using chiral SFC (Chiralpak AD-H, 45% EtOH/CO₂+0.5% Dimethylethylamine). The faster moving isomer (Isomer I) was obtained as a white solid (87.6 mg). The slower moving isomer (Isomer 2) was isolated as a white solid (87.6 mg).

Isomer I: LC/MS (M+1) m/z: 465. ¹H NMR (300 MHz, DMSO) δ 9.66 (s, 1H), 7.61 (td, J=9.3, 2.7 Hz, 4H), 6.30 (s, 2H), 4.65 (dd, J=9.2, 3.1 Hz, 1H), 3.81 (t, J=7.0 Hz, 2H), 3.66 (s, 3H), 3.14-2.89 (m, 4H), 2.89-2.64 (m, 2H), 2.48-2.40 (m, 6H), 2.29-2.20 (m, 1H), 2.19 (s, 3H), 2.05 (quintet, J=7.6 Hz, 2H), 1.98-1.92 (m, 1H). Chiral SFC RT=3.81 min, >99.0%.

Isomer II: LC/MS (M+1) m/z: 465. ¹H NMR (300 MHz, DMSO) δ 9.66 (s, 1H), 7.61 (td, J=9.3, 2.7 Hz, 4H), 6.30 (s, 2H), 4.65 (dd, J=9.2, 3.1 Hz, 1H), 3.81 (t, J=7.0 Hz, 2H), 3.66 (s, 3H), 3.14-2.89 (m, 4H), 2.89-2.64 (m, 2H), 2.48-2.40 (m, 6H), 2.29-2.20 (m, 1H), 2.19 (s, 3H), 2.05 (quintet, J=7.6 Hz, 2H), 1.98-1.92 (m, 1H). Chiral SFC RT=4.84 min, >99.0%.

### Example 4

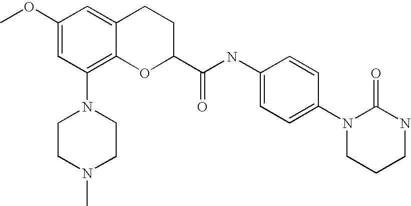

### 6-Methoxy-8-(4-methyl-piperazin-1-yl)-N-[4-(2-oxo-tetrahydro-pyrimidin-1-yl)-phenyl]chroman-2-carboxamide

(rac)-6-Methoxy-8-(4-methyl-piperazin-1-yl)chroman-2-carboxylic acid hydrochloride (Reference Example 14, 1.02 mmol) was suspended in anhydrous N,N-dimethylformamide (5 mL) and the following were added in order: N,N-diisopropylethylamine (0.530 ml, 3.04 mmol) and TBTU (0.353 g, 1.10 mmol). After stirring for 5 min at room temperature, 1-(4-aminophenyl)-tetrahydro-pyrimidin-2-one hydrochloride (Reference Example 1, 0.229 g, 1.00 mmol) was added, and the reaction was stirred overnight at room temperature. The reaction mixture was quenched with 5% aqueous potassium carbonate (10 mL), and extracted with ethyl acetate (2×50 mL), then 20% isopropyl alcohol/chloroform (2×50 mL). Combined organic extracts were washed with water (3×20 mL), dried (Na₂SO₄), filtered, and concentrated in vacuo to give the product. Trituration from 1:1:1 ethyl acetate/ether/methylene chloride gave 0.337 g (70%) of the title compound as a white solid.

LC/MS (M+1) m/z: 480. ¹H NMR (300 MHz, DMSO) δ 9.62 (s, 1H), 7.52 (d, J=8.9 Hz, 2H), 7.24 (d, J=8.8 Hz, 2H), 6.50 (s, 1H), 6.31 (s, 2H), 4.65 (dd, J=9.3, 3.1 Hz, 1H), 3.67 (s, 3H), 3.59 (t, J=5.7 Hz, 2H), 3.22 (td, J=5.7, 2.2 Hz, 2H), 3.14-2.90 (m, 4H), 2.90-2.64 (m, 2H), 2.58-2.38 [m (obscured by DMSO), 4H], 2.27-2.17 (m, 4H), 2.07-1.87 (m, 3H).

### Examples 4A and 4B

The enantiomers could be separated using any of the following methods and columns including: Preparative HPLC using 21×250 mm columns (˜20 mL/min flow rates) with column packings including Chiralpak AD, Chiralpak OD or Chiralpak OJ and mixtures of either EtOH and Hexane or isopropanol and hexane or supercritical fluid chromatography using columns such as Chiracel OD or Chiracel OD-H with either methanol/CO2/dimethylethylamine or ethanol/CO2/dimethylethylamine yielding (+)-6-methoxy-8-(4-methylpiperazin-1-yl)-N-(4-(2-oxotetrahydropyrimidin-1(2H)-yl)phenyl)chroman-2-carboxamide and (−)-6-methoxy-8-(4-methylpiperazin-1-yl)-N-(4-(2-oxotetrahydropyrimidin-1(2H)-yl)phenyl)chroman-2-carboxamide.

### Example 5

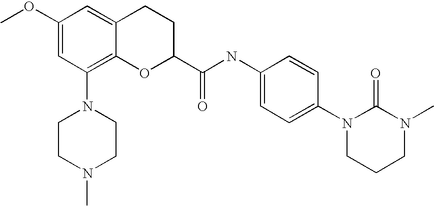

### 6-Methoxy-8-(4-methyl-piperazin-1-yl)-N-[4-(3-methyl-2-oxo-tetrahydro-pyrimidin-1-yl)-phenyl]chroman-2-carboxamide

(rac)-6-Methoxy-8-(4-methyl-piperazin-1-yl)chroman-2-carboxylic acid hydrochloride (Reference Example 14, 1.17 mmol) was suspended in anhydrous N,N-dimethylformamide (5 mL) and the following were added in order: N,N-diisopropylethylamine (0.410 ml, 2.35 mmol) and TBTU (0.418 g, 1.30 mmol). After stirring for 5 min at room temperature, 1-(4-amino-phenyl)-3-methyl-tetrahydro-pyrimidin-2-one (Reference Example 2, 0.242 g, 1.18 mmol) was added, and the reaction was stirred overnight at room temperature. The reaction mixture was quenched with 5% aqueous potassium carbonate (10 mL), and extracted with ethyl acetate (2×20 mL). Combined organic extracts were washed with water (3×15 mL), dried (Na₂SO₄), filtered, and concentrated in vacuo to give the product. Trituration from 1:10:10 ethyl acetate/ether/hexane gave 0.288 g (47%) of the title compound as a pale yellow solid.

LC/MS (M+1) m/z: 494. ¹H NMR (300 MHz, DMSO) δ 9.62 (s, 1H), 7.52 (d, J=8.8 Hz, 2H), 7.20 (d, J=8.8 Hz, 2H), 6.30 (s, 2H), 4.64 (dd, J=9.3, 3.1 Hz, 1H), 3.66 (s, 3H), 3.61 (t, J=5.7 Hz, 2H), 3.33 (t, J=6.1 Hz, 2H), 3.14-2.90 (m, 4H), 2.84 (s, 3H), 2.82-2.65 (m, 2H), 2.56-2.39 (m, 4H), 2.29-2.15 (m, 4H), 2.07-1.93 (m, 3H).

The enantiomers (210 mg) were separated using chiral SFC (Chiralpak AD, 50% IPA/CO₂+0.5% Dimethylethylamine). The faster moving isomer (Isomer I) was obtained as a white solid (100 mg). The slower moving isomer (Isomer 2) was isolated as a white solid (103 mg).

Isomer I: LC/MS (M+1) m/z: 494. ¹H NMR (300 MHz, DMSO) δ 9.61 (s, 1H), 7.51 (d, J=8.9 Hz, 2H), 7.20 (d, J=8.9 Hz, 2H), 6.30 (s, 2H), 4.64 (dd, J=9.3, 3.2 Hz, 1H), 3.66 (s, 3H), 3.61 (t, J=5.7 Hz, 2H), 3.33 (t, J=6.1 Hz, 2H), 3.13-3.01 (m, 2H), 3.01-2.91 (m, 2H), 2.84 (s, 3H), 2.82-2.64 (m, 2H), 2.48-2.40 (m, 4H), 2.28-2.15 (m, 4H), 2.08-1.92 (m, 3H). Chiral SFC RT=2.44 min, >99.0%.

Isomer II: LC/MS (M+1) m/z: 494. ¹H NMR (300 MHz, DMSO) δ 9.61 (s, 1H), 7.52 (d, J=8.9 Hz, 2H), 7.20 (d, J=8.8 Hz, 2H), 6.30 (s, 2H), 4.64 (dd, J=9.3, 3.2 Hz, 1H), 3.66 (s, 3H), 3.61 (t, J=5.7 Hz, 2H), 3.33 (t, J=6.1 Hz, 2H), 3.14-3.01 (m, 2H), 3.01-2.90 (m, 2H), 2.84 (s, 3H), 2.82-2.64 (m, 2H), 2.48-2.39 (m, 4H), 2.29-2.15 (m, 4H), 2.07-1.93 (m, 3H). Chiral SFC RT=3.94 min, >99.0%.

### Example 6

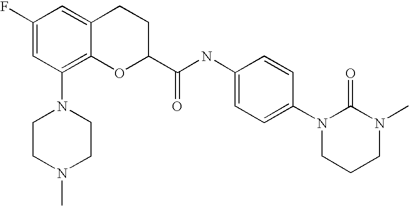

### 6-Fluoro-8-(4-methyl-piperazin-1-yl)-N-[4-(3-methyl-2-oxo-tetrahydro-pyrimidin-1-yl)-phenyl]chroman-2-carboxamide

(rac)-6-Fluoro-8-(4-methyl-piperazin-1-yl)chroman-2-carboxylic acid hydrochloride (Reference Example 15, 1.18 mmol) was suspended in anhydrous N,N-dimethylformamide (5 mL) and the following were added in order: N,N-diisopropylethylamine (0.410 ml, 2.35 mmol) and TBTU (0.417 g, 1.30 mmol). After stirring for 5 min at room temperature, 1-(4-amino-phenyl)-3-methyl-tetrahydro-pyrimidin-2-one (Reference Example 2, 0.243 g, 1.18 mmol) was added, and the reaction was stirred overnight at room temperature. The reaction mixture was quenched with 5% aqueous potassium carbonate (10 mL), and extracted with ethyl acetate (2×20 mL). Combined organic extracts were washed with water (3×15 mL), dried (Na₂SO₄), filtered, and concentrated in vacuo to give the product. The product was purified by silica gel chromatography, eluting with 98:2 methylene chloride/methanol containing 0.1% ammonium hydroxide then 95:5 methylene chloride/methanol containing 0.1% ammonium hydroxide. The product was collected as a tacky semi-solid. This residue was crystallized from 1:1:10 hexane/ethyl acetate/ether to give the title compound as a pale yellow solid (0.288 g, 51%).

LC/MS (M+1) m/z: 206. ¹H NMR (300 MHz, DMSO) δ 9.72 (s, 1H), 7.52 (d, J=8.7 Hz, 2H), 7.20 (d, J=8.7 Hz, 2H), 6.57 (d, J=4.1 Hz, 1H), 6.54 (d, J=3.6 Hz, 1H), 4.71 (dd, J=8.6, 2.7 Hz, 1H), 3.61 (t, J=5.6 Hz, 2H), 3.32 (t, J=5.9 Hz, 2H), 3.19-2.89 (m, 4H), 2.88-2.65 (m, 5H), 2.61-2.35 [m (obscured by DMSO), 4H), 2.29-2.12 (m, 4H), 2.10-1.92 (m, 3H).

The title compound was also synthesized in non-racemic form from resolved 6-fluoro-8-(4-methyl-piperazin-1-yl)chroman-2-carboxylic acid hydrochloride (Reference Example 12) using the above procedure. The product was isolated as a pale yellow, foamy solid. Crystallization from 1:1:10 hexane/ethyl acetate/ether gave the title compound as a pale yellow solid (0.289 g, 60%).

LC/MS (M+1) m/z: 206. ¹H NMR (300 MHz, DMSO) δ 9.71 (s, 1H), 7.52 (d, J=8.8 Hz, 2H), 7.20 (d, J=8.8 Hz, 2H), 6.56 (dd, J=7.2, 3.2 Hz, 1H), 6.53 (d, J=2.0 Hz, 1H), 4.71 (dd, J=9.0, 3.2 Hz, 1H), 3.60 (t, J=5.7 Hz, 2H), 3.33 (t, J=6.1 Hz, 2H), 3.18-3.04 (m, 2H), 3.02-2.88 (m, 2H), 2.84 (s, 3H), 2.82-2.65 (m, 2H), 2.47-2.38 (m, 4H), 2.29-2.15 (m, 4H), 2.08-1.94 (m, 3H).

### Example 7

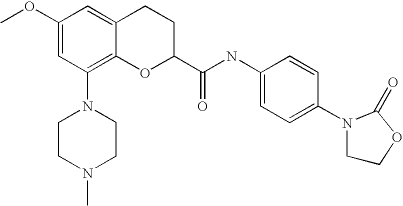

### 6-Methoxy-8-(4-methyl-piperazin-1-yl)-N-[4-(2-oxo-oxazolidin-3-yl)-phenyl]chroman-2-carboxamide

(rac)-6-Methoxy-8-(4-methyl-piperazin-1-yl)chroman-2-carboxylic acid hydrochloride (Reference Example 14, 0.990 mmol) was suspended in anhydrous N,N-dimethylformamide (3 mL) and the following were added in order: N,N-diisopropylethylamine (0.350 ml, 2.01 mmol) and TBTU (0.351 g, 1.09 mmol). After stirring for 5 min at room temperature, 3-(4-aminophenyl)-oxazolidin-2-one (Reference Example 4, 0.176 g, 0.990 mmol) was added, and the reaction was stirred overnight at room temperature. The reaction mixture was quenched with 5% aqueous potassium carbonate (10 mL), and extracted with ethyl acetate (2×20 mL). Combined organic extracts were washed with water (3×15 mL), dried (Na₂SO₄), filtered, and concentrated in vacuo to give the product. Purification by flash column chromatography (10 g SiO₂, eluting with 0.5% NH₄OH in 2-5% methanol/methylene chloride gradient) yielded a tacky solid. Crystallization from 1:10 ethyl acetate/ether gave the title compound as a white solid (0.276 g, 57%).

LC/MS (M+1) m/z: 467. ¹H NMR (300 MHz, DMSO) δ 9.69 (s, 1H), 7.62 (d, J=9.1 Hz, 2H), 7.53 (d, J=9.1 Hz, 2H), 6.30 (s, 2H), 4.65 (dd, J=9.2, 3.2 Hz, 1H), 4.43 (t, J=8.0 Hz, 2H), 4.04 (t, J=7.9 Hz, 2H), 3.67 (s, 3H), 3.15-2.90 (m, 4H), 2.89-2.63 (m, 2H), 2.57-2.38 (m, 4H), 2.29-2.13 (m, 4H), 2.07-1.91 (m, 1H).

### Examples 7A and 7B

The enantiomers could be separated using any of the following methods and columns including: Preparative HPLC using 21×250 mm columns (˜20 mL/min flow rates) with column packings including Chiralpak AD, Chiralpak OD or Chiralpak OJ and mixtures of either EtOH and Hexane or isopropanol and hexane or supercritical fluid chromatography using columns such as Chiracel OD or Chiracel OD-H with either methanol/CO2/dimethylethylamine or ethanol/CO2/dimethylethylamine yielding (+)-6-Methoxy-8-(4-methyl-piperazin-1-yl)-N-[4-(2-oxo-oxazolidin-3-yl)-phenyl]chroman-2-carboxamide and (−)-6-Methoxy-8-(4-methyl-piperazin-1-yl)-N-[4-(2-oxo-oxazolidin-3-yl)-phenyl]chroman-2-carboxamide

### Example 8

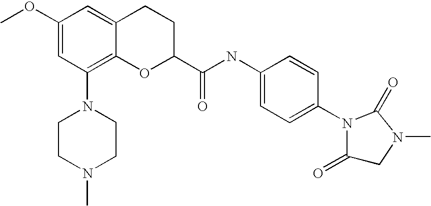

### 6-Methoxy-8-(4-methyl-piperazin-1-yl)-N-[4-(3-methyl-2,5-dioxo-imidazolidin-1-yl)-phenyl]chroman-2-carboxamide

(rac)-6-Methoxy-8-(4-methyl-piperazin-1-yl)chroman-2-carboxylic acid hydrochloride (Reference Example 14, 1.00 mmol) was suspended in anhydrous N,N-dimethylformamide (5 mL) and the following were added in order: N,N-diisopropylethylamine (0.435 ml, 2.49 mmol) and TBTU (0.357 g, 1.11 mmol). After stirring for 5 min at room temperature, 3-(4-aminophenyl)-1-methyl-imidazolidine-2,4-dione (Reference Example 5, 0.220 g, 1.01 mmol) was added, and the reaction was stirred overnight at room temperature. The reaction mixture was quenched with 5% aqueous potassium carbonate (10 mL), and extracted with ethyl acetate (2×20 mL). Combined organic extracts were washed with water (3×15 mL), dried (Na₂SO₄), filtered, and concentrated in vacuo to give the product as a red-brown solid. Purification by flash column chromatography (10 g SiO₂, eluting with 0.5% NH₄OH in 2-5% methanol/methylene chloride gradient) yielded an orange solid. Trituration from 10:1 ethyl acetate/hexane gave the title compound (0.225 g, 46%).

LC/MS (M+1) m/z: 494. ¹H NMR (300 MHz, DMSO) δ 9.87 (s, 1H), 7.69 (d, J=8.9 Hz, 2H), 7.31 (d, J=8.8 Hz, 2H), 6.30 (s, 2H), 4.68 (dd, J=9.2, 3.2 Hz, 1H), 4.10 (s, 2H), 3.67 (s, 3H), 3.16-3.02 (m, 2H), 3.00-2.88 (m, 5H), 2.85-2.77 (m, 1H), 2.76-2.65 (m, 1H), 2.55-2.39 (m [obscured by DMSO], 4H), 2.30-2.14 (m, 4H), 2.10-1.93 (m, 1H).

### Examples 8A and 8B

The enantiomers could be separated using any of the following methods and columns including: Preparative HPLC using 21×250 mm columns (˜20 mL/min flow rates) with column packings including Chiralpak AD, Chiralpak OD or Chiralpak OJ and mixtures of either EtOH and Hexane or isopropanol and hexane or supercritical fluid chromatography using columns such as Chiracel OD or Chiracel OD-H with either methanol/CO2/dimethylethylamine or ethanol/CO2/dimethylethylamine yielding (+)-6-Methoxy-8-(4-methyl-piperazin-1-yl)-N-[4-(3-methyl-2,5-dioxo-imidazolidin-1-yl)-phenyl]chroman-2-carboxamide and (−)-6-Methoxy-8-(4-methyl-piperazin-1-yl)-N-[4-(3-methyl-2,5-dioxo-imidazolidin-1-yl)-phenyl]chroman-2-carboxamide.

### Example 9

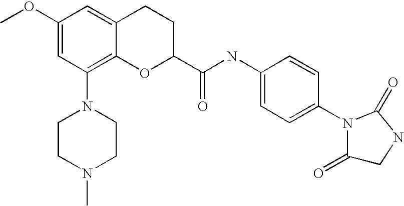

### 6-Methoxy-8-(4-methyl-piperazin-1-yl)-N-[4-(2,5-dioxo-imidazolidin-1-yl)-phenyl]-chroman-2-carboxamide

(rac)-6-Methoxy-8-(4-methyl-piperazin-1-yl)chroman-2-carboxylic acid hydrochloride (Reference Example 14, 1.20 mmol) was suspended in anhydrous N,N-dimethylformamide (6 mL) and the following were added in order: N,N-diisopropylethylamine (0.530 ml, 3.04 mmol) and TBTU (0.431 g, 1.34 mmol). After stirring for 5 min at room temperature, 3-(4-aminophenyl)-imidazolidine-2,4-dione (Reference Example 6, 0.230 g, 1.20 mmol) was added, and the reaction was stirred overnight at room temperature. The reaction mixture was quenched with 10% aqueous potassium carbonate (10 mL), and extracted with ethyl acetate (2×20 mL). Combined organic extracts were washed with water (3×15 mL), dried (Na₂SO₄), filtered, and concentrated in vacuo to give the product. Trituration from 1:1:10 methylene chloride/ethyl acetate/ether gave the title compound (0.242 g, 42%).

LC/MS (M+1) m/z: 480. ¹H NMR (300 MHz, DMSO) δ 9.84 (s, 1H), 9.84 (s, 1H), 7.69 (d, J=8.9 Hz, 2H), 7.31 (d, J=8.8 Hz, 2H), 6.30 (s, 2H), 4.68 (dd, J=9.3, 3.2 Hz, 1H), 4.05 (s, 2H), 3.67 (s, 3H), 3.15-3.01 (m, 2H), 3.01-2.90 (m, 2H), 2.90-2.65 (m, 2H), 2.48-2.38 (m, 4H), 2.29-2.15 (m, 4H), 2.10-1.94 (m, 1H).

### Examples 9A and 9B

The enantiomers could be separated using any of the following methods and columns including: Preparative HPLC using 21×250 mm columns (˜20 mL/min flow rates) with column packings including Chiralpak AD, Chiralpak OD or Chiralpak OJ and mixtures of either EtOH and Hexane or isopropanol and hexane or supercritical fluid chromatography using columns such as Chiracel OD or Chiracel OD-H with either methanol/CO2/dimethylethylamine or ethanol/CO2/dimethylethylamine yielding (+)-6-Methoxy-8-(4-methyl-piperazin-1-yl)-N-[4-(2,5-dioxo-imidazolidin-1-yl)-phenyl]-chroman-2-carboxamide and (−)-6-Methoxy-8-(4-methyl-piperazin-1-yl)-N-[4-(2,5-dioxo-imidazolidin-1-yl)-phenyl]-chroman-2-carboxamide.

### Example 10

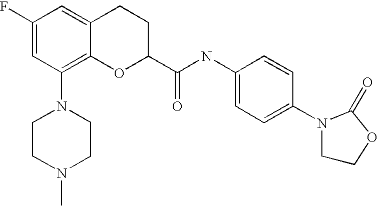

### 6-Fluoro-8-(4-methyl-piperazin-1-yl)-N-[4-(2-oxo-oxazolidin-3-yl)-phenyl]chroman-2-carboxamide

(+)-6-fluoro-8-(4-methyl-piperazin-1-yl)chroman-2-carboxylic acid hydrochloride (Reference Example 12, 1.01 mmol) was suspended in anhydrous N,N-dimethylformamide (5 mL) and the following were added in order: N,N-diisopropylethylamine (0.355 ml, 2.04 mmol) and TBTU (0.351 g, 1.09 mmol). After stirring for 5 min at room temperature, 3-(4-aminophenyl)-oxazolidin-2-one (Reference Example 4, 0.183 g, 1.03 mmol) was added, and the reaction was stirred overnight at room temperature. The reaction mixture was quenched with 5% aqueous potassium carbonate (10 mL), and extracted with ethyl acetate (2×20 mL). Combined organic extracts were washed with water (3×15 mL), dried (Na₂SO₄), filtered, and concentrated in vacuo to give the product. Purification by flash column chromatography (10 g SiO₂, eluting with 0.5% NH₄OH in 2-5% methanol/methylene chloride gradient) yielded an off-white solid. Trituration from 1:10 ethyl acetate/ether gave the title compound as a white solid (0.320 g, 70%).

LC/MS (M+1) m/z: 455. ¹H NMR (300 MHz, DMSO) δ 9.77 (s, 1H), 7.63 (d, J=9.1 Hz, 2H), 7.53 (d, J=9.2 Hz, 2H), 6.57 (dd, J=7.0, 3.1 Hz, 1H), 6.53 (d, J=1.9 Hz, 1H), 4.71 (dd, J=9.0, 3.2 Hz, 1H), 4.43 (t, J=8.0 Hz, 2H), 4.04 (t, J=8.0 Hz, 2H), 3.18-3.03 (m, 2H), 3.00-2.88 (m, 2H), 2.88-2.65 (m, 2H), 2.47-2.38 (m, 4H), 2.28-2.16 (m, 4H), 2.10-1.94 (m, 1H). ¹⁹F NMR (282 MHz, CDCl₃)δ −117.24.

### Example 11

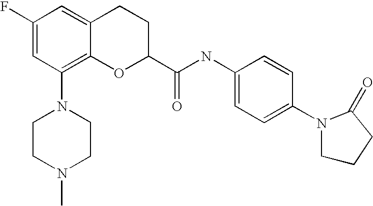

### 6-Fluoro-8-(4-methyl-piperazin-1-yl)-N-[4-(2-oxo-pyrrolidin-1-yl)-phenyl]-chroman-2-carboxamide

(+)-6-fluoro-8-(4-methyl-piperazin-1-yl)chroman-2-carboxylic acid hydrochloride (Reference Example 12, 1.01 mmol) was suspended in anhydrous N,N-dimethylformamide (5 mL) and the following were added in order: N,N-diisopropylethylamine (0.355 ml, 2.04 mmol) and TBTU (0.356 g, 1.11 mmol). After stirring for 5 min at room temperature, 1-(4-amino-phenyl)-pyrrolidin-2-one (Reference Example 3, 0.180 g, 1.02 mmol) was added, and the reaction was stirred overnight at room temperature. The reaction mixture was quenched with 5% aqueous potassium carbonate (10 mL), and extracted with ethyl acetate (2×20 mL). Combined organic extracts were washed with water (3×15 mL), dried (Na₂SO₄), filtered, and concentrated in vacuo to give the product. Purification by flash column chromatography (10 g SiO₂, eluting with 0.5% NH₄OH in 2-5% methanol/methylene chloride gradient) yielded an off-white solid. Trituration from 1:10 ethyl acetate/ether gave the title compound as a white solid (0.283 g, 62%).

LC/MS (M+1) m/z: 453. ¹H NMR (300 MHz, DMSO) δ 9.76 (s, 1H), 7.70-7.53 (m, 4H), 6.60-6.51 (m, 2H), 4.71 (dd, J=9.0, 3.3 Hz, 1H), 3.81 (t, J=7.0 Hz, 2H), 3.19-3.04 (m, 2H), 3.01-2.89 (m, 2H), 2.89-2.65 (m, 2H), 2.54-2.39 [m (obscured by DMSO), 6H), 2.29-2.16 (m, 4H), 2.12-1.94 (m, 3H).

### Example 12

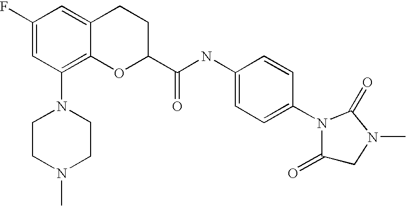

### 6-Fluoro-8-(4-methyl-piperazin-1-yl)-N-[4-(3-methyl-2,5-dioxo-imidazolidin-1-yl)-phenyl]chroman-2-carboxamide

(+)-6-fluoro-8-(4-methyl-piperazin-1-yl)chroman-2-carboxylic acid hydrochloride (Reference Example 12, 1.01 mmol) was suspended in anhydrous N,N-dimethylformamide (5 mL) and the following were added in order: N,N-diisopropylethylamine (0.355 ml, 2.04 mmol) and TBTU (0.354 g, 1.10 mmol). After stirring for 5 min at room temperature, 3-(4-aminophenyl)-1-methyl-imidazolidine-2,4-dione (Reference Example 5, 0.209 g, 1.02 mmol) was added, and the reaction was stirred overnight at room temperature. The reaction mixture was quenched with 5% aqueous potassium carbonate (10 mL), and extracted with ethyl acetate (2×20 mL). Combined organic extracts were washed with water (3×15 mL), dried (Na₂SO₄), filtered, and concentrated in vacuo to give the product as a pale orange solid. Trituration from 10:1 ethyl acetate/hexane gave the title compound (0.284 g, 58%).

LC/MS (M+1) m/z: 482.11H NMR (300 MHz, DMSO) δ 9.96 (s, 1H), 7.70 (d, J=8.9 Hz, 2H), 7.31 (d, J=8.8 Hz, 2H), 6.61-6.50 (m, 2H), 4.74 (dd, J=8.9, 3.2 Hz, 1H), 4.10 (s, 2H), 3.20-3.08 (m, 2H), 2.99-2.88 (m, 5H), 2.88-2.67 (m, 2H), 2.48-2.41 (m, 4H), 2.29-2.16 (m, 4H), 2.12-1.97 (m, 1H).

### Example 13

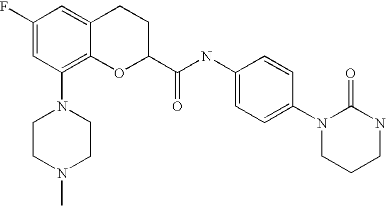

### 6-Fluoro-8-(4-methyl-piperazin-1-yl)-N-[4-(2,5-dioxo-imidazolidin-1-yl)-phenyl]-chroman-2-carboxamide

(+)-6-fluoro-8-(4-methyl-piperazin-1-yl)chroman-2-carboxylic acid hydrochloride (Reference Example 12, 1.01 mmol) was suspended in anhydrous N,N-dimethylformamide (5 mL) and the following were added in order: N,N-diisopropylethylamine (0.355 ml, 2.04 mmol) and TBTU (0.356 g, 1.11 mmol). After stirring for 5 min at room temperature, 3-(4-aminophenyl)-imidazolidine-2,4-dione (Reference Example 6, 0.196 g, 1.03 mmol) was added, and the reaction was stirred overnight at room temperature. The reaction mixture was quenched with 10% aqueous potassium carbonate (10 mL), and extracted with ethyl acetate (2×20 mL). Combined organic extracts were washed with water (3×15 mL), dried (Na₂SO₄), filtered, and concentrated in vacuo to give the product. Trituration from 1:1:10 methylene chloride/ethyl acetate/ether gave the title compound (0.305 g, 65%).

LC/MS (M+1) m/z: 468. ¹H NMR (300 MHz, DMSO) δ 9.95 (s, 1H), 8.25 (s, 1H), 7.70 (d, J=8.9 Hz, 2H), 7.31 (d, J=8.8 Hz, 2H), 6.57 (dd, J=7.0, 3.2 Hz, 1H), 6.54 (d, J=1.7 Hz, 1H), 4.74 (dd, J=8.9, 3.2 Hz, 1H), 4.05 (s, 2H), 3.21-3.08 (m, 2H), 3.01-2.89 (m, 2H), 2.88-2.66 (m, 2H), 2.55-2.40 [m (obscured by DMSO), 4H), 2.30-2.17 (m, 4H), 2.12-1.97 (m, 1H).

### Example 14

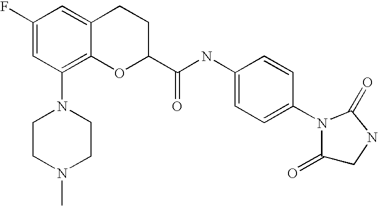

### 6-Fluoro-8-(4-methyl-piperazin-1-yl)-N-[4-(2-oxo-tetrahydro-pyrimidin-1-yl)-phenyl]chroman-2-carboxamide

(+)-6-fluoro-8-(4-methyl-piperazin-1-yl)chroman-2-carboxylic acid hydrochloride (Reference Example 12, 1.02 mmol) was suspended in anhydrous N,N-dimethylformamide (5 mL) and the following were added in order: N,N-diisopropylethylamine (0.530 ml, 3.04 mmol) and TBTU (0.357 g, 1.11 mmol). After stirring for 5 min at room temperature, 1-(4-aminophenyl)-tetrahydro-pyrimidin-2-one hydrochloride (Reference Example 1, 0.231 g, 1.01 mmol) was added, and the reaction was stirred overnight at room temperature. The reaction mixture was quenched with 10% aqueous potassium carbonate (10 mL), and extracted with ethyl acetate (2×40 mL). Combined organic extracts were washed with water (3×20 mL), dried (Na₂SO₄), filtered, and concentrated in vacuo to give the product. Trituration from 1:1:1 ethyl acetate/ether/methylene chloride gave 0.255 g (54%) of the title compound as a white solid.

LC/MS (M+1) m/z: 468. ¹H NMR (300 MHz, DMSO) δ 9.71 (s, 1H), 7.52 (d, J=8.8 Hz, 2H), 7.24 (d, J=8.8 Hz, 2H), 6.60-6.46 (m, 3H), 4.71 (dd, J=9.1, 3.1 Hz, 1H), 3.59 (t, J=5.7 Hz, 2H), 3.25-3.18 (m, 2H), 3.17-3.04 (m, 2H), 3.01-2.88 (m, 2H), 2.88-2.66 (m, 2H), 2.47-2.38 (m, 4H), 2.28-2.15 (m, 4H), 2.09-1.88 (m, 3H).

### Example 15

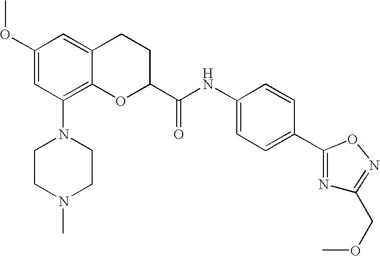

### 6-Methoxy-N-{4-[3-(methoxymethyl)-1,2,4-oxadiazol-5-yl]phenyl}-8-(4-methylpiperazin-1-yl)chroman-2-carboxamide

The title compound was prepared in 95% yield in the same manner as Example 9, but using 6-methoxy-8-(4-methyl-piperazin-1-yl)chroman-2-carboxylic acid hydrochloride (Reference Example 14) and 4-[3-(methoxymethyl)-1,2,4-oxadiazol-5-yl]aniline (Reference Example 7). LC/MS (M+1) m/z: 494. ¹H-NMR (300 MHz, CDCl₃) δ 9.06 (s, 1H) 8.18 (d, 2H) 7.78(d, 2H), 6.47 (d, 1H), 6.31 (d, 1H), 4.72 (dd, 1H), 4.63 (s, 2H), 3.75 (s, 3H), 3.52 (s, 3H), 3.36 (bm, 2H), 2.99-2.52 (m, 7H), 2.39 (s, 3H), 2.06 (m, 1H).

### Examples 15A and 15B

The enantiomers could be separated using any of the following methods and columns including: Preparative HPLC using 21×250 mm columns (˜20 mL/min flow rates) with column packings including Chiralpak AD, Chiralpak OD or Chiralpak OJ and mixtures of either EtOH and Hexane or isopropanol and hexane or supercritical fluid chromatography using columns such as Chiracel OD or Chiracel OD-H with either methanol/CO2/dimethylethylamine or ethanol/CO2/dimethylethylamine yielding (+)-6-Methoxy-N-{4-[3-(methoxymethyl)-1,2,4-oxadiazol-5-yl]phenyl}-8-(4-methylpiperazin-1-yl)chroman-2-carboxamide and (−)-6-Methoxy-N-{4-[3-(methoxymethyl)-1,2,4-oxadiazol-5-yl]phenyl}-8-(4-methylpiperazin-1-yl)chroman-2-carboxamide.

### Example 16

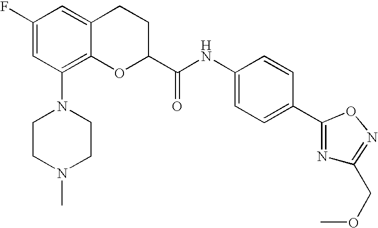

### (+)-6-Fluoro-N-{4-[3-(methoxymethyl)-1,2,4-oxadiazol-5-yl]phenyl}-8-(4-methylpiperazin-1-yl)chroman-2-carboxamide

The title compound was prepared in 49% yield in the same manner as Example 15, but using (+)-6-fluoro-8-(4-methyl-piperazin-1-yl)chroman-2-carboxylic acid hydrochloride lithium chloride (Reference Example 12) and 4-[3-(methoxymethyl)-1,2,4-oxadiazol-5-yl]aniline (Reference Example 7). LC/MS (M+1) m/z: 482. ¹H-NMR (300 MHz, CDCl₃) δ 9.00 (s, 1H) 8.18 (d, 2H) 7.78(d, 2H), 6.60 (d, 1H), 6.50 (d, 1H), 4.74 (dd, 1H), 4.64 (s, 2H), 3.52 (s, 3H), 3.34 (bm, 2H), 2.99-2.53 (m, 7H), 2.39 (s, 3H), 2.03 (m, 1H).

### Example 17

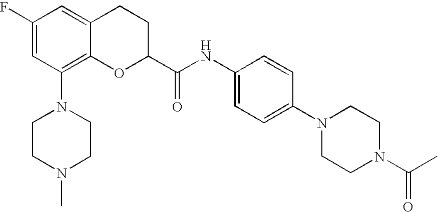

### (+)-N-[4-(4-acetylpiperazin-1-yl)phenyl]-6-fluoro-8-(4-methylpiperazin-1-yl)chroman-2-carboxamide (isomer 1)

The title compound was prepared in 87% yield in the same manner as Example 16, but using 4-(4-acetylpiperazin-1-yl)aniline instead of 4-[3-(methoxymethyl)-1,2,4-oxadiazol-5-yl]aniline (Reference Example 7). LC/MS (M+1) m/z: 496. ¹H-NMR (300 MHz, CDCl₃) δ 8.63 (s, 1H) 7.49 (d, 2H) 6.92(d, 2H), 6.57 (dd, 1H), 6.48 (dd, 1H), 4.68 (dd, 1H), 3.77 (t, 2H), 3.62 (t, 2H), 3.31 (bm, 2H), 3.14 (m, 4H), 2.99-2.54 (m, 9H), 2.38 (s, 3H), 2.14 (s, 3H), 2.02 (m, 1H).

### Example 18

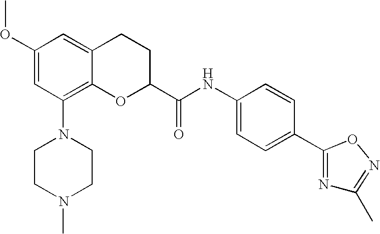

### 6-Methoxy-N-[4-(3-methyl-1,2,4-oxadiazol-5-yl)phenyl]-8-(4-methylpiperazin-1-yl)chroman-2-carboxamide

The title compound was prepared in 22% yield in the same manner as Example 17, but using (rac)-6-methoxy-8-(4-methyl-piperazin-1-yl)chroman-2-carboxylic acid hydrochloride (Reference Example 14) and 4-(3-methyl-1,2,4-oxadiazol-5-yl)aniline (Reference Example 8). LC/MS (M+1) m/z: 464. ¹H-NMR (300 MHz, CDCl₃) δ 9.05 (s, 1H) 8.11 (d, 2H) 7.78(d, 2H), 6.47 (d, 1H), 6.31 (d, 1H), 4.72 (dd, 1H), 3.75 (s, 3H), 3.32 (bm, 2H), 2.98-2.53 (m, 7H), 2.46 (s, 3H), 2.39 (s, 3H), 2.07 (m, 1H).

### Examples 18A and 18B

The enantiomers could be separated using any of the following methods and columns including: Preparative HPLC using 21×250 mm columns (˜20 mL/min flow rates) with column packings including Chiralpak AD, Chiralpak OD or Chiralpak OJ and mixtures of either EtOH and Hexane or isopropanol and hexane or supercritical fluid chromatography using columns such as Chiracel OD or Chiracel OD-H with either methanol/CO2/dimethylethylamine or ethanol/CO2/dimethylethylamine yielding (+)-6-Methoxy-N-[4-(3-methyl-1,2,4-oxadiazol-5-yl)phenyl]-8-(4-methylpiperazin-1-yl)chroman-2-carboxamide and (−)-6-Methoxy-N-[4-(3-methyl-1,2,4-oxadiazol-5-yl)phenyl]-8-(4-methylpiperazin-1-yl)chroman-2-carboxamide.

### Example 19

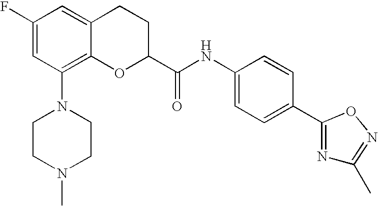

### (+)-6-Fluoro-N-[4-(3-methyl-1,2,4-oxadiazol-5-yl)phenyl]-8-(4-methylpiperazin-1-yl)chroman-2-carboxamide

The title compound was prepared in 15% yield in the same manner as Example 18, but using (+)-6-fluoro-8-(4-methyl-piperazin-1-yl)chroman-2-carboxylic acid lithium chloride hydrochloride (Reference Example 12) and 4-(3-methyl-1,2,4-oxadiazol-5-yl)aniline (Reference Example 8). LC/MS (M+1) m/z: 452. ¹H-NMR (300 MHz, CDCl₃) δ 98.98 (s, 1H) 8.12 (d, 2H) 7.78(d, 2H), 6.60 (d, 1H), 6.50 (d, 1H), 4.73 (dd, 1H), 3.35 (bm, 2H), 2.98-2.53 (m, 7H), 2.46 (s, 3H), 2.39 (s, 3H), 2.07 (m, 1H).

### Example 20

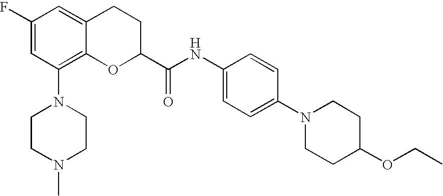

### N-[4-(4-ethoxypiperidin-1-yl)phenyl]-6-fluoro-8-(4-methylpiperazin-1-yl)chroman-2-carboxamide

The title compound was prepared in 80% yield in the same manner as Example 19, but using (rac)-6-fluoro-8-(4-methyl-piperazin-1-yl)chroman-2-carboxylic acid hydrochloride (Reference Example 15) and 4-(4-ethoxypiperidin-1-yl)aniline (Reference Example 48).

LC/MS (M+1) m/z: 497. ¹H-NMR (300 MHz, CDCl₃) δ 8.58 (s, 1H), 7.45 (d, 2H) 6.93(d, 2H), 6.57 (d, 1H), 6.48 (d, 1H), 4.67 (dd, 1H), 3.56 (m, 4H), 3.31 (bm, 2H), 2.99-2.55 (m, 10H), 2.38 (s, 3H), 2.04 (m, 3H), 1.70 (m, 2H), 1.20 (t, 3H).

### Examples 20A and 20B

The enantiomers could be separated using any of the following methods and columns including: Preparative HPLC using 21×250 mm columns (˜20 mL/min flow rates) with column packings including Chiralpak AD, Chiralpak OD or Chiralpak OJ and mixtures of either EtOH and Hexane or isopropanol and hexane or supercritical fluid chromatography using columns such as Chiracel OD or Chiracel OD-H with either methanol/CO2/dimethylethylamine or ethanol/CO2/dimethylethylamine yielding (+)-N-[4-(4-ethoxypiperidin-1-yl)phenyl]-6-fluoro-8-(4-methylpiperazin-1-yl)chroman-2-carboxamide and (−)-N-[4-(4-ethoxypiperidin-1-yl)phenyl]-6-fluoro-8-(4-methylpiperazin-1-yl)chroman-2-carboxamide.

### Example 58

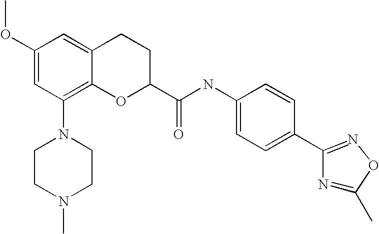

### 6-methoxy-N-(4-(5-methyl-1,2,4-oxadiazol-3-yl)phenyl)-8-(4-methylpiperazin-1-yl)chroman-2-carboxamide

To a stirred solution of 4-(5-methyl-[1,2,4]oxadiazol-3-yl)-phenylamine (Reference Example 25, 158 mg, 0.902 mmol) and (rac)-6-methoxy-8-(4-methyl-piperazin-1-yl)-chroman-2-carboxylic acid hydrochloride (Reference Example 14, 275 mg, 0.898 mmol) in DMF (10 mL) containing Hunigs base (0.35 mL, 2.0 mmol) was added TBTU (322 mg, 0.977 mmol) all in one portion. Reaction was stirred for 18 h, DMF was evaporated under reduced pressure, and residue was mixed with 20% aqueous K₂CO₃. This was extracted with 9:1 (EtOAc:DCM) (2×30 mL). Extracts were combined, dried over Na₂SO₄, filtered, and evaporated under reduced pressure. Product was purified by flash chromatography on silica using a gradient 100% DCM to 10:1 DCM: MeOH to give 128 mg (31%) of solid product. MS: m/z 464 (M+H); ¹H NMR (300.132 MHz, CDCl₃) δ 8.99 (s, 1H), 8.06 (d, J=8.5 Hz, 2H), 7.73 (d, J=8.5 Hz, 2H), 6.46 (d, J=2.7 Hz, 1H), 6.31 (d, J=2.5 Hz, 1H), 4.71 (dd, J=9.9, 3.8 Hz, 1H), 3.75 (s, 3H), 3.39-3.25 (m, 2H), 3.05-2.50 (m, 9H), 2.43 (s, 3H), 2.38 (s, 3H), 2.13-1.96 (m, 1H).

### Examples 58A and 58B

The enantiomers could be separated using any of the following methods and columns including: Preparative HPLC using 21×250 mm columns (˜20 mL/min flow rates) with column packings including Chiralpak AD, Chiralpak OD or Chiralpak OJ and mixtures of either EtOH and Hexane or isopropanol and hexane or supercritical fluid chromatography using columns such as Chiracel OD or Chiracel OD-H with either methanol/CO2/dimethylethylamine or ethanol/CO2/dimethylethylamine yielding (+)-6-methoxy-N-(4-(5-methyl-1,2,4-oxadiazol-3-yl)phenyl)-8-(4-methylpiperazin-1-yl)chroman-2-carboxamide and (−)-6-methoxy-N-(4-(5-methyl-1,2,4-oxadiazol-3-yl)phenyl)-8-(4-methylpiperazin-1-yl)chroman-2-carboxamide.

### Example 59

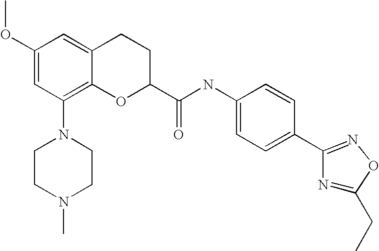

### N-(4-(5-ethyl-1,2,4-oxadiazol-3-yl)phenyl)-6-methoxy-8-(4-methylpiperazin-1-yl)chroman-2=carboxamide

4-(5-Ethyl-[1,2,4]oxadiazol-3-yl)-phenylamine (Reference Example 26, 170 mg, 0.898 mmol) was combined with (rac)-6-methoxy-8-(4-methyl-piperazin-1-yl)-chroman-2-carboxylic acid hydrochloride (Reference Example 14, 320 mg, 0.997 mmol) in an analogous fashion to that described in Example 58 to give 198 mg (46%) of product. MS: m/z 478 (M+H); ¹HNMR (300.132 MHz, CDCl₃) δ 8.98 (s, 1H), 8.07 (d, J=8.5 Hz, 2H), 7.73 (d, J=8.5 Hz, 2H), 6.46 (d, J=2.7 Hz, 1H), 6.31 (d, J=2.5 Hz, 1H), 4.71 (dd, J=9.9, 3.8 Hz, 1H), 3.75 (s, 3H), 3.39-3.25 (m, 2H), 2.97 (q, J=7.6 Hz, 2H), 3.05-2.50 (m, 9H), 2.37 (s, 3H), 2.13-1.96 (m, 1H), 1.45 (t, J=7.6 Hz, 3H).

### Examples 59A and 59B

The enantiomers could be separated using any of the following methods and columns including: Preparative HPLC using 21×250 mm columns (˜20 mL/min flow rates) with column packings including Chiralpak AD, Chiralpak OD or Chiralpak OJ and mixtures of either EtOH and Hexane or isopropanol and hexane or supercritical fluid chromatography using columns such as Chiracel OD or Chiracel OD-H with either methanol/CO2/dimethylethylamine or ethanol/CO2/dimethylethylamine yielding (+)-N-(4-(5-ethyl-1,2,4-oxadiazol-3-yl)phenyl)-6-methoxy-8-(4-methylpiperazin-1-yl)chroman-2-carboxamide and (−)-N-(4-(5-ethyl-1,2,4-oxadiazol-3-yl)phenyl)-6-methoxy-8-(4-methylpiperazin-1-yl)chroman-2-carboxamide.

### Example 60

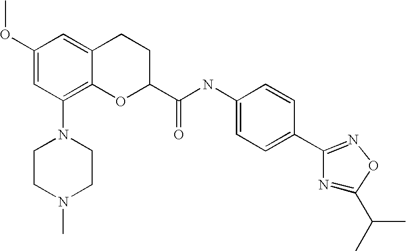

### N-(4-(5-isopropyl-1,2,4-oxadiazol-3-yl)phenyl)-6-methoxy-8-(4-methylpiperazin-1-yl)chroman-2-carboxamide

4-(5-isopropyl-[1,2,4]oxadiazol-3-yl)-phenylamine (Reference Example 27, 266 mg, 1.31 mmol) was combined with (rac)-6-methoxy-8-(4-methyl-piperazin-1-yl)-chroman-2-carboxylic acid hydrochloride (Reference Example 14, 466 mg, 1.36 mmol) in an analogous fashion to that described in Example 58 to give 177 mg (46%) of product. MS: m/z 492 (M+H); ¹H NMR (300.132 MHz, CDCl₃) δ 8.97 (s, 1H), 8.08 (d, J=8.5 Hz, 2H), 7.73 (d, J=8.5 Hz, 2H), 6.46 (d, J=2.7 Hz, 1H), 6.31 (d, J=2.5 Hz, 1H), 4.70 (dd, J=9.9, 3.8 Hz, 1H), 3.75 (s, 3H), 3.39-3.20 (m, 3H), 3.05-2.50 (m, 9H), 2.38 (s, 3H), 2.13-1.96 (m, 1H), 1.46 (d, J=7.2 Hz, 6H).

The enantiomers were separated using chiral SFC (Chiralpak AD, 30% IPA/CO₂+0.5% Dimethylethylamine) yielding N-(4-(5-isopropyl-1,2,4-oxadiazol-3-yl)phenyl)-6-methoxy-8-(4-methylpiperazin-1-yl)chroman-2-carboxamide (Isomer 1, rt=4.06 min) and N-(4-(5-isopropyl-1,2,4-oxadiazol-3-yl)phenyl)-6-methoxy-8-(4-methylpiperazin-1-yl)chroman-2-carboxamide (Isomer 2, rt=4.93 min).

Isomer 1: MS: m/z 492 (M+H). ¹H NMR (300.132 MHz, CDCl₃) δ 8.97 (s, 1H), 8.08 (d, J=8.5 Hz, 2H), 7.73 (d, J=8.5 Hz, 2H), 6.46 (d, J=2.7 Hz, 1H), 6.31 (d, J=2.5 Hz, 1H), 4.70 (dd, J=9.9, 3.8 Hz, 1H), 3.75 (s, 3H), 3.39-3.20 (m, 3H), 3.05-2.50 (m, 9H), 2.38 (s, 3H), 2.13-1.96 (m, 1H), 1.46 (d, J=7.2 Hz, 6H).

Isomer 2: MS: m/z 492 (M+H). ¹H NMR (300.132 MHz, CDCl₃) δ 8.97 (s, 1H), 8.08 (d, J=8.5 Hz, 2H), 7.73 (d, J=8.5 Hz, 2H), 6.46 (d, J=2.7 Hz, 1H), 6.31 (d, J=2.5 Hz, 1H), 4.70 (dd, J=9.9, 3.8 Hz, 1H), 3.75 (s, 3H), 3.39-3.20 (m, 3H), 3.05-2.50 (m, 9H), 2.38 (s, 3H), 2.13-1.96 (m, 1H), 1.46 (d, J=7.2 Hz, 6H).

### Example 61

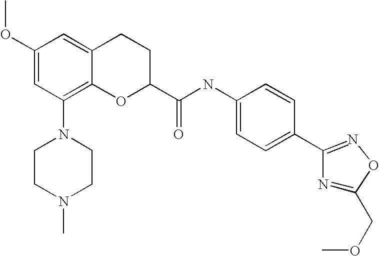

### 6-methoxy-N-(4-(5-(methoxymethyl)-1,2,4-oxadiazol-3-yl)phenyl)-8-(4-methylpiperazin-1-yl)chroman-2-carboxamide

4-(5-Methoxymethyl-[1,2,4]oxadiazol-3-yl)-phenylamine (Reference Example 28, 266 mg, 1.30 mmol) was combined with (rac)-6-methoxy-8-(4-methyl-piperazin-1-yl)-chroman-2-carboxylic acid hydrochloride (Reference Example 14, 397 mg, 1.30 mmol) in an analogous fashion to that described in Example 58 to give 227 mg (36%) of product. MS: m/z 494 (M+H); ¹H NMR (300.132 MHz, CDCl₃) δ 9.00 (s, 1H), 8.10 (d, J=8.7 Hz, 2H), 7.75 (d, J=8.7 Hz, 2H), 6.47 (d, J=2.7 Hz, 1H), 6.31 (d, J=2.5 Hz, 1H), 4.74 (s, 2H), 4.71 (dd, J=9.9, 3.8 Hz, 1H), 3.75 (s, 3H), 3.56 (s, 3H), 3.39-3.26 (m, 2H), 3.05-2.53 (m, 9H), 2.38 (s, 3H), 2.13-1.96 (m, 1H).

### Examples 61A and 61B

The enantiomers could be separated using any of the following methods and columns including: Preparative HPLC using 21×250 mm columns (˜20 mL/min flow rates) with column packings including Chiralpak AD, Chiralpak OD or Chiralpak OJ and mixtures of either EtOH and Hexane or isopropanol and hexane or supercritical fluid chromatography using columns such as Chiracel OD or Chiracel OD-H with either methanol/CO2/dimethylethylamine or ethanol/CO2/dimethylethylamine yielding (+)-6-methoxy-N-(4-(5-(methoxymethyl)-1,2,4-oxadiazol-3-yl)phenyl)-8-(4-methylpiperazin-1-yl)chroman-2-carboxamide and (−)-6-methoxy-N-(4-(5-(methoxymethyl)-1,2,4-oxadiazol-3-yl)phenyl)-8-(4-methylpiperazin-1-yl)chroman-2-carboxamide.

### Example 62

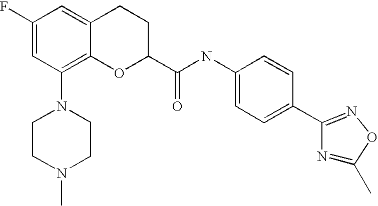

### 6-Fluoro-N-(4-(5-methyl-1,2,4-oxadiazol-3-yl)phenyl)-8-(4-methylpiperazin-1-yl)chroman-2-carboxamide

4-(5-Methyl-[1,2,4]oxadiazol-3-yl)-phenylamine (Reference Example 25, 143 mg, 0.815 mmol) was combined with (rac)-6-fluoro-8-(4-methyl-piperazin-1-yl)-chroman-2-carboxylic acid hydrochloride (Reference Example 15, 270 mg, 0.816 mmol) in an analogous fashion to that described in Example 58 to give 203 mg (55%) of product. MS: m/z 452 (M+H); ¹H NMR (300.132 MHz, CDCl₃) δ 8.92 (s, 1H), 8.07 (d, J=8.7 Hz, 2H), 7.74 (d, J=8.7 Hz, 2H), 6.59 (dm, J=10.2 Hz, 1H), 6.50 (dm, J=10.2 Hz, 1H), 4.72 (dd, J=9.9, 3.6 Hz, 1H), 3.45-3.25 (m, 2H), 2.64 (s, 3H), 3.10-2.50 (m, 9H), 2.38 (s, 3H), 2.14-1.94 (m, 1H); ¹⁹F NMR (282.384 MHz, CDCl₃)δ −121.055.

### Examples 62A and 62B

The enantiomers could be separated using any of the following methods and columns including: Preparative HPLC using 21×250 mm columns (˜20 mL/min flow rates) with column packings including Chiralpak AD, Chiralpak OD or Chiralpak OJ and mixtures of either EtOH and Hexane or isopropanol and hexane or supercritical fluid chromatography using columns such as Chiracel OD or Chiracel OD-H with either methanol/CO2/K dimethylethylamine or ethanol/CO2/dimethylethylamine yielding (+)-6-Fluoro-N-(4-(5-methyl-1,2,4-oxadiazol-3-yl)phenyl)-8-(4-methylpiperazin-1-yl)chroman-2-carboxamide and (−)-6-Fluoro-N-(4-(5-methyl-1,2,4-oxadiazol-3-yl)phenyl)-8-(4-methylpiperazin-1-yl)chroman-2-carboxamide.

### Example 63

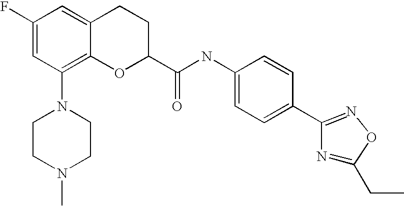

### N-(4-(5-ethyl-1,2,4-oxadiazol-3-yl)phenyl)-6-fluoro-8-(4-methylpiperazin-1-yl)chroman-2-carboxamide

4-(5-Ethyl-[1,2,4]oxadiazol-3-yl)-phenylamine (Reference Example 26, 170 mg, 0.90 mmol) was combined with (rac)-6-fluoro-8-(4-methyl-piperazin-1-yl)-chroman-2-carboxylic acid hydrochloride (Reference Example 15, 297 mg, 0.898 mmol) in an analogous fashion to that described in Example 58 to give 145 mg (35%) of product. MS: m/z 466 (M+H); ¹H NMR (300.132 MHz, CDCl₃) δ 8.92 (s, 1H), 8.08 (d, J=8.7 Hz, 2H), 7.73 (d, J=8.7 Hz, 2H), 6.59 (dm, J=10.2 Hz, 1H), 6.50 (dm, J=10.2 Hz, 1H), 4.72 (dd, J=9.9, 3.6 Hz, 1H), 3.45-3.22 (m, 2H), 3.10-2.50 (m, 11H), 2.38 (s, 3H), 2.14-1.94 (m, 1H), 1.45 (t, J=7.8, 3H); ¹⁹F NMR (282.384 MHz, CDCl₃)δ −121.064

### Examples 63A and 63B

The enantiomers could be separated using any of the following methods and columns including: Preparative HPLC using 21×250 mm columns (˜20 mL/min flow rates) with column packings including Chiralpak AD, Chiralpak OD or Chiralpak OJ and mixtures of either EtOH and Hexane or isopropanol and hexane or supercritical fluid chromatography using columns such as Chiracel OD or Chiracel OD-H with either methanol/CO2/dimethylethylamine or ethanol/CO2/dimethylethylamine yielding (+)-N-(4-(5-ethyl-1,2,4-oxadiazol-3-yl)phenyl)-6-fluoro-8-(4-methylpiperazin-1-yl)chroman-2-carboxamide and (−)-N-(4-(5-ethyl-1,2,4-oxadiazol-3-yl)phenyl)-6-fluoro-8-(4-methylpiperazin-1-yl)chroman-2-carboxamide.

### Example 64

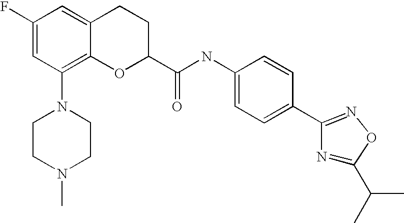

### N-(4-(5-isopropyl-1,2,4-oxadiazol-3-yl)phenyl)-6-fluoro-8-(4-methylpiperazin-1-yl)chroman-2-carboxamide

4-(5-Isopropyl-[1,2,4]oxadiazol-3-yl)-phenylamine (Reference Example 27, 266 mg, 1.31 mmol) was combined with 6-fluoro-8-(4-methyl-piperazin-1-yl)-chroman-2-carboxylic acid (Reference Example 15, 488 mg, 1.31 mmol) in an analogous fashion to that described in Example 58 to give 227 mg (36%) of product. MS: m/z 480 (M+H); ¹H NMR (300.132 MHz, CDCl₃) δ 8.82 (s, 1H), 8.08 (d, J=8.4 Hz, 2H), 7.71 (d, J=8.4 Hz, 2H), 6.59 (dd, J=9.9, 2.7 Hz, 1H), 6.50 (dm, J=9.9, 2.7 Hz, 1H), 4.73 (dd, J=9.9, 3.6 Hz, 1H), 3.45-3.10 (m, 2H), 3.28 (septet, J=6.9 Hz, 1H), 3.10-2.35 (m, 11H), 2.14-1.97 (m, 1H), 1.46 (d, J=6.9 Hz, 6H); ¹⁹F NMR (282.384 MHz, CDCl₃)δ −120.893.

### Examples 64A and 64B

The enantiomers could be separated using any of the following methods and columns including: Preparative HPLC using 21×250 mm columns (˜20 mL/min flow rates) with column packings including Chiralpak AD, Chiralpak OD or Chiralpak OJ and mixtures of either EtOH and Hexane or isopropanol and hexane or supercritical fluid chromatography using columns such as Chiracel OD or Chiracel OD-H with either methanol/CO2/dimethylethylamine or ethanol/CO2/dimethylethylamine yielding (+)-N-(4-(5-isopropyl-1,2,4-oxadiazol-3-yl)phenyl)-6-fluoro-8-(4-methylpiperazin-1-yl)chroman-2-carboxamide and (−)-N-(4-(5-isopropyl-1,2,4-oxadiazol-3-yl)phenyl)-6-fluoro-8-(4-methylpiperazin-1-yl)chroman-2-carboxamide.

### Example 65

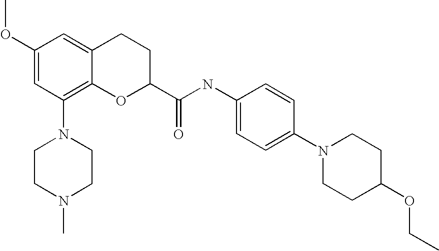

### N-(4-(4-ethoxypiperidin-1-yl)phenyl)-6-methoxy-8-(4-methylpiperazin-1-yl)chroman-2-carboxamide

4-(4-ethoxy-piperidin-1-yl)phenylamine (Reference Example 48, 302 mg, 1.37 mmol) was combined with (rac)-6-methoxy-8-(4-methyl-piperazin-1-yl)-chroman-2-carboxylic acid hydrochloride (Reference Example 14, 382 mg, 1.25 mmol) in an analogous fashion to that described in Example 58 to give 390 mg (62%) of product. MS: m/z 509 (M+H); ¹H NMR (300.132 MHz, CDCl₃) δ 8.64 (s, 1H), 7.45 (d, J=8.7 Hz, 2H), 6.92 (d, J=8.7 Hz, 2H), 6.44 (d, J=3.0 Hz, 1H), 6.29 (d, J=3.0 Hz, 1H), 4.65 (dd, J=9.9, 3.6 Hz, 1H), 3.75 (s, 3H), 3.54 (q, 2H), 3.60-3.38 (m, 3H), 3.35-3.22 (m, 2H), 3.05-2.48 (m, 11H), 2.37 (s, 3H), 2.13-1.92 (m, 3H), 1.80-1.62 (m, 2H), 1.22 (t, J=6.9 Hz, 3H).

The enantiomers were separated using chiral SFC (Chiralpak AD, 50% IPA/CO₂+0.5% Dimethylethylamine) yielding N-(4-(4-ethoxypiperidin-1-yl)phenyl)-6-methoxy-8-(4-methylpiperazin-1-yl)chroman-2-carboxamide (Isomer 1, rt=2.96 min) and N-(4-(4-ethoxypiperidin-1-yl)phenyl)-6-methoxy-8-(4-methylpiperazin-1-yl)chroman-2-carboxamide (Isomer 2, rt=4.57 min).

Isomer 1: MS: m/z 509 (M+H); ¹H NMR (300.132 MHz, CDCl₃) δ 8.64 (s, 1H), 7.45 (d, J=8.7 Hz, 2H), 6.92 (d, J=8.7 Hz, 2H), 6.44 (d, J=3.0 Hz, 1H), 6.29 (d, J=3.0 Hz, 1H), 4.65 (dd, J=9.9, 3.6 Hz, 1H), 3.75 (s, 3H), 3.54 (q, 2H), 3.60-3.38 (m, 3H), 3.35-3.22 (m, 2H), 3.05-2.48 (m, 1H), 2.37 (s, 3H), 2.13-1.92 (m, 3H), 1.80-1.62 (m, 2H), 1.22 (t, J=6.9 Hz, 3H).

Isomer 2: MS: m/z 509 (M+H); ¹H NMR (300.132 MHz, CDCl₃) δ 8.64 (s, 1H), 7.45 (d, J=8.7 Hz, 2H), 6.92 (d, J=8.7 Hz, 2H), 6.44 (d, J=3.0 Hz, 1H), 6.29 (d, J=3.0 Hz, 1H), 4.65 (dd, J=9.9, 3.6 Hz, 1H), 3.75 (s, 3H), 3.54 (q, 2H), 3.60-3.38 (m, 3H), 3.35-3.22 (m, 2H), 3.05-2.48 (m, 11H), 2.37 (s, 3H), 2.13-1.92 (m, 3H), 1.80-1.62 (m, 2H), 1.22 (t, J=6.9 Hz, 3H).

### Example 67

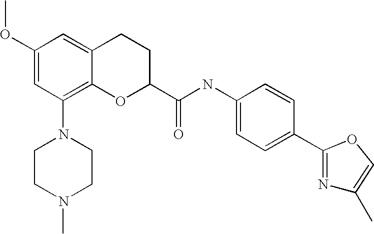

### 6-methoxy-N-(4-(4-methyloxazol-2-yl)phenyl)-8-(4-methylpiperazin-1-yl)chroman-2-carboxamide

4-(4-Methyl-oxazol-2-yl)-phenylamine (Reference Example 30, 155 mg, 0.89 mmol) was combined with (rac)-6-methoxy-8-(4-methyl-piperazin-1-yl)-chroman-2-carboxylic acid hydrochloride (Reference Example 14, 273 mg, 0.89 mmol) in an analogous fashion to that described in Example 58 to give 33 mg (8%) of product. MS: m/z 463 (M+H); ¹H NMR (300.132 MHz, CDCl₃) δ 8.96 (s, 1H), 8.01 (d, J=8.4 Hz, 2H), 7.70 (d, J=8.4 Hz, 2H), 7.41 (s, 1H), 6.46 (d, J=2.7 Hz, 1H), 6.30 (d, J=2.7 Hz, 1H), 4.70 (dd, J=9.9, 3.9 Hz, 1H), 3.75 (s, 3H), 3.40-3.25 (m, 2H), 3.05-2.50 (m, 9H), 2.37 (s, 3H), 2.24 (s, 3H), 2.13-1.94 (m, 1H).

### Examples 67A and 67B

The enantiomers could be separated using any of the following methods and columns including: Preparative HPLC using 21×250 mm columns (˜20 mL/min flow rates) with column packings including Chiralpak AD, Chiralpak OD or Chiralpak OJ and mixtures of either EtOH and Hexane or isopropanol and hexane or supercritical fluid chromatography using columns such as Chiracel OD or Chiracel OD-H with either methanol/CO2/dimethylethylamine or ethanol/CO2/dimethylethylamine yielding (+)-6-methoxy-N-(4-(4-methyloxazol-2-yl)phenyl)-8-(4-methylpiperazin-1-yl)chroman-2-carboxamide and (−)-6-methoxy-N-(4-(4-methyloxazol-2-yl)phenyl)-8-(4-methylpiperazin-1-yl)chroman-2-carboxamide.

### Example 72

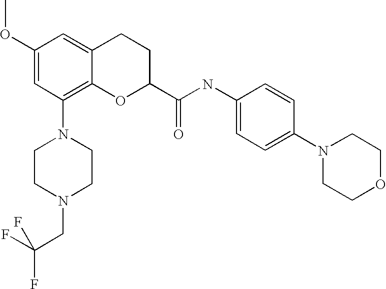

### 6-methoxy-N-(4-morpholinophenyl)-8-(4-(2,2,2-trifluoroethyl)piperazin-1-yl)chroman-2-carboxamide

To a stirred solution of 6-methoxy-8-[4-(2,2,2-trifluoro-ethyl)-piperazin-1-yl]-chroman-2-carboxylic acid (Reference Example 35, 200 mg, 0.52 mmol), 4-morpholin-4-yl-phenylamine (110 mg, 0.62 mmol), and triethylamine (0.30 mL, 2.15 mmol) in 10 mL DCM was added HATU (280 mg, 0.74 mmol). After 18 h the reaction was quenched with 20 mL aqueous 20% K₂CO₃ and extracted with DCM (3×30 mL). Organic extracts were combined, dried over Na₂SO₄, filtered, and evaporated under reduced pressure to give an amber oil that was purified by Prep RPHPLC. [Conditions: 25-90% MeCN (w/0.1% TFA) over 20 minutes, 7 min equilibration at 25% MeCN, 5 min flush at 90% MeCN. Used a 2″ diameter radial compression C8 dynamax 60 Å column]. Residue after evaporation was mixed with 15 mL 20% aqueous K₂CO₃ and extracted with DCM (3×30 mL). Extracts were combined, dried over Na₂SO₄, filtered, and evaporated under reduced pressure to give a solid (31 mg, 12%) product. MS: m/z 535 (M+H). ¹H NMR (300.132 MHz, CDCl₃) δ 8.64 (s, 1H), 7.47 (d, J=8.9 Hz, 2H), 6.90 (d, J=8.9 Hz, 2H), 6.43 (d, J=2.6 Hz, 1H), 6.31 (d, J=2.4 Hz, 1H), 4.94 (dd, J=10.0, 3.7 Hz, 1H), 3.91-3.81 (m, 4H), 3.75 (s, 3H), 3.35-3.23 (m, 2H), 3.00-2.70 (m, 14H), 2.62-2.48 (m, 1H), 2.12-1.93 (m, 1H).

### Examples 72A and 72B

The enantiomers could be separated using any of the following methods and columns including: Preparative HPLC using 21×250 mm columns (˜20 mL/min flow rates) with column packings including Chiralpak AD, Chiralpak OD or Chiralpak OJ and mixtures of either EtOH and Hexane or isopropanol and hexane or supercritical fluid chromatography using columns such as Chiracel OD or Chiracel OD-H with either methanol/CO2/dimethylethylamine or ethanol/CO2/dimethylethylamine yielding (+)-6-methoxy-N-(4-morpholinophenyl)-8-(4-(2,2,2-trifluoroethyl)piperazin-1-yl)chroman-2-carboxamide and (−)-6-methoxy-N-(4-morpholinophenyl)-8-(4-(2,2,2-trifluoroethyl)piperazin-1-yl)chroman-2-carboxamide.

### Example 73

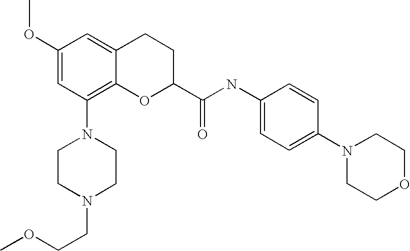

### 6-methoxy-8-(4-(2-methoxyethyl)piperazin-1-yl)-N-(4-morpholinophenyl)chroman-2-carboxamide

4-Morpholin-4-yl-phenylamine (140 mg, 0.77 mmol) was combined with 6-methoxy-8-[4-(2-methoxy-ethyl)-piperazin-1-yl]-chroman-2-carboxylic acid (Reference Example 38, 270 mg, 0.77 mmol) in an analogous fashion to that described in Example 72 to give 41 mg (11%) of product. MS: m/z 469 (M+H). ¹H-NMR (300.132 MHz, CDCl₃) δ 8.68 (s, 1H), 7.48 (d, J=8.9 Hz, 2H), 6.89 (d, J=8.9 Hz, 2H), 6.44 (d, J=2.8 Hz, 1H), 6.29 (d, J=2.7 Hz, 1H), 4.66 (dd, J=10.0, 3.7 Hz, 1H), 3.91-3.81 (m, 4H), 3.74 (s, 3H), 3.55 (t, J=5.5 Hz, 2H), 3.37 (s, 3H), 3.39-3.26 (m, 2H), 3.16-3.08 (m, 4H), 3.05-2.48 (m, 11H), 2.12-1.92 (m, 1H).

### Examples 73A and 73B

The enantiomers could be separated using any of the following methods and columns including: Preparative HPLC using 21×250 mm columns (˜20 mL/min flow rates) with column packings including Chiralpak AD, Chiralpak OD or Chiralpak OJ and mixtures of either EtOH and Hexane or isopropanol and hexane or supercritical fluid chromatography using columns such as Chiracel OD or Chiracel OD-H with either methanol/CO2/dimethylethylamine or ethanol/CO2/dimethylethylamine yielding (+)-6-methoxy-8-(4-(2-methoxyethyl)piperazin-1-yl)-N-(4-morpholinophenyl)chroman-2-carboxamide and (−)-6-methoxy-8-(4-(2-methoxyethyl)piperazin-1-yl)-N-(4-morpholinophenyl)chroman-2-carboxamide.

### Example 75

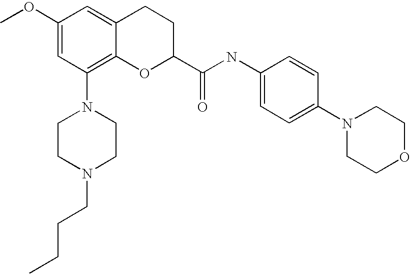

### 8-(4-butylpiperazine-1-yl)-6-methoxy-N-(4-morpholinophenyl)chroman-2-carboxamide

4-Morpholin-4-yl-phenylamine (76 mg, 0.43 mmol) was combined with 8-(4-butyl-piperazin-1-yl)-6-methoxy-chroman-2-carboxylic acid (Reference Example 40, 151 mg, 0.43 mmol) in an analogous fashion to that described in Example 58. Compound needed additional purification by Prep RPHPLC. [Conditions: 25-90% MeCN (w/0.1% TFA) over 20 minutes, 7 min equilibration at 25% MeCN, 5 min flush at 90% MeCN. Used a 2″ diameter radial compression C8 dynamax 60 Å column]. Residue after evaporation was mixed with 15 mL 20% aqueous K₂CO₃ and extracted with DCM (3×30 mL). Extracts were combined, dried over Na₂SO₄, filtered, and evaporated under reduced pressure to give 33 mg (15%) of product.

MS: m/z 509 (M+H); ¹H NMR (300.132 MHz, CD₃CN) δ 8.69 (s, 1H), 7.47 (d, J=9.1 Hz, 2H), 6.94 (d, J=9.0 Hz, 2H), 6.43 (d, J=2.8 Hz, 1H), 6.36 (d, J=2.7 Hz, 1H), 4.64 (dd, J=10.0, 4.0 Hz, 1H), 3.85-3.75 (m, 4H), 3.73 (s, 3H), 3.28-3.16 (m, 2H), 3.16-3.04 (m, 4H), 3.01-2.69 (m, 3H), 2.68-2.51 (m, 4H), 2.50-2.34 (m, 1H), 2.38 (t, J=7.3 Hz, 2H), 2.19-2.08 (m, 2H), 1.56-1.27 (m, 4H), 0.95 (t, J=7.2 Hz, 3H).

### Examples 75A and 75B

The enantiomers could be separated using any of the following methods and columns including: Preparative HPLC using 21×250 mm columns (˜20 mL/min flow rates) with column packings including Chiralpak AD, Chiralpak OD or Chiralpak OJ and mixtures of either EtOH and Hexane or isopropanol and hexane or supercritical fluid chromatography using columns such as Chiracel OD or Chiracel OD-H with either methanol/CO2/dimethylethylamine or ethanol/CO2/dimethylethylamine yielding (+)-8-(4-butylpiperazine-1-yl)-6-methoxy-N-(4-morpholinophenyl)chroman-2-carboxamide and (−)-8-(4-butylpiperazine-1-yl)-6-methoxy-N-(4-morpholinophenyl)chroman-2-carboxamide.

### Example 76

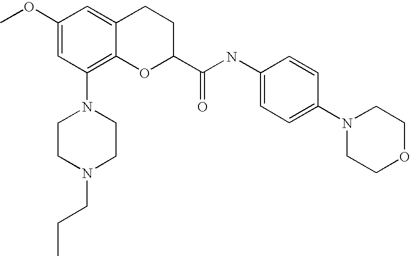

### 8-(4-propylpiperazin-1-yl)-6-methoxy-N-(4-morpholinophenyl)chroman-2-carboxamide

4-Morpholin-4-yl-phenylamine (53 mg, 0.30 mmol) was combined with 6-methoxy-8-(4-propyl-piperazin-1-yl)-chroman-2-carboxylic acid (Reference Example 41, 104 mg, 0.31 mmol) in an analogous fashion to that described in Example 75 to give 31 mg (21%) of product. MS: m/z 495 (M+H); ¹H NMR (300.132 MHz, CDCl₃) δ 8.69 (s, 1H), 7.49 (d, J=8.9 Hz, 2H), 6.90 (d, J=8.9 Hz, 2H), 6.44 (d, J=2.8 Hz, 1H), 6.29 (d, J=2.7 Hz, 1H), 4.66 (dd, J=10.0, 3.8 Hz, 1H), 3.86 (t, J=4.7 Hz, 4H), 3.75 (s, 3H), 3.40-3.23 (m, 2H), 3.19-3.07 (m, 4H), 3.05-2.49 (m, 9H), 2.38 (t, J=7.6 Hz, 2H), 2.15-1.89 (m, 1H), 1.55 (sextet, J=7.4 Hz, 2H), 0.93 (t, J=7.3 Hz, 3H).

### Examples 76A and 76B

The enantiomers could be separated using any of the following methods and columns including: Preparative HPLC using 21×250 mm columns (˜20 mL/min flow rates) with column packings including Chiralpak AD, Chiralpak OD or Chiralpak OJ and mixtures of either EtOH and Hexane or isopropanol and hexane or supercritical fluid chromatography using columns such as Chiracel OD or Chiracel OD-H with either methanol/CO2/dimethylethylamine or ethanol/CO2/dimethylethylamine yielding (+)-8-(4-propylpiperazin-1-yl)-6-methoxy-N-(4-morpholinophenyl)chroman-2-carboxamide and (−)-8-(4-propylpiperazin-1-yl)-6-methoxy-N-(4-morpholinophenyl)chroman-2-carboxamide.

### Example 77

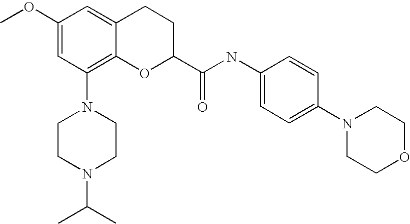

### 8-(4-Isopropylpiperazin-1-yl)-6-methoxy-N-(4-morpholinophenyl)chroman-2-carboxamide

4-Morpholin-4-yl-phenylamine (125 mg, 0.70 mmol) was combined with 8-(4-isopropyl-piperazin-1-yl)-6-methoxy-chroman-2-carboxylic acid (Reference Example 42, 242 mg, 0.72 mmol) in an analogous fashion to that described in Example 58 to give 99 mg (29%) of product. MS: m/z 495 (M+H); ¹H NMR (300.132 MHz, CDCl₃) δ 8.72 (s, 1H), 7.50 (d, J=8.7 Hz, 2H), 6.89 (d, J=8.7 Hz, 2H), 6.44 (d, J=2.5 Hz, 1H), 6.29 (d, J=2.5 Hz, 1H), 4.66 (dd, J=10.2, 3.7 Hz, 1H), 3.94-3.79 (m, 4H), 3.75 (s, 3H), 3.39-3.24 (m, 2H), 3.20-3.06 (m, 4H), 3.05-2.66 (m, 9H), 2.65-2.49 (m, 1H), 2.11-1.90 (m, 1H), 1.10 (d, J=6.4 Hz, 6H).

### Examples 77A and 77B

The enantiomers could be separated using any of the following methods and columns including: Preparative HPLC using 21×250 mm columns (˜20 mL/min flow rates) with column packings including Chiralpak AD, Chiralpak OD or Chiralpak OJ and mixtures of either EtOH and Hexane or isopropanol and hexane or supercritical fluid chromatography using columns such as Chiracel OD or Chiracel OD-H with either methanol/CO2/dimethylethylamine or ethanol/CO2/dimethylethylamine yielding (+)-8-(4-isopropylpiperazin-1-yl)-6-methoxy-N-(4-morpholinophenyl)chroman-2-carboxamide and (−)-8-(4-Isopropylpiperazin-1-yl)-6-methoxy-N-(4-morpholinophenyl)chroman-2-carboxamide.

### Example 78

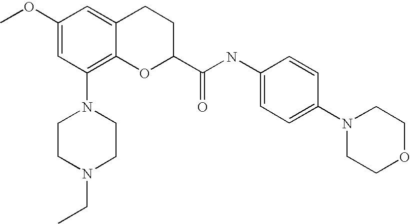

### 8-(4-Ethylpiperazin-1-yl)-6-methoxy-N-(4-morpholinophenyl)chroman-2-carboxamide

4-Morpholin-4-yl-phenylamine (130 mg, 0.73 mmol) was combined with 8-(4-ethyl-piperazin-1-yl)-6-methoxy-chroman-2-carboxylic acid (Reference Example 43, 231 mg, 0.72 mmol) in an analogous fashion to that described in Example 58 to give 70 mg (20%) of product. MS: m/z 481 (M+H); ¹H NMR (300.132 MHz, CDCl₃) δ 8.69 (s, 1H), 7.49 (d, J=8.9 Hz, 2H), 6.89 (d, J=8.9 Hz, 2H), 6.45 (d, J=2.6 Hz, 1H), 6.29 (d, J=2.6 Hz, 1H), 4.66 (dd, J=9.9, 3.6 Hz, 1H), 3.92-3.81 (m, 4H), 3.75 (s, 3H), 3.41-3.24 (m, 2H), 3.19-3.07 (m, 4H), 3.06-2.43 (m, 1H), 2.13-1.91 (m, 1H), 1.13 (t, J=7.1 Hz, 3H).

### Examples 78A and 78B

The enantiomers could be separated using any of the following methods and columns including: Preparative HPLC using 21×250 mm columns (˜20 mL/min flow rates) with column packings including Chiralpak AD, Chiralpak OD or Chiralpak OJ and mixtures of either EtOH and Hexane or isopropanol and hexane or supercritical fluid chromatography using columns such as Chiracel OD or Chiracel OD-H with either methanol/CO2/dimethylethylamine or ethanol/CO2/dimethylethylamine yielding (+)-8-(4-Ethylpiperazin-1-yl)-6-methoxy-N-(4-morpholinophenyl)chroman-2-carboxamide and (−)-8-(4-Ethylpiperazin-1-yl)-6-methoxy-N-(4-morpholinophenyl)chroman-2-carboxamide.

### Example 79

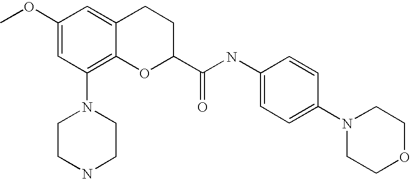

### 8-(piperazin-1-yl)-6-methoxy-N-(4-morpholinophenyl)chroman-2-carboxamide

### Example 79A

### 4-[6-Methoxy-2-(4-morpholin-4-yl-phenylcarbamoyl)-chroman-8-yl]-piperazine-1-carboxylic acid tert-butyl ester

4-Morpholin-4-yl-phenylamine (85 mg, 0.48 mmol) was combined with 4-(2-carboxy-6-methoxy-chroman-8-yl)-piperazine-1-carboxylic acid tert-butyl ester (Reference Example 44, 190 mg, 0.48 mmol) in an analogous fashion (except PyAOP was substituted for TBTU) to that described in Example 58 to give 180 mg (67%) of product; ¹H NMR (300.132 MHz, CDCl₃) δ 10.21 (s, 1H), 8.01 (d, J=8.7 Hz, 2H), 7.50 (d, J=8.7 Hz, 2H), 6.81 (d, J=2.7 Hz, 1H), 6.69 (d, J=2.7 Hz, 1H), 5.12-5.05 (m, 1H), 4.63 (d, J=9.2 Hz, 1H), 4.04-3.87 (m, 4H), 3.87-3.29 (m, 8H), 3.75 (s, 3H), 3.01-2.75 (m, 6H), 2.28-1.82 (m, 1H), 1.49 (s, 9H).

### Example 79B

### 8-(piperazin-1-yl)-6-methoxy-N-(4-morpholinophenyl)chroman-2-carboxamide

To a solution of 4-[6-methoxy-2-(4-morpholin-4-yl-phenylcarbamoyl)-chroman-8-yl]-piperazine-1-carboxylic acid tert-butyl ester (Example 79A, 80 mg, 0.14 mmol) in dichloromethane (5 mL) was added trifluoroacetic acid (1 mL). Reaction was stirred for 1 h. Solvents were evaporated under reduced pressure and product was purified by Prep RPHPLC. [Conditions: 25-90% MeCN (w/0.1% TFA) over 20 minutes, 7 min equilibration at 25% MeCN, 5 min flush at 90% MeCN. Used a 2″ diameter radial compression C8 dynamax 60 Å column]. Residue after evaporation was mixed with 15 mL 20% K₂CO₃ (aq) and extracted with DCM (3×30 mL). Extracts were combined, dried over Na₂SO₄, filtered, and evaporated under reduced pressure to give 17 mg (26%) of product. MS: m/z 453 (M+H); ¹H NMR (300.132 MHz, CD₃CN) δ 8.61 (s, 1H), 7.46 (d, J=9.0 Hz, 1H), 6.94 (d, J=9.0 Hz, 1H), 6.43 (d, J=2.9 Hz, 1H), 6.39 (d, J=2.9 Hz, 1H), 4.64 (dd, J=9.5, 3.6 Hz, 1H), 3.84-3.76 (m, 4H), 3.73 (s, 3H), 3.35-2.91 (m, 14H), 2.88 (dd, J=10.2, 5.2 Hz, 1H), 2.77 (dt, J=16.4, 4.9 Hz, 1H), 2.50-2.36 (m, 1H), 2.11-1.96 (m, 1H).

### Examples 79C and 79D

The enantiomers could be separated using any of the following methods and columns including: Preparative HPLC using 21×250 mm columns (˜20 mL/min flow rates) with column packings including Chiralpak AD, Chiralpak OD or Chiralpak OJ and mixtures of either EtOH and Hexane or isopropanol and hexane or supercritical fluid chromatography using columns such as Chiracel OD or Chiracel OD-H with either methanol/CO2/dimethylethylamine or ethanol/CO2/dimethylethylamine yielding (+)-8-(piperazin-1-yl)-6-methoxy-N-(4-morpholinophenyl)chroman-2-carboxamide and (−)-8-(piperazin-1-yl)-6-methoxy-N-(4-morpholinophenyl)chroman-2-carboxamide.

### Example 80

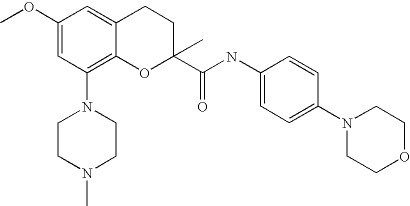

### 6-methoxy-2-methyl-8-(4-methylpiperazin-1-yl)-N-(4-morpholinophenyl)chroman-2-carboxamide

To a stirred solution of 6-methoxy-2-methyl-8-(4-methyl-piperazin-1-yl)-chroman-2-carboxylic acid HCL/LiCl salt (Reference Example 45, 400 mg, 1.01 mmol), 4-morpholin-4-yl-phenylamine (200 mg, 1.12 mmol), and diisopropylethylamine (0.62 mL, 3.6 mmol) in 10 mL DCM was added HATU (420 mg, 1.11 mmol). After 18 h the reaction was concentrated under reduced pressure and residue was purified by chromatography on silica 100% DCM to 98:2 to 96:4 DCM:MeOH (w/0.1% aq NH₃). Purified product was triturated in diethyl ether to give an off-white solid (325 mg, 64%). MS: m/z 481 (M+H); ¹H NMR (300.132 MHz, CDCl₃) δ 8.78 (s, 1H), 7.37 (d, J=9.3 Hz, 2H), 6.85 (d, J=9.3 Hz, 2H), 6.41 (d, J=2.8 Hz, 1H), 6.26 (d, J=2.8 Hz, 1H), 3.89-3.78 (m, 4H), 3.73 (s, 3H), 3.52-3.39 (m, 2H), 3.13-3.04 (m, 4H), 2.96-2.82 (m, 2H), 2.74 (t, J=6.6 Hz, 2H), 2.79-2.56 (m, 4H), 2.38 (s, 3H), 2.45-2.31 (m, 1H), 2.06 (dt, J=13.9, 7.0 Hz, 1H), 1.65 (s, 3H).

The enantiomers were separated using chiral SFC (Chiralpak AD-H, 40% EtOH/CO₂+0.5% Dimethylethylamine) yielding 6-methoxy-2-methyl-8-(4-methylpiperazin-1-yl)-N-(4-morpholinophenyl)chroman-2-carboxamide (Isomer 1, rt=2.82 min) and 6-methoxy-2-methyl-8-(4-methylpiperazin-1-yl)-N-(4-morpholinophenyl)chroman-2-carboxamide (Isomer 2, rt=4.23 min).

Isomer 1: MS: m/z 480 (M+). ¹H NMR (300.132 MHz, CDCl₃) δ 8.78 (s, 1H), 7.37 (d, J=9.3 Hz, 2H), 6.85 (d, J=9.3 Hz, 2H), 6.41 (d, J=2.8 Hz, 1H), 6.26 (d, J=2.8 Hz, 1H), 3.89-3.78 (m, 4H), 3.73 (s, 3H), 3.52-3.39 (m, 2H), 3.13-3.04 (m, 4H), 2.96-2.82 (m, 2H), 2.74 (t, J=6.6 Hz, 2H), 2.79-2.56 (m, 4H), 2.38 (s, 3H), 2.45-2.31 (m, 1H), 2.06 (dt, J=13.9, 7.0 Hz, 1H), 1.65 (s, 3H).

Isomer 2: MS: m/z 480 (M+). ¹H NMR (300.132 MHz, CDCl₃) δ 8.78 (s, 1H), 7.37 (d, J=9.3 Hz, 2H), 6.85 (d, J=9.3 Hz, 2H), 6.41 (d, J=2.8 Hz, 1H), 6.26 (d, J=2.8 Hz, 1H), 3.89-3.78 (m, 4H), 3.73 (s, 3H), 3.52-3.39 (m, 2H), 3.13-3.04 (m, 4H), 2.96-2.82 (m, 2H), 2.74 (t, J=6.6 Hz, 2H), 2.79-2.56 (m, 4H), 2.38 (s, 3H), 2.45-2.31 (m, 1H), 2.06 (dt, J=13.9, 7.0 Hz, 1H), 1.65 (s, 3H).

### Example 105

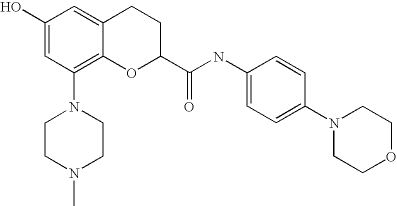

### 6-Hydroxy-8-(4-methylpiperazin-1-yl)-N-(4-morpholinophenyl)chroman-2-carboxamide

This compound was prepared in 77% yield from (+/−)-6-Methoxy-8-(4-methyl-piperazin-1-yl)-N-(4-morpholin-4-yl-phenyl)chroman-2-carboxamide (Example 2) and boron tribromide as described in AstraZeneca patent application WO2003037872A1, Example 86.

LC/MS: 453 (M+H, 100%), ¹H-NMR (300 MHz, DMSO-d₆) δ 8.97 (bs, 1H), 7.26 (d, J=9.3 Hz, 2H), 6.60 (d, J=9.3 Hz, 2H), 6.37 (s, 1H), 6.04 (s, 1H), 4.59 (m, 1H), 3.69 (m, 4H), 3.22 (m, 4H), 3.13 (m, 4H), 2.92 (m, 2H), 2.73 (m, 2H), 2.53 (m, 4H), 2.34 (s, 3H).

### Examples 105A and 105B

The enantiomers could be separated using any of the following methods and columns including: Preparative HPLC using 21×250 mm columns (˜20 mid min flow rates) with column packings including Chiralpak AD, Chiralpak OD or Chiralpak OJ and mixtures of either EtOH and Hexane or isopropanol and hexane or supercritical fluid chromatography using columns such as Chiracel OD or Chiracel OD-H with either methanol/CO2/dimethylethylamine or ethanol/CO2/dimethylethylamine yielding (+)-6-Hydroxy-8-(4-methylpiperazin-1-yl)-N-(4-morpholinophenyl)chroman-2-carboxamide and (−)-6-Hydroxy-8-(4-methylpiperazin-1-yl)-N-(4-morpholinophenyl)chroman-2-carboxamide.

Compounds of the present invention include, but are not limited to, the following compositions listed in Table 1 on the following pages.

TABLE 1
Compounds.

| Ex. | | |
|---|---|---|
| No. | Structure | Compound Name |
| 3 | 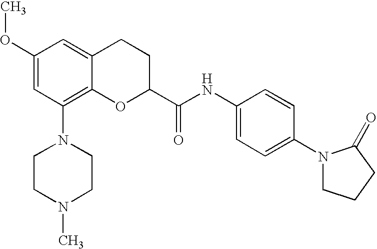 | 6-methoxy-8-(4- methylpiperazin-1-yl)-N- (4-(2-oxopyrrolidin-1- yl)phenyl)chroman-2- carboxamide |
| 3 | 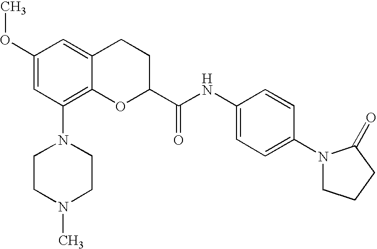 | (+)-6-methoxy-8-(4- methylpiperazin-1-yl)-N- (4-(2-oxopyrrolidin-1- yl)phenyl)chroman-2- carboxamide (Isomer 1) |
| 3 | 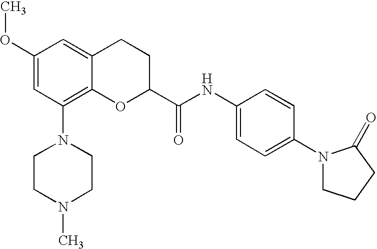 | (−)-6-methoxy-8-(4- methylpiperazin-1-yl)-N- (4-(2-oxopyrrolidin-1- yl)phenyl)chroman-2- carboxamide (Isomer 2) |
| 4 | 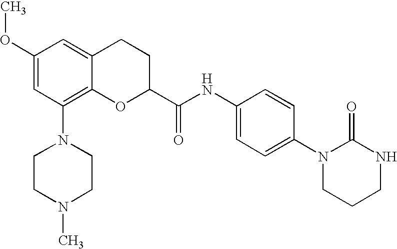 | 6-methoxy-8-(4- methylpiperazin-1-yl)-N- (4-(2- oxotetrahydropyrimidin- 1(2H)-yl)phenyl)chroman- 2-carboxamide |
| 4A | 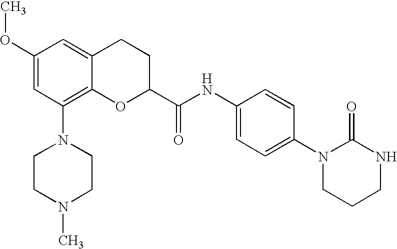 | (+)-6-methoxy-8-(4- methylpiperazin-1-yl)-N- (4-(2- oxotetrahydropyrimidin- 1(2H)-yl)phenyl)chroman- 2-carboxamide (Isomer 1) |
| 4B | 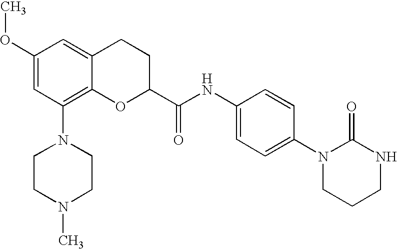 | (−)-6-methoxy-8-(4- methylpiperazin-1-yl)-N- (4-(2- oxotetrahydropyrimidin- 1(2H)-yl)phenyl)chroman- 2-carboxamide (Isomer 2) |
| 5 | 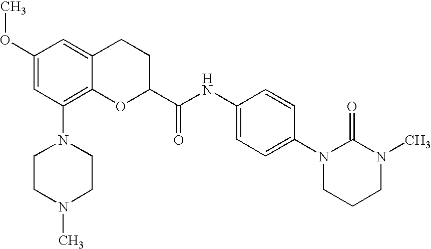 | 6-methoxy-N-(4-(3- methyl-2- oxotetrahydropyrimidin- 1(2H)-yl)phenyl)-8-(4- methylpiperazin-1- 1)chroman-2- carboxamide |
| 5 | 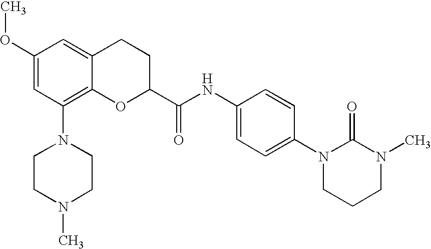 | (+)-6-methoxy-N-(4-(3- methyl-2- oxotetrahydropyrimidin- 1(2H)-yl)phenyl)-8-(4- methylpiperazin-1- 1)chroman-2- carboxamide (Isomer 1) |
| 5 | 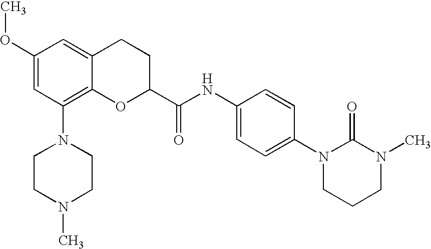 | (−)-6-methoxy-N-(4-(3- methyl-2- oxotetrahydropyrimidin- 1(2H)-yl)phenyl)-8-(4- methylpiperazin-1- yl)chroman-2- carboxamide (Isomer 2) |
| 6 | 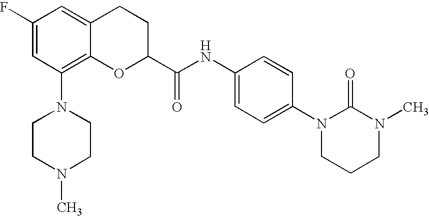 | 6-fluoro-N-(4-(3-methyl- 2-oxotetrahydropyrimidin- 1(2H)-yl)phenyl)-8-(4- methylpiperazin-1- yl)chroman-2- carboxamide |
| 6A | 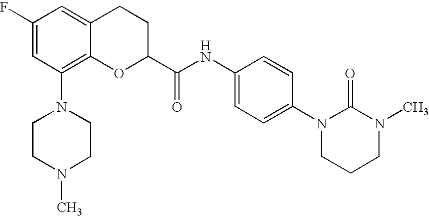 | (+)-6-fluoro-N-(4-(3- methyl-2- oxotetrahydropyrimidin- 1(2H)-yl)phenyl)-8-(4- methylpiperazin-1- yl)chroman-2- carboxamide (Isomer 1) |
| 6B | 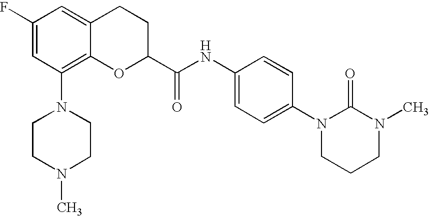 | (−)-6-fluoro-N-(4-(3- ethyl-2- oxotetrahydropyrimidin- 1(2H)-yl)phenyl)-8-(4- methylpiperazin-1- yl)chroman-2- carboxamide (Isomer 2) |
| 7 | 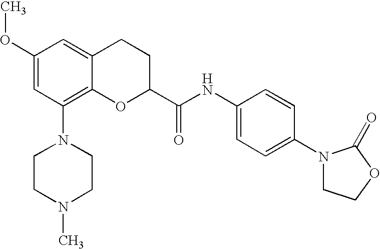 | 6-methoxy-8-(4- methylpiperazin-1-yl)-N- (4-(2-oxooxazolidin-3- yl)phenyl)chroman-2- carboxamide |
| 7A | 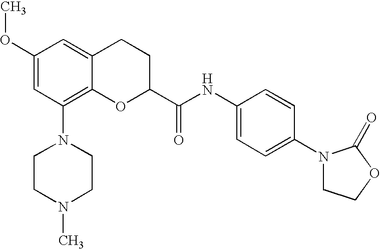 | (+)-6-methoxy-8-(4- methylpiperazin-1-yl)-N- (4-(2-oxooxazolidin-3- yl)phenyl)ohroman-2- carboxamide (Isomer 1) |
| 7B | 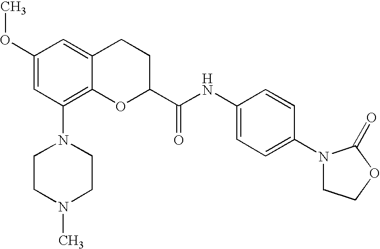 | (−)-6-methoxy-8-(4- ethylpiperazin-1-yl)-N- (4-(2-oxooxazolidin-3- yl)penyl)chroman-2- carboxamide (Isomer 2) |
| 8 | 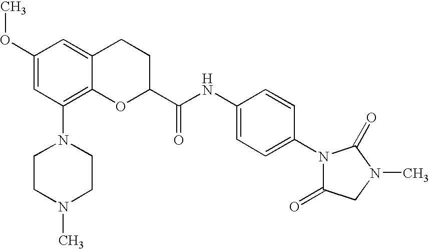 | 6-methoxy-N-(4-(3- methyl-2,5- dioxoimidazolidin-1- yl)phenyl)-8-(4- methylpiperazin-1- yl)chroman-2- carboxamide |
| 8A | 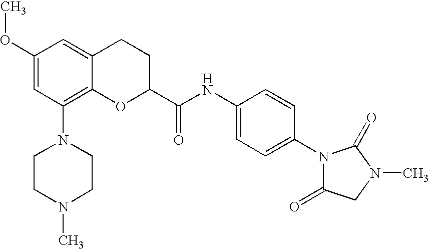 | (+)-6-methoxy-N-(4-(3- methyl-2,5- dioxoimidazolidin-1- yl)phenyl)-8-(4- methylpiperazin-1- yl)chroman-2- carboxamide (Isomer 1) |
| 8B | 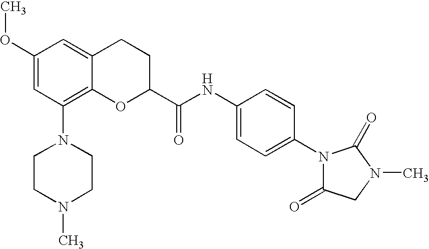 | (−)-6-methoxy-N-(4-(3- methyl-2,5- dioxoimidazolidin-1- yl)phenyl)-8-(4- methylpiperazin-1- yl)chroman-2- carboxamide (Isomer 2) |
| 9 | 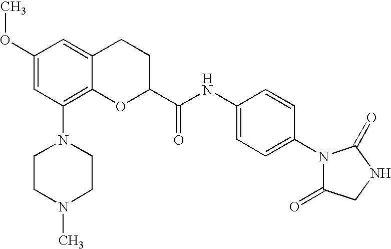 | N-(4-(2,5- dioxoimidazolidin-1- yl)phenyl)-6-methoxy-8- (4-methylpiperazin-1- yl)chroman-2- carboxamide |
| 9A | 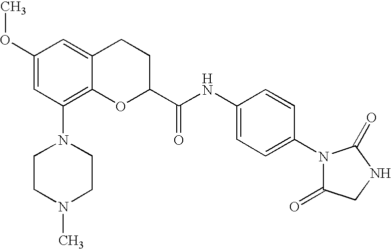 | (+)-N-(4-(2,5- dioxoimidazolidin-1- yl)phenyl)-6-methoxy-8- (4-methylpiperazin-1- yl)chroman-2- carboxamide (Isomer 1) |
| 9B | 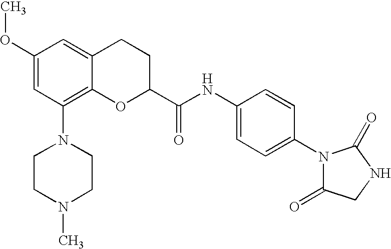 | (−)-N-(4-(2,5- dioxoimidazolidin-1- yl)phenyl)-6-methoxy-8- (4-methylpiperazin-1- yl)chroman-2- carboxamide (Isomer 2) |
| 10 | 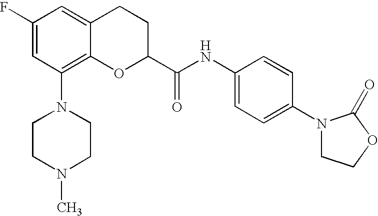 | 6-fluoro-8-(4- methylpiperazine-1-yl)-N- (4-(2-oxooxazolidin-3- yl)phenyl)chroman-2- carboxamide |
| 10A | 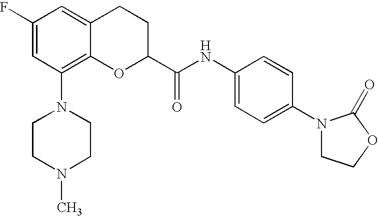 | (+)-6-fluoro-8-(4- methylpiperazin-1-yl)-N- (4-(2-oxooxazolidin-3- yl)phenyl)chroman-2- carboxamide (Isomer 1) |
| 10B | 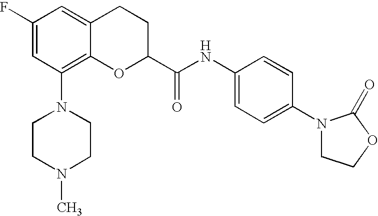 | (−)-6-fluoro-8-(4- methylpiperazin-1-yl)-N- (4-(2-oxooxazolidin-3- yl)phenyl)chroman-2- carboxamide (Isomer 2) |
| 11 | 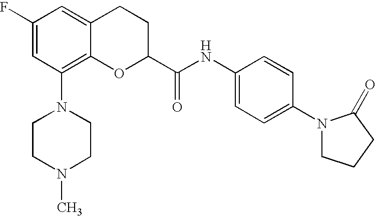 | 6-fluoro-8-(4- methylpiperazin-1-yl)-N- (4-(2-oxopyrrolidin-1- yl)phenyl)chroman-2- carboxamide |
| 11A | 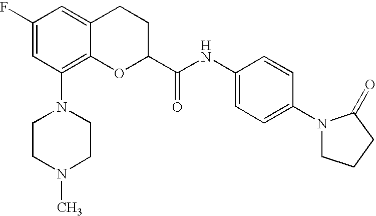 | (+)-6-fluoro-8-(4- methylpiperazin-1-yl)-N- oxopyrrolidin-1- yl)phenyl)chroman-2- carboxamide (Isomer 1) |
| 11B | 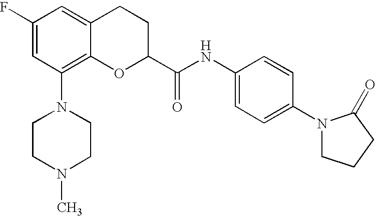 | (−)-6-fluoro-8-(4- methylpiperazin-1-yl)-N- (4-(2-oxopyrrolidin-1- yl)phenyl)chroman-2- carboxamide (Isomer 2) |
| 12 | 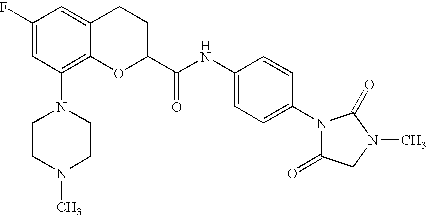 | 6-fluoro-N-(4-(3-methyl- 2,5-dioxoimidazolidin-1- yl)phenyl)-8-(4- methylpiperazin-1- yl)chroman-2- carboxamide |
| 12A | 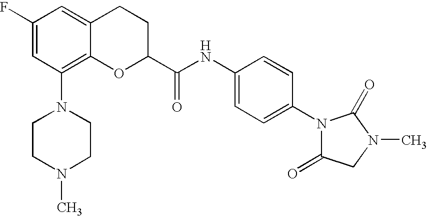 | (+)-6-fluoro-N-(4-(3- methyl-2,5- dioxoimidazolidin-1- yl)phenyl)-8-(4- methylpiperazin-1- yl)chroman-2- carboxamide (Isomer 1) |
| 12B | 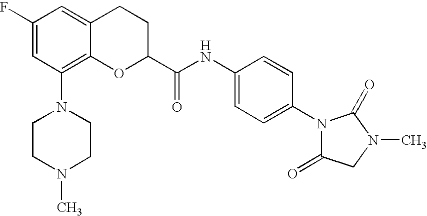 | (−)-6-fluoro-N-(4-(3- methyl-2,5- dioxoimidazolidin-1- yl)phenyl)-8-(4- methylpiperazin-1- yl)chroman-2- carboxamide (Isomer 2) |
| 13 | 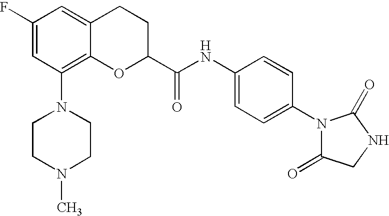 | N-(4-(2,5- dioxoimidazolidin-1- yl)phenyl)-6-fluoro-8-(4- methylpiperazin-1- yl)chroman-2- carboxamide |
| 13A | 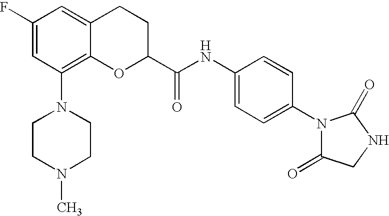 | (+)-N-(4-(2,5- dioxoimidazolidin-1- yl)phenyl)-6-fluoro-8-(4- methylpiperazin-1- yl)chroman-2- carboxamide (Isomer 1) |
| 13B | 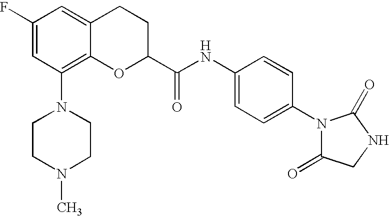 | (−)-N-(4-(2,5- dioxoimidazolidin-1- yl)phenyl)-6-fluoro-8-(4- methylpiperazin-1- yl)chroman-2- carboxamide (Isomer 2) |
| 14 | 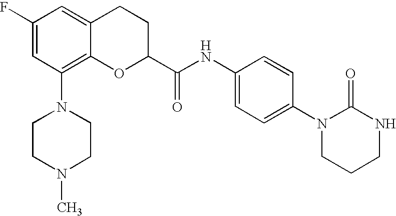 | 6-fluoro-8-(4- methylpiperazin-1-yl)-N- (4-(2- oxotetrahydropyrimidin- 1(2H)-yl)phenyl)chroman- 2-carboxamide |
| 14A | 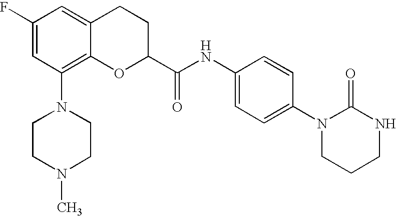 | (+)-6-fluoro-8-(4- methylpiperazin-1-yl)-N- (4-(2- oxotetrahydropyrimidin- 1(2H)-yl)phenyl)chroman- 2-carboxamide (Isomer 1) |
| 14B | 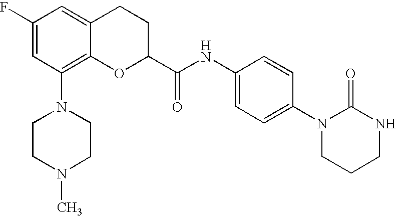 | (−)-6-fluoro-8-(4- methylpiperazin-1-yl)-N- (4-(2- oxotetrabydropyrimidin- 1(2H)-yl)phenyl)chroman- 2-carboxamide (Isomer 2) |
| 15 | 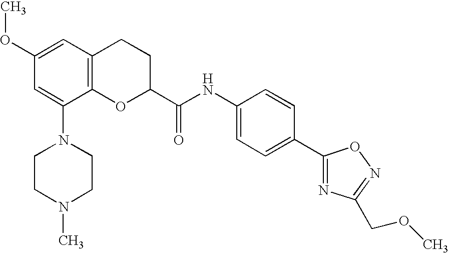 | 6-methoxy-N-(4-(3- (methoxymethyl)-1,2,4- oxadiazol-5-yl)phenyl)-8- (4-methylpiperazin-1- yl)chroman-2- carboxamide |
| 15A | 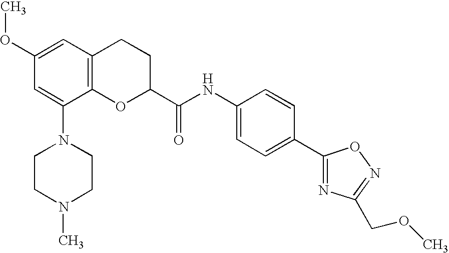 | (+)-6-methoxy-N-(4-(3- (methoxymethyl)-1,2,4- oxadiazol-5-yl)phenyl)-8- (4-methylpiperazin-1- yl)chroman-2- carboxamide (Isomer 1) |
| 15B | 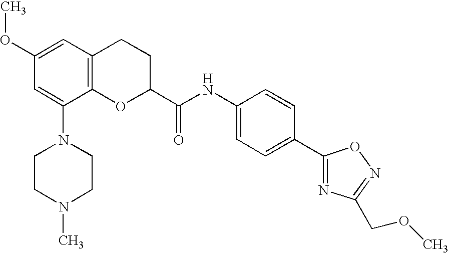 | (−)-6-methoxy-N-(4-(3- (methoxymethyl)-1,2,4- oxadiazol-5-yl)phenyl)-8- (4-methylpiperazin-1- yl)chroman-2- carboxamide (Isomer 2) |
| 16 | 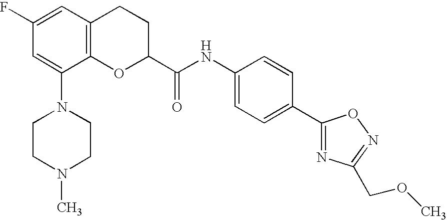 | 6-fluoro-N-(4-(3- (methoxymethyl)-1,2,4- oxadiazol-5-yl)phenyl)-8- (4-methylpiperazin-1- yl)chroman-2- carboxamide |
| 16A | 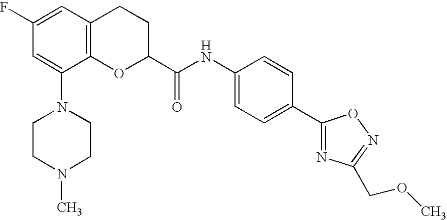 | (+)-6-fluoro-N-(4-(3- (methoxymethyl)-1,2,4- oxadiazol-5-yl)phenyl)-8- (4-methylpiperazin-1- yl)chroman-2- carboxamide (Isomer 1) |
| 16B | 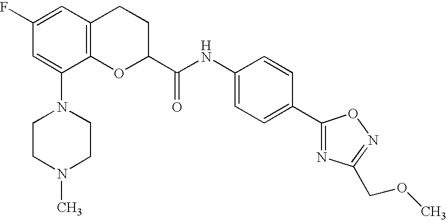 | (−)-6-fluoro-N-(4-(3- (methoxymethyl)-1,2,4- oxadiazol-5-yl)phenyl)-8- (4-methylpiperazin-1- yl)chroman-2- carboxamide (Isomer 2) |
| 18 | 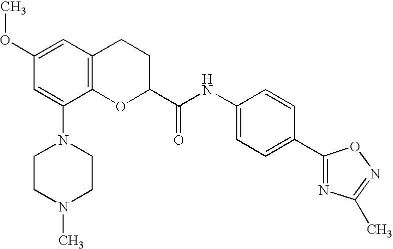 | 6-methoxy-N-(4-(3- methyl-1,2,4-oxadiazol-5- yl)phenyl)-8-(4- methylpiperazin-1- yl)chroman-2- carboxamide |
| 18A | 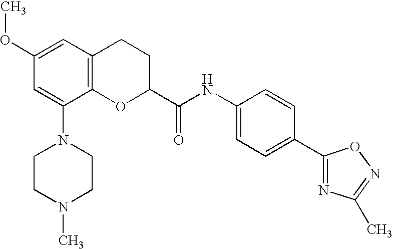 | (+)-6-methoxy-N-(4-(3- methyl-1,2,4-oxadiazol-5- yl)phenyl)-8-(4- methylpiperazin-1- yl)chroman-2- carboxamide (Isomer 1) |
| 18B | 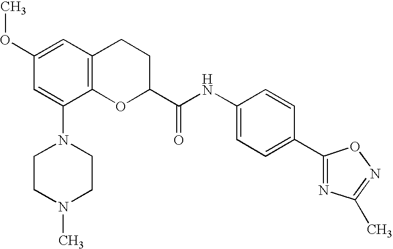 | (−)-6-methoxy-N-(4-(3- methyl-1,2,4-oxadiazol-5- yl)phenyl)-8-(4- methylpiperazin-1- yl)chroman-2- carboxamide (Isomer 2) |
| 19 | 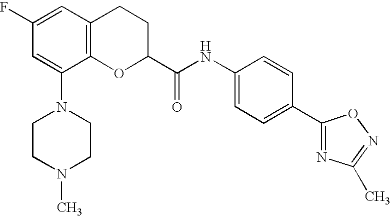 | 6-fluoro-N-(4-(3-methyl- 1,2,4-oxadiazol-5- yl)phenyl)-8-(4- methylpiperazin-1- yl)chroman-2- carboxamide |
| 19A | 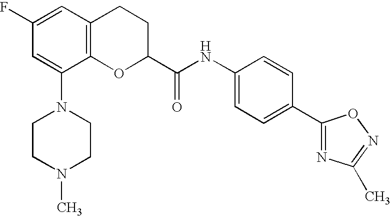 | (+)-6-fluoro-N-(4-(3- methyl-1,2,4-oxadiazol-5- yl)phenyl)-8-(4- methylpiperazin-1- yl)chroman-2- carboxamide(Isomer 1) |
| 19B | 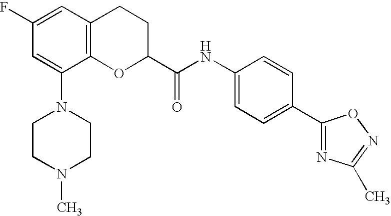 | (−)-6-fluoro-N-(4-(3- methyl-1,2,4-oxadiazol-5- yl)phenyl)-8-(4- methylpiperazin-1- yl)chroman-2- carboxamide (Isomer 2) |
| 20 | 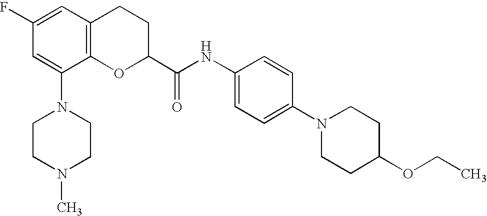 | N-(4-(4-ethoxypiperidin- 1-yl)phenyl)-6-fluoro-8- yl)chroman-2- (4-methylpiperazin-1- carboxamide |
| 20A | 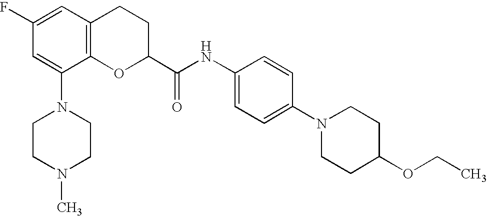 | (+)-N-(4-(4- ethoxypiperidin-1- yl)phenyl)-6-fluoro-8-(4- methylpiperazin-1- yl)chroman-2- carboxamide (Isomer 1) |
| 20B | 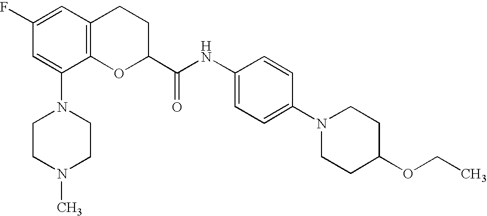 | (−)-N-(4-(4- ethoxypiperidin-1- yl)phenyl)-6-fluoro-8-(4- methylpiperazin-1- yl)chroman-2- carboxamide (Isomer 2) |
| 58 | 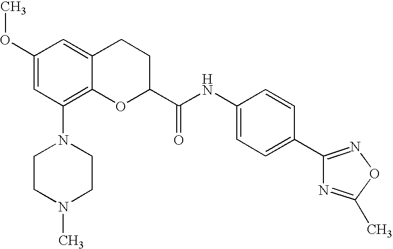 | 6-methoxy-N-(4-(5- methyl-1,2,4-oxadiazol-3- yl)phenyl)-8-(4- methylpiperazin-1- yl)chroman-2- carboxamide |
| 58A | 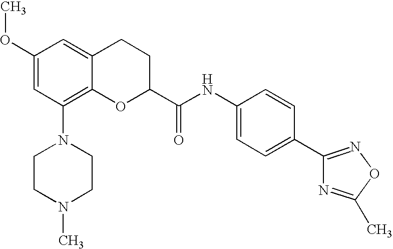 | (+)-6-methoxy-N-(4-(5- methyl-1,2,4-oxadiazol-3- yl)phenyl)-8-(4- methylpiperazin-1- yl)chroman-2- carboxamide (Isomer 1) |
| 58B | 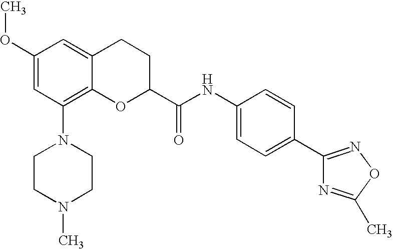 | (−)-6-methoxy-N-(4-(5- methyl-1,2,4-oxadiazol-3- yl)phenyl)-8-(4- methylpiperazin-1- yl)chroman-2- carboxamide (Isomer 2) |
| 59 | 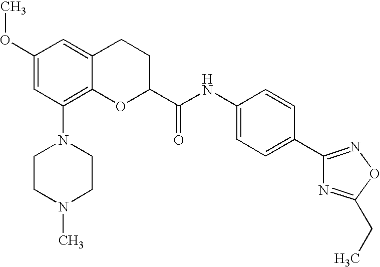 | N-(4-(5-ethyl-1,2,4- oxadiazol-3-yl)phenyl)-6- methoxy-8-(4- methylpiperazin-1- yl)chroman-2- carboxamide |
| 59A | 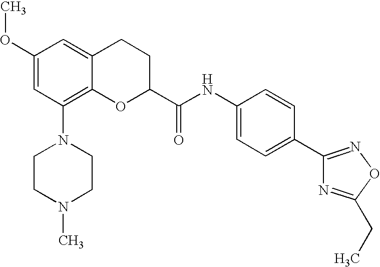 | (+)-N-(4-(5-ethyl-1,2,4- oxadiazol-3-yl)phenyl)-6- methoxy-8-(4- methylpiperazin-1- yl)chroman-2- carboxamide (Isomer 1) |
| 59B | 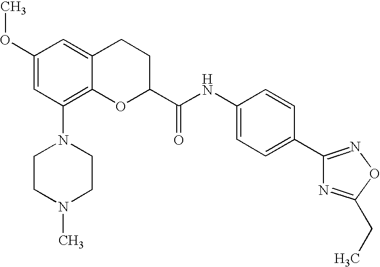 | (−)-N-(4-(5-ethyl-1,2,4- oxadiazol-3-yl)phenyl)-6- methoxy-8-(4- methylpiperazin-1- yl)chroman-2- carboxamide (Isomer 2) |
| 60 | 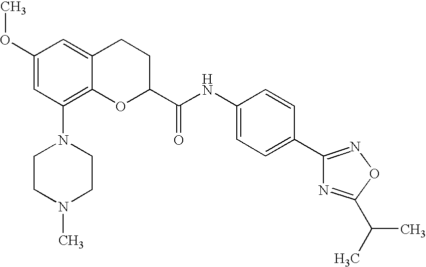 | N-(4-(5-isopropyl-1,2,4- oxadiazol-3-yl)phenyl)-6- ethoxy-8-(4- methylpiperazin-1- yl)chroman-2- carboxamide |
| 60A | 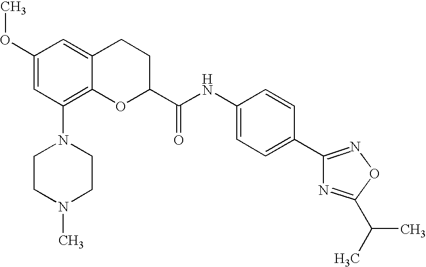 | (−)-N-(4-(5-isopropyl- 1,2,4-oxadiazol-3- yl)phenyl)-6-methoxy-8- (4-methylpiperazin-1- yl)chroman-2- carboxamide (Isomer 2) |
| 60B | 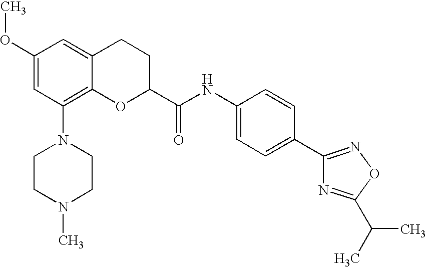 | (+)-N-(4-(5-isopropyl- 1,2,4-oxadiazol-3- yl)phenyl)-6-methoxy-8- (4-methylpiperazin-1- yl)chroman-2- carboxamide (Isomer 1) |
| 61 | 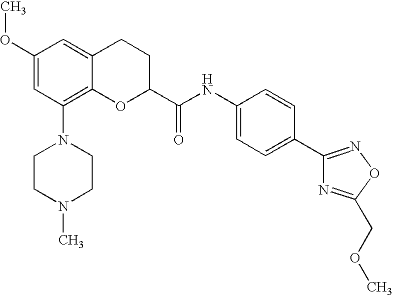 | 6-methoxy-N-(4-(5- (methoxymethyl)-1,2,4- oxadiazol-3-yl)phenyl)-8- (4-methylpiperazin-1- yl)chroman-2- carboxamide |
| 61A | 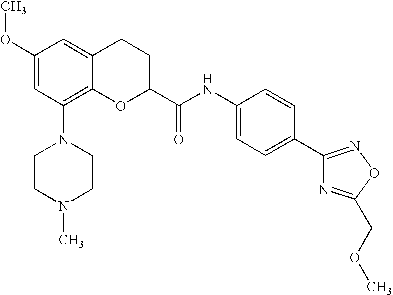 | (+)-6-methoxy-N-(4-(5- (methoxymethyl)-1,2,4- oxadiazol-3-yl)phenyl)-8- (4-methylpiperazin-1- yl)chroman-2- carboxamide (Isomer 1) |
| 61B | 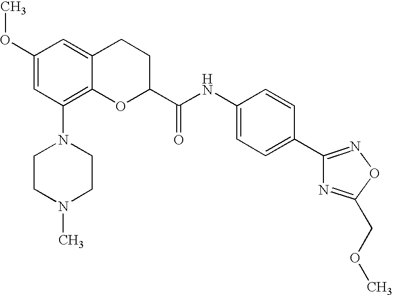 | (−)-6-methoxy-N-(4-(5- (methoxymethy yl)-1,2,4- oxadiazol-3-yl)phenyl)-8- (4-methylpiperazin-1- yl)chroman-2- carboxamide (Isomer 2) |
| 62 | 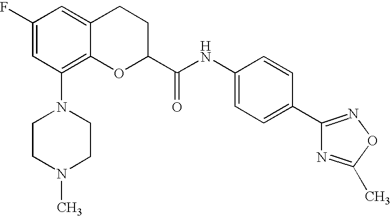 | 6-fluoro-N-(4-(5-methyl- 1,2,4-oxadiazol-3- yl)phenyl)-8-(4- methylpiperazin-1- yl)chroman-2- carboxamide |
| 62A | 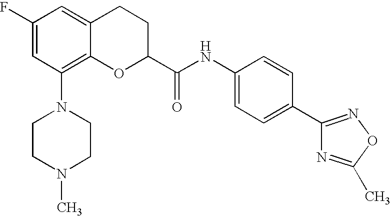 | (+)-6-fluoro-N-(4-(5- methyl-1,2,4-oxadiazol-3- yl)phenyl)-8-(4- methylpiperazin-1- yl)chroman-2- carboxamide (Isomer 1) |
| 62B | 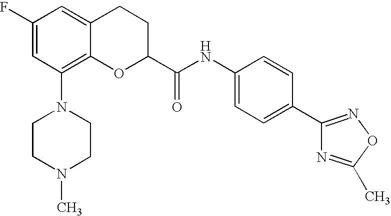 | (−)-6-fluoro-N-(4-(5- methyl-1,2,4-oxadiazol-3- yl)pbenyl)-8-(4- methylpiperazin-1- yl)chroman-2- carboxamide (Isomer 2) |
| 63 | 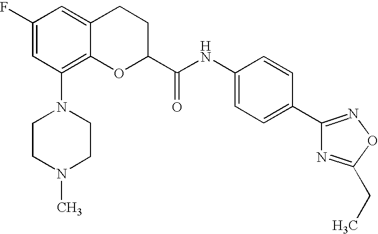 | N-(4-(5-ethyl-1,2,4- oxadiazol-3-yl)phenyl)-6- fluoro-8-(4- methylpiperazin-1- carboxamide |
| 63A | 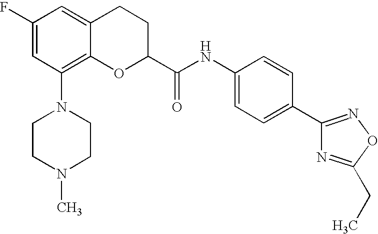 | (+)-N-(4-(5-ethyl-1,2,4- oxadiazol-3-yl)phenyl)-6- fluoro-8-(4- methylpiperazin-1- yl)chroman-2- carboxamide (Isomer 1) |
| 63B | 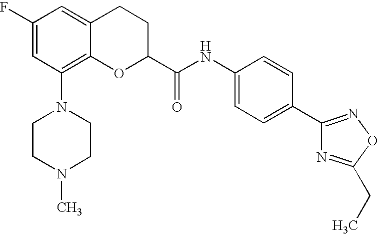 | (−)-N-(4-(5-ethyl-1,2,4- oxadiazol-3-yl)phenyl)-6- fluoro-8-(4- methylpiperazin-1- yl)chroman-2- carboxamide (Isomer 2) |
| 64 | 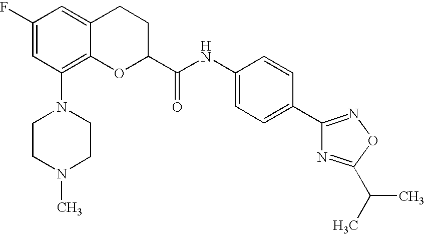 | 6-fluoro-N-(4-(5- isopropyl-1,2,4-oxadiazol- 3-yl)phenyl)-8-(4- methylpiperazin-1- yl)chroman-2- carboxamide |
| 64A | 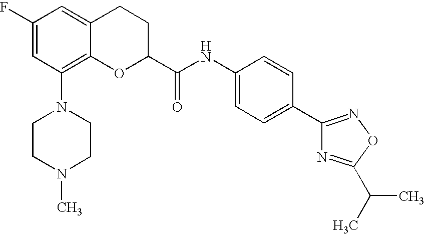 | (+)-6-fluoro-N-(4-(5- isopropyl-1,2,4-oxadiazol- 3-yl)phenyl)-8-(4- methylpiperazin-1- yl)chroman-2- carboxamide (Isomer 1) |
| 64B | 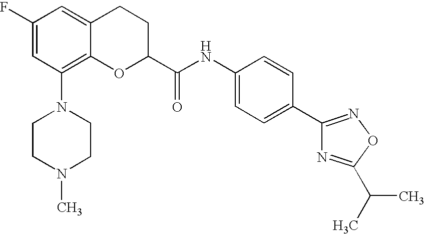 | (−)-6-fluoro-N-(4-(5- sopropyl-1,2,4-oxadiazol- 3-yl)phenyl)-8-(4- yl)chroman-2- carboxamide (Isomer 2) |
| 65 | 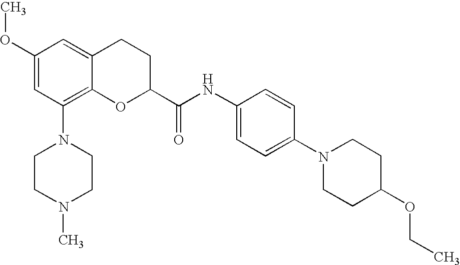 | N-(4-(4-ethoxypiperidin- 1-yl)phenyl)-6-methoxy-8- (4-methylpiperazin-1- yl)chroman-2- carboxamide |
| 65B | 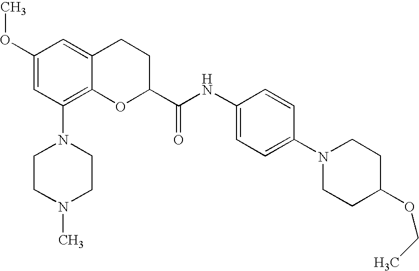 | (−)-N-(4-(4- ethoxypiperidin-1- yl)phenyl)-6-methoxy-8- (4-methylpiperazin-1- yl)chroman-2- carboxamide (Isomer 2) |
| 65A | 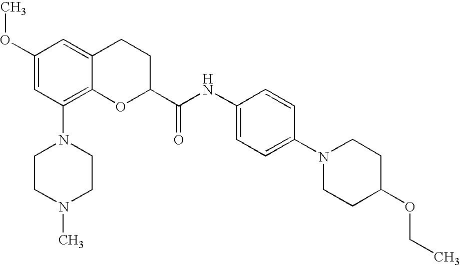 | (+)-N-(4-(4- ethoxypiperidin-1- yl)phenyl)-6-methoxy-8- (4-methylpiperazin-1- yl)chroman-2- carboxamide (Isomer 1) |
| 67 | 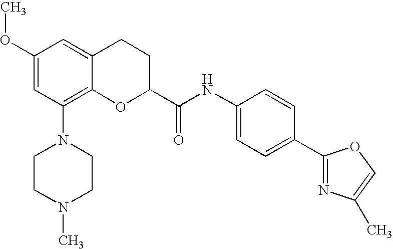 | 6-methoxy-N-(4-(4- methyloxazol-2- yl)phenyl)-8-(4- methylpiperazin-1- yl)chroman-2- carboxamide |
| 67A | 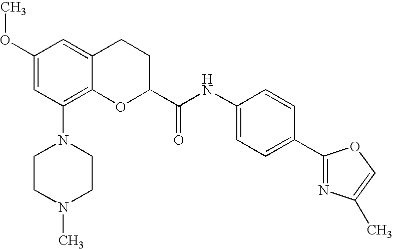 | (+)-6-methoxy-N-(4-(4- methyloxazol-2- yl)phenyl)-8-(4- methylpiperazin-1- yl)chroman-2- carboxamide (Isomer 1) |
| 67B | 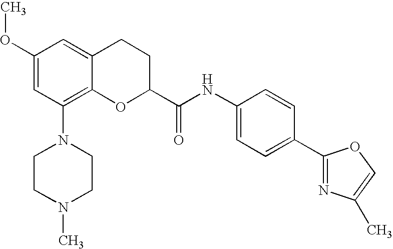 | (−)-6-methoxy-N-(4-(4- methyloxazol-2- yl)phenyl)-8-(4- methylpiperazin-1- yl)chroman-2- carboxamide (Isomer 2) |
| 72 | 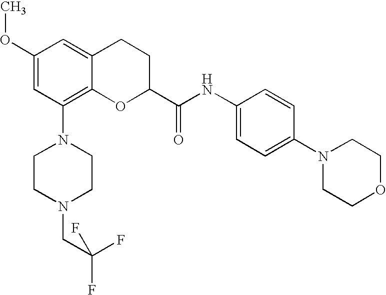 | 6-methoxy-N-(4- morpholinophenyl)-8-(4- (2,2,2- trifluoroethyl)piperazin-1- yl)chroman-2- carboxamide |
| 72A | 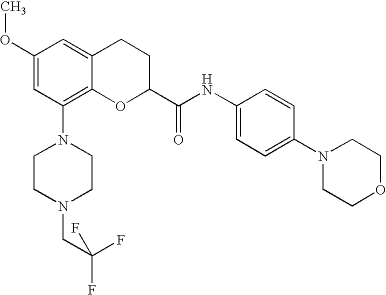 | (+)-6-methoxy-N-(4- morpholinophenyl)-8-(4- (2,2,2- yl)chroman-2- trifluoroethyl)piperazin-1- carboxamide (Isomer 1) |
| 72B | 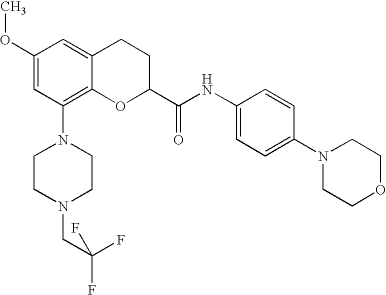 | (−)-6-methoxy-N-(4- morpholinophenyl)-8-(4- (2,2,2- trifluoroethyl)piperazin-1- yl)chroman-2- carboxamide (Isomer 2) |
| 73 | 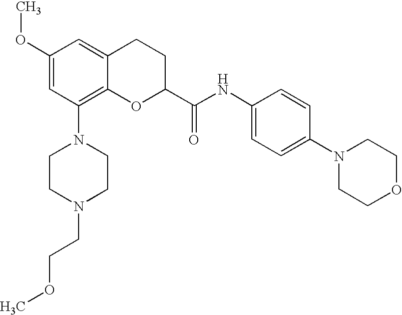 | 6-methoxy-8-(4-(2- ethoxyethyl)piperazin-1- morpholinophenyl)chroman- 2-carboxamide |
| 73A | 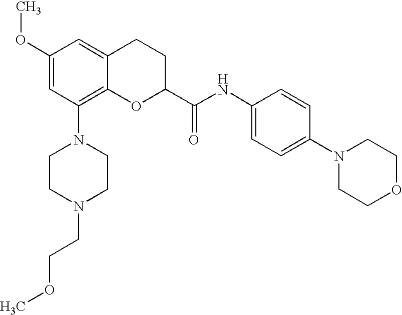 | (+)-6-methoxy-8-(4-(2- methoxyethyl)piperazin-1- yl)-N-(4- morpholinophenyl)chroman- 2-carboxamide (Isomer 1) |
| 73B | 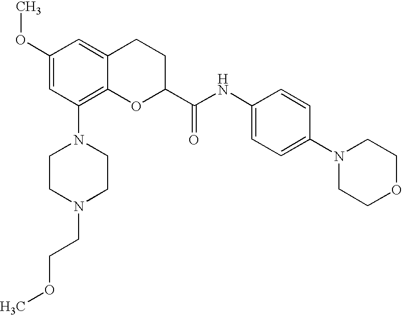 | (−)-6-methoxy-8-(4-(2- methoxyethyl)piperazin-1- yl)-N-(4- morpholinophenyl)chroman- 2-carboxamide (Isomer 2) |
| 75 | 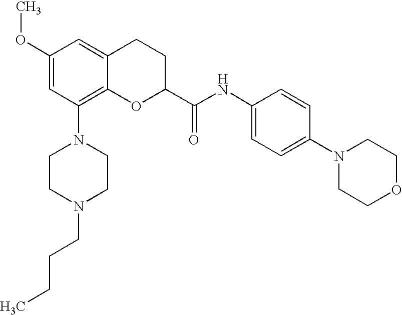 | 8-(4-butylpiperazin-1-yl)- 6-methoxy-N-(4- morpholinophenyl)chroman- 2-carboxamide |
| 75A | 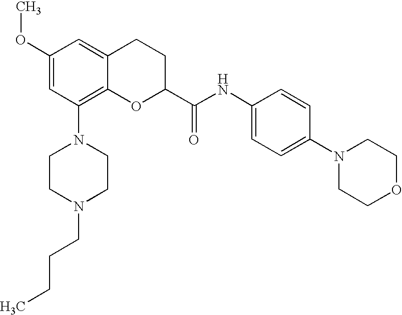 | (+)-8-(4-butylpiperazin-1- yl)-6-methoxy-N-(4- morpholinophenyl)chroman- 2-carboxamide (Isomer 1) |
| 75B | 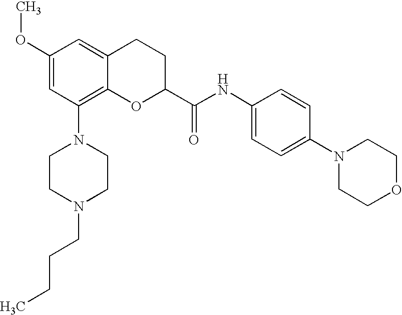 | (−)-8-(4-butylpiperazin-1- yl)-6-methoxy-N-(4- morpholinophenyl)chroman- 2-carboxamide (Isomer 2) |
| 76 | 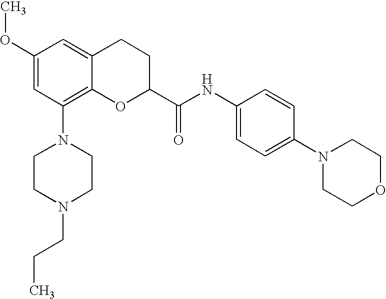 | 6-methoxy-N-(4- morpholinophenyl)-8-(4- propylpiperazin-1- yl)chroman-2- carboxamide |
| 76A | 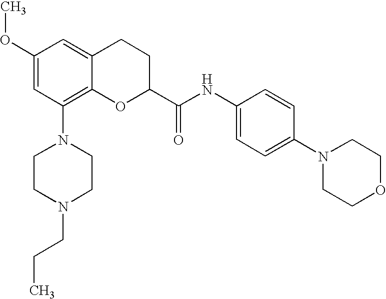 | (+)-6-methoxy-N-(4- morpholinophenyl)-8-(4- propylpiperazin-1- yl)chroman-2- carboxamide (Isomer 1) |
| 76B | 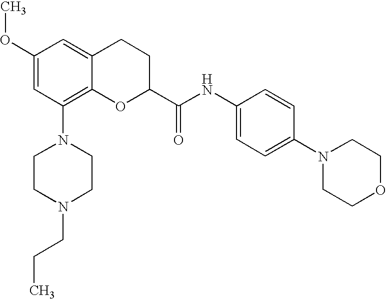 | (−)-6-methoxy-N-(4- morpholinophenyl)-8-(4- propylpiperazin-1- yl)chroman-2- carboxamide (Isomer 2) |
| 77 | 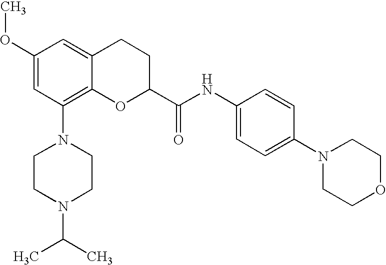 | 8-(4-isopropylpiperazin-1- yl)-6-methoxy-N-(4- morpholinophenyl)chroman- 2-carboxamide |
| 77A | 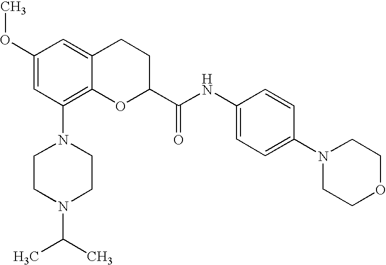 | (+)-8-(4- isopropylpiperazin-1-yl)- 6-methoxy-N-(4- morpholinophenyl)chroman- 2-carboxamide |
| 77B | 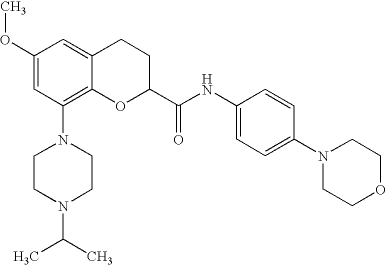 | (−)-8-(4- isopropylpiperazin-1-yl)- 6-methoxy-N-(4- morpholinophenyl)chroman- 2-carboxamide (Isomer 2) |
| 78 | 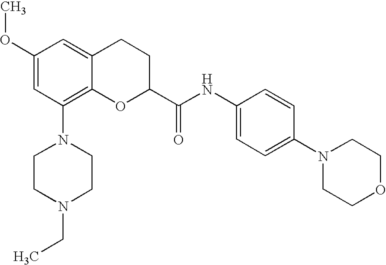 | 8-(4-ethylpiperazin-l-yl)- 6-methoxy-N-(4- morpholinophenyl)chroman- 2-carboxamide |
| 78A | 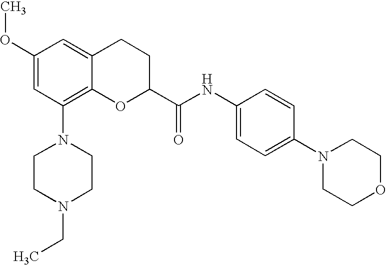 | (+)-8-(4-ethylpiperazin-1- yl)-6-methoxy-N-(4- morpholinophenyl)chroman- 2-carboxamide (Isomer 1) |
| 78B | 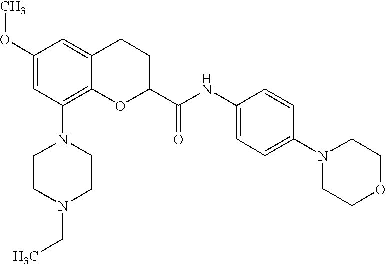 | (−)-8-(4-ethylpiperazin-1- yl)-6-methoxy-N-(4- morpholinophenyl)chroman- 2-carboxamide (Isomer 2) |
| 79 | 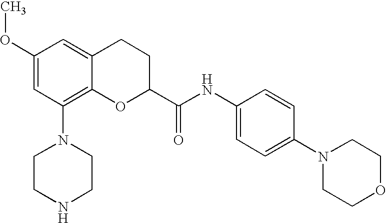 | 6-methoxy-N-(4- morpholinophenyl)-8- (piperazin-1-yl)chroman- 2-carboxamide |
| 79C | 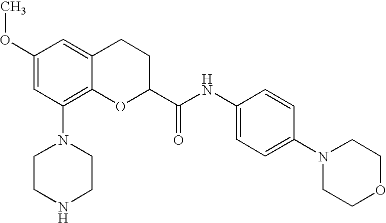 | (+)-6-methoxy-N-(4- morpholinophenyl)-8- (piperazin-1-yl)chroman- 2-carboxamide (Isomer 1) |
| 79D | 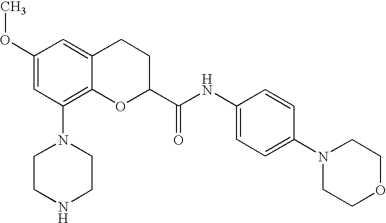 | (−)-6-methoxy-N-(4- morpholinophenyl)-8- (piperazin-1-yl)chroman- 2-carboxamide (Isomer 2) |
| 80 | 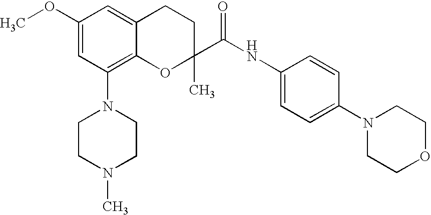 | 6-methoxy-2-methyl-8-(4- methylpiperazin-1-yl)-N- (4- morpholinophenyl)chroman- 2-carboxamide |
| 80A | 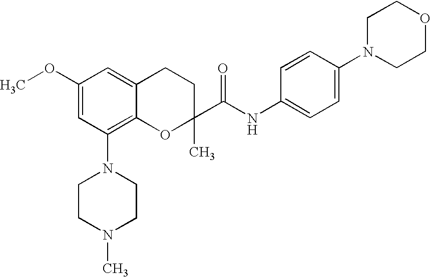 | (+)-6-methoxy-2-methyl- 8-(4-methylpiperazin-1- morpholinophenyl)chroman- 2-carboxamide (Isomer 1) |
| 80B | 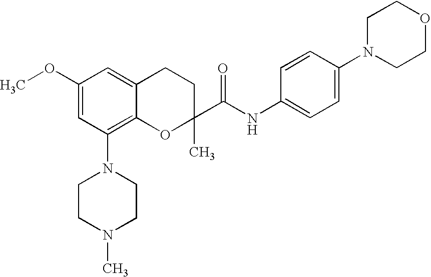 | (−)-6-methoxy-2-methyl-8- (4-methylpiperazin-1-yl)- N-(4- morpholinophenyl)chroman- 2-carboxamide (Isomer 2) |
| 105 | 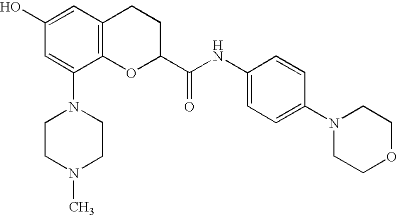 | 6-hydroxy-8-(4- methylpiperazin-1-yl)-N- (4- morpholinophenyl)chroman- 2-carboxamide |
| 105A | 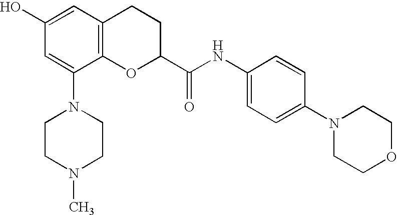 | (+)-6-hydroxy-8-(4- methylpiperazin-1-yl)-N- (4- morpholinophenyl)chroman- 2-carboxamide (Isomer 1) |
| 105B | 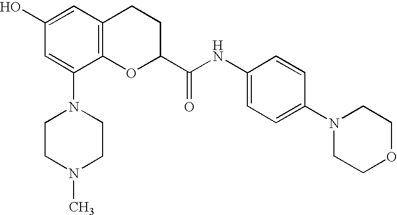 | (−)-6-hydroxy-8-(4- methylpiperazin-1-yl)-N- (4- morpholinophenyl)chroman- 2-carboxamide (Isomer 2) |

We claim:

1. A compound in accord with Formula I: 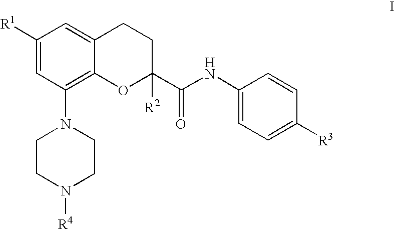
wherein:
R¹ is F or OCH₃ or OH;
R² is H, or CH₃;
R³ is a heterocycle optionally substituted with one of the following —CH₃, —CH₂CH₃, —CH₂CH₂CH₃, —CH(CH₃)₂, —CH₂OCH₃, or —OCH₂CH₃; and
R⁴ is H, —CH₃, CH₂CF₃, —CH₂CH₂OCH₃, CH₂CH₂CH₂CH₃, —CH₂CH₂CH₃, —CH(CH₃)₂, or —CH₂CH₃
or a pharmaceutically-acceptable salt thereof.

2. A compound according to claim 1 wherein R¹ is F.

3. A compound according to claim 1 wherein R¹ is OCH₃.

4. A compound according to claim 1 wherein R¹ is OH.

5. A compound according to claims 1-4 wherein R² is H.

6. A compound according to claims 1-4 wherein R² is CH₃.

7. A compound according to claims 1-6 wherein R³ is oxadiazole.

8. A compound according to claims 1-6 wherein R³ is oxooxazolidine.

9. A compound according to claims 1-6 wherein R³ is oxotetrahydorpyrimidine optionally substituted with —CH₃.

10. A compound according to claims 1-6 wherein R³ is dioxoimidazolidin optionally substituted with —CH₃.

11. A compound according to claims 1-6 wherein R³ is oxopyrrolidin.

12. A compound according to claims 1-6 wherein R³ is piperidine optionally substituted with —OCH₂CH₃.

13. A compound according to claims 1-6 wherein R³ is oxazole.

14. A compound according to claims 1-6 wherein R³ is morpholine.

15. A compound according to claim 1 wherein:
R¹ is F or OCH₃;
R² is H;
R³ is a heterocycle optionally substituted with one of the following —CH₃, —CH₂CH₃, —CH₂CH₂CH₃, —CH(CH₃)₂, —CH₂OCH₃, or —OCH₂CH₃; and
R⁴ is —CH₃;
or a pharmaceutically-acceptable salt thereof.

16. A compound according to claim 15 wherein R¹ is F.

17. A compound according to claim 15 wherein R¹ is OCH₃.

18. A compound according to claims 15, 16, or 17 wherein R³ is oxadiazole optionally substituted with one of the following —CH₃, —CH₂CH₃, CH(CH₃)₂, or —CH₂OCH₃.

19. A compound according to claims 15, 16, or 17 wherein R³ is oxooxazolidine.

20. A compound according to claims 15, 16, or 17 wherein R³ is oxotetrahydropyrimidine optionally substituted with —CH₃.

21. A compound according to claims 15, 16, or 17 wherein R³ is dioxoimidazolidin optionally substituted with CH₃.

22. A compound according to claims 15, 16, or 17 wherein R³ is oxopyrrolidin.

23. A compound according to claims 15, 16, or 17 wherein R³ is piperidine optionally substituted with —OCH₂CH₃.

24. A compound according to claims 15, 16, or 17 wherein R³ is oxazole.

25. A compound according to claims 15, 16, or 17 wherein R³ is morpholine.

26. A compound selected from:
6-methoxy-8-(4-methylpiperazin-1-yl)-N-(4-(2-oxopyrrolidin-1-yl)phenyl)chroman-2-carboxamide;
(+)-6-methoxy-8-(4-methylpiperazin-1-yl)-N-(4-(2-oxopyrrolidin-1-yl)phenyl)chroman-2-carboxamide; (Isomer 1)
(−)-6-methoxy-8-(4-methylpiperazin-1-yl)-N-(4-(2-oxopyrrolidin-1-yl)phenyl)chroman-2-carboxamide; (Isomer 2)
6-methoxy-8-(4-methylpiperazin-1-yl)-N-(4-(2-oxotetrahydropyrimidin-1(2H)-yl)phenyl)chroman-2-carboxamide;
(+)-6-methoxy-8-(4-methylpiperazin-1-yl)-N-(4-(2-oxotetrahydropyrimidin-1(2H)-yl)phenyl)chroman-2-carboxamide; (Isomer 1)
(−)-6-methoxy-8-(4-methylpiperazin-1-yl)-N-(4-(2-oxotetrahydropyrimidin-1(2H)-yl)phenyl)chroman-2-carboxamide; (Isomer 2)
6-methoxy-N-(4-(3-methyl-2-oxotetrahydropyrimidin-1(2H)-yl)phenyl)-8-(4-methylpiperazin-1-yl)chroman-2-carboxamide;
(+)-6-methoxy-N-(4-(3-methyl-2-oxotetrahydropyrimidin-1(2H)-yl)phenyl)-8-(4-methylpiperazin-1-yl)chroman-2-carboxamide; (Isomer 1)
(−)-6-methoxy-N-(4-(3-methyl-2-oxotetrahydropyrimidin-1(2H)-yl)phenyl)-8-(4-methylpiperazin-1-yl)chroman-2-carboxamide; (Isomer 2)
6-fluoro-N-(4-(3-methyl-2-oxotetrahydropyrimidin-1(2H)-yl)phenyl)-8-(4-methylpiperazin-1-yl)chroman-2-carboxamide;
(+)-6-fluoro-N-(4-(3-methyl-2-oxotetrahydropyrimidin-1 (2H)-yl)phenyl)-8-(4-methylpiperazin-1-yl)chroman-2-carboxamide; (Isomer 1)
(−)-6-fluoro-N-(4-(3-methyl-2-oxotetrahydropyrimidin-1(2H)-yl)phenyl)-8-(4-methylpiperazin-1-yl)chroman-2-carboxamide; (Isomer 2)
6-methoxy-8-(4-methylpiperazin-1-yl)-N-(4-(2-oxooxazolidin-3-yl)phenyl)chroman-2-carboxamide;
(+)-6-methoxy-8-(4-methylpiperazin-1-yl)-N-(4-(2-oxooxazolidin-3-yl)phenyl)chroman-2-carboxamide; (Isomer 1)
(−)-6-methoxy-8-(4-methylpiperazin-1-yl)-N-(4-(2-oxooxazolidin-3-yl)phenyl)chroman-2-carboxamide; (Isomer 2)
6-methoxy-N-(4-(3-methyl-2,5-dioxoimidazolidin-1-yl)phenyl)-8-(4-methylpiperazin-1-yl)chroman-2-carboxamide;
(+)-6-methoxy-N-(4-(3-methyl-2,5-dioxoimidazolidin-1-yl)phenyl)-8-(4-methylpiperazin-1-yl)chroman-2-carboxamide; (Isomer 1)
(−)-6-methoxy-N-(4-(3-methyl-2,5-dioxoimidazolidin-1-yl)phenyl)-8-(4-methylpiperazin-1-yl)chroman-2-carboxamide; (Isomer 2)
N-(4-(2,5-dioxoimidazolidin-1-yl)phenyl)-6-methoxy-8-(4-methylpiperazin-1-yl)chroman-2-carboxamide;
(+)-N-(4-(2,5-dioxoimidazolidin-1-yl)phenyl)-6-methoxy-8-(4-methylpiperazin-1-yl)chroman-2-carboxamide; (Isomer 1)
(−)-N-(4-(2,5-dioxoimidazolidin-1-yl)phenyl)-6-methoxy-8-(4-methylpiperazin-1-yl)chroman-2-carboxamide; (Isomer 2)
6-fluoro-8-(4-methylpiperazin-1-yl)-N-(4-(2-oxooxazolidin-3-yl)phenyl)chroman-2-carboxamide;
(+)-6-fluoro-8-(4-methylpiperazin-1-yl)-N-(4-(2-oxooxazolidin-3-yl)phenyl)chroman-2-carboxamide; (Isomer 1)
(−)-6-fluoro-8-(4-methylpiperazin-1-yl)-N-(4-(2-oxooxazolidin-3-yl)phenyl)chroman-2-carboxamide; (Isomer 2)
6-fluoro-8-(4-methylpiperazin-1-yl)-N-(4-(2-oxopyrrolidin-1-yl)phenyl)chroman-2-carboxamide;
(+)-6-fluoro-8-(4-methylpiperazin-1-yl)-N-(4-(2-oxopyrrolidin-1-yl)phenyl)chroman-2-carboxamide; (Isomer 1);
(−)-6-fluoro-8-(4-methylpiperazin-1-yl)-N-(4-(2-oxopyrrolidin-1-yl)phenyl)chroman-2-carboxamide; (Isomer 2)
6-fluoro-N-(4-(3-methyl-2,5-dioxoimidazolidin-1-yl)phenyl)-8-(4-methylpiperazin-1-yl)chroman-2-carboxamide;
(+)-6-fluoro-N-(4-(3-methyl-2,5-dioxoimidazolidin-1-yl)phenyl)-8-(4-methylpiperazin-1-yl)chroman-2-carboxamide; (Isomer 1)
(−)-6-fluoro-N-(4-(3-methyl-2,5-dioxoimidazolidin-1-yl)phenyl)-8-(4-methylpiperazin-1-yl)chroman-2-carboxamide; (Isomer 2)
N-(4-(2,5-dioxoimidazolidin-1-yl)phenyl)-6-fluoro-8-(4-methylpiperazin-1-yl)chroman-2-carboxamide;
(+)-N-(4-(2,5-dioxoimidazolidin-1-yl)phenyl)-6-fluoro-8-(4-methylpiperazin-1-yl)chroman-2-carboxamide; (Isomer 1)
(−)-N-(4-(2,5-dioxoimidazolidin-1-yl)phenyl)-6-fluoro-8-(4-methylpiperazin-1-yl)chroman-2-carboxamide; (Isomer 2)
6-fluoro-8-(4-methylpiperazin-1-yl)-N-(4-(2-oxotetrahydropyrimidin-1(2H)-yl)phenyl)chroman-2-carboxamide;
(+)-6-fluoro-8-(4-methylpiperazin-1-yl)-N-(4-(2-oxotetrahydropyrimidin-1(2H)-yl)phenyl)chroman-2-carboxamide; (Isomer 1)
(−)-6-fluoro-8-(4-methylpiperazin-1-yl)-N-(4-(2-oxotetrahydropyrimidin-1(2H)-yl)phenyl)chroman-2-carboxamide; (Isomer 2)
6-methoxy-N-(4-(3-(methoxymethyl)-1,2,4-oxadiazol-5-yl)phenyl)-8-(4-methylpiperazin-1-yl)chroman-2-carboxamide;
(+)-6-methoxy-N-(4-(3-(methoxymethyl)-1,2,4-oxadiazol-5-yl)phenyl)-8-(4-methylpiperazin-1-yl)chroman-2-carboxamide; (Isomer 1)
(−)-6-methoxy-N-(4-(3-(methoxymethyl)-1,2,4-oxadiazol-5-yl)phenyl)-8-(4-methylpiperazin-1-yl)chroman-2-carboxamide; (Isomer 2)
6-fluoro-N-(4-(3-(methoxymethyl)-1,2,4-oxadiazol-5-yl)phenyl)-8-(4-methylpiperazin-1-yl)chroman-2-carboxamide;
(+)-6-fluoro-N-(4-(3-(methoxymethyl)-1,2,4-oxadiazol-5-yl)phenyl)-8-(4-methylpiperazin-1-yl)chroman-2-carboxamide; (Isomer 1)
(−)-6-fluoro-N-(4-(3-(methoxymethyl)-1,2,4-oxadiazol-5-yl)phenyl)-8-(4-methylpiperazin-1-yl)chroman-2-carboxamide; (Isomer 2)
6-methoxy-N-(4-(3-methyl-1,2,4-oxadiazol-5-yl)phenyl)-8-(4-methylpiperazin-1-yl)chroman-2-carboxamide;
(+)-6-methoxy-N-(4-(3-methyl-1,2,4-oxadiazol-5-yl)phenyl)-8-(4-methylpiperazin-1-yl)chroman-2-carboxamide; (Isomer 1)
(−)-6-methoxy-N-(4-(3-methyl-1,2,4-oxadiazol-5-yl)phenyl)-8-(4-methylpiperazin-1-yl)chroman-2-carboxamide; (Isomer 2)
6-fluoro-N-(4-(3-methyl-1,2,4-oxadiazol-5-yl)phenyl)-8-(4-methylpiperazin-1-yl)chroman-2-carboxamide;
(+)-6-fluoro-N-(4-(3-methyl-1,2,4-oxadiazol-5-yl)phenyl)-8-(4-methylpiperazin-1-yl)chroman-2-carboxamide; (Isomer 1)
(−)-6-fluoro-N-(4-(3-methyl-1,2,4-oxadiazol-5-yl)phenyl)-8-(4-methylpiperazin-1-yl)chroman-2-carboxamide; (Isomer 2)
N-(4-(4-ethoxypiperidin-1-yl)phenyl)-6-fluoro-8-(4-methylpiperazin-1-yl)chroman-2-carboxamide;
(+)-N-(4-(4-ethoxypiperidin-1-yl)phenyl)-6-fluoro-8-(4-methylpiperazin-1-yl)chroman-2-carboxamide; (Isomer 1)
(−)-N-(4-(4-ethoxypiperidin-1-yl)phenyl)-6-fluoro-8-(4-methylpiperazin-1-yl)chroman-2-carboxamide; (Isomer 2)
6-methoxy-N-(4-(5-methyl-1,2,4-oxadiazol-3-yl)phenyl)-8-(4-methylpiperazin-1-yl)chroman-2-carboxamide;
(+)-6-methoxy-N-(4-(5-methyl-1,2,4-oxadiazol-3-yl)phenyl)-8-(4-methylpiperazin-1-yl)chroman-2-carboxamide; (Isomer 1)
(−)-6-methoxy-N-(4-(5-methyl-1,2,4-oxadiazol-3-yl)phenyl)-8-(4-methylpiperazin-1-yl)chroman-2-carboxamide; (Isomer 2)
N-(4-(5-ethyl-1,2,4-oxadiazol-3-yl)phenyl)-6-methoxy-8-(4-methylpiperazin-1-yl)chroman-2-carboxamide;
(+)-N-(4-(5-ethyl-1,2,4-oxadiazol-3-yl)phenyl)-6-methoxy-8-(4-methylpiperazin-1-yl)chroman-2-carboxamide; (Isomer 1)
(−)-N-(4-(5-ethyl-1,2,4-oxadiazol-3-yl)phenyl)-6-methoxy-8-(4-methylpiperazin-1-yl)chroman-2-carboxamide; (Isomer 2)
N-(4-(5-isopropyl-1,2,4-oxadiazol-3-yl)phenyl)-6-methoxy-8-(4-methylpiperazin-1-yl)chroman-2-carboxamide;
(−)-N-(4-(5-isopropyl-1,2,4-oxadiazol-3-yl)phenyl)-6-methoxy-8-(4-methylpiperazin-1-yl)chroman-2-carboxamide; (Isomer 2)
(+)-N-(4-(5-isopropyl-1,2,4-oxadiazol-3-yl)phenyl)-6-methoxy-8-(4-methylpiperazin-1-yl)chroman-2-carboxamide; (Isomer 1)
6-methoxy-N-(4-(5-(methoxymethyl)-1,2,4-oxadiazol-3-yl)phenyl)-8-(4-methylpiperazin-1-yl)chroman-2-carboxamide;
(+)-6-methoxy-N-(4-(5-(methoxymethyl)-1,2,4-oxadiazol-3-yl)phenyl)-8-(4-methylpiperazin-1-yl)chroman-2-carboxamide; (Isomer 1)
(−)-6-methoxy-N-(4-(5-(methoxymethyl)-1,2,4-oxadiazol-3-yl)phenyl)-8-(4-methylpiperazin-1-yl)chroman-2-carboxamide; (Isomer 2)
6-fluoro-N-(4-(5-methyl-1,2,4-oxadiazol-3-yl)phenyl)-8-(4-methylpiperazin-1-yl)chroman-2-carboxamide;
(+)-6-fluoro-N-(4-(5-methyl-1,2,4-oxadiazol-3-yl)phenyl)-8-(4-methylpiperazin-1-yl)chroman-2-carboxamide; (Isomer 1)
(−)-6-fluoro-N-(4-(5-methyl-1,2,4-oxadiazol-3-yl)phenyl)-8-(4-methylpiperazin-1-yl)chroman-2-carboxamide; (Isomer 2)
N-(4-(5-ethyl-1,2,4-oxadiazol-3-yl)phenyl)-6-fluoro-8-(4-methylpiperazin-1-yl)chroman-2-carboxamide;
(+)-N-(4-(5-ethyl-1,2,4-oxadiazol-3-yl)phenyl)-6-fluoro-8-(4-methylpiperazin-1-yl)chroman-2-carboxamide; (Isomer 1)
(−)-N-(4-(5-ethyl-1,2,4-oxadiazol-3-yl)phenyl)-6-fluoro-8-(4-methylpiperazin-1-yl)chroman-2-carboxamide; (Isomer 2)
6-fluoro-N-(4-(5-isopropyl-1,2,4-oxadiazol-3-yl)phenyl)-8-(4-methylpiperazin-1-yl)chroman-2-carboxamide;
(+)-6-fluoro-N-(4-(5-isopropyl-1,2,4-oxadiazol-3-yl)phenyl)-8-(4-methylpiperazin-1-yl)chroman-2-carboxamide; (Isomer 1)
(−)-6-fluoro-N-(4-(5-isopropyl-1,2,4-oxadiazol-3-yl)phenyl)-8-(4-methylpiperazin-1-yl)chroman-2-carboxamide; (Isomer 2)
N-(4-(4-ethoxypiperidin-1-yl)phenyl)-6-methoxy-8-(4-methylpiperazin-1-yl)chroman-2-carboxamide;
(−)-N-(4-(4-ethoxypiperidin-1-yl)phenyl)-6-methoxy-8-(4-methylpiperazin-1-yl)chroman-2-carboxamide; (Isomer 2)
(+)-N-(4-(4-ethoxypiperidin-1-yl)phenyl)-6-methoxy-8-(4-methylpiperazin-1-yl)chroman-2-carboxamide; (Isomer 1)
6-methoxy-N-(4-(4-methyloxazol-2-yl)phenyl)-8-(4-methylpiperazin-1-yl)chroman-2-carboxamide;
(+)-6-methoxy-N-(4-(4-methyloxazol-2-yl)phenyl)-8-(4-methylpiperazin-1-yl)chroman-2-carboxamide; (Isomer 1)
(−)-6-methoxy-N-(4-(4-methyloxazol-2-yl)phenyl)-8-(4-methylpiperazin-1-yl)chroman-2-carboxamide; (Isomer 2)
6-methoxy-N-(4-morpholinophenyl)-8-(4-(2,2,2-trifluoroethyl)piperazin-1-yl)chroman-2-carboxamide;
(+)-6-methoxy-N-(4-morpholinophenyl)-8-(4-(2,2,2-trifluoroethyl)piperazin-1-yl)chroman-2-carboxamide; (Isomer 1)
(−)-6-methoxy-N-(4-morpholinophenyl)-8-(4-(2,2,2-trifluoroethyl)piperazin-1-yl)chroman-2-carboxamide; (Isomer 2)
6-methoxy-8-(4-(2-methoxyethyl)piperazin-1-yl)-N-(4-morpholinophenyl)chroman-2-carboxamide;
(+)-6-methoxy-8-(4-(2-methoxyethyl)piperazin-1-yl)-N-(4-morpholinophenyl)chroman-2-carboxamide; (Isomer 1)
(−)-6-methoxy-8-(4-(2-methoxyethyl)piperazin-1-yl)-N-(4-morpholinophenyl)chroman-2-carboxamide; (Isomer 2)
8-(4-butylpiperazine-1-yl)-6-methoxy-N-(4-morpholinophenyl)chroman-2-carboxamide;
(+)-8-(4-butylpiperazine-1-yl)-6-methoxy-N-(4-morpholinophenyl)chroman-2-carboxamide; (Isomer 1)
(−)-8-(4-butylpiperazine-1-yl)-6-methoxy-N-(4-morpholinophenyl)chroman-2-carboxamide; (Isomer 2)
6-methoxy-N-(4-morpholinophenyl)-8-(4-propylpiperazin-1-yl)chroman-2-carboxamide;
(+)-6-methoxy-N-(4-morpholinophenyl)-8-(4-propylpiperazin-1-yl)chroman-2-carboxamide; (Isomer 1)
(−)-6-methoxy-N-(4-morpholinophenyl)-8-(4-propylpiperazin-1-yl)chroman-2-carboxamide; (Isomer 2)
8-(4-isopropylpiperazin-1-yl)-6-methoxy-N-(4-morpholinophenyl)chroman-2-carboxamide;
(+)-8-(4-isopropylpiperazin-1-yl)-6-methoxy-N-(4-morpholinophenyl)chroman-2-carboxamide; (Isomer 1)
(−)-8-(4-isopropylpiperazin-1-yl)-6-methoxy-N-(4-morpholinophenyl)chroman-2-carboxamide; (Isomer 2)
8-(4-ethylpiperazin-1-yl)-6-methoxy-N-(4-morpholinophenyl)chroman-2-carboxamide;
(+)-8-(4-ethylpiperazin-1-yl)-6-methoxy-N-(4-morpholinophenyl)chroman-2-carboxamide; (Isomer 1)
(−)-8-(4-ethylpiperazin-1-yl)-6-methoxy-N-(4-morpholinophenyl)chroman-2-carboxamide; (Isomer 2)
6-methoxy-N-(4-morpholinophenyl)-8-(piperazin-1-yl)chroman-2-carboxamide;
(+)-6-methoxy-N-(4-morpholinophenyl)-8-(piperazin-1-yl)chroman-2-carboxamide; (Isomer 1)
(−)-6-methoxy-N-(4-morpholinophenyl)-8-(piperazin-1-yl)chroman-2-carboxamide; (Isomer 2)
6-methoxy-2-methyl-8-(4-methylpiperazin-1-yl)-N-(4-morpholinophenyl)chroman-2-carboxamide;
(+)-6-methoxy-2-methyl-8-(4-methylpiperazin-1-yl)-N-(4-morpholinophenyl)chroman-2-carboxamide; (Isomer 1)
(−)-6-methoxy-2-methyl-8-(4-methylpiperazin-1-yl)-N-(4-morpholinophenyl)chroman-2-carboxamide; (Isomer 2)
6-hydroxy-8-(4-methylpiperazin-1-yl)-N-(4-morpholinophenyl)chroman-2-carboxamide;
(+)-6-hydroxy-8-(4-methylpiperazin-1-yl)-N-(4-morpholinophenyl)chroman-2-carboxamide; (Isomer 1), and
(−)-6-hydroxy-8-(4-methylpiperazin-1-yl)-N-(4-morpholinophenyl)chroman-2-carboxamide; (Isomer 2)
or a pharmaceutically-acceptable salt thereof.

27. A compound selected from:
(+)-6-methoxy-8-(4-methylpiperazin-1-yl)-N-(4-(2-oxopyrrolidin-1-yl)phenyl)chroman-2-carboxamide; (Isomer 1)
(+)-6-methoxy-8-(4-methylpiperazin-1-yl)-N-(4-(2-oxotetrahydropyrimidin-1(2H)-yl)phenyl)chroman-2-carboxamide; (Isomer 1)
(+)-6-methoxy-N-(4-(3-methyl-2-oxotetrahydropyrimidin-1(2H)-yl)phenyl)-8-(4-methylpiperazin-1-yl)chroman-2-carboxamide; (Isomer 1)
(+)-6-fluoro-N-(4-(3-methyl-2-oxotetrahydropyrimidin-1(2H)-yl)phenyl)-8-(4-methylpiperazin-1-yl)chroman-2-carboxamide; (Isomer 1)
(+)-6-methoxy-8-(4-methylpiperazin-1-yl)-N-(4-(2-oxooxazolidin-3-yl)phenyl)chroman-2-carboxamide; (Isomer 1)
(+)-6-methoxy-N-(4-(3-methyl-2,5-dioxoimidazolidin-1-yl)phenyl)-8-(4-methylpiperazin-1-yl)chroman-2-carboxamide; (Isomer 1)
(+)-N-(4-(2,5-dioxoimidazolidin-1-yl)phenyl)-6-methoxy-8-(4-methylpiperazin-1-yl)chroman-2-carboxamide; (Isomer 1)
(+)-6-fluoro-8-(4-methylpiperazin-1-yl)-N-(4-(2-oxooxazolidin-3-yl)phenyl)chroman-2-carboxamide; (Isomer 1)
(+)-6-fluoro-8-(4-methylpiperazin-1-yl)-N-(4-(2-oxopyrrolidin-1-yl)phenyl)chroman-2-carboxamide; (Isomer 1)
(+)-6-fluoro-N-(4-(3-methyl-2,5-dioxoimidazolidin-1-yl)phenyl)-8-(4-methylpiperazin-1-yl)chroman-2-carboxamide; (Isomer 1)
(+)-N-(4-(2,5-dioxoimidazolidin-1-yl)phenyl)-6-fluoro-8-(4-methylpiperazin-1-yl)chroman-2-carboxamide; (Isomer 1)
(+)-6-fluoro-8-(4-methylpiperazin-1-yl)-N-(4-(2-oxotetrahydropyrimidin-1(2H)-yl)phenyl)chroman-2-carboxamide; (Isomer 1)
(+)-6-methoxy-N-(4-(3-(methoxymethyl)-1,2,4-oxadiazol-5-yl)phenyl)-8-(4-methylpiperazin-1-yl)chroman-2-carboxamide; (Isomer 1)
(+)-6-fluoro-N-(4-(3-(methoxymethyl)-1,2,4-oxadiazol-5-yl)phenyl)-8-(4-methylpiperazin-1-yl)chroman-2-carboxamide; (Isomer 1)
(+)-6-methoxy-N-(4-(3-methyl-1,2,4-oxadiazol-5-yl)phenyl)-8-(4-methylpiperazin-1-yl)chroman-2-carboxamide; (Isomer 1)
(+)-6-fluoro-N-(4-(3-methyl-1,2,4-oxadiazol-5-yl)phenyl)-8-(4-methylpiperazin-1-yl)chroman-2-carboxamide; (Isomer 1)
(+)-N-(4-(4-ethoxypiperidin-1-yl)phenyl)-6-fluoro-8-(4-methylpiperazin-1-yl)chroman-2-carboxamide; (Isomer 1)
(+)-6-methoxy-N-(4-(5-methyl-1,2,4-oxadiazol-3-yl)phenyl)-8-(4-methylpiperazin-1-yl)chroman-2-carboxamide; (Isomer 1)
(+)-N-(4-(5-ethyl-1,2,4-oxadiazol-3-yl)phenyl)-6-methoxy-8-(4-methylpiperazin-1-yl)chroman-2-carboxamide; (Isomer 1)
(+)-N-(4-(5-isopropyl-1,2,4-oxadiazol-3-yl)phenyl)-6-methoxy-8-(4-methylpiperazin-1-yl)chroman-2-carboxamide; (Isomer 1)
(+)-6-methoxy-N-(4-(5-(methoxymethyl)-1,2,4-oxadiazol-3-yl)phenyl)-8-(4-methylpiperazin-1-yl)chroman-2-carboxamide; (Isomer 1)
(+)-6-fluoro-N-(4-(5-methyl-1,2,4-oxadiazol-3-yl)phenyl)-8-(4-methylpiperazin-1-yl)chroman-2-carboxamide; (Isomer 1)
(+)-N-(4-(5-ethyl-1,2,4-oxadiazol-3-yl)phenyl)-6-fluoro-8-(4-methylpiperazin-1-yl)chroman-2-carboxamide; (Isomer 1)
(+)-6-fluoro-N-(4-(5-isopropyl-1,2,4-oxadiazol-3-yl)phenyl)-8-(4-methylpiperazin-1-yl)chroman-2-carboxamide; (Isomer 1)
(+)-N-(4-(4-ethoxypiperidin-1-yl)phenyl)-6-methoxy-8-(4-methylpiperazin-1-yl)chroman-2-carboxamide; (Isomer 1)
(+)-6-methoxy-N-(4-(4-methyloxazol-2-yl)phenyl)-8-(4-methylpiperazin-1-yl)chroman-2-carboxamide; (Isomer 1)
(+)-6-methoxy-N-(4-morpholinophenyl)-8-(4-(2,2,2-trifluoroethyl)piperazin-1-yl)chroman-2-carboxamide; (Isomer 1)
(+)-6-methoxy-8-(4-(2-methoxyethyl)piperazin-1-yl)-N-(4-morpholinophenyl)chroman-2-carboxamide; (Isomer 1)
(+)-8-(4-butylpiperazine-1-yl)-6-methoxy-N-(4-morpholinophenyl)chroman-2-carboxamide; (Isomer 1)
(+)-6-methoxy-N-(4-morpholinophenyl)-8-(4-propylpiperazin-1-yl)chroman-2-carboxamide; (Isomer 1)
(+)-8-(4-isopropylpiperazin-1-yl)-6-methoxy-N-(4-morpholinophenyl)chroman-2-carboxamide; (Isomer 1)
(+)-8-(4-ethylpiperazin-1-yl)-6-methoxy-N-(4-morpholinophenyl)chroman-2-carboxamide; (Isomer 1)
(+)-6-methoxy-N-(4-morpholinophenyl)-8-(piperazin-1-yl)chroman-2-carboxamide; (Isomer 1)
(+)-6-methoxy-2-methyl-8-(4-methylpiperazin-1-yl)-N-(4-morpholinophenyl)chroman-2-carboxamide; (Isomer 1), and
(+)-6-hydroxy-8-(4-methylpiperazin-1-yl)-N-(4-morpholinophenyl)chroman-2-carboxamide; (Isomer 1)
or a pharmaceutically-acceptable salt thereof.

28. A method of treating anxiety disorders in a mammal comprising administering to such mammal an effective amount of a compound of formula I 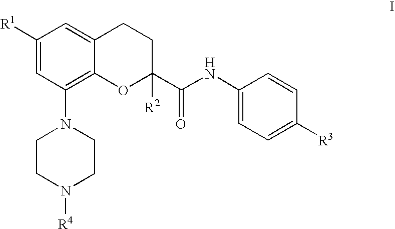
wherein:
R¹ is F or OCH₃ or OH;
R² is H, or CH₃;
R³ is a heterocycle optionally substituted with one of the following —CH₃, —CH₂CH₃, —CH₂CH₂CH₃, —CH(CH₃)₂, —CH₂OCH₃, or —OCH₂CH₃; and
R⁴ is H, —CH₃, CH₂CF₃, —CH₂CH₂OCH₃, CH₂CH₂CH₂CH₃, —CH₂CH₂CH₃, —CH(CH₃)₂, —CH₂CH₃;
or a pharmaceutically-acceptable salt thereof.

29. The method of claim 28 wherein the disorder is a mood disorder.

30. The method of claim 28 wherein the disorder is a cognitive disorder.

31. A method of treating anxiety disorders in a mammal comprising administering to such mammal an effective amount of a compound selected from:
6-methoxy-8-(4-methylpiperazin-1-yl)-N-(4-(2-oxopyrrolidin-1-yl)phenyl)chroman-2-carboxamide;
(+)-6-methoxy-8-(4-methylpiperazin-1-yl)-N-(4-(2-oxopyrrolidin-1-yl)phenyl)chroman-2-carboxamide; (Isomer 1)
(−)-6-methoxy-8-(4-methylpiperazin-1-yl)-N-(4-(2-oxopyrrolidin-1-yl)phenyl)chroman-2-carboxamide; (Isomer 2)
6-methoxy-8-(4-methylpiperazin-1-yl)-N-(4-(2-oxotetrahydropyrimidin-1(2H)-yl)phenyl)chroman-2-carboxamide;
(+)-6-methoxy-8-(4-methylpiperazin-1-yl)-N-(4-(2-oxotetrahydropyrimidin-1(2H)-yl)phenyl)chroman-2-carboxamide; (Isomer 1)
(−)-6-methoxy-8-(4-methylpiperazin-1-yl)-N-(4-(2-oxotetrahydropyrimidin-1(2H)-yl)phenyl)chroman-2-carboxamide; (Isomer 2)
6-methoxy-N-(4-(3-methyl-2-oxotetrahydropyrimidin-1(2H)-yl)phenyl)-8-(4-methylpiperazin-1-yl)chroman-2-carboxamide;
(+)-6-methoxy-N-(4-(3-methyl-2-oxotetrahydropyrimidin-1(2H)-yl)phenyl)-8-(4-methylpiperazin-1-yl)chroman-2-carboxamide; (Isomer 1)
(−)-6-methoxy-N-(4-(3-methyl-2-oxotetrahydropyrimidin-1(2H)-yl)phenyl)-8-(4-methylpiperazin-1-yl)chroman-2-carboxamide; (Isomer 2)
6-fluoro-N-(4-(3-methyl-2-oxotetrahydropyrimidin-1(2H)-yl)phenyl)-8-(4-methylpiperazin-1-yl)chroman-2-carboxamide;
(+)-6-fluoro-N-(4-(3-methyl-2-oxotetrahydropyrimidin-1(2H)-yl)phenyl)-8-(4-methylpiperazin-1-yl)chroman-2-carboxamide; (Isomer 1)
6-methoxy-8-(4-methylpiperazin-1-yl)-N-(4-(2-oxooxazolidin-3-yl)phenyl)chroman-2-carboxamide;
(+)-6-methoxy-8-(4-methylpiperazin-1-yl)-N-(4-(2-oxooxazolidin-3-yl)phenyl)chroman-2-carboxamide; (Isomer 1)
(−)-6-methoxy-8-(4-methylpiperazin-1-yl)-N-(4-(2-oxooxazolidin-3-yl)phenyl)chroman-2-carboxamide; (Isomer 2)
6-methoxy-N-(4-(3-methyl-2,5-dioxoimidazolidin-1-yl)phenyl)-8-(4-methylpiperazin-1-yl)chroman-2-carboxamide;
(+)-6-methoxy-N-(4-(3-methyl-2,5-dioxoimidazolidin-1-yl)phenyl)-8-(4-methylpiperazin-1-yl)chroman-2-carboxamide; (Isomer 1)
(−)-6-methoxy-N-(4-(3-methyl-2,5-dioxoimidazolidin-1-yl)phenyl)-8-(4-methylpiperazin-1-yl)chroman-2-carboxamide; (Isomer 2)
N-(4-(2,5-dioxoimidazolidin-1-yl)phenyl)-6-methoxy-8-(4-methylpiperazin-1-yl)chroman-2-carboxamide;
(+)-N-(4-(2,5-dioxoimidazolidin-1-yl)phenyl)-6-methoxy-8-(4-methylpiperazin-1-yl)chroman-2-carboxamide; (Isomer 1)
(−)-N-(4-(2,5-dioxoimidazolidin-1-yl)phenyl)-6-methoxy-8-(4-methylpiperazin-1-yl)chroman-2-carboxamide; (Isomer 2)
6-fluoro-8-(4-methylpiperazin-1-yl)-N-(4-(2-oxooxazolidin-3-yl)phenyl)chroman-2-carboxamide;
(+)-6-fluoro-8-(4-methylpiperazin-1-yl)-N-(4-(2-oxooxazolidin-3-yl)phenyl)chroman-2-carboxamide; (Isomer 1)
(−)-6-fluoro-8-(4-methylpiperazin-1-yl)-N-(4-(2-oxooxazolidin-3-yl)phenyl)chroman-2-carboxamide; (Isomer 2)
6-fluoro-8-(4-methylpiperazin-1-yl)-N-(4-(2-oxopyrrolidin-1-yl)phenyl)chroman-2-carboxamide;
(+)-6-fluoro-8-(4-methylpiperazin-1-yl)-N-(4-(2-oxopyrrolidin-1-yl)phenyl)chroman-2-carboxamide; (Isomer 1)
(−)-6-fluoro-8-(4-methylpiperazin-1-yl)-N-(4-(2-oxopyrrolidin-1-yl)phenyl)chroman-2-carboxamide; (Isomer 2)
6-fluoro-N-(4-(3-methyl-2,5-dioxoimidazolidin-1-yl)phenyl)-8-(4-methylpiperazin-1-yl)chroman-2-carboxamide;
(+)-6-fluoro-N-(4-(3-methyl-2,5-dioxoimidazolidin-1-yl)phenyl)-8-(4-methylpiperazin-1-yl)chroman-2-carboxamide; (Isomer 1)
(−)-6-fluoro-N-(4-(3-methyl-2,5-dioxoimidazolidin-1-yl)phenyl)-8-(4-methylpiperazin-1-yl)chroman-2-carboxamide; (Isomer 2)
N-(4-(2,5-dioxoimidazolidin-1-yl)phenyl)-6-fluoro-8-(4-methylpiperazin-1-yl)chroman-2-carboxamide;
(+)-N-(4-(2,5-dioxoimidazolidin-1-yl)phenyl)-6-fluoro-8-(4-methylpiperazin-1-yl)chroman-2-carboxamide; (Isomer 1)
(−)-N-(4-(2,5-dioxoimidazolidin-1-yl)phenyl)-6-fluoro-8-(4-methylpiperazin-1-yl)chroman-2-carboxamide; (Isomer 2)
6-fluoro-8-(4-methylpiperazin-1-yl)-N-(4-(2-oxotetrahydropyrimidin-1(2H)-yl)phenyl)chroman-2-carboxamide;
(+)-6-fluoro-8-(4-methylpiperazin-1-yl)-N-(4-(2-oxotetrahydropyrimidin-1(2H)-yl)phenyl)chroman-2-carboxamide; (Isomer 1)
(−)-6-fluoro-8-(4-methylpiperazin-1-yl)-N-(4-(2-oxotetrahydropyrimidin-1(2H)-yl)phenyl)chroman-2-carboxamide; (Isomer 2)
6-methoxy-N-(4-(3-(methoxymethyl)-1,2,4-oxadiazol-5-yl)phenyl)-8-(4-methylpiperazin-1-yl)chroman-2-carboxamide;
(+)-6-methoxy-N-(4-(3-(methoxymethyl)-1,2,4-oxadiazol-5-yl)phenyl)-8-(4-methylpiperazin-1-yl)chroman-2-carboxamide; (Isomer 1)
(−)-6-methoxy-N-(4-(3-(methoxymethyl)-1,2,4-oxadiazol-5-yl)phenyl)-8-(4-methylpiperazin-1-yl)chroman-2-carboxamide; (Isomer 2)
6-fluoro-N-(4-(3-(methoxymethyl)-1,2,4-oxadiazol-5-yl)phenyl)-8-(4-methylpiperazin-1-yl)chroman-2-carboxamide;
(+)-6-fluoro-N-(4-(3-(methoxymethyl)-1,2,4-oxadiazol-5-yl)phenyl)-8-(4-methylpiperazin-1-yl)chroman-2-carboxamide; (Isomer 1)
(−)-6-fluoro-N-(4-(3-(methoxymethyl)-1,2,4-oxadiazol-5-yl)phenyl)-8-(4-methylpiperazin-1-yl)chroman-2-carboxamide; (Isomer 2)
6-methoxy-N-(4-(3-methyl-1,2,4-oxadiazol-5-yl)phenyl)-8-(4-methylpiperazin-1-yl)chroman-2-carboxamide;
(+)-6-methoxy-N-(4-(3-methyl-1,2,4-oxadiazol-5-yl)phenyl)-8-(4-methylpiperazin-1-yl)chroman-2-carboxamide; (Isomer 1)
(−)-6-methoxy-N-(4-(3-methyl-1,2,4-oxadiazol-5-yl)phenyl)-8-(4-methylpiperazin-1-yl)chroman-2-carboxamide; (Isomer 2)
6-fluoro-N-(4-(3-methyl-1,2,4-oxadiazol-5-yl)phenyl)-8-(4-methylpiperazin-1-yl)chroman-2-carboxamide;
(+)-6-fluoro-N-(4-(3-methyl-1,2,4-oxadiazol-5-yl)phenyl)-8-(4-methylpiperazin-1-yl)chroman-2-carboxamide; (Isomer 1)
(−)-6-fluoro-N-(4-(3-methyl-1,2,4-oxadiazol-5-yl)phenyl)-8-(4-methylpiperazin-1-yl)chroman-2-carboxamide; (Isomer 2)
N-(4-(4-ethoxypiperidin-1-yl)phenyl)-6-fluoro-8-(4-methylpiperazin-1-yl)chroman-2-carboxamide;
(+)-N-(4-(4-ethoxypiperidin-1-yl)phenyl)-6-fluoro-8-(4-methylpiperazin-1-yl)chroman-2-carboxamide; (Isomer 1)
(−)-N-(4-(4-ethoxypiperidin-1-yl)phenyl)-6-fluoro-8-(4-methylpiperazin-1-yl)chroman-2-carboxamide; (Isomer 2)
6-methoxy-N-(4-(5-methyl-1,2,4-oxadiazol-3-yl)phenyl)-8-(4-methylpiperazin-1-yl)chroman-2-carboxamide;
(+)-6-methoxy-N-(4-(5-methyl-1,2,4-oxadiazol-3-yl)phenyl)-8-(4-methylpiperazin-1-yl)chroman-2-carboxamide; (Isomer-1)
(−)-6-methoxy-N-(4-(5-methyl-1,2,4-oxadiazol-3-yl)phenyl)-8-(4-methylpiperazin-1-yl)chroman-2-carboxamide; (Isomer 2)
N-(4-(5-ethyl-1,2,4-oxadiazol-3-yl)phenyl)-6-methoxy-8-(4-methylpiperazin-1-yl)chroman-2-carboxamide;
(+)-N-(4-(5-ethyl-1,2,4-oxadiazol-3-yl)phenyl)-6-methoxy-8-(4-methylpiperazin-1-yl)chroman-2-carboxamide; (Isomer 1)
(−)-N-(4-(5-ethyl-1,2,4-oxadiazol-3-yl)phenyl)-6-methoxy-8-(4-methylpiperazin-1-yl)chroman-2-carboxamide; (Isomer 2)
N-(4-(5-isopropyl-1,2,4-oxadiazol-3-yl)phenyl)-6-methoxy-8-(4-methylpiperazin-1-yl)chroman-2-carboxamide;
(−)-N-(4-(5-isopropyl-1,2,4-oxadiazol-3-yl)phenyl)-6-methoxy-8-(4-methylpiperazin-1-yl)chroman-2-carboxamide; (Isomer 2)
(+)-N-(4-(5-isopropyl-1,2,4-oxadiazol-3-yl)phenyl)-6-methoxy-8-(4-methylpiperazin-1-yl)chroman-2-carboxamide; (Isomer 1)
6-methoxy-N-(4-(5-(methoxymethyl)-1,2,4-oxadiazol-3-yl)phenyl)-8-(4-methylpiperazin-1-yl)chroman-2-carboxamide;
(+)-6-methoxy-N-(4-(5-(methoxymethyl)-1,2,4-oxadiazol-3-yl)phenyl)-8-(4-methylpiperazin-1-yl)chroman-2-carboxamide; (Isomer 1)
(−)-6-methoxy-N-(4-(5-(methoxymethyl)-1,2,4-oxadiazol-3-yl)phenyl)-8-(4-methylpiperazin-1-yl)chroman-2-carboxamide; (Isomer 2)
6-fluoro-N-(4-(5-methyl-1,2,4-oxadiazol-3-yl)phenyl)-8-(4-methylpiperazin-1-yl)chroman-2-carboxamide;
(+)-6-fluoro-N-(4-(5-methyl-1,2,4-oxadiazol-3-yl)phenyl)-8-(4-methylpiperazin-1-yl)chroman-2-carboxamide; (Isomer 1)
(−)-6-fluoro-N-(4-(5-methyl-1,2,4-oxadiazol-3-yl)phenyl)-8-(4-methylpiperazin-1-yl)chroman-2-carboxamide; (Isomer 2)
N-(4-(5-ethyl-1,2,4-oxadiazol-3-yl)phenyl)-6-fluoro-8-(4-methylpiperazin-1-yl)chroman-2-carboxamide;
(+)-N-(4-(5-ethyl-1,2,4-oxadiazol-3-yl)phenyl)-6-fluoro-8-(4-methylpiperazin-1-yl)chroman-2-carboxamide; (Isomer 1)
(−)-N-(4-(5-ethyl-1,2,4-oxadiazol-3-yl)phenyl)-6-fluoro-8-(4-methylpiperazin-1-yl)chroman-2-carboxamide; (Isomer 2)
6-fluoro-N-(4-(5-isopropyl-1,2,4-oxadiazol-3-yl)phenyl)-8-(4-methylpiperazin-1-yl)chroman-2-carboxamide;
(+)-6-fluoro-N-(4-(5-isopropyl-1,2,4-oxadiazol-3-yl)phenyl)-8-(4-methylpiperazin-1-yl)chroman-2-carboxamide; (Isomer 1)
(−)-6-fluoro-N-(4-(5-isopropyl-1,2,4-oxadiazol-3-yl)phenyl)-8-(4-methylpiperazin-1-yl)chroman-2-carboxamide; (Isomer 2)
N-(4-(4-ethoxypiperidin-1-yl)phenyl)-6-methoxy-8-(4-methylpiperazin-1-yl)chroman-2-carboxamide;
(−)-N-(4-(4-ethoxypiperidin-1-yl)phenyl)-6-methoxy-8-(4-methylpiperazin-1-yl)chroman-2-carboxamide; (Isomer 2)
(+)-N-(4-(4-ethoxypiperidin-1-yl)phenyl)-6-methoxy-8-(4-methylpiperazin-1-yl)chroman-2-carboxamide; (Isomer 1)
6-methoxy-N-(4-(4-methyloxazol-2-yl)phenyl)-8-(4-methylpiperazin-1-yl)chroman-2-carboxamide;
(+)-6-methoxy-N-(4-(4-methyloxazol-2-yl)phenyl)-8-(4-methylpiperazin-1-yl)chroman-2-carboxamide; (Isomer 1)
(−)-6-methoxy-N-(4-(4-methyloxazol-2-yl)phenyl)-8-(4-methylpiperazin-1-yl)chroman-2-carboxamide; (Isomer 2)
6-methoxy-N-(4-morpholinophenyl)-8-(4-(2,2,2-trifluoroethyl)piperazin-1-yl)chroman-2-carboxamide;
(+)-6-methoxy-N-(4-morpholinophenyl)-8-(4-(2,2,2-trifluoroethyl)piperazin-1-yl)chroman-2-carboxamide; (Isomer 1)
(−)-6-methoxy-N-(4-morpholinophenyl)-8-(4-(2,2,2-trifluoroethyl)piperazin-1-yl)chroman-2-carboxamide; (Isomer 2)
6-methoxy-8-(4-(2-methoxyethyl)piperazin-1-yl)-N-(4-morpholinophenyl)chroman-2-carboxamide;
(+)-6-methoxy-8-(4-(2-methoxyethyl)piperazin-1-yl)-N-(4-morpholinophenyl)chroman-2-carboxamide; (Isomer 1)
(−)-6-methoxy-8-(4-(2-methoxyethyl)piperazin-1-yl)-N-(4-morpholinophenyl)chroman-2-carboxamide; (Isomer 2)
8-(4-butylpiperazine-1-yl)-6-methoxy-N-(4-morpholinophenyl)chroman-2-carboxamide;
(+)-8-(4-butylpiperazine-1-yl)-6-methoxy-N-(4-morpholinophenyl)chroman-2-carboxamide; (Isomer 1)
(−)-8-(4-butylpiperazine-1-yl)-6-methoxy-N-(4-morpholinophenyl)chroman-2-carboxamide; (Isomer 2)
6-methoxy-N-(4-morpholinophenyl)-8-(4-propylpiperazin-1-yl)chroman-2-carboxamide;
(+)-6-methoxy-N-(4-morpholinophenyl)-8-(4-propylpiperazin-1-yl)chroman-2-carboxamide; (Isomer 1)
(−)-6-methoxy-N-(4-morpholinophenyl)-8-(4-propylpiperazin-1-yl)chroman-2-carboxamide; (Isomer 2)
8-(4-isopropylpiperazin-1-yl)-6-methoxy-N-(4-morpholinophenyl)chroman-2-carboxamide;
(+)-8-(4-isopropylpiperazin-1-yl)-6-methoxy-N-(4-morpholinophenyl)chroman-2-carboxamide; (Isomer 1)
(−)-8-(4-isopropylpiperazin-1-yl)-6-methoxy-N-(4-morpholinophenyl)chroman-2-carboxamide; (Isomer 2)
8-(4-ethylpiperazin-1-yl)-6-methoxy-N-(4-morpholinophenyl)chroman-2-carboxamide;
(+)-8-(4-ethylpiperazin-1-yl)-6-methoxy-N-(4-morpholinophenyl)chroman-2-carboxamide; (Isomer 1)
(−)-8-(4-ethylpiperazin-1-yl)-6-methoxy-N-(4-morpholinophenyl)chroman-2-carboxamide; (Isomer 2)
6-methoxy-N-(4-morpholinophenyl)-8-(piperazin-1-yl)chroman-2-carboxamide;
(+)-6-methoxy-N-(4-morpholinophenyl)-8-(piperazin-1-yl)chroman-2-carboxamide; (Isomer 1)
(−)-6-methoxy-N-(4-morpholinophenyl)-8-(piperazin-1-yl)chroman-2-carboxamide; (Isomer 2)
6-methoxy-2-methyl-8-(4-methylpiperazin-1-yl)-N-(4-morpholinophenyl)chroman-2-carboxamide;
(+)-6-methoxy-2-methyl-8-(4-methylpiperazin-1-yl)-N-(4-morpholinophenyl)chroman-2-carboxamide; (Isomer 1)
(−)-6-methoxy-2-methyl-8-(4-methylpiperazin-1-yl)-N-(4-morpholinophenyl)chroman-2-carboxamide; (Isomer 2)
6-hydroxy-8-(4-methylpiperazin-1-yl)-N-(4-morpholinophenyl)chroman-2-carboxamide;
(+)-6-hydroxy-8-(4-methylpiperazin-1-yl)-N-(4-morpholinophenyl)chroman-2-carboxamide; (Isomer 1), and
(−)-6-hydroxy-8-(4-methylpiperazin-1-yl)-N-(4-morpholinophenyl)chroman-2-carboxamide; (Isomer 2)
or a pharmaceutically-acceptable salt thereof.

32. The method of claim 31 wherein the disorder is a mood disorder.

33. The method of claim 31 wherein the disorder is a cognitive disorder.

34. A method of treating anxiety disorders in a mammal comprising administering to such mammal an effective amount of a compound selected from:
(+)-6-methoxy-8-(4-methylpiperazin-1-yl)-N-(4-(2-oxopyrrolidin-1-yl)phenyl)chroman-2-carboxamide; (Isomer 1)
(+)-6-methoxy-8-(4-methylpiperazin-1-yl)-N-(4-(2-oxotetrahydropyrimidin-1(2H)-yl)phenyl)chroman-2-carboxamide; (Isomer 1)
(+)-6-methoxy-N-(4-(3-methyl-2-oxotetrahydropyrimidin-1(2H)-yl)phenyl)-8-(4-methylpiperazin-1-yl)chroman-2-carboxamide; (Isomer 1)
(+)-6-fluoro-N-(4-(3-methyl-2-oxotetrahydropyrimidin-1(2H)-yl)phenyl)-8-(4-methylpiperazin-1-yl)chroman-2-carboxamide; (Isomer 1)
(+)-6-methoxy-8-(4-methylpiperazin-1-yl)-N-(4-(2-oxooxazolidin-3-yl)phenyl)chroman-2-carboxamide; (Isomer 1)
(+)-6-methoxy-N-(4-(3-methyl-2,5-dioxoimidazolidin-1-yl)phenyl)-8-(4-methylpiperazin-1-yl)chroman-2-carboxamide; (Isomer 1)
(+)-N-(4-(2,5-dioxoimidazolidin-1-yl)phenyl)-6-methoxy-8-(4-methylpiperazin-1-yl)chroman-2-carboxamide; (Isomer 1)
(+)-6-fluoro-8-(4-methylpiperazin-1-yl)-N-(4-(2-oxooxazolidin-3-yl)phenyl)chroman-2-carboxamide; (Isomer 1)
(+)-6-fluoro-8-(4-methylpiperazin-1-yl)-N-(4-(2-oxopyrrolidin-1-yl)phenyl)chroman-2-carboxamide; (Isomer 1)
(+)-6-fluoro-N-(4-(3-methyl-2,5-dioxoimidazolidin-1-yl)phenyl)-8-(4-methylpiperazin-1-yl)chroman-2-carboxamide; (Isomer 1)
(+)-N-(4-(2,5-dioxoimidazolidin-1-yl)phenyl)-6-fluoro-8-(4-methylpiperazin-1-yl)chroman-2-carboxamide; (Isomer 1)
(+)-6-fluoro-8-(4-methylpiperazin-1-yl)-N-(4-(2-oxotetrahydropyrimidin-1(2H)-yl)phenyl)chroman-2-carboxamide; (Isomer 1)
(+)-6-methoxy-N-(4-(3-(methoxymethyl)-1,2,4-oxadiazol-5-yl)phenyl)-8-(4-methylpiperazin-1-yl)chroman-2-carboxamide; (Isomer 1)
(+)-6-fluoro-N-(4-(3-(methoxymethyl)-1,2,4-oxadiazol-5-yl)phenyl)-8-(4-methylpiperazin-1-yl)chroman-2-carboxamide; (Isomer 1)
(+)-6-methoxy-N-(4-(3-methyl-1,2,4-oxadiazol-5-yl)phenyl)-8-(4-methylpiperazin-1-yl)chroman-2-carboxamide; (Isomer 1)
(+)-6-fluoro-N-(4-(3-methyl-1,2,4-oxadiazol-5-yl)phenyl)-8-(4-methylpiperazin-1-yl)chroman-2-carboxamide; (Isomer 1)
(+)-N-(4-(4-ethoxypiperidin-1-yl)phenyl)-6-fluoro-8-(4-methylpiperazin-1-yl)chroman-2-carboxamide; (Isomer 1)
(+)-6-methoxy-N-(4-(5-methyl-1,2,4-oxadiazol-3-yl)phenyl)-8-(4-methylpiperazin-1-yl)chroman-2-carboxamide; (Isomer 1)
(+)-N-(4-(5-ethyl-1,2,4-oxadiazol-3-yl)phenyl)-6-methoxy-8-(4-methylpiperazin-1-yl)chroman-2-carboxamide; (Isomer 1)
(+)-N-(4-(5-isopropyl-1,2,4-oxadiazol-3-yl)phenyl)-6-methoxy-8-(4-methylpiperazin-1-yl)chroman-2-carboxamide; (Isomer 1)
(+)-6-methoxy-N-(4-(5-(methoxymethyl)-1,2,4-oxadiazol-3-yl)phenyl)-8-(4-methylpiperazin-1-yl)chroman-2-carboxamide; (Isomer 1)
(+)-6-fluoro-N-(4-(5-methyl-1,2,4-oxadiazol-3-yl)phenyl)-8-(4-methylpiperazin-1-yl)chroman-2-carboxamide; (Isomer 1)
(+)-N-(4-(5-ethyl-1,2,4-oxadiazol-3-yl)phenyl)-6-fluoro-8-(4-methylpiperazin-1-yl)chroman-2-carboxamide; (Isomer 1)
(+)-6-fluoro-N-(4-(5-isopropyl-1,2,4-oxadiazol-3-yl)phenyl)-8-(4-methylpiperazin-1-yl)chroman-2-carboxamide; (Isomer 1)
(+)-N-(4-(4-ethoxypiperidin-1-yl)phenyl)-6-methoxy-8-(4-methylpiperazin-1-yl)chroman-2-carboxamide; (Isomer 1)
(+)-6-methoxy-N-(4-(4-methyloxazol-2-yl)phenyl)-8-(4-methylpiperazin-1-yl)chroman-2-carboxamide; (Isomer 1)
(+)-6-Methoxy-N-(4-morpholinophenyl)-8-(4-(2,2,2-trifluoroethyl)piperazin-1-yl)chroman-2-carboxamide; (Isomer 1)
(+)-6-methoxy-8-(4-(2-methoxyethyl)piperazin-1-yl)-N-(4-morpholinophenyl)chroman-2-carboxamide; (Isomer 1)
(+)-8-(4-butylpiperazine-1-yl)-6-methoxy-N-(4-morpholinophenyl)chroman-2-carboxamide; (Isomer 1)
(+)-6-methoxy-N-(4-morpholinophenyl)-8-(4-propylpiperazin-1-yl)chroman-2-carboxamide; (Isomer 1)
(+)-8-(4-isopropylpiperazin-1-yl)-6-methoxy-N-(4-morpholinophenyl)chroman-2-carboxamide; (Isomer 1)
(+)-8-(4-ethylpiperazin-1-yl)-6-methoxy-N-(4-morpholinophenyl)chroman-2-carboxamide; (Isomer 1)
(+)-6-methoxy-N-(4-morpholinophenyl)-8-(piperazin-1-yl)chroman-2-carboxamide; (Isomer 1)
(+)-6-methoxy-2-methyl-8-(4-methylpiperazin-1-yl)-N-(4-morpholinophenyl)chroman-2-carboxamide; (Isomer 1), and
(+)-6-hydroxy-8-(4-methylpiperazin-1-yl)-N-(4-morpholinophenyl)chroman-2-carboxamide; (Isomer 1)
or a pharmaceutically-acceptable salt thereof.

35. The method of claim 34 wherein the disorder is a mood disorder.

36. The method of claim 34 wherein the disorder is a cognitive disorder.

37. The use of a compound according to any one of claims 1 to 25 in the preparation of a medicament for the treatment of anxiety disorders or mood disorders or cognitive disorders.

38. A pharmaceutical composition comprising a compound of any one of claims 1 to 25 or a pharmaceutically acceptable salt thereof and a pharmaceutically acceptable carrier.

39. A process for the preparation of a compound according to claim 1 or a pharmaceutically acceptable salt thereof, which process comprises reacting a compound of formula (II): 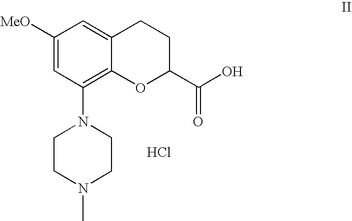
with a compound of formula (III): 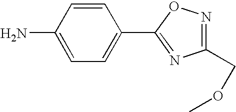
and isolating the desired isomer thereof.
